(19) United States
(12) Patent Application Publication
(10) Pub. No.: US 2025/0228882 A1
(43) Pub. Date: Jul. 17, 2025

(54) ORALLY-BIOAVAILABLE NUCLEOSIDE ANALOGS

(71) Applicant: Venatorx Pharmaceuticals, Inc., Malvern, PA (US)

(72) Inventors: Steven A. BOYD, Chester Springs, PA (US); Stephen M. CONDON, Glenmoore, PA (US); Glen A. COBURN, Bethel, CT (US)

(21) Appl. No.: 18/853,929

(22) PCT Filed: Apr. 6, 2023
(86) PCT No.: PCT/US2023/017669

§ 371 Date: Oct. 3, 2024

Related U.S. Application Data
(63) Continuation-in-part of application No. PCT/US2022/033196, filed on Jun. 13, 2022.
(60) Provisional application No. 63/328,106, filed on Apr. 6, 2022.
(60) Provisional application No. 63/387,361, filed on Dec. 14, 2022.

(51) Int. Cl. A61K 31/706; A61K 9/00; A61K 9/20; A61K 9/48; A61K 45/06; C07D 487/04

(52) U.S. Cl. CPC A61K 31/706; A61K 9/0053; A61K 9/2054; A61K 9/2059; A61K 9/4825; A61K 9/4858; A61K 45/06; C07D 487/04

(57) ABSTRACT

Described herein are orally-bioavailable nucleoside analogs and pharmaceutical compositions comprising said compounds. The subject compounds and compositions are useful for the treatment of coronavirus infections, including SARS-CoV-2 infection.

### CROSS-REFERENCE

This application claims the benefit of U.S. Provisional Application Ser. No. 63/328,106 filed Apr. 6, 2022; U.S. Provisional Application Ser. No. 63/387,361 filed Dec. 14, 2022; and is a continuation-in-part of International Application Serial No. PCT/US2022/033 196 filed Jun. 13, 2022; each of which are hereby incorporated by reference in their entirety.

### BACKGROUND

Remdesivir is a parenterally administered prodrug that was previously developed for the treatment of Ebola virus disease and more recently received emergency use authorization for the treatment of severe acute respiratory syndrome-coronavirus 2 (SARS-CoV-2) infection (Eastman, R. T., Roth, J. S., Brimacombe, K. R., Simeonov, A., Shen, M., Patnaik, S., and Hall, M. D. (2020). Remdesivir: A Review of Its Discovery and Development Leading to Emergency Use Authorization for Treatment of COVID-19. ACS CentSci 6, 672-683). The parent compound is a 1′-cyano modified adenosine C-nucleoside analog (GS-441524) that is phosphorylated by intracellular nucleotide kinases to the active tri-phosphorylated form (GS-443902). GS-443902 inhibits viral replication by competing with adenosine 5′-triphosphate for incorporation into nascent RNA chains by the virally-encoded RNA-dependent RNA polymerase leading to a delayed chain termination that can occur three nucleotides downstream from its position (Kokic et a., 2021; Nature Communications 12:279). In full-length read-through transcripts, the presence of GS-443902 residues in the template strand significantly compromises the incorporation of uridine residues during second-strand synthesis (Tchesnokov et al. 2020 Journal of Biological Chemistry 295(47):16156-16165) Remdesivir (GS-5734) is a monophosphoramidate prodrug of GS-441524 and demonstrates broad spectrum antiviral activity against a wide range of different RNA virus families including: filoviruses (e.g. Ebola viruses, Marburg virus), paramyxoviruses (e.g. parainfluenza type II virus, Nipah virus, Hendra virus, measles morbillivirus, mumps virus), coronaviruses (e.g. SARS-CoV, SARS-CoV-2, MERS-CoV), and pneumoviruses (e.g. respiratory syncytial virus) (Cho, A., Saunders, O. L., Butler, T., Zhang, L., Xu, J., Vela, J. E., Feng, J. Y., Ray, A. S., and Kim, C. U. (2012). Synthesis and antiviral activity of a series of 1′-substituted 4-aza-7,9-dideazaadenosine C-nucleosides. Bioorg Med Chem Lett 22, 2705-2707).

Coronaviruses (CoV) are a large family of viruses that typically infect the upper respiratory tract causing mild to moderate disease in humans. Infections with human coronaviruses: 229E, NL63, OC43 and HKU1 account for approximately 5-30% of “common” colds. There are several hundred coronaviruses that circulate in animals such as pigs, camels, bats, and cats. In the past twenty years, at least three coronaviruses have jumped from one or more of these animal reservoirs into humans in what is known as a spillover event. One example is “Middle East Respiratory Syndrome Coronavirus” (MERS-CoV or MERS) which was first reported in Saudi Arabia in 2012. Another example is SARS-CoV, the coronavirus responsible for “Severe Acute Respiratory Syndrome” (SARS) that was first identified in China in 2002. Still another example is SARS-CoV-2, the causative agent of COVID-19 (Coronavirus Disease of 2019). All three of these beta-coronaviruses: SARS-CoV, MERS-CoV, and SARS-CoV-2, have shown the potential to replicate in the lower respiratory tract and cause more serious illnesses in humans giving rise to a viral pneumonia that can be fatal. Other clinical manifestations and organ system complications (i.e., cardiac, neurologic, hematologic) have also been described in patients who are infected with SARS-CoV-2.

### SUMMARY

The present disclosure relates to small-molecule compounds that block coronavirus replication with the potential to be used as a monotherapy or in combination with additional antivirals and/or other agents that are useful for the treatment of coronavirus infection.

Disclosed herein is a compound of Formula (VIa), (VIb), or (VIc), or a pharmaceutically acceptable salt, solvate, or stereoisomer thereof:

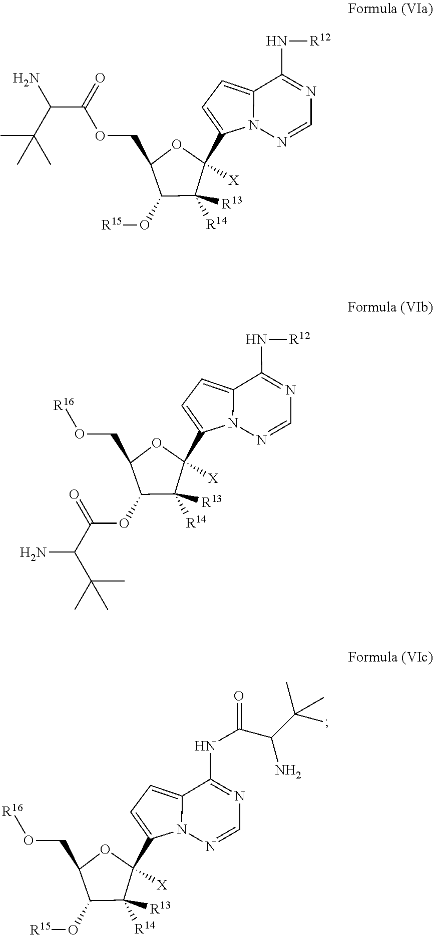

wherein:
X is hydrogen or —CN;
R¹² is hydrogen, —C(═O)R²², —C(═O)OR²², or C₁-C₆alkyl optionally substituted with one or more R^{12a};
each R^{12a} is independently halogen, —CN, —OH, —OR^{a}, —NR^{c}R^{d}, cycloalkyl, or heterocycloalkyl;
or two R^{12a} on the same atom are taken together to form an oxo;
R²² is C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R²²;
each R²² is independently halogen, —CN, —NO₂, —OH, —OR^{a}, —OC(═O)R^{a}, —OC(═O)OR^{b}, —OC(═O)NR^{c}R^{d}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, cycloalkyl, heterocycloalkyl, aryl, and heteroaryl is optionally and independently substituted with one or more R;
or two R²² on the same atom are taken together to form an oxo;
or two R²² are taken together to form a cycloalkyl or heterocycloalkyl; each optionally and independently substituted with one or more R;
R¹³ is hydrogen or C₁-C₆alkyl;
R¹⁴ is —OH or fluoro;
R¹⁵ is hydrogen, —C(═O)R²⁵, —C(═O)OR²⁵, —CH₂—O—C(═O)R²⁵, or —CH₂—O—C(═O)OR²⁵;
R²⁵ is C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{25a};
each R^{25a} is independently halogen, —CN, —NO₂, —OH, —OR^{a}, —OC(═O)R^{a}, —OC(═O)OR^{b}, —OC(═O)NR^{c}R^{d}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, cycloalkyl, heterocycloalkyl, aryl, and heteroaryl is optionally and independently substituted with one or more R;
or two R^{25a} on the same atom are taken together to form an oxo;
or two R^{25a} are taken together to form a cycloalkyl or heterocycloalkyl; each optionally and independently substituted with one or more R;
R¹⁶ is hydrogen, —C(═O)R²⁶, —C(═O)OR²⁶, —CH₂—O—C(═O)R²⁶, —CH₂—O—C(═O)OR²⁶, or C₁-C₆alkyl optionally substituted with one or more R^{16a};
each R^{16a} is independently halogen, —CN, —OH, —OR^{a}, —NR^{c}R^{d}, cycloalkyl, or heterocycloalkyl;
or two R^{16a} on the same atom are taken together to form an oxo;
R²⁶ is C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{26a};
each R^{26a} is independently halogen, —CN, —NO₂, —OH, —OR^{a}, —OC(═O)R^{a}, —OC(═O)OR^{b}, —OC(═O)NR^{c}R^{d}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, cycloalkyl, heterocycloalkyl, aryl, and heteroaryl is optionally and independently substituted with one or more R;
or two R^{26a} on the same atom are taken together to form an oxo;
or two R^{26a} are taken together to form a cycloalkyl or heterocycloalkyl; each optionally and independently substituted with one or more R;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, and C₁-C₆heteroalkyl; or two R on the same atom are taken together to form an oxo.

Also disclosed herein is a pharmaceutical composition comprising the compound disclosed herein, or a pharmaceutically acceptable salt, solvate, or stereoisomer thereof, and at least one pharmaceutically acceptable carrier.

Also disclosed herein is a method of treating a viral infection, comprising administering to a subject in need thereof a therapeutically effective amount of the compound disclosed herein, or a pharmaceutically acceptable salt, solvate, or stereoisomer thereof, or a pharmaceutical composition disclosed herein.

In some embodiments, the method further comprises administering at least one antiviral agent in combination with the compound disclosed herein, or a pharmaceutically acceptable salt, solvate, or stereoisomer thereof, or a pharmaceutical composition disclosed herein.

In some embodiments, the viral infection is caused by a virus selected from the group consisting of coronavirus disease 2019 (SARS-CoV-2), YellowFever, Eastern Equine Encephalitis virus, Human Immunodeficiency virus (HIV), “African Swine Fever Viruses,” Arbovirus, Adenoviridae, Arenaviridae, Arterivirus, Astroviridae, Baculoviridae, Bimaviridae, Bimaviridae, Bunyaviridae, Caliciviridae, Caulimoviridae, Circoviridae, Coronaviridae, Cystoviridae, Ebolavirus, Deltaviridae, Filviridae, Filoviridae, Flaviviridae, Iridoviridae, Mononegavirus, Myoviridae, Papiloma virus, Papovaviridae, Paramyxoviridae, Prions, Parvoviridae, Phycodnaviridae, Poxviridae, Potyviridae, Reoviridae, Retroviridae, Rhabdoviridae, Tectiviridae, Togaviridae, pox, papilloma, corona, influenza, sendai virus (SeV), sindbis virus (SINV), vaccinia viruses, West Nile, Hanta, viruses which cause the common cold, and any combination thereof.

In some embodiments, the viral infection is caused by coronavirus disease 2019 (SARS-CoV-2).

In some embodiments, the viral infection is caused by the Middle East respiratory syndrome coronavirus (MERS-CoV).

In some embodiments, the viral infection is caused by an Ebolavirus.

In some embodiments, the Ebolavirus is Zaira Ebolavirus (Ebola virus).

In some embodiments, the at least one antiviral agent is abacavir, acyclovir, adefovir, brivudine, cidofovir, didanosine, edoxudine, emtricitabine, entecavir, famciclovir, favipiravir, ganciclovir, idoxuridine, lamivudine, molnupiravir, penciclovir, remdesivir, ribavirin, sofosbuvir, ST-193, stavudine, T-705 diphosphate,T-705 monophosphate, T-705 triphosphate, telbivudine, tenofovir alafenamide, tenofovir disoproxil, trifluridine, valaciclovir, valganciclovir, vidarabine, zalcitabine, zidovudine, or any combinations thereof.

In some embodiments, the at least one antiviral agent is molnupiravir.

In some embodiments, the at least one antiviral agent is a ribonucleic acid (RNA)-dependent RNA polymerase inhibitor, a checkpoint inhibitor or PD-1/PD-L1 inhibitor, a therapeutic vaccine, an RNA interference (RNAi) therapeutic, an antisense-based therapeutic, an coronavirus entry inhibitor, a TLR agonist, an RIG-I agonist, or an interferon.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### Definitions

In the following description, certain specific details are set forth to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to —COOH.

“Cyano” refers to —CN.

“Alkyl” refers to a straight-chain or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁-6alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁-C₆alkyl. In some embodiments, the alkyl is a C₁₋₅alkyl. In some embodiments, the alkyl is a C₁-4alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain or branched-chain hydrocarbon monoradical having one or more carbon-carbon double-bonds and having from two to about ten carbon atoms, more preferably two to about six carbon atoms. The group may be in either the cis or trans conformation about the double bond(s) and should be understood to include both isomers. Examples include, but are not limited to ethenyl (—CH═CH₂), 1-propenyl (—CH₂CH═CH₂), isopropenyl [—C(CH₃)—CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula -Oalkyl where alkyl is as defined above. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (e.g., C₃-C₁₅ fully saturated cycloalkyl or C₃-C₁₅ cycloalkenyl), from three to ten carbon atoms (e.g., C₃-C₁ fully saturated cycloalkyl or C₃-C₁₀ cycloalkenyl), from three to eight carbon atoms (e.g., C₃-C₈ fully saturated cycloalkyl or C₃-C₈ cycloalkenyl), from three to six carbon atoms (e.g., C₃-C₆ fully saturated cycloalkyl or C₃-C₆ cycloalkenyl), from three to five carbon atoms (e.g., C₃-C₅ fully saturated cycloalkyl or C₃-C₅ cycloalkenyl), or three to four carbon atoms (e.g., C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (e.g., C₂-C₁₅ fully saturated heterocycloalkyl or C₂-C₁₅ heterocycloalkenyl), from two to ten carbon atoms (e.g., C₂-C₁₀ fully saturated heterocycloalkyl or C₂-C₁₀ heterocycloalkenyl), from two to eight carbon atoms (e.g., C₂-C₈ fully saturated heterocycloalkyl or C₂-C₈ heterocycloalkenyl), fromtwo to seven carbon atoms (e.g., C₂-C₇ fully saturated heterocycloalkyl or C₂-C₇ heterocycloalkenyl), fromtwo to six carbon atoms (e.g., C₂-C₆ fully saturated heterocycloalkyl or C₂-C₆ heterocycloalkenyl), fromtwo to five carbon atoms (e.g., C₂-C₅ fully saturated heterocycloalkyl or C₂-C₅ heterocycloalkenyl), or two to four carbon atoms (e.g., C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns that are sterically impractical and/or synthetically non-feasible. Thus, any substituents described should generally be understood as having a maximum molecular weight of about 1,000 daltons, and more typically, up to about 500 daltons.

The term “one or more” when referring to an optional substituent means that the subject group is optionally substituted with one, two, three, four, or more substituents. In some embodiments, the subject group is optionally substituted with one, two, three or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition.

“Coronavirus infection” refers to any and all conditions deriving from infection with coronaviruses, including but not limited to SARS-CoV, SARS-CoV-2, and MERS-CoV, preferably SARS-CoV-2.

### Compounds

Described herein are compounds disclosed herein, or a pharmaceutically acceptable salt, solvate, or stereoisomer thereof useful in the treatment of viral infections. In some embodiments, the viral infection is a coronavirus infection.

Disclosed herein is a compound of Formula (VIa), (VIb), or (VIc), or a pharmaceutically acceptable salt, solvate, or stereoisomer thereof.

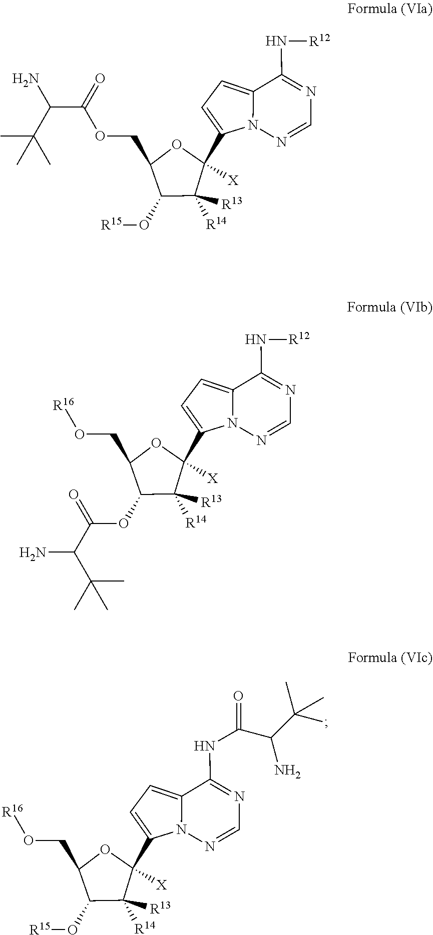

wherein:
X is hydrogen or —CN;
R¹² is hydrogen, —C(═O)R²², —C(═O)OR²², —C(═O)NR²²R²³, or C₁-C₆alkyl optionally substituted with one or more R^{12a};
each R^{12a} is independently halogen, —CN, —OH, —OR^{a}, —NR^{c}R^{d}, cycloalkyl, or heterocycloalkyl;
or two R^{12a} on the same atom are taken together to form an oxo;
R²² is C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R²²;
R²³ is hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{22a};
or R²² and R²³ are taken together to form a heterocycloalkyl optionally substituted with one or more R^{22a};
each R²² is independently halogen, —CN, —NO₂, —OH, —OR^{a}, —OC(═O)R^{a}, —OC(═O)OR^{b}, —OC(═O)NR^{c}R^{d}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, cycloalkyl, heterocycloalkyl, aryl, and heteroaryl is optionally and independently substituted with one or more R;
or two R^{22a} on the same atom are taken together to form an oxo;
or two R^{22a} are taken together to form a cycloalkyl or heterocycloalkyl; each optionally and independently substituted with one or more R;
R¹³ is hydrogen or C₁-C₆alkyl;
R¹⁴ is —OH or fluoro;
R¹⁵ is hydrogen, —C(═O)R²⁵, —C(═O)OR²⁵, —CH₂—O—C(═O)R²⁵, or —CH₂—O—C(═O)OR²⁵;
R²⁵ is C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{25a};
each R^{25a} is independently halogen, —CN, —NO₂, —OH, —OR^{a}, —OC(═O)R^{a}, —OC(═O)OR^{b}, —OC(═O)NR^{c}R^{d}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, cycloalkyl, heterocycloalkyl, aryl, and heteroaryl is optionally and independently substituted with one or more R;
or two R^{25a} on the same atom are taken together to form an oxo;
or two R^{25a} are taken together to form a cycloalkyl or heterocycloalkyl; each optionally and independently substituted with one or more R;
R¹⁶ is hydrogen, —C(═O)R²⁶, —C(═O)OR²⁶, —CH₂—O—C(═O)R²⁶, —CH₂—O—C(═O)OR²⁶, or C₁-C₆alkyl optionally substituted with one or more R^{16a};
each R^{16a} is independently halogen, —CN, —OH, —OR^{a}, —NR^{c}R^{d}, cycloalkyl, or heterocycloalkyl;
or two R^{16a} on the same atom are taken together to form an oxo;
R²⁶ is C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{26a};
each R^{26a} is independently halogen, —CN, —NO₂, —OH, —OR^{a}, —OC(═O)R^{a}, —OC(═O)OR^{b}, —OC(═O)NR^{c}R^{d}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, cycloalkyl, heterocycloalkyl, aryl, and heteroaryl is optionally and independently substituted with one or more R;
or two R^{26a} on the same atom are taken together to form an oxo;
or two R^{26a} are taken together to form a cycloalkyl or heterocycloalkyl; each optionally and independently substituted with one or more R;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, and C₁-C₆heteroalkyl;
or two R on the same atom are taken together to form an oxo.

Disclosed herein is a compound of Formula (VIa), (VIb), or (VIc), or a pharmaceutically acceptable salt, solvate, or stereoisomer thereof:

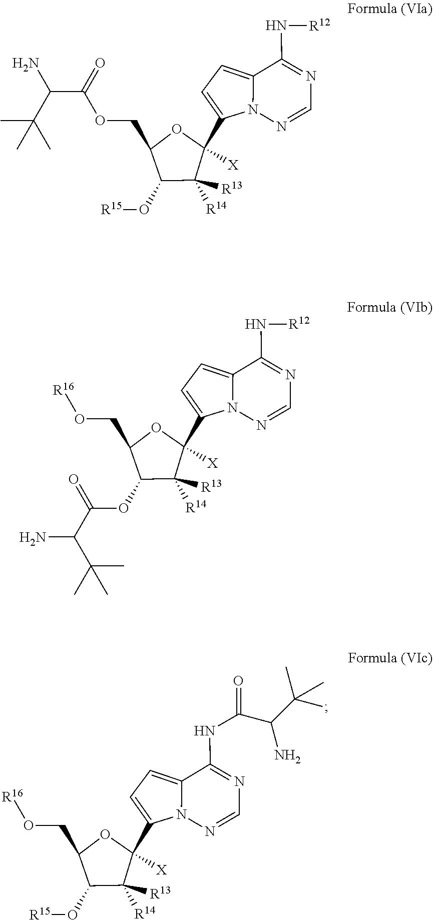

wherein:
X is hydrogen or —CN;
R¹² is hydrogen, —C(═O)R²², —C(═O)OR²², or C₁-C₆alkyl optionally substituted with one or more R^{12a};
each R^{12a} is independently halogen, —CN, —OH, —OR^{a}, —NR^{c}R^{d}, cycloalkyl, or heterocycloalkyl;
or two R^{12a} on the same atom are taken together to form an oxo;
R²² is C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R²²;
each R^{22a} is independently halogen, —CN, —NO₂, —OH, —OR^{a}, —OC(═O)R^{a}, —OC(═O)OR^{b}, —OC(═O)NR^{c}R^{d}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, cycloalkyl, heterocycloalkyl, aryl, and heteroaryl is optionally and independently substituted with one or more R;
or two R²² on the same atom are taken together to form an oxo;
or two R²² are taken together to form a cycloalkyl or heterocycloalkyl; each optionally and independently substituted with one or more R;
R¹³ is hydrogen or C₁-C₆alkyl;
R¹⁴ is —OH or fluoro;
R¹⁵ is hydrogen, —C(═O)R²⁵, —C(═O)OR²⁵, —CH₂—O—C(═O)R²⁵, or —CH₂—O—C(═O)OR²⁵;
R²⁵ is C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{25a};
each R^{25a} is independently halogen, —CN, —NO₂, —OH, —OR^{a}, —OC(═O)R^{a}, —OC(═O)OR^{b}, —OC(═O)NR^{c}R^{d}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, cycloalkyl, heterocycloalkyl, aryl, and heteroaryl is optionally and independently substituted with one or more R;
or two R^{25a} on the same atom are taken together to form an oxo;
or two R^{25a} are taken together to form a cycloalkyl or heterocycloalkyl; each optionally and independently substituted with one or more R;
R¹⁶ is hydrogen, —C(═O)R²⁶, —C(═O)OR²⁶, —CH₂—O—C(═O)R²⁶, —CH₂—O—C(═O)OR²⁶, or C₁-C₆alkyl optionally substituted with one or more R^{16a};
each R^{16a} is independently halogen, —CN, —OH, —OR^{a}, —NR^{c}R^{d}, cycloalkyl, or heterocycloalkyl;
or two R^{16a} on the same atom are taken together to form an oxo;
R²⁶ is C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{26a};
each R^{26a} is independently halogen, —CN, —NO₂, —OH, —OR^{a}, —OC(═O)R^{a}, —OC(═O)OR^{b}, —OC(═O)NR^{c}R^{d}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, cycloalkyl, heterocycloalkyl, aryl, and heteroaryl is optionally and independently substituted with one or more R;
or two R^{26a} on the same atom are taken together to form an oxo;
or two R^{26a} are taken together to form a cycloalkyl or heterocycloalkyl; each optionally and independently substituted with one or more R;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, and C₁-C₆heteroalkyl;
or two R on the same atom are taken together to form an oxo.

In some embodiments, the compound is a compound of Formula (VIa):

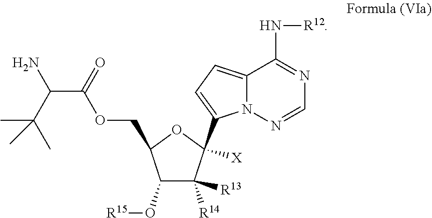

In some embodiments, the compound is a compound of Formula (VIa-1):

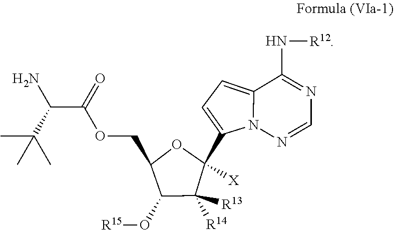

In some embodiments, the compound is a compound of Formula (VIa-2):

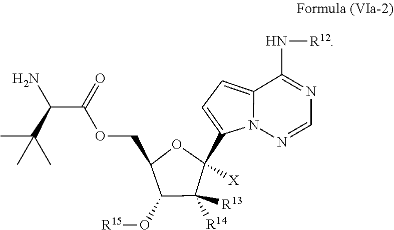

In some embodiments, the compound is a compound of Formula (VIb):

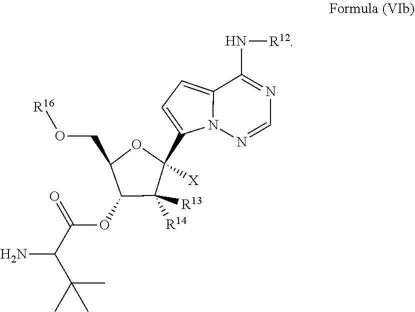

In some embodiments, the compound is a compound of Formula (VIb-1):

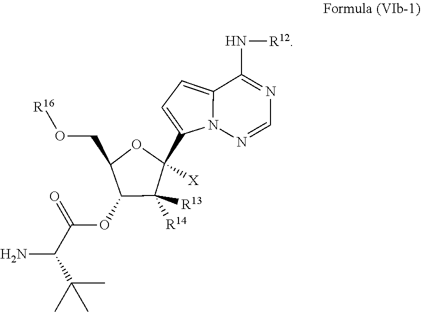

In some embodiments, the compound is a compound of Formula (VIb-2):

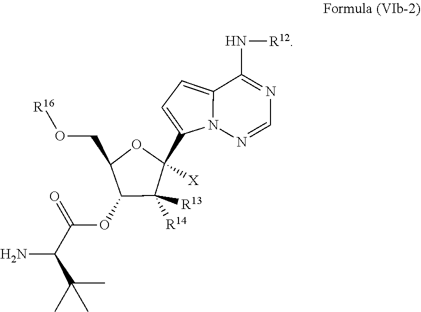

In some embodiments, the compound is a compound of Formula (VIc):

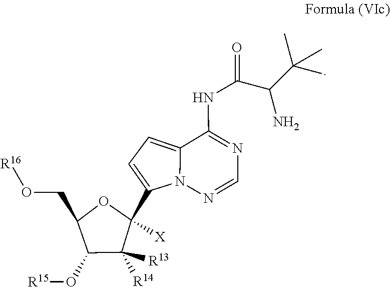

In some embodiments, the compound is a compound of Formula (VIc-1):

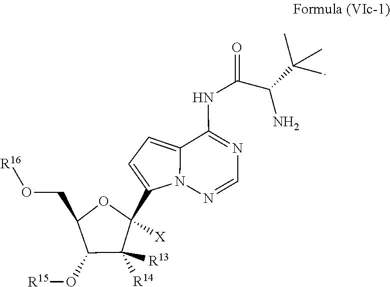

In some embodiments, the compound is a compound of Formula (VIc-2):

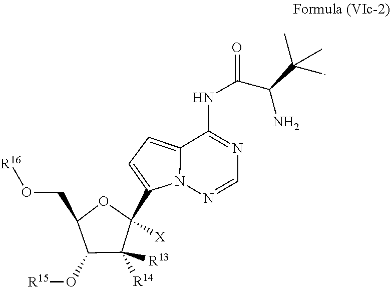

In some embodiments of a compound of Formula (VIa), (VIa-1), (VIa-2), (VIb), (VIb-1), (VIb-2), (VIc), (VIc-1), or (VIc-2), X is hydrogen. In some embodiments of a compound of Formula (VIa), (VIa-1), (VIa-2), (VIb), (VIb-1), (VIb-2), (VIc), (VIc-1), or (VIc-2), X is —CN.

In some embodiments of a compound of Formula (VIa), (VIa-1), (VIa-2), (VIb), (VIb-1), (VIb-2), (VIc), (VIc-1), or (VIc-2), R¹³ is hydrogen. In some embodiments of a compound of Formula (VIa), (VIa-1), (VIa-2), (VIb), (VIb-1), (VIb-2), (VIc), (VIc-1), or (VIc-2), R¹³ is C₁-C₆alkyl.

In some embodiments of a compound of Formula (VIa), (VIa-1), (VIa-2), (VIb), (VIb-1), (VIb-2), (VIc), (VIc-1), or (VIc-2), R¹⁴ is —OH. In some embodiments of a compound of Formula (VIa), (VIa-1), (VIa-2), (VIb), (VIb-1), (VIb-2), (VIc), (VIc-1), or (VIc-2), R¹⁴ is fluoro.

In some embodiments of a compound of Formula (VIa), (VIa-1), (VIa-2), (VIb), (VIb-1), or (VIb-2), R¹² is hydrogen, —C(═O)R²², —C(═O)OR²², or C₁-C₆alkyl. In some embodiments of a compound of Formula (VIa), (VIa-1), (VIa-2), (VIb), (VIb-1), or (VIb-2), R¹² is —C(═O)R²², —C(═O)OR²², or C₁-C₆alkyl. In some embodiments of a compound of Formula (VIa), (VIa-1), (VIa-2), (VIb), (VIb-1), or (VIb-2), R¹² is —C(═O)R²². In some embodiments of a compound of Formula (VIa), (VIa-1), (VIa-2), (VIb), (VIb-1), or (VIb-2), R¹² is —C(═O)OR²². In some embodiments of a compound of Formula (VIa), (VIa-1), (VIa-2), (VIb), (VIb-1), or (VIb-2), R¹² is C₁-C₆alkyl. In some embodiments of a compound of Formula (VIa), (VIa-1), (VIa-2), (VIb), (VIb-1), or (VIb-2), R¹² is hydrogen.

In some embodiments of a compound of Formula (VIa), (VIa-1), (VIa-2), (VIb), (VIb-1), or (VIb-2), R¹² is —C(═O)NR²²R²³.

In some embodiments of a compound of Formula (VIa), (VIa-1), (VIa-2), (VIb), (VIb-1), or (VIb-2), R²² is C₁-C₆alkyl, C₁-C₆aminoalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{22a}.

In some embodiments of a compound of Formula (VIa), (VIa-1), (VIa-2), (VIb), (VIb-1), or (VIb-2), R²² is C₁-C₆alkyl, C₁-C₆aminoalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl).

In some embodiments of a compound of Formula (VIa), (VIa-1), (VIa-2), (VIb), (VIb-1), or (VIb-2), R²² is C₁-C₆alkyl, C₁-C₆aminoalkyl, aryl, C₁-C₆alkylene(cycloalkyl), C₁-C₆alkylene(heterocycloalkyl), C₁-C₆alkylene(aryl), or C₁-C₆alkylene(heteroaryl).

In some embodiments of a compound of Formula (VIa), (VIa-1), (VIa-2), (VIb), (VIb-1), or (VIb-2), R²² is C₁-C₆alkyl, C₁-C₆aminoalkyl, aryl, C₁-C₆alkylene(cycloalkyl), or C₁-C₆alkylene(aryl).

In some embodiments of a compound of Formula (VIa), (VIa-1), (VIa-2), (VIb), (VIb-1), or (VIb-2), R²² is C₁-C₆alkyl or C₁-C₆alkylene(aryl); wherein the alkyl, alkylene, and aryl is optionally and independently substituted with one or more R^{22a}.

In some embodiments of a compound of Formula (VIa), (VIa-1), (VIa-2), (VIb), (VIb-1), or (VIb-2), R²² is C₁-C₆alkyl optionally and independently substituted with one or more R^{22a}.

In some embodiments of a compound of Formula (VIa), (VIa-1), (VIa-2), (VIb), (VIb-1), or (VIb-2), R²³ is hydrogen, C₁-C₆alkyl, C₁-C₆aminoalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R²². In some embodiments of a compound of Formula (VIa), (VIa-1), (VIa-2), (VIb), (VIb-1), or (VIb-2), R²³ is hydrogen, C₁-C₆alkyl, C₁-C₆aminoalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl). In some embodiments of a compound of Formula (VIa), (VIa-1), (VIa-2), (VIb), (VIb-1), or (VIb-2), R²³ is hydrogen, C₁-C₆alkyl, C₁-C₆aminoalkyl, aryl, C₁-C₆alkylene(cycloalkyl), C₁-C₆alkylene(heterocycloalkyl), C₁-C₆alkylene(aryl), or C₁-C₆alkylene(heteroaryl). In some embodiments of a compound of Formula (VIa), (VIa-1), (VIa-2), (VIb), (VIb-1), or (VIb-2), R²³ is hydrogen, C₁-C₆alkyl, C₁-C₆aminoalkyl, aryl, C₁-C₆alkylene(cycloalkyl), or C₁-C₆alkylene(aryl).

In some embodiments of a compound of Formula (VIa), (VIa-1), (VIa-2), (VIb), (VIb-1), or (VIb-2), R²³ is hydrogen, C₁-C₆alkyl, or C₁-C₆alkylene(aryl); wherein the alkyl, alkylene, and aryl is optionally and independently substituted with one or more R²². In some embodiments of a compound of Formula (VIa), (VIa-1), (VIa-2), (VIb), (VIb-1), or (VIb-2), R²³ is hydrogen or C₁-C₆alkyl optionally and independently substituted with one or more R²². In some embodiments of a compound of Formula (VIa), (VIa-1), (VIa-2), (VIb), (VIb-1), or (VIb-2), R²³ is C₁-C₆alkyl optionally and independently substituted with one or more R²². In some embodiments of a compound of Formula (VIa), (VIa-1), (VIa-2), (VIb), (VIb-1), or (VIb-2), R²³ is C₁-C₆alkyl. In some embodiments of a compound of Formula (VIa), (VIa-1), (VIa-2), (VIb), (VIb-1), or (VIb-2), R²³ is hydrogen.

In some embodiments of a compound of Formula (VIa), (VIa-1), (VIa-2), (VIb), (VIb-1), or (VIb-2), each R²² is independently halogen, —CN, —OH, —OR^{a}, —OC(═O)R^{a}, —OC(═O)OR^{b}, —OC(═O)NR^{c}R^{d}, —NR^{c}R^{d}, —C(═O)R^{a}, —C(═O)OR^{b}, —C(═O)NR^{c}R^{d}, C₁-C₆alkyl, or C₁-C₆haloalkyl.

In some embodiments of a compound of Formula (VIa), (VIa-1), (VIa-2), (VIb), (VIb-1), or (VIb-2), each R²² is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, or C₁-C₆haloalkyl.

In some embodiments of a compound of Formula (VIa), (VIa-1), (VIa-2), (VIb), (VIb-1), or (VIb-2), each R²² is independently halogen, —OH, —OR^{a}, —NR^{c}R^{d}, C₁-C₆alkyl, or C₁-C₆haloalkyl.

In some embodiments of a compound of Formula (VIa), (VIa-1), (VIa-2), (VIb), (VIb-1), or (VIb-2), each R²² is independently halogen, —OC(═O)R^{a}, or —NR^{c}R^{d}. In some embodiments of a compound of Formula (VIa), (VIa-1), (VIa-2), (VIb), (VIb-1), or (VIb-2), each R²² is independently —OC(═O)R^{a} or —NR^{c}R^{d}. In some embodiments of a compound of Formula (VIa), (VIa-1), (VIa-2), (VIb), (VIb-1), or (VIb-2), each R²² is independently —OC(═O)R^{a}.

In some embodiments of a compound of Formula (VIa), (VIa-1), (VIa-2), (VIb), (VIb-1), or (VIb-2), R¹² is hydrogen,

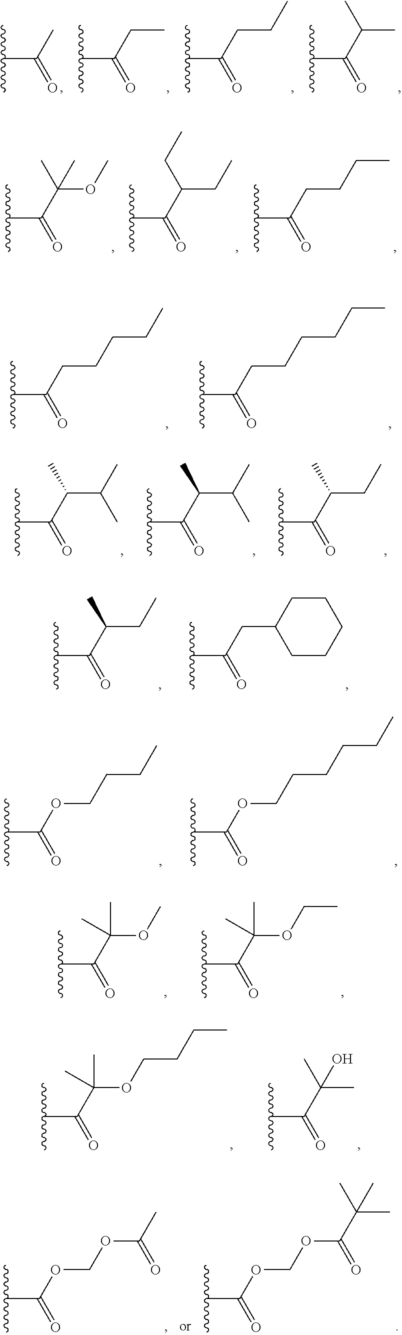

In some embodiments of a compound of Formula (VIa), (VIa-1), (VIa-2), (VIb), (VIb-1), or (VIb-2), R¹² is

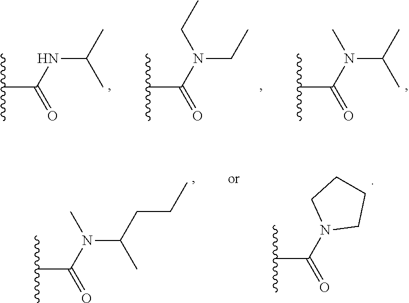

In some embodiments of a compound of Formula (VIa), (VIa-1), (VIa-2), (VIc), (VIc-1), or (VIc-2), R¹⁵ is —C(═O)R²⁵ or —C(═O)OR²⁵, or —CH₂—O—C(═O)R²⁵. In some embodiments of a compound of Formula (VIa), (VIa-1), (VIa-2), (VIc), (VIc-1), or (VIc-2), R¹⁵ is hydrogen. In some embodiments of a compound of Formula (VIa), (VIa-1), (VIa-2), (VIc), (VIc-1), or (VIc-2), R¹⁵ is —C(═O)R²⁵. In some embodiments of a compound of Formula (VIa), (VIa-1), (VIa-2), (VIc), (VIc-1), or (VIc-2), R¹⁵ is —C(═O)OR²⁵. In some embodiments of a compound of Formula (VIa), (VIa-1), (VIa-2), (VIc), (VIc-1), or (VIc-2), R¹⁵ is hydrogen.

In some embodiments of a compound of Formula (VIa), (VIa-1), (VIa-2), (VIc), (VIc-1), or (VIc-2), R²⁵ is C₁-C₆alkylene(cycloalkyl) or C₁-C₆alkylene(aryl); wherein the alkylene, cycloalkyl, and aryl is optionally and independently substituted with one or more R^{25a}.

In some embodiments of a compound of Formula (VIa), (VIa-1), (VIa-2), (VIc), (VIc-1), or (VIc-2), R²⁵ is C₁-C₆alkylene(cycloalkyl); wherein the alkylene and cycloalkyl is optionally and independently substituted with one or more R^{25a}.

In some embodiments of a compound of Formula (VIa), (VIa-1), (VIa-2), (VIc), (VIc-1), or (VIc-2), R²⁵ is C₁-C₆alkylene(aryl); wherein the alkylene, and aryl is optionally and independently substituted with one or more R^{25a}.

In some embodiments of a compound of Formula (VIa), (VIa-1), (VIa-2), (VIc), (VIc-1), or (VIc-2), each R^{25a} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, or C₁-C₆haloalkyl.

In some embodiments of a compound of Formula (VIa), (VIa-1), (VIa-2), (VIc), (VIc-1), or (VIc-2), each R^{25a} is independently halogen, —OH, —OR^{a}, —NR^{c}R^{d}, C₁-C₆alkyl, or C₁-C₆haloalkyl.

In some embodiments of a compound of Formula (VIa), (VIa-1), (VIa-2), (VIc), (VIc-1), or (VIc-2), R¹⁵ is hydrogen,

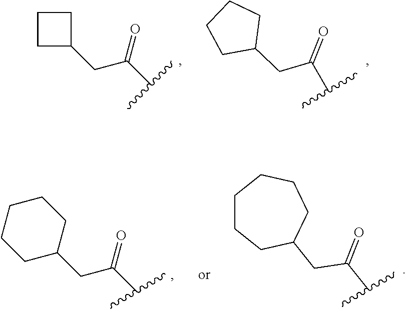 [SMILES: *C(=O)CC1CCC1 ; "," ; *C(=O)CC1CCCC1 ; "," ; *C(=O)CC1CCCCC1 ; *C(=O)CC1CCCCCC1]

In some embodiments of a compound of Formula (VIa), (VIa-1), (VIa-2), (VIc), (VIc-1), or (VIc-2), R¹⁵ is hydrogen,

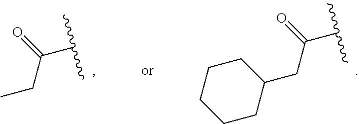

In some embodiments of a compound of Formula (VIb), (VIb-1), (VIb-2), (VIc), (VIc-1), or (VIc-2), R¹⁶ is hydrogen, —C(═O)R²⁶, —C(═O)OR²⁶, or C₁-C₆alkyl optionally substituted with one or more R^{16a}. In some embodiments of a compound of Formula (VIb), (VIb-1), (VIb-2), (VIc), (VIc-1), or (VIc-2), R¹⁶ is hydrogen, —C(═O)R²⁶, or —C(═O)OR²⁶. In some embodiments of a compound of Formula (VIb), (VIb-1), (VIb-2), (VIc), (VIc-1), or (VIc-2), R¹⁶ is —C(═O)R²⁶ or —C(═O)OR²⁶. In some embodiments of a compound of Formula (VIb), (VIb-1), (VIb-2), (VIc), (VIc-1), or (VIc-2), R¹⁶ is hydrogen. In some embodiments of a compound of Formula (VIb), (VIb-1), (VIb-2), (VIc), (VIc-1), or (VIc-2), R¹⁶ is —C(═O)R²⁶. In some embodiments of a compound of Formula (VIb), (VIb-1), (VIb-2), (VIc), (VIc-1), or (VIc-2), R¹⁶ is —C(═O)OR²⁶.

In some embodiments of a compound of Formula (VIb), (VIb-1), (VIb-2), (VIc), (VIc-1), or (VIc-2), R²⁶ is C₁-C₆alkyl, C₁-C₆aminoalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{26a}.

In some embodiments of a compound of Formula (VIb), (VIb-1), (VIb-2), (VIc), (VIc-1), or (VIc-2), R²⁶ is C₁-C₆alkyl, C₁-C₆aminoalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl).

In some embodiments of a compound of Formula (VIb), (VIb-1), (VIb-2), (VIc), (VIc-1), or (VIc-2), R²⁶ is C₁-C₆alkyl, C₁-C₆aminoalkyl, aryl, C₁-C₆alkylene(cycloalkyl), C₁-C₆alkylene(heterocycloalkyl), C₁-C₆alkylene(aryl), or C₁-C₆alkylene(heteroaryl).

In some embodiments of a compound of Formula (VIb), (VIb-1), (VIb-2), (VIc), (VIc-1), or (VIc-2), R²⁶ is C₁-C₆alkyl, C₁-C₆aminoalkyl, aryl, C₁-C₆alkylene(cycloalkyl), or C₁-C₆alkylene(aryl). In some embodiments of a compound of Formula (VIb), (VIb-1), (VIb-2), (VIc), (VIc-1), or (VIc-2), R²⁶ is C₁-C₆alkylene(cycloalkyl) or C₁-C₆alkylene(aryl).

In some embodiments of a compound of Formula (VIb), (VIb-1), (VIb-2), (VIc), (VIc-1), or (VIc-2), R²⁶ is C₁-C₆alkyl or C₁-C₆aminoalkyl.

In some embodiments of a compound of Formula (VIb), (VIb-1), (VIb-2), (VIc), (VIc-1), or (VIc-2), each R^{26a} is independently halogen, —CN, —OH, —OR^{a}, —OC(═O)R^{a}, —OC(═O)OR^{b}, —OC(═O)NR^{c}R^{d}, —NR^{c}R^{d}, —C(═O)R^{a}, —C(═O)OR^{b}, —C(═O)NR^{c}R^{d}, C₁-C₆alkyl, or C₁-C₆haloalkyl.

In some embodiments of a compound of Formula (VIb), (VIb-1), (VIb-2), (VIc), (VIc-1), or (VIc-2), each R^{26a} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, or C₁-C₆haloalkyl.

In some embodiments of a compound of Formula (VIb), (VIb-1), (VIb-2), (VIc), (VIc-1), or (VIc-2), each R^{26a} is independently halogen, —OH, —OR^{a}, —NR^{c}R^{d}, C₁-C₆alkyl, or C₁-C₆haloalkyl.

In some embodiments of a compound of Formula (VIb), (VIb-1), (VIb-2), (VIc), (VIc-1), or (VIc-2), each R^{26a} is independently —OC(═O)R^{a}, or —NR^{c}R^{d}.

In some embodiments of a compound of Formula (VIb), (VIb-1), (VIb-2), (VIc), (VIc-1), or (VIc-2), R¹⁶ is hydrogen,

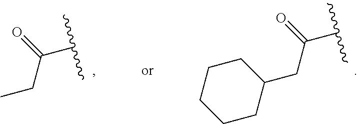

In some embodiments of a compound of Formula (Vb-1), (VIb-1), (VIb-2), (VIc), (VIc-1), or (VIc-2), R¹⁶ is hydrogen,

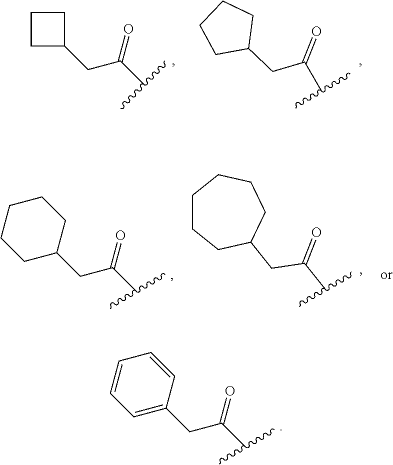

Disclosed herein is a compound of Formula (VIIa) or (VIIb), or a pharmaceutically acceptable salt, solvate, or stereoisomer thereof:

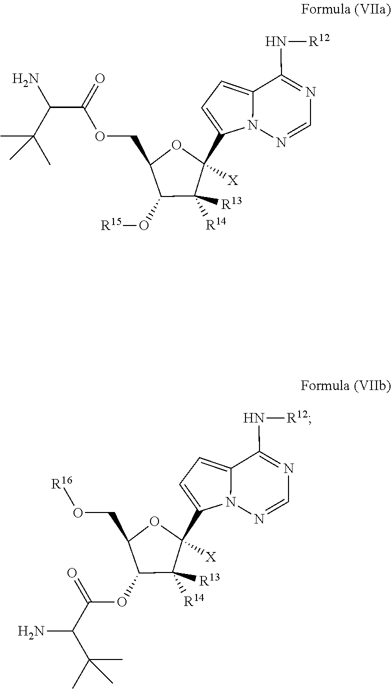

wherein:
X is hydrogen or —CN;
R¹² is —C(═O)NR²²R²³;
R²² is C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R²²;
R²³ is hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{22a};
or R²² and R²³ are taken together to form a heterocycloalkyl optionally substituted with one or more R^{22a};
each R²² is independently halogen, —CN, —NO₂, —OH, —OR^{a}, —OC(═O)R^{a}, —OC(═O)OR^{b}, —OC(═O)NR^{c}R^{d}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, cycloalkyl, heterocycloalkyl, aryl, and heteroaryl is optionally and independently substituted with one or more R;
or two R²² on the same atom are taken together to form an oxo;
or two R²² are taken together to form a cycloalkyl or heterocycloalkyl; each optionally and independently substituted with one or more R;
R¹³ is hydrogen or C₁-C₆alkyl;
R¹⁴ is —OH or fluoro;
R¹⁵ is hydrogen, —C(═O)R²⁵, —C(═O)OR²⁵, —CH₂—O—C(═O)R²⁵, or —CH₂—O—C(═O)OR²⁵;
R²⁵ is C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{25a};
each R^{25a} is independently halogen, —CN, —NO₂, —OH, —OR^{a}, —OC(═O)R^{a}, —OC(═O)OR^{b}, —OC(═O)NR^{c}R^{d}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, cycloalkyl, heterocycloalkyl, aryl, and heteroaryl is optionally and independently substituted with one or more R;
or two R^{25a} on the same atom are taken together to form an oxo;
or two R^{25a} are taken together to form a cycloalkyl or heterocycloalkyl; each optionally and independently substituted with one or more R;
R¹⁶ is hydrogen, —C(═O)R²⁶, —C(═O)OR²⁶, —CH₂—O—C(═O)R²⁶, —CH₂—O—C(═O)OR²⁶, or C₁-C₆alkyl optionally substituted with one or more R^{16a};
each R^{16a} is independently halogen, —CN, —OH, —OR^{a}, —NR^{c}R^{d}, cycloalkyl, or heterocycloalkyl;
or two R^{16a} on the same atom are taken together to form an oxo;
R²⁶ is C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{26a};
each R^{26a} is independently halogen, —CN, —NO₂, —OH, —OR^{a}, —OC(═O)R^{a}, —OC(═O)OR^{b}, —OC(═O)NR^{c}R^{d}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, cycloalkyl, heterocycloalkyl, aryl, and heteroaryl is optionally and independently substituted with one or more R;
or two R^{26a} on the same atom are taken together to form an oxo;
or two R^{26a} are taken together to form a cycloalkyl or heterocycloalkyl; each optionally and independently substituted with one or more R;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, and C₁-C₆heteroalkyl;
or two R on the same atom are taken together to form an oxo.

In some embodiments, the compound is a compound of Formula (VIIa):

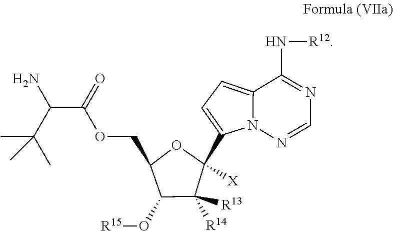

In some embodiments, the compound is a compound of Formula (VIIa-1):

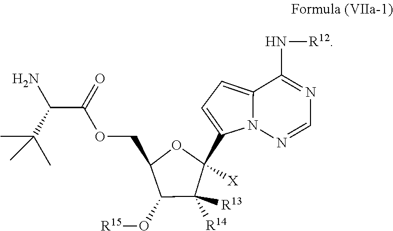

In some embodiments, the compound is a compound of Formula (VIIa-2):

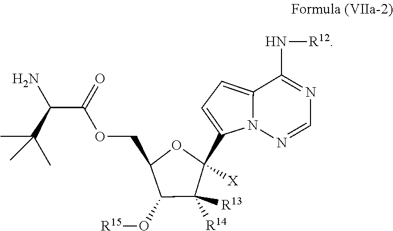

In some embodiments, the compound is a compound of Formula (VIIb):

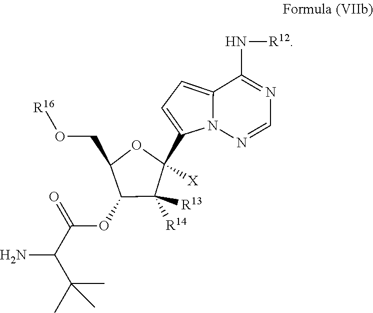

In some embodiments, the compound is a compound of Formula (VIIb-1):

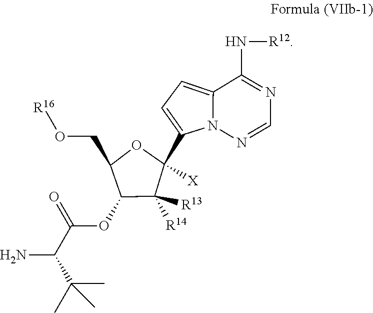

In some embodiments, the compound is a compound of Formula (VIIb-2):

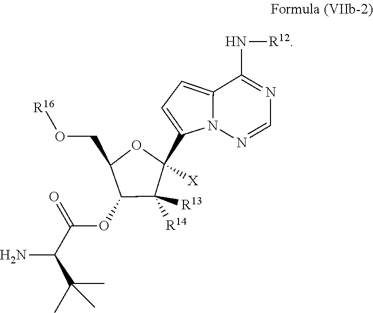

In some embodiments of a compound of Formula (VIIa), (VIIa-1), (VIIa-2), (VIIb), (VIIb-1), or (VIIb-2), X is hydrogen. In some embodiments of a compound of Formula (VIIa), (VIIa-1), (VIIa-2), (VIIb), (VIIb-1), or (VIIb-2), X is —CN.

In some embodiments of a compound of Formula (VIIa), (VIIa-1), (VIIa-2), (VIIb), (VIIb-1), or (VIIb-2), R¹³ is hydrogen. In some embodiments of a compound of Formula (VIIa), (VIIa-1), (VIIa-2), (VIIb), (VIIb-1), or (VIIb-2), R¹³ is C₁-C₆alkyl.

In some embodiments of a compound of Formula (VIIa), (VIIa-1), (VIIa-2), (VIIb), (VIIb-1), or (VIIb-2), R¹⁴ is —OH. In some embodiments of a compound of Formula (VIIa), (VIIa-1), (VIIa-2), (VIIb), (VIIb-1), or (VIIb-2), R¹⁴ is fluoro.

In some embodiments of a compound of Formula (VIIa), (VIIa-1), (VIIa-2), (VIIb), (VIIb-1), or (VIIb-2), R¹² is hydrogen, —C(═O)R²², —C(═O)OR²², or C₁-C₆alkyl. In some embodiments of a compound of Formula (VIIa), (VIIa-1), (VIIa-2), (VIIb), (VIIb-1), or (VIIb-2), R¹² is —C(═O)R²², —C(═O)OR²², or C₁-C₆alkyl. In some embodiments of a compound of Formula (VIIa), (VIIa-1), (VIIa-2), (VIIb), (VIIb-1), or (VIIb-2), R¹² is —C(═O)R²². In some embodiments of a compound of Formula (VIIa), (VIIa-1), (VIIa-2), (VIIb), (VIIb-1), or (VIIb-2), R¹² is —C(═O)OR²². In some embodiments of a compound of Formula (VIIa), (VIIa-1), (VIIa-2), (VIIb), (VIIb-1), or (VIIb-2), R¹² is C₁-C₆alkyl. In some embodiments of a compound of Formula (VIIa), (VIIa-1), (VIIa-2), (VIIb), (VIIb-1), or (VIIb-2), R¹² is hydrogen.

In some embodiments of a compound of Formula (VIIa), (VIIa-1), (VIIa-2), (VIIb), (VIIb-1), or (VIIb-2), R¹² is —C(═O)NR²²R²³.

In some embodiments of a compound of Formula (VIIa), (VIIa-1), (VIIa-2), (VIIb), (VIIb-1), or (VIIb-2), R²² is C₁-C₆alkyl, C₁-C₆aminoalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{22a}.

In some embodiments of a compound of Formula (VIIa), (VIIa-1), (VIIa-2), (VIIb), (VIIb-1), or (VIIb-2), R²² is C₁-C₆alkyl, C₁-C₆aminoalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl).

In some embodiments of a compound of Formula (VIIa), (VIIa-1), (VIIa-2), (VIIb), (VIIb-1), or (VIIb-2), R²² is C₁-C₆alkyl, C₁-C₆aminoalkyl, aryl, C₁-C₆alkylene(cycloalkyl), C₁-C₆alkylene(heterocycloalkyl), C₁-C₆alkylene(aryl), or C₁-C₆alkylene(heteroaryl).

In some embodiments of a compound of Formula (VIIa), (VIIa-1), (VIIa-2), (VIIb), (VIIb-1), or (VIIb-2), R²² is C₁-C₆alkyl, C₁-C₆aminoalkyl, aryl, C₁-C₆alkylene(cycloalkyl), or C₁-C₆alkylene(aryl).

In some embodiments of a compound of Formula (VIIa), (VIIa-1), (VIIa-2), (VIIb), (VIIb-1), or (VIIb-2), R²² is C₁-C₆alkyl or C₁-C₆alkylene(aryl); wherein the alkyl, alkylene, and aryl is optionally and independently substituted with one or more R^{22a}.

In some embodiments of a compound of Formula (VIIa), (VIIa-1), (VIIa-2), (VIIb), (VIIb-1), or (VIIb-2), R²² is C₁-C₆alkyl optionally and independently substituted with one or more R^{22a}.

In some embodiments of a compound of Formula (VIIa), (VIIa-1), (VIIa-2), (VIIb), (VIIb-1), or (VIIb-2), R²³ is hydrogen, C₁-C₆alkyl, C₁-C₆aminoalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R²². In some embodiments of a compound of Formula (VIIa), (VIIa-1), (VIIa-2), (VIIb), (VIIb-1), or (VIIb-2), R²³ is hydrogen, C₁-C₆alkyl, C₁-C₆aminoalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl). In some embodiments of a compound of Formula (VIIa), (VIIa-1), (VIIa-2), (VIIb), (VIIb-1), or (VIIb-2), R²³ is hydrogen, C₁-C₆alkyl, C₁-C₆aminoalkyl, aryl, C₁-C₆alkylene(cycloalkyl), C₁-C₆alkylene(heterocycloalkyl), C₁-C₆alkylene(aryl), or C₁-C₆alkylene(heteroaryl). In some embodiments of a compound of Formula (VIIa), (VIIa-1), (VIIa-2), (VIIb), (VIIb-1), or (VIIb-2), R²³ is hydrogen, C₁-C₆alkyl, C₁-C₆aminoalkyl, aryl, C₁-C₆alkylene(cycloalkyl), or C₁-C₆alkylene(aryl).

In some embodiments of a compound of Formula (VIIa), (VIIa-1), (VIIa-2), (VIIb), (VIIb-1), or (VIIb-2), R²³ is hydrogen, C₁-C₆alkyl, or C₁-C₆alkylene(aryl); wherein the alkyl, alkylene, and aryl is optionally and independently substituted with one or more R²². In some embodiments of a compound of Formula (VIIa), (VIIa-1), (VIIa-2), (VIIb), (VIIb-1), or (VIIb-2), R²³ is hydrogen or C₁-C₆alkyl optionally and independently substituted with one or more R²². In some embodiments of a compound of Formula (VIIa), (VIIa-1), (VIIa-2), (VIIb), (VIIb-1), or (VIIb-2), R²³ is C₁-C₆alkyl optionally and independently substituted with one or more R²². In some embodiments of a compound of Formula (VIIa), (VIIa-1), (VIIa-2), (VIIb), (VIIb-1), or (VIIb-2), R²³ is C₁-C₆alkyl. In some embodiments of a compound of Formula (VIIa), (VIIa-1), (VIIa-2), (VIIb), (VIIb-1), or (VIIb-2), R²³ is hydrogen.

In some embodiments of a compound of Formula (VIIa), (VIIa-1), (VIIa-2), (VIIb), (VIIb-1), or (VIIb-2), each R²² is independently halogen, —CN, —OH, —OR^{a}, —OC(═O)R^{a}, —OC(═O)OR^{b}, —OC(═O)NR^{c}R^{d}, —NR^{c}R^{d}, —C(═O)R^{a}, —C(═O)OR^{b}, —C(═O)NR^{c}R^{d}, C₁-C₆alkyl, or C₁-C₆haloalkyl.

In some embodiments of a compound of Formula (VIIa), (VIIa-1), (VIIa-2), (VIIb), (VIIb-1), or (VIIb-2), each R²² is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, or C₁-C₆haloalkyl.

In some embodiments of a compound of Formula (VIIa), (VIIa-1), (VIIa-2), (VIIb), (VIIb-1), or (VIIb-2), each R²² is independently halogen, —OH, —OR^{a}, —NR^{c}R^{d}, C₁-C₆alkyl, or C₁-C₆haloalkyl.

In some embodiments of a compound of Formula (VIIa), (VIIa-1), (VIIa-2), (VIIb), (VIIb-1), or (VIIb-2), each R²² is independently halogen, —OC(═O)R^{a}, or —NR^{c}R^{d}. In some embodiments of a compound of Formula (VIIa), (VIIa-1), (VIIa-2), (VIIb), (VIIb-1), or (VIIb-2), each R²² is independently —OC(═O)R^{a} or —NR^{c}R^{d}. In some embodiments of a compound of Formula (VIIa), (VIIa-1), (VIIa-2), (VIIb), (VIIb-1), or (VIIb-2), each R²² is independently —OC(═O)R^{a}.

In some embodiments of a compound of Formula (VIIa), (VIIa-1), (VIIa-2), (VIIb), (VIIb-1), or (VIIb-2), R¹² is

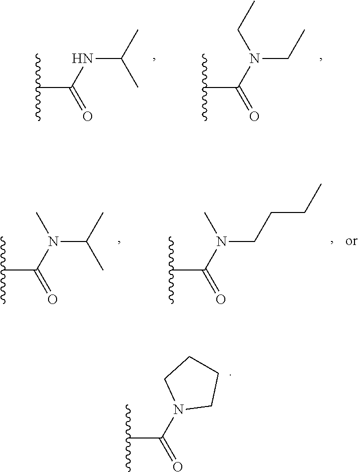

In some embodiments of a compound of Formula (VIIa), (VIIa-1), or (VIIa-2), R¹⁵ is —C(═O)R²⁵ or —C(═O)OR²⁵, or —CH₂—O—C(═O)R²⁵. In some embodiments of a compound of Formula (VIIa), (VIIa-1), or (VIIa-2), R¹⁵ is hydrogen. In some embodiments of a compound of Formula (VIIa), (VIIa-1), or (VIIa-2), R¹⁵ is —C(═O)R²⁵. In some embodiments of a compound of Formula (VIIa), (VIIa-1), or (VIIa-2), R¹⁵ is —C(═O)OR²⁵.

In some embodiments of a compound of Formula (VIIa), (VIIa-1), or (VIIa-2), R²⁵ is C₁-C₆alkylene(cycloalkyl) or C₁-C₆alkylene(aryl); wherein the alkylene, cycloalkyl, and aryl is optionally and independently substituted with one or more R^{25a}.

In some embodiments of a compound of Formula (VIIa), (VIIa-1), or (VIIa-2), R²⁵ is C₁-C₆alkylene(cycloalkyl); wherein the alkylene and cycloalkyl is optionally and independently substituted with one or more R^{25a}.

In some embodiments of a compound of Formula (VIIa), (VIIa-1), or (VIIa-2), R²⁵ is C₁-C₆alkylene(aryl); wherein the alkylene, and aryl is optionally and independently substituted with one or more R^{25a}.

In some embodiments of a compound of Formula (VIIa), (VIIa-1), or (VIIa-2), each R^{25a} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, or C₁-C₆haloalkyl.

In some embodiments of a compound of Formula (VIIa), (VIIa-1), or (VIIa-2), each R^{25a} is independently halogen, —OH, —OR^{a}, —NR^{c}R^{d}, C₁-C₆alkyl, or C₁-C₆haloalkyl.

In some embodiments of a compound of Formula (VIIa), (VIIa-1), or (VIIa-2), R¹⁵ is hydrogen,

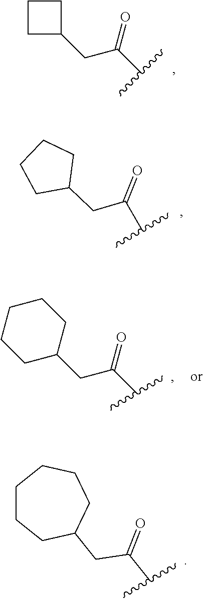 [SMILES: *C(=O)CC1CCC1 ; "," ; *C(=O)CC1CCCC1 ; *C(=O)CC1CCCCC1 ; *C(=O)CC1CCCCCC1]

In some embodiments of a compound of Formula (VIIa), (VIIa-1), or (VIIa-2), R¹⁵ is hydrogen,

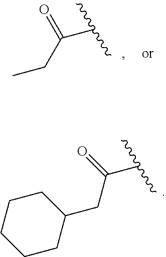

In some embodiments of a compound of Formula (VIIb), (VIIb-1), or (VIIb-2), R¹⁶ is hydrogen, —C(═O)R²⁶, —C(═O)OR²⁶, or C₁-C₆alkyl optionally substituted with one or more R^{16a}. In some embodiments of a compound of Formula (VIIb), (VIIb-1), or (VIIb-2), R¹⁶ is hydrogen, —C(═O)R²⁶, or —C(═O)OR²⁶. In some embodiments of a compound of Formula (VIIb), (VIIb-1), or (VIIb-2), R¹⁶ is —C(═O)R²⁶ or —C(═O)OR²⁶. In some embodiments of a compound of Formula (VIIb), (VIIb-1), or (VIIb-2), R¹⁶ is hydrogen. In some embodiments of a compound of Formula (VIIb), (VIIb-1), or (VIIb-2), R¹⁶ is —C(═O)R²⁶. In some embodiments of a compound of Formula (VIIb), (VIIb-1), or (VIIb-2), R¹⁶ is —C(═O)OR²⁶.

In some embodiments of a compound of Formula (VIIb), (VIIb-1), or (VIIb-2), R²⁶ is C₁-C₆alkyl, C₁-C₆aminoalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{26a}.

In some embodiments of a compound of Formula (VIIb), (VIIb-1), or (VIIb-2), R²⁶ is C₁-C₆alkyl, C₁-C₆aminoalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl).

In some embodiments of a compound of Formula (VIIb), (VIIb-1), or (VIIb-2), R²⁶ is C₁-C₆alkyl, C₁-C₆aminoalkyl, aryl, C₁-C₆alkylene(cycloalkyl), C₁-C₆alkylene(heterocycloalkyl), C₁-C₆alkylene(aryl), or C₁-C₆alkylene(heteroaryl).

In some embodiments of a compound of Formula (VIIb), (VIIb-1), or (VIIb-2), R²⁶ is C₁-C₆alkyl, C₁-C₆aminoalkyl, aryl, C₁-C₆alkylene(cycloalkyl), or C₁-C₆alkylene(aryl). In some embodiments of a compound of Formula (VIIb), (VIIb-1), or (VIIb-2), R²⁶ is C₁-C₆alkylene(cycloalkyl) or C₁-C₆alkylene(aryl).

In some embodiments of a compound of Formula (VIIb), (VIIb-1), or (VIIb-2), R²⁶ is C₁-C₆alkyl or C₁-C₆aminoalkyl.

In some embodiments of a compound of Formula (VIIb), (VIIb-1), or (VIIb-2), each R^{26a} is independently halogen, —CN, —OH, —OR^{a}, —OC(═O)R^{a}, —OC(═O)OR^{b}, —OC(═O)NR^{c}R^{d}, —NR^{c}R^{d}, —C(═O)R^{a}, —C(═O)OR^{b}, —C(═O)NR^{c}R^{d}, C₁-C₆alkyl, or C₁-C₆haloalkyl.

In some embodiments of a compound of Formula (VIIb), (VIIb-1), or (VIIb-2), each R^{26a} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, or C₁-C₆haloalkyl.

In some embodiments of a compound of Formula (VIIb), (VIIb-1), or (VIIb-2), each R^{26a} is independently halogen, —OH, —OR^{a}, —NR^{c}R^{d}, C₁-C₆alkyl, or C₁-C₆haloalkyl.

In some embodiments of a compound of Formula (VIIb), (VIIb-1), or (VIIb-2), each R^{26a} is independently —OC(═O)R^{a}, or —NR^{c}R^{d}.

In some embodiments of a compound of Formula (VIIb), (VIIb-1), or (VIIb-2), R¹⁶ is hydrogen,

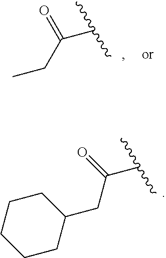

In some embodiments of a compound of Formula (VIIb), (VIIb-1), or (VIIb-2), R¹⁶ is hydrogen,

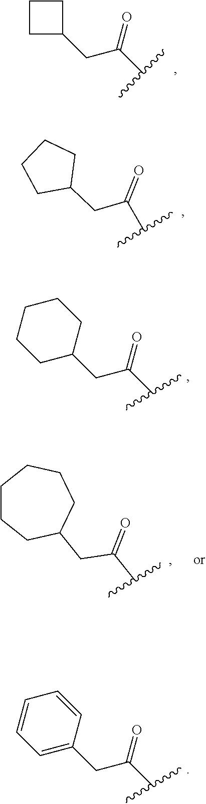 [SMILES: *C(=O)CC1CCC1 ; "," ; *C(=O)CC1CCCC1 ; *C(=O)CC1CCCCC1 ; *C(=O)CC1CCCCCC1 ; *C(=O)Cc1ccccc1]

In some embodiments of a compound disclosed herein, or a pharmaceutically acceptable salt, solvate, or stereoisomer thereof, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkyl(cycloalkyl), C₁-C₆alkylene(heterocycloalkyl), C₁-C₆alkyl(aryl), or C₁-C₆alkyl(heteroaryl); wherein each alkyl, cycloalkyl, heterocycloalkyl, aryl, and heteroaryl is independently optionally substituted with one or more R. In some embodiments of a compound disclosed herein, or a pharmaceutically acceptable salt, solvate, or stereoisomer thereof, each R^{a} is independently C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound disclosed herein, or a pharmaceutically acceptable salt, solvate, or stereoisomer thereof, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl). In some embodiments of a compound disclosed herein, or a pharmaceutically acceptable salt, solvate, or stereoisomer thereof, each R^{a} is independently C₁-C₆alkyl, C₁-C₆haloalkyl, or cycloalkyl, heterocycloalkyl. In some embodiments of a compound disclosed herein, or a pharmaceutically acceptable salt, solvate, or stereoisomer thereof, each R^{a} is independently C₁-C₆alkyl or C₁-C₆haloalkyl. In some embodiments of a compound disclosed herein, or a pharmaceutically acceptable salt, solvate, or stereoisomer thereof, each R^{a} is independently C₁-C₆alkyl.

In some embodiments of a compound disclosed herein, or a pharmaceutically acceptable salt, solvate, or stereoisomer thereof, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, cycloalkyl, heterocycloalkyl, aryl, and heteroaryl is independently optionally substituted with one or more R. In some embodiments of a compound disclosed herein, or a pharmaceutically acceptable salt, solvate, or stereoisomer thereof, each R^{b} is independently hydrogen, C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound disclosed herein, or a pharmaceutically acceptable salt, solvate, or stereoisomer thereof, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl). In some embodiments of a compound disclosed herein, or a pharmaceutically acceptable salt, solvate, or stereoisomer thereof, each R^{b} is independently hydrogen, C₁-C₆alkyl, C₁-C₆haloalkyl, or cycloalkyl, heterocycloalkyl. In some embodiments of a compound disclosed herein, or a pharmaceutically acceptable salt, solvate, or stereoisomer thereof, each R^{b} is independently hydrogen, C₁-C₆alkyl or C₁-C₆haloalkyl. In some embodiments of a compound disclosed herein, or a pharmaceutically acceptable salt, solvate, or stereoisomer thereof, each R^{b} is independently hydrogen or C₁-C₆alkyl. In some embodiments of a compound disclosed herein, or a pharmaceutically acceptable salt, solvate, or stereoisomer thereof, each R^{b} is hydrogen. In some embodiments of a compound disclosed herein, or a pharmaceutically acceptable salt, solvate, or stereoisomer thereof, each R^{b} is independently C₁-C₆alkyl.

In some embodiments of a compound disclosed herein, or a pharmaceutically acceptable salt, solvate, or stereoisomer thereof, each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, cycloalkyl, heterocycloalkyl, aryl, and heteroaryl is independently optionally substituted with one or more R. In some embodiments of a compound disclosed herein, or a pharmaceutically acceptable salt, solvate, or stereoisomer thereof, each R^{c} and R^{d} are independently hydrogen, C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound disclosed herein, or a pharmaceutically acceptable salt, solvate, or stereoisomer thereof, each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkyl(heteroaryl). In some embodiments of a compound disclosed herein, or a pharmaceutically acceptable salt, solvate, or stereoisomer thereof, each R^{c} and R^{d} are independently hydrogen, C₁-C₆alkyl, C₁-C₆haloalkyl, or cycloalkyl, heterocycloalkyl. In some embodiments of a compound disclosed herein, or a pharmaceutically acceptable salt, solvate, or stereoisomer thereof, each R^{c} and R^{d} are independently hydrogen, C₁-C₆alkyl or C₁-C₆haloalkyl. In some embodiments of a compound disclosed herein, or a pharmaceutically acceptable salt, solvate, or stereoisomer thereof, each R^{c} and R^{d} are independently hydrogen or C₁-C₆alkyl. In some embodiments of a compound disclosed herein, or a pharmaceutically acceptable salt, solvate, or stereoisomer thereof, each R^{c} and R^{d} are hydrogen. In some embodiments of a compound disclosed herein, or a pharmaceutically acceptable salt, solvate, or stereoisomer thereof, each R^{c} and R^{d} are independently C₁-C₆alkyl.

In some embodiments of a compound disclosed herein, or a pharmaceutically acceptable salt, solvate, or stereoisomer thereof, R^{c} and R^{d} are taken together with the atom to which they are attached to form a heterocycloalkyl optionally substituted with one or more R.

In some embodiments of a compound disclosed herein, or a pharmaceutically acceptable salt, solvate, or stereoisomer thereof, each R is independently halogen, —CN, —OH, —OC₁-C₆alkyl, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, or C₁-C₆haloalkyl. In some embodiments of a compound disclosed herein, or a pharmaceutically acceptable salt, solvate, or stereoisomer thereof, each R is independently halogen, —CN, —OH, —OC₁-C₆alkyl, —NH₂, —C(═O)C₁-C₆alkyl, —C(═O)OH, —C(═O)OC₁-C₆alkyl, —C(═O)NH₂, C₁-C₆alkyl, or C₁-C₆haloalkyl. In some embodiments of a compound disclosed herein, or a pharmaceutically acceptable salt, solvate, or stereoisomer thereof, each R is independently halogen, —CN, —OH, —OC₁-C₆alkyl, —NH₂, C₁-C₆alkyl, or C₁-C₆haloalkyl.

Any combination of the groups described above for the various variables is contemplated herein. Throughout the specification, groups and substituents thereof are chosen by one skilled in the field to provide stable moieties and compounds.

Described herein is a compound, or a pharmaceutically acceptable salt, solvate, or stereoisomer thereof, selected from a compound in Table 1.

TABLE 1
Exemplary compounds

| | | | ESI-MS |
|---|---|---|---|
| Example | | | (M + H)⁺ |
| No. | Structure | Chemical Name | (m/z) |
| 1 | 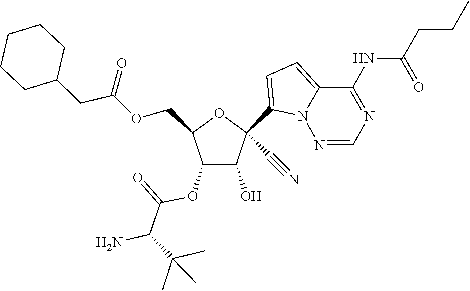 [SMILES: CCCC(=O)Nc1ncnn2c([C@]3(C#N)O[C@H](COC(=O)CC4CCCCC4)[C@@H](OC(=O)[C@@H](N)C(C)(C)C)[C@H]3O)ccc12] | (2R,3S,4R,5R)-5-(4- butyramidopyrrolo[2,1-f][1,2,4]triazin- 7-yl)-5-cyano-2-((2- cyclohexylacetoxy)methyl)-4- hydroxytetrahydrofuran-3-yl (S)-2- amino-3,3-dimethylbutanoate | 599.25 |
| 2 | 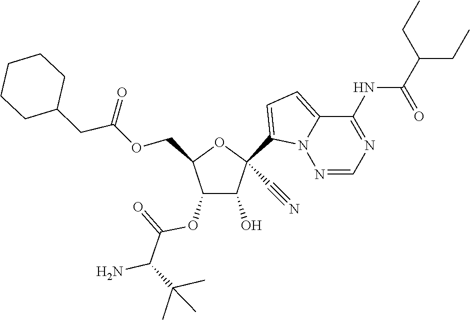 [SMILES: CCC(CC)C(=O)Nc1ncnn2c([C@]3(C#N)O[C@H](COC(=O)CC4CCCCC4)[C@@H](OC(=O)[C@@H](N)C(C)(C)C)[C@H]3O)ccc12] | (2R,3S,4R,5R)-5-cyano-2-((2- cyclohexylacetoxy)methyl)-5-(4-(2- ethylbutanamido)pyrrolo[2,1- f][1,2,4]triazin-7-yl)-4- hydroxytetrahydrofuran-3-yl (S)-2- amino-3,3-dimethylbutanoate | 627.15 |
| 3 | 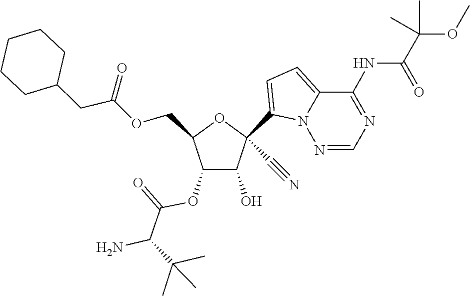 [SMILES: COC(C)(C)C(=O)Nc1ncnn2c([C@]3(C#N)O[C@H](COC(=O)CC4CCCCC4)[C@@H](OC(=O)[C@@H](N)C(C)(C)C)[C@H]3O)ccc12] | (2R,3S,4R,5R)-5-cyano-2-((2- cyclohexylacetoxy)methyl)-4-hydroxy- 5-(4-(2-methoxy-2- methylpropanamido)pyrrolo[2,1- f][1,2,4]triazin-7-yl)tetrahydrofuran-3- yl (S)-2-amino-3,3-dimethylbutanoate | 629.15 |
| 4 | 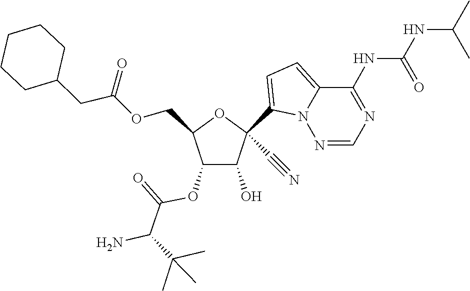 [SMILES: CC(C)NC(=O)Nc1ncnn2c([C@]3(C#N)O[C@H](COC(=O)CC4CCCCC4)[C@@H](OC(=O)[C@@H](N)C(C)(C)C)[C@H]3O)ccc12] | (2R,3S,4R,5R)-5-cyano-2-((2- cyclohexylacetoxy)methyl)-4-hydroxy- 5-(4-(3-isopropylureido)pyrrolo[2,1- f][1,2,4]triazin-7-yl)tetrahydrofuran-3- yl (S)-2-amino-3,3-dimethylbutanoate | 614.15 |
| 5 | 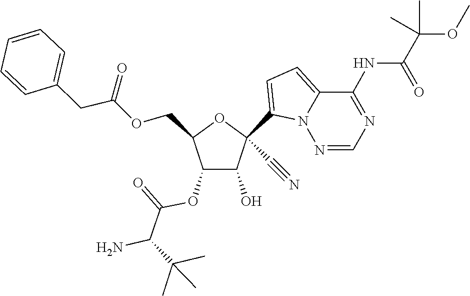 [SMILES: COC(C)(C)C(=O)Nc1ncnn2c([C@]3(C#N)O[C@H](COC(=O)Cc4ccccc4)[C@@H](OC(=O)[C@@H](N)C(C)(C)C)[C@H]3O)ccc12] | (2R,3S,4R,5R)-5-cyano-4-hydroxy-5- (4-(2-methoxy-2- methylpropanamido)pyrrolo[2,1- f][1,2,4]triazin-7-yl)-2-((2- phenylacetoxy)methyl)tetrahydrofuran- 3-yl (S)-2-amino-3,3-dimethylbutanoate | 623.15 |
| 6 | 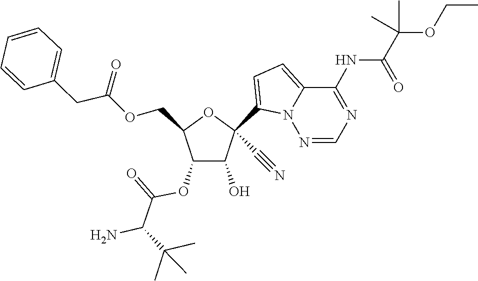 [SMILES: CCOC(C)(C)C(=O)Nc1ncnn2c([C@]3(C#N)O[C@H](COC(=O)Cc4ccccc4)[C@@H](OC(=O)[C@@H](N)C(C)(C)C)[C@H]3O)ccc12] | (2R,3S,4R,5R)-5-cyano-5-(4-(2-ethoxy- 2-methylpropanamido)pyrrolo[2,1- f][1,2,4]triazin-7-yl)-4-hydroxy-2-((2- phenylacetoxy)methyl)tetrahydrofuran- 3-yl (S)-2-amino-3,3-dimethylbutanoate | 637.15 |
| 7 | 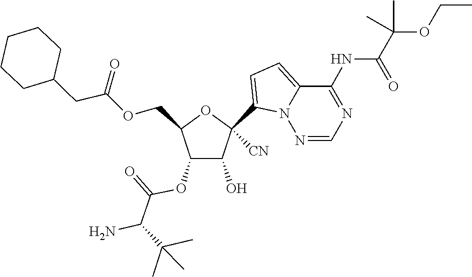 [SMILES: CCOC(C)(C)C(=O)Nc1ncnn2c([C@]3(C#N)O[C@H](COC(=O)CC4CCCCC4)[C@@H](OC(=O)[C@@H](N)C(C)(C)C)[C@H]3O)ccc12] | (2R,3S,4R,5R)-5-cyano-2-((2- cyclohexylacetoxy)methyl)-5-(4-(2- ethoxy-2- methylpropanamido)pyrrolo[2,1- f][1,2,4]triazin-7-yl)-4- hydroxytetrahydrofuran-3-yl (S)-2- amino-3,3-dimethylbutanoate | 643.25 |
| 8 | 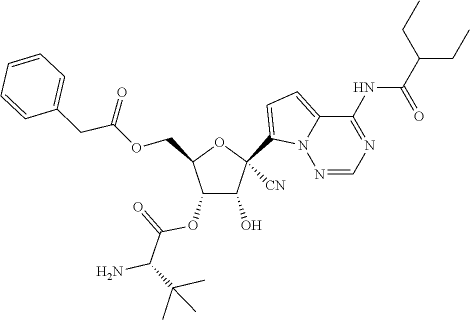 [SMILES: CCC(CC)C(=O)Nc1ncnn2c([C@]3(C#N)O[C@H](COC(=O)Cc4ccccc4)[C@@H](OC(=O)[C@@H](N)C(C)(C)C)[C@H]3O)ccc12] | (2R,3S,4R,5R)-5-cyano-5-(4-(2- ethylbutanamido)pyrrolo[2,1- f][1,2,4]triazin-7-yl)-4-hydroxy-2-((2- phenylacetoxy)methyl)tetrahydrofuran- 3-yl (S)-2-amino-3,3-dimethylbutanoate | 621.10 |
| 9 | 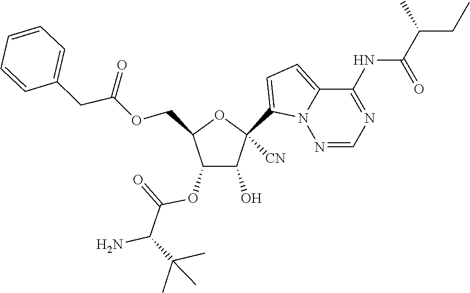 [SMILES: CC[C@@H](C)C(=O)Nc1ncnn2c([C@]3(C#N)O[C@H](COC(=O)Cc4ccccc4)[C@@H](OC(=O)[C@@H](N)C(C)(C)C)[C@H]3O)ccc12] | (2R,3S,4R,5R)-5-cyano-4-hydroxy-5- (4-((R)-2- methylbutanamido)pyrrolo[2,1- f][1,2,4]triazin-7-yl)-2-((2- phenylacetoxy)methyl)tetrahydrofuran- 3-yl (S)-2-amino-3,3-dimethylbutanoate | 607.10 |
| 10 | 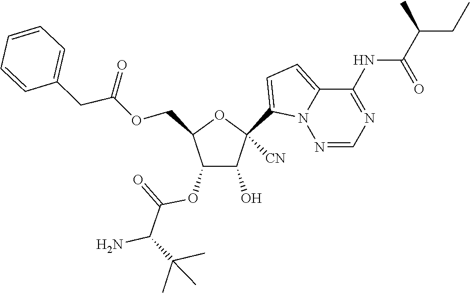 [SMILES: CC[C@H](C)C(=O)Nc1ncnn2c([C@]3(C#N)O[C@H](COC(=O)Cc4ccccc4)[C@@H](OC(=O)[C@@H](N)C(C)(C)C)[C@H]3O)ccc12] | (2R,3S,4R,5R)-5-cyano-4-hydroxy-5- (4-((S)-2- methylbutanamido)pyrrolo[2,1- f][1,2,4]triazin-7-yl)-2-((2- phenylacetoxy)methyl)tetrahydrofuran- 3-yl (S)-2-amino-3,3-dimethylbutanoate | 607.10 |
| 11 | 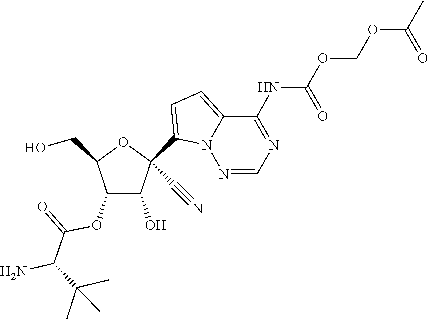 [SMILES: CC(=O)OCOC(=O)Nc1ncnn2c([C@]3(C#N)O[C@H](CO)[C@@H](OC(=O)[C@@H](N)C(C)(C)C)[C@H]3O)ccc12] | (2R,3S,4R,5R)-5-(4- (((acetoxymethoxy)carbonyl)amino) pyrrolo[2,1-f][1,2,4]triazin-7-yl)-5-cyano- 4-hydroxy-2- (hydroxymethyl)tetrahydrofuran-3-yl (S)-2-amino-3,3-dimethylbutanoate | 521.10 |
| 12 | 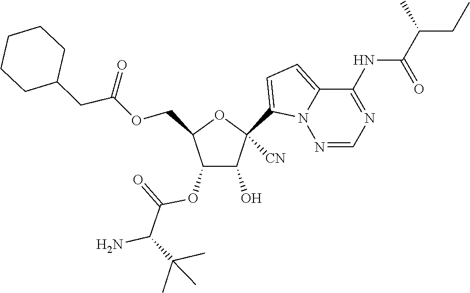 [SMILES: CC[C@@H](C)C(=O)Nc1ncnn2c([C@]3(C#N)O[C@H](COC(=O)CC4CCCCC4)[C@@H](OC(=O)[C@@H](N)C(C)(C)C)[C@H]3O)ccc12] | (2R,3S,4R,5R)-5-cyano-2-((2- cyclohexylacetoxy)methyl)-4-hydroxy- 5-(4-((R)-2- methylbutanamido)pyrrolo[2,1- f][1,2,4]triazin-7-yl)tetrahydrofuran-3- yl (S)-2-amino-3,3-dimethylbutanoate | 613.25 |
| 13 | 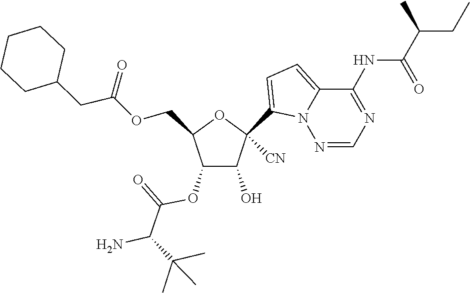 [SMILES: CC[C@H](C)C(=O)Nc1ncnn2c([C@]3(C#N)O[C@H](COC(=O)CC4CCCCC4)[C@@H](OC(=O)[C@@H](N)C(C)(C)C)[C@H]3O)ccc12] | (2R,3S,4R,5R)-5-cyano-2-((2- cyclohexylacetoxy)methyl)-4-hydroxy- 5-(4-((S)-2- methylbutanamido)pyrrolo[2,1- f][1,2,4]triazin-7-yl)tetrahydrofuran-3- yl (S)-2-amino-3,3-dimethylbutanoate | 613.15 |
| 14 | 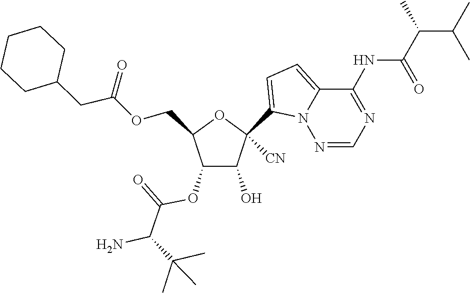 [SMILES: CC(C)[C@@H](C)C(=O)Nc1ncnn2c([C@]3(C#N)O[C@H](COC(=O)CC4CCCCC4)[C@@H](OC(=O)[C@@H](N)C(C)(C)C)[C@H]3O)ccc12] | (2R,3S,4R,5R)-5-cyano-2-((2- cyclohexylacetoxy)methyl)-5-(4-((R)- 2,3-dimethylbutanamido)pyrrolo[2,1- f][1,2,4]triazin-7-yl)-4- hydroxytetrahydrofuran-3-yl (S)-2- amino-3,3-dimethylbutanoate | 627.15 |
| 15 | 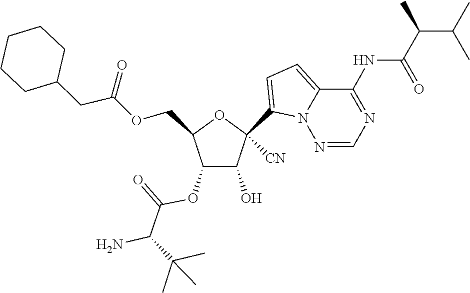 [SMILES: CC(C)[C@H](C)C(=O)Nc1ncnn2c([C@]3(C#N)O[C@H](COC(=O)CC4CCCCC4)[C@@H](OC(=O)[C@@H](N)C(C)(C)C)[C@H]3O)ccc12] | (2R,3S,4R,5R)-5-cyano-2-((2- cyclohexylacetoxy)methyl)-5-(4-((S)- 2,3-dimethylbutanamido)pyrrolo[2,1- f][1,2,4]triazin-7-yl)-4- hydroxytetrahydrofuran-3-yl (S)-2- amino-3,3-dimethylbutanoate | 627.20 |
| 16 | 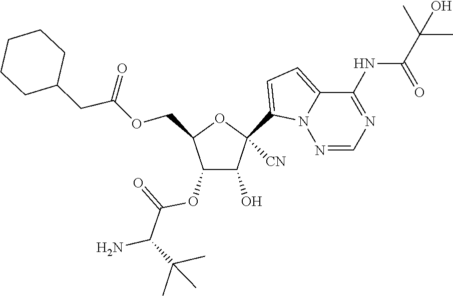 [SMILES: CC(C)(O)C(=O)Nc1ncnn2c([C@]3(C#N)O[C@H](COC(=O)CC4CCCCC4)[C@@H](OC(=O)[C@@H](N)C(C)(C)C)[C@H]3O)ccc12] | (2R,3S,4R,5R)-5-cyano-2-((2- cyclohexylacetoxy)methyl)-4-hydroxy- 5-(4-(2-hydroxy-2- methylpropanamido)pyrrolo[2,1- f][1,2,4]triazin-7-yl)tetrahydrofuran-3- yl (S)-2-amino-3,3-dimethylbutanoate | |
| 17 | 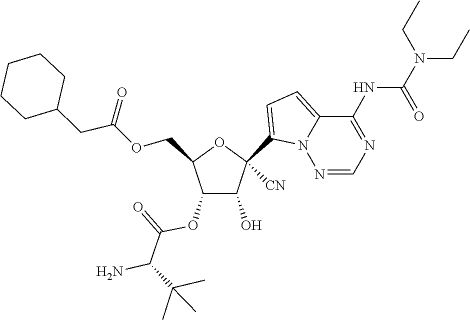 [SMILES: CCN(CC)C(=O)Nc1ncnn2c([C@]3(C#N)O[C@H](COC(=O)CC4CCCCC4)[C@@H](OC(=O)[C@@H](N)C(C)(C)C)[C@H]3O)ccc12] | (2R,3S,4R,5R)-5-cyano-2-((2- cyclohexylacetoxy)methyl)-5-(4-(3,3- diethylureido)pyrrolo[2,1- f][1,2,4]triazin-7-yl)-4- hydroxytetrahydrofuran-3-yl (S)-2- amino-3,3-dimethylbutanoate | |
| 18 | 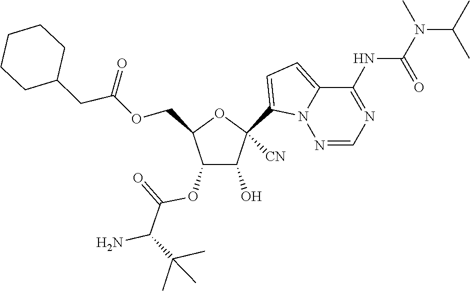 [SMILES: CC(C)N(C)C(=O)Nc1ncnn2c([C@]3(C#N)O[C@H](COC(=O)CC4CCCCC4)[C@@H](OC(=O)[C@@H](N)C(C)(C)C)[C@H]3O)ccc12] | (2R,3S,4R,5R)-5-cyano-2-((2- cyclohexylacetoxy)methyl)-4-hydroxy- 5-(4-(3-isopropyl-3- methylureido)pyrrolo[2,1- f][1,2,4]triazin-7-yl)tetrahydrofuran-3- yl (S)-2-amino-3,3-dimethylbutanoate | |
| 19 | 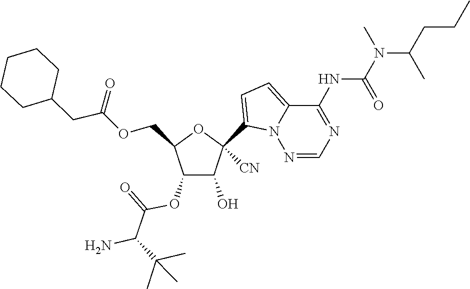 [SMILES: CCCC(C)N(C)C(=O)Nc1ncnn2c([C@]3(C#N)O[C@H](COC(=O)CC4CCCCC4)[C@@H](OC(=O)[C@@H](N)C(C)(C)C)[C@H]3O)ccc12] | (2R,3S,4R,5R)-5-cyano-2-((2- cyclohexylacetoxy)methyl)-4-hydroxy- 5-(4-(3-methyl-3-(pentan-2- yl)ureido)pyrrolo[2,1-f][1,2,4]triazin-7- yl)tetrahydrofuran-3-yl (2S)-2-amino- 3,3-dimethylbutanoate | |
| 20 | 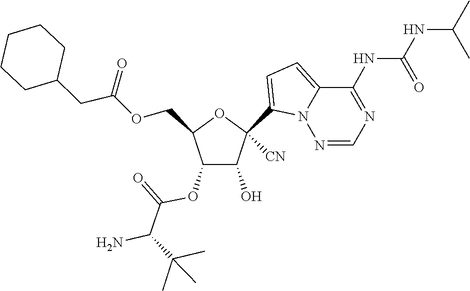 [SMILES: CC(C)NC(=O)Nc1ncnn2c([C@]3(C#N)O[C@H](COC(=O)CC4CCCCC4)[C@@H](OC(=O)[C@@H](N)C(C)(C)C)[C@H]3O)ccc12] | (2R,3S,4R,5R)-5-cyano-2-((2- cyclohexylacetoxy)methyl)-4-hydroxy- 5-(4-(3-isopropylureido)pyrrolo[2,1- f][1,2,4]triazin-7-yl)tetrahydrofuran-3- yl (S)-2-amino-3,3-dimethylbutanoate | 614.15 |
| 21 | 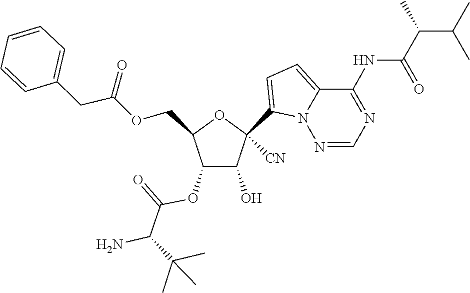 [SMILES: CC(C)[C@@H](C)C(=O)Nc1ncnn2c([C@]3(C#N)O[C@H](COC(=O)Cc4ccccc4)[C@@H](OC(=O)[C@@H](N)C(C)(C)C)[C@H]3O)ccc12] | (2R,3S,4R,5R)-5-cyano-5-(4-((R)-2,3- dimethylbutanamido)pyrrolo[2,1- f][1,2,4]triazin-7-yl)-4-hydroxy-2-((2- phenylacetoxy)methyl)tetrahydrofuran- 3-yl (S)-2-amino-3,3-dimethylbutanoate | 621.15 |
| 22 | 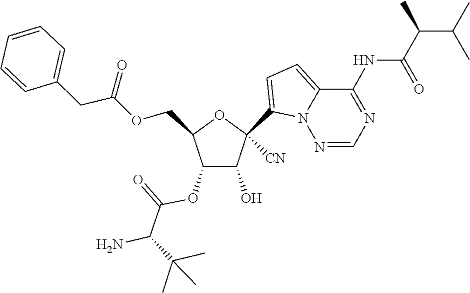 [SMILES: CC(C)[C@H](C)C(=O)Nc1ncnn2c([C@]3(C#N)O[C@H](COC(=O)Cc4ccccc4)[C@@H](OC(=O)[C@@H](N)C(C)(C)C)[C@H]3O)ccc12] | (2R,3S,4R,5R)-5-cyano-5-(4-((S)-2,3- dimethylbutanamido)pyrrolo[2,1- f][1,2,4]triazin-7-yl)-4-hydroxy-2-((2- phenylacetoxy)methyl)tetrahydrofuran- 3-yl (S)-2-amino-3,3-dimethylbutanoate | 621.20 |
| 23 | 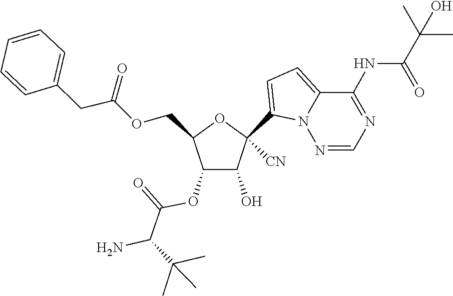 [SMILES: CC(C)(O)C(=O)Nc1ncnn2c([C@]3(C#N)O[C@H](COC(=O)Cc4ccccc4)[C@@H](OC(=O)[C@@H](N)C(C)(C)C)[C@H]3O)ccc12] | (2R,3S,4R,5R)-5-cyano-4-hydroxy-5- (4-(2-hydroxy-2- methylpropanamido)pyrrolo[2,1- f][1,2,4]triazin-7-yl)-2-((2- phenylacetoxy)methyl)tetrahydrofuran- 3-yl (S)-2-amino-3,3-dimethylbutanoate | |
| 24 | 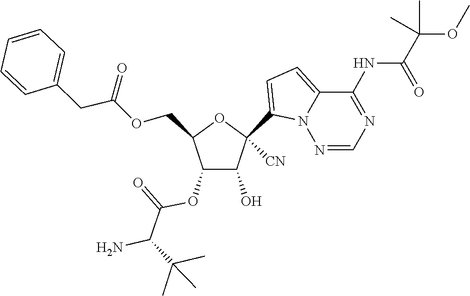 [SMILES: COC(C)(C)C(=O)Nc1ncnn2c([C@]3(C#N)O[C@H](COC(=O)Cc4ccccc4)[C@@H](OC(=O)[C@@H](N)C(C)(C)C)[C@H]3O)ccc12] | (2R,3S,4R,5R)-5-cyano-4-hydroxy-5- (4-(2-methoxy-2- methylpropanamido)pyrrolo[2,1- f][1,2,4]triazin-7-yl)-2-((2- phenylacetoxy)methyl)tetrahydrofuran- 3-yl (S)-2-amino-3,3-dimethylbutanoate | 623.15 |
| 25 | 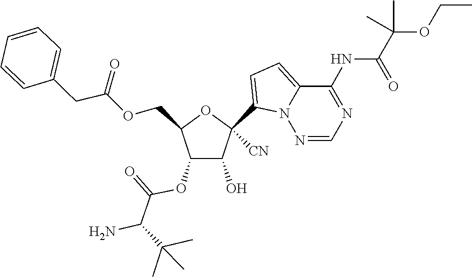 [SMILES: CCOC(C)(C)C(=O)Nc1ncnn2c([C@]3(C#N)O[C@H](COC(=O)Cc4ccccc4)[C@@H](OC(=O)[C@@H](N)C(C)(C)C)[C@H]3O)ccc12] | (2R,3S,4R,5R)-5-cyano-5-(4-(2-ethoxy- 2-methylpropanamido)pyrrolo[2,1- f][1,2,4]triazin-7-yl)-4-hydroxy-2-((2- phenylacetoxy)methyl)tetrahydrofuran- 3-yl (S)-2-amino-3,3-dimethylbutanoate | 637.15 |
| 26 | 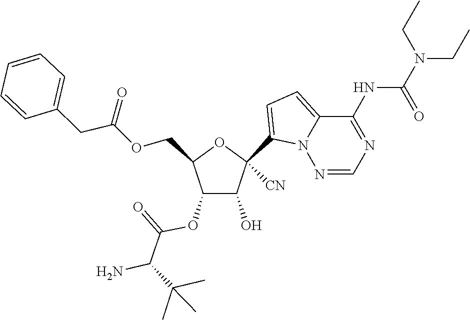 [SMILES: CCN(CC)C(=O)Nc1ncnn2c([C@]3(C#N)O[C@H](COC(=O)Cc4ccccc4)[C@@H](OC(=O)[C@@H](N)C(C)(C)C)[C@H]3O)ccc12] | (2R,3S,4R,5R)-5-cyano-5-(4-(3,3- diethylureido)pyrrolo[2,1- f][1,2,4]triazin-7-yl)-4-hydroxy-2-((2- phenylacetoxy)methyl)tetrahydrofuran- 3-yl (S)-2-amino-3,3-dimethylbutanoate | |
| 27 | 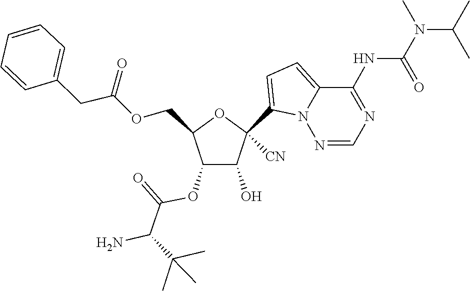 [SMILES: CC(C)N(C)C(=O)Nc1ncnn2c([C@]3(C#N)O[C@H](COC(=O)Cc4ccccc4)[C@@H](OC(=O)[C@@H](N)C(C)(C)C)[C@H]3O)ccc12] | (2R,3S,4R,5R)-5-cyano-4-hydroxy-5- (4-(3-isopropyl-3- methylureido)pyrrolo[2,1- f][1,2,4]triazin-7-yl)-2-((2- phenylacetoxy)methyl)tetrahydrofuran- 3-yl (S)-2-amino-3,3-dimethylbutanoate | |
| 28 | 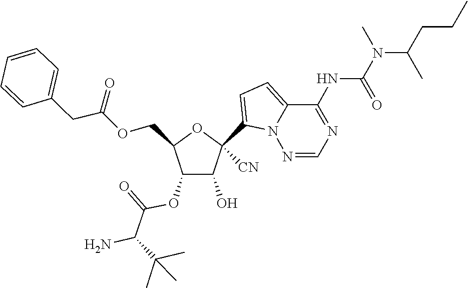 [SMILES: CCCC(C)N(C)C(=O)Nc1ncnn2c([C@]3(C#N)O[C@H](COC(=O)Cc4ccccc4)[C@@H](OC(=O)[C@@H](N)C(C)(C)C)[C@H]3O)ccc12] | (2R,3S,4R,5R)-5-cyano-4-hydroxy-5- (4-(3-methyl-3-(pentan-2- yl)ureido)pyrrolo[2,1-f][1,2,4]triazin-7- yl)-2-((2- phenylacetoxy)methyl)tetrahydrofuran- 3-yl (2S)-2-amino-3,3- dimethylbutanoate | |
| 29 | 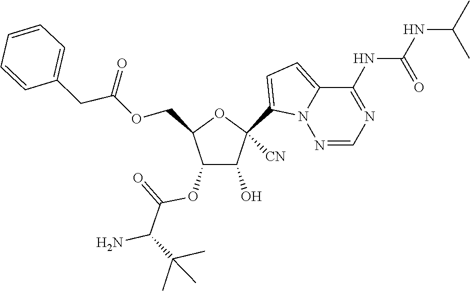 [SMILES: CC(C)NC(=O)Nc1ncnn2c([C@]3(C#N)O[C@H](COC(=O)Cc4ccccc4)[C@@H](OC(=O)[C@@H](N)C(C)(C)C)[C@H]3O)ccc12] | (2R,3S,4R,5R)-5-cyano-4-hydroxy-5- (4-(3-isopropylureido)pyrrolo[2,1- f][1,2,4]triazin-7-yl)-2-((2- phenylacetoxy)methyl)tetrahydrofuran- 3-yl (S)-2-amino-3,3-dimethylbutanoate | 608.15 |
| 30 | 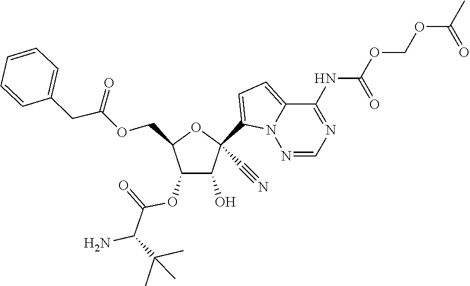 [SMILES: CC(=O)OCOC(=O)Nc1ncnn2c([C@]3(C#N)O[C@H](COC(=O)Cc4ccccc4)[C@@H](OC(=O)[C@@H](N)C(C)(C)C)[C@H]3O)ccc12] | (2R,3S,4R,5R)-5-(4- (((acetoxymethoxy)carbonyl)amino) pyrrolo[2,1-f][1,2,4]triazin-7-yl)-5-cyano- 4-hydroxy-2-((2- phenylacetoxy)methyl)tetrahydrofuran- 3-yl (S)-2-amino-3,3-dimethylbutanoate | 639.3 |
| 31 | 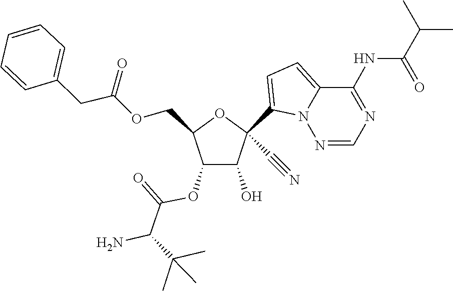 [SMILES: CC(C)C(=O)Nc1ncnn2c([C@]3(C#N)O[C@H](COC(=O)Cc4ccccc4)[C@@H](OC(=O)[C@@H](N)C(C)(C)C)[C@H]3O)ccc12] | (2R,3S,4R,5R)-5-cyano-4-hydroxy-5- (4-isobutyramidopyrrolo[2,1- f][1,2,4]triazin-7-yl)-2-((2- phenylacetoxy)methyl)tetrahydrofuran- 3-yl (S)-2-amino-3,3-dimethylbutanoate | 593.3 |
| 32 | 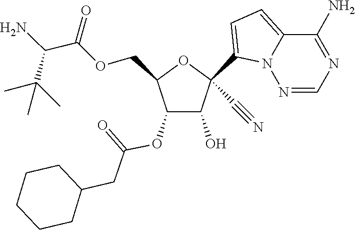 [SMILES: CC(C)(C)[C@H](N)C(=O)OC[C@H]1O[C@@](C#N)(c2ccc3c(N)ncnn23)[C@H](O)[C@@H]1OC(=O)CC1CCCCC1] | ((2R,3S,4R,5R)-5-(4-aminopyrrolo[2,1- f][1,2,4]triazin-7-yl)-5-cyano-3-(2- cyclohexylacetoxy)-4- hydroxytetrahydrofuran-2-yl)methyl (S)-2-amino-3,3-dimethylbutanoate | 529.3 |
| 33 | 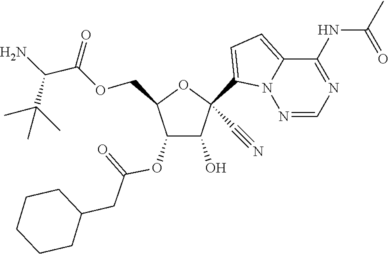 [SMILES: CC(=O)Nc1ncnn2c([C@]3(C#N)O[C@H](COC(=O)[C@@H](N)C(C)(C)C)[C@@H](OC(=O)CC4CCCCC4)[C@H]3O)ccc12] | ((2R,3S,4R,5R)-5-(4- acetamidopyrrolo[2,1-f][1,2,4]triazin-7- yl)-5-cyano-3-(2-cyclohexylacetoxy)-4- hydroxytetrahydrofuran-2-yl)methyl (S)-2-amino-3,3-dimethylbutanoate | 571.3 |
| 34 | 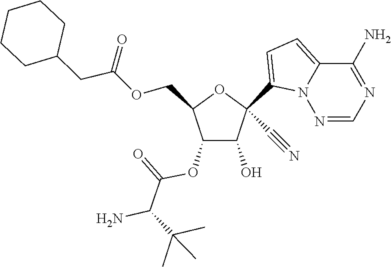 [SMILES: CC(C)(C)[C@H](N)C(=O)O[C@H]1[C@@H](O)[C@](C#N)(c2ccc3c(N)ncnn23)O[C@@H]1COC(=O)CC1CCCCC1] | (2R,3S,4R,5R)-5-(4-aminopyrrolo[2,1- f][1,2,4]triazin-7-yl)-5-cyano-2-((2- cyclohexylacetoxy)methyl)-4- hydroxytetrahydrofuran-3-yl (S)-2- amino-3,3-dimethylbutanoate | 529.2 |
| 35 | 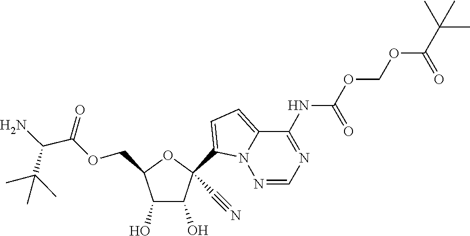 [SMILES: CC(C)(C)C(=O)OCOC(=O)Nc1ncnn2c([C@]3(C#N)O[C@H](COC(=O)[C@@H](N)C(C)(C)C)[C@@H](O)[C@H]3O)ccc12] | ((2R,3S,4R,5R)-5-cyano-3,4- dihydroxy-5-(4- ((((pivaloyloxy)methoxy)carbonyl)amino) pyrrolo[2,1-f][1,2,4]triazin-7- yl)tetrahydrofuran-2-yl)methyl (S)-2- amino-3,3-dimethylbutanoate | 563.2 |
| 36 | 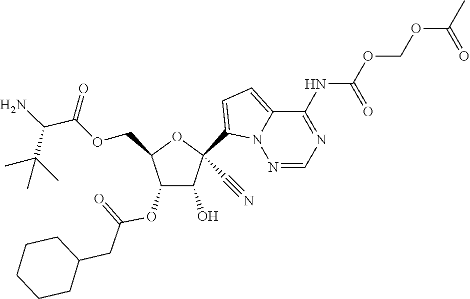 [SMILES: CC(=O)OCOC(=O)Nc1ncnn2c([C@]3(C#N)O[C@H](COC(=O)[C@@H](N)C(C)(C)C)[C@@H](OC(=O)CC4CCCCC4)[C@H]3O)ccc12] | ((2R,3S,4R,5R)-5-(4- (((acetoxymethoxy)carbonyl)amino) pyrrolo[2,1-f][1,2,4]triazin-7-yl)-5-cyano- 3-(2-cyclohexylacetoxy)-4- hydroxytetrahydrofuran-2-yl)methyl (S)-2-amino-3,3-dimethylbutanoate | 645.3 |
| 37 | 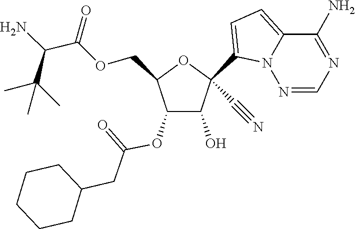 [SMILES: CC(C)(C)[C@@H](N)C(=O)OC[C@H]1O[C@@](C#N)(c2ccc3c(N)ncnn23)[C@H](O)[C@@H]1OC(=O)CC1CCCCC1] | ((2R,3S,4R,5R)-5-(4-aminopyrrolo[2,1- f][1,2,4]triazin-7-yl)-5-cyano-3-(2- cyclohexylacetoxy)-4- hydroxytetrahydrofuran-2-yl)methyl (R)-2-amino-3,3-dimethylbutanoate | 529.3 |
| 38 | 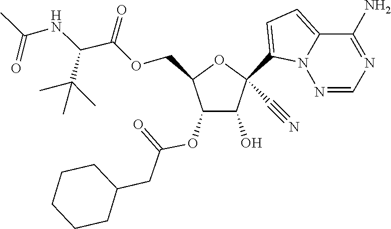 [SMILES: CC(=O)N[C@H](C(=O)OC[C@H]1O[C@@](C#N)(c2ccc3c(N)ncnn23)[C@H](O)[C@@H]1OC(=O)CC1CCCCC1)C(C)(C)C] | ((2R,3S,4R,5R)-5-(4-aminopyrrolo[2,1- f][1,2,4]triazin-7-yl)-5-cyano-3-(2- cyclohexylacetoxy)-4- hydroxytetrahydrofuran-2-yl)methyl (S)-2-acetamido-3,3-dimethylbutanoate | 571.2 |
| 39 | 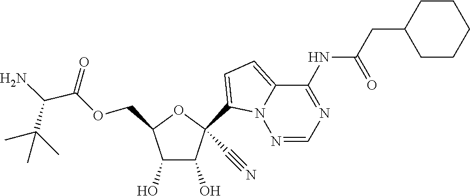 [SMILES: CC(C)(C)[C@H](N)C(=O)OC[C@H]1O[C@@](C#N)(c2ccc3c(NC(=O)CC4CCCCC4)ncnn23)[C@H](O)[C@@H]1O] | ((2R,3S,4R,5R)-5-cyano-5-(4-(2- cyclohexylacetamido)pyrrolo[2,1- f][1,2,4]triazin-7-yl)-3,4- dihydroxytetrahydrofuran-2-yl)methyl (S)-2-amino-3,3-dimethylbutanoate | 529.2 |
| 40 | 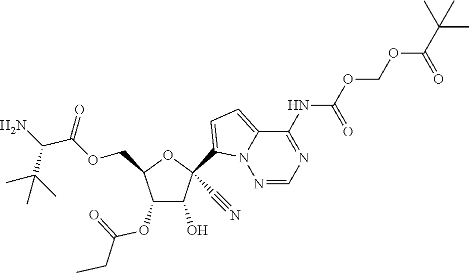 [SMILES: CCC(=O)O[C@H]1[C@@H](O)[C@](C#N)(c2ccc3c(NC(=O)OCOC(=O)C(C)(C)C)ncnn23)O[C@@H]1COC(=O)[C@@H](N)C(C)(C)C] | ((2R,3S,4R,5R)-5-cyano-4-hydroxy-5- (4- ((((pivaloyloxy)methoxy)carbonyl)amino) pyrrolo[2,1-f][1,2,4]triazin-7-yl)-3- (propionyloxy)tetrahydrofuran-2- yl)methyl (S)-2-amino-3,3- dimethylbutanoate | 619.3 |
| 41 | 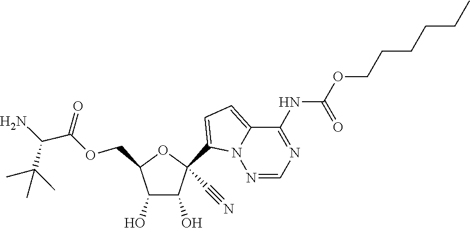 [SMILES: CCCCCCOC(=O)Nc1ncnn2c([C@]3(C#N)O[C@H](COC(=O)[C@@H](N)C(C)(C)C)[C@@H](O)[C@H]3O)ccc12] | ((2R,3S,4R,5R)-5-cyano-5-(4- (((hexyloxy)carbonyl)amino)pyrrolo[2,1- f][1,2,4]triazin-7-yl)-3,4- dihydroxytetrahydrofuran-2-yl)methyl (S)-2-amino-3,3-dimethylbutanoate | 533.3 |
| 42 | 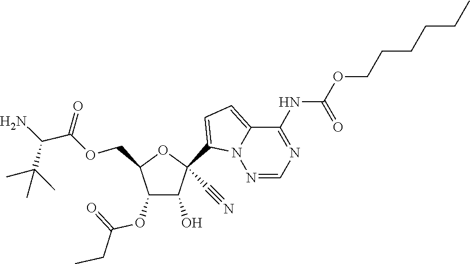 [SMILES: CCCCCCOC(=O)Nc1ncnn2c([C@]3(C#N)O[C@H](COC(=O)[C@@H](N)C(C)(C)C)[C@@H](OC(=O)CC)[C@H]3O)ccc12] | ((2R,3S,4R,5R)-5-cyano-5-(4- (((hexyloxy)carbonyl)amino)pyrrolo[2,1- f][1,2,4]triazin-7-yl)-4-hydroxy-3- (propionyloxy)tetrahydrofuran-2- yl)methyl (S)-2-amino-3,3- dimethylbutanoate | 589.05 |
| 43 | 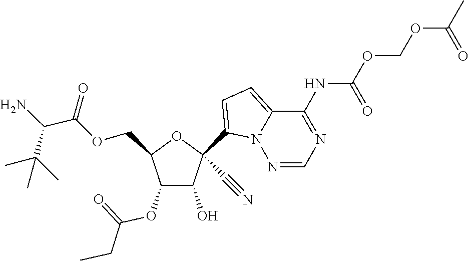 [SMILES: CCC(=O)O[C@H]1[C@@H](O)[C@](C#N)(c2ccc3c(NC(=O)OCOC(C)=O)ncnn23)O[C@@H]1COC(=O)[C@@H](N)C(C)(C)C] | ((2R,3S,4R,5R)-5-(4- (((acetoxymethoxy)carbonyl)amino) pyrrolo[2,1-f][1,2,4]triazin-7-yl)-5-cyano- 4-hydroxy-3- (propionyloxy)tetrahydrofuran-2- yl)methyl (S)-2-amino-3,3- dimethylbutanoate | 577.15 |
| 44 | 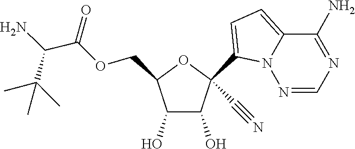 [SMILES: CC(C)(C)[C@H](N)C(=O)OC[C@H]1O[C@@](C#N)(c2ccc3c(N)ncnn23)[C@H](O)[C@@H]1O] | ((2R,3S,4R,5R)-5-(4-aminopyrrolo[2,1- f][1,2,4]triazin-7-yl)-5-cyano-3,4- dihydroxytetrahydrofuran-2-yl)methyl (S)-2-amino-3,3-dimethylbutanoate | 405.2 |
| 45 | 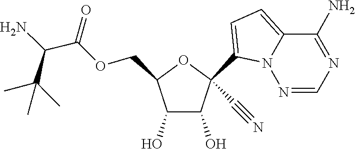 [SMILES: CC(C)(C)[C@@H](N)C(=O)OC[C@H]1O[C@@](C#N)(c2ccc3c(N)ncnn23)[C@H](O)[C@@H]1O] | ((2R,3S,4R,5R)-5-(4-aminopyrrolo[2,1- f][1,2,4]triazin-7-yl)-5-cyano-3,4- dihydroxytetrahydrofuran-2-yl)methyl (R)-2-amino-3,3-dimethylbutanoate | 405.2 |
| 46 | 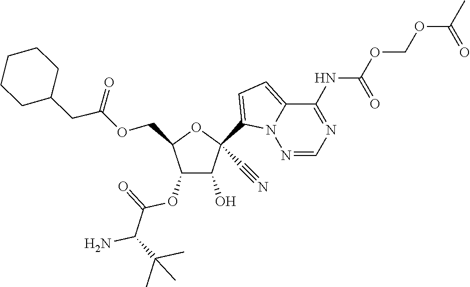 [SMILES: CC(=O)OCOC(=O)Nc1ncnn2c([C@]3(C#N)O[C@H](COC(=O)CC4CCCCC4)[C@@H](OC(=O)[C@@H](N)C(C)(C)C)[C@H]3O)ccc12] | (2R,3S,4R,5R)-5-(4- (((acetoxymethoxy)carbonyl)amino) pyrrolo[2,1-f][1,2,4]triazin-7-yl)-5-cyano- 2-((2-cyclohexylacetoxy)methyl)-4- hydroxytetrahydrofuran-3-yl (S)-2- amino-3,3-dimethylbutanoate | 645.20 |
| 47 | 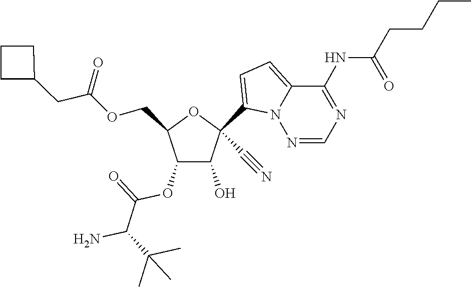 [SMILES: CCCCC(=O)Nc1ncnn2c([C@]3(C#N)O[C@H](COC(=O)CC4CCC4)[C@@H](OC(=O)[C@@H](N)C(C)(C)C)[C@H]3O)ccc12] | (2R,3S,4R,5R)-5-cyano-2-((2- cyclobutylacetoxy)methyl)-4-hydroxy- 5-(4-pentanamidopyrrolo[2,1- f][1,2,4]triazin-7-yl)tetrahydrofuran-3- yl (S)-2-amino-3,3-dimethylbutanoate | 585.00 |
| 48 | 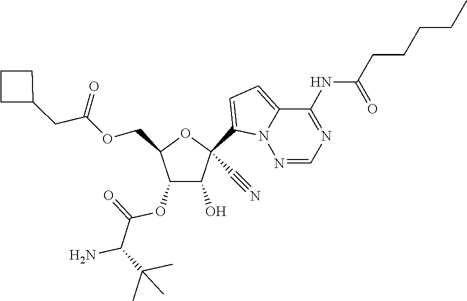 [SMILES: CCCCCC(=O)Nc1ncnn2c([C@]3(C#N)O[C@H](COC(=O)CC4CCC4)[C@@H](OC(=O)[C@@H](N)C(C)(C)C)[C@H]3O)ccc12] | (2R,3S,4R,5R)-5-cyano-2-((2- cyclobutylacetoxy)methyl)-5-(4- hexanamidopyrrolo[2,1-f][1,2,4]triazin- 7-yl)-4-hydroxytetrahydrofuran-3-yl (S)-2-amino-3,3-dimethylbutanoate | 599.20 |
| 49 | 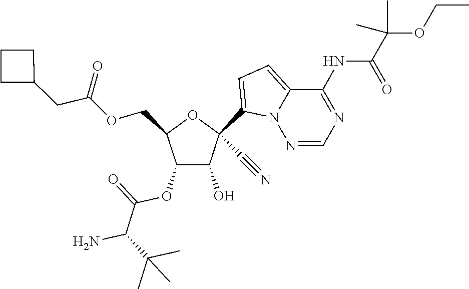 [SMILES: CCOC(C)(C)C(=O)Nc1ncnn2c([C@]3(C#N)O[C@H](COC(=O)CC4CCC4)[C@@H](OC(=O)[C@@H](N)C(C)(C)C)[C@H]3O)ccc12] | (2R,3S,4R,5R)-5-cyano-2-((2- cyclobutylacetoxy)methyl)-5-(4-(2- ethoxy-2- methylpropanamido)pyrrolo[2,1- f][1,2,4]triazin-7-yl)-4- hydroxytetrahydrofuran-3-yl (S)-2- amino-3,3-dimethylbutanoate | 615.10 |
| 50 | 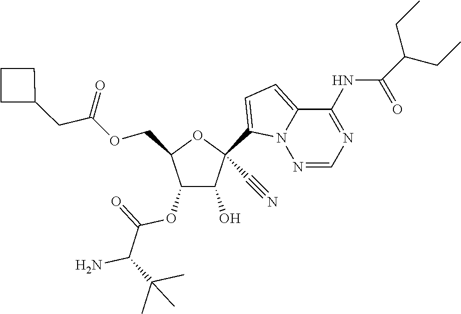 [SMILES: CCC(CC)C(=O)Nc1ncnn2c([C@]3(C#N)O[C@H](COC(=O)CC4CCC4)[C@@H](OC(=O)[C@@H](N)C(C)(C)C)[C@H]3O)ccc12] | (2R,3S,4R,5R)-5-cyano-2-((2- cyclobutylacetoxy)methyl)-5-(4-(2- ethylbutanamido)pyrrolo[2,1- f][1,2,4]triazin-7-yl)-4- hydroxytetrahydrofuran-3-yl (S)-2- amino-3,3-dimethylbutanoate | 599.05 |
| 51 | 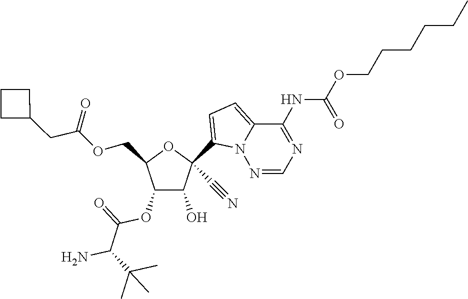 [SMILES: CCCCCCOC(=O)Nc1ncnn2c([C@]3(C#N)O[C@H](COC(=O)CC4CCC4)[C@@H](OC(=O)[C@@H](N)C(C)(C)C)[C@H]3O)ccc12] | (2R,3S,4R,5R)-5-cyano-2-((2- cyclobutylacetoxy)methyl)-5-(4- (((hexyloxy)carbonyl)amino)pyrrolo[2,1- f][1,2,4]triazin-7-yl)-4- hydroxytetrahydrofuran-3-yl (S)-2- amino-3,3-dimethylbutanoate | |
| 52 | 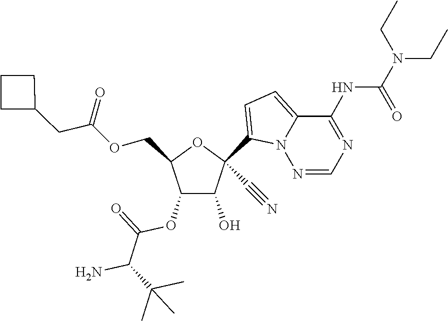 [SMILES: CCN(CC)C(=O)Nc1ncnn2c([C@]3(C#N)O[C@H](COC(=O)CC4CCC4)[C@@H](OC(=O)[C@@H](N)C(C)(C)C)[C@H]3O)ccc12] | (2R,3S,4R,5R)-5-cyano-2-((2- cyclobutylacetoxy)methyl)-5-(4-(3,3- diethylureido)pyrrolo[2,1- f][1,2,4]triazin-7-yl)-4- hydroxytetrahydrofuran-3-yl (S)-2- amino-3,3-dimethylbutanoate | |
| 53 | 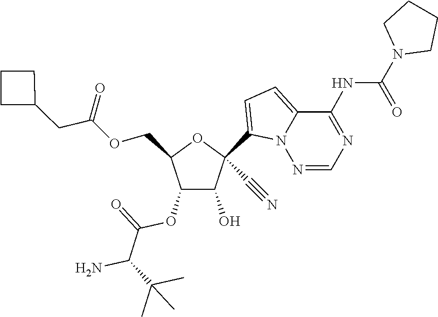 [SMILES: CC(C)(C)[C@H](N)C(=O)O[C@H]1[C@@H](O)[C@](C#N)(c2ccc3c(NC(=O)N4CCCC4)ncnn23)O[C@@H]1COC(=O)CC1CCC1] | (2R,3S,4R,5R)-5-cyano-2-((2- cyclobutylacetoxy)methyl)-4-hydroxy- 5-(4-(pyrrolidine-1- carboxamido)pyrrolo[2,1- f][1,2,4]triazin-7-yl)tetrahydrofuran-3- yl (S)-2-amino-3,3-dimethylbutanoate | |
| 54 | 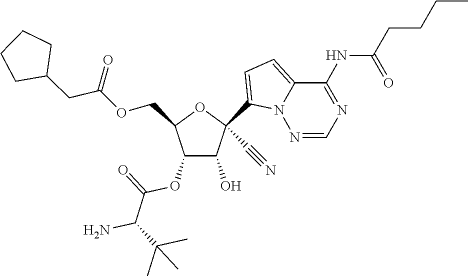 [SMILES: CCCCC(=O)Nc1ncnn2c([C@]3(C#N)O[C@H](COC(=O)CC4CCCC4)[C@@H](OC(=O)[C@@H](N)C(C)(C)C)[C@H]3O)ccc12] | (2R,3S,4R,5R)-5-cyano-2-((2- cyclopentylacetoxy)methyl)-4-hydroxy- 5-(4-pentanamidopyrrolo[2,1- f][1,2,4]triazin-7-yl)tetrahydrofuran-3- yl (S)-2-amino-3,3-dimethylbutanoate | 599.05 |
| 55 | 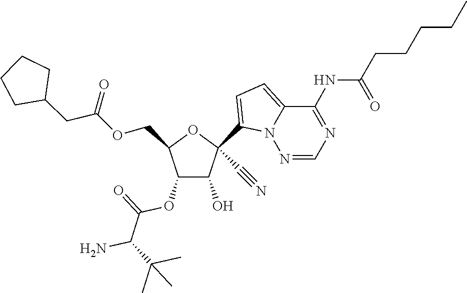 [SMILES: CCCCCC(=O)Nc1ncnn2c([C@]3(C#N)O[C@H](COC(=O)CC4CCCC4)[C@@H](OC(=O)[C@@H](N)C(C)(C)C)[C@H]3O)ccc12] | (2R,3S,4R,5R)-5-cyano-2-((2- cyclopentylacetoxy)methyl)-5-(4- hexanamidopyrrolo[2,1-f][1,2,4]triazin- 7-yl)-4-hydroxytetrahydrofuran-3-yl (S)-2-amino-3,3-dimethylbutanoate | 613.05 |
| 56 | 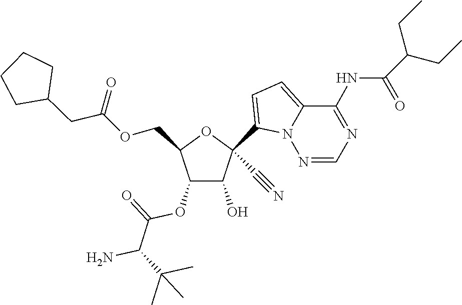 [SMILES: CCC(CC)C(=O)Nc1ncnn2c([C@]3(C#N)O[C@H](COC(=O)CC4CCCC4)[C@@H](OC(=O)[C@@H](N)C(C)(C)C)[C@H]3O)ccc12] | (2R,3S,4R,5R)-5-cyano-2-((2- cyclopentylacetoxy)methyl)-5-(4-(2- ethylbutanamido)pyrrolo[2,1- f][1,2,4]triazin-7-yl)-4- hydroxytetrahydrofuran-3-yl (S)-2- amino-3,3-dimethylbutanoate | 613.10 |
| 57 | 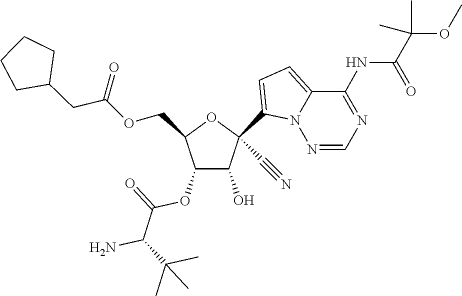 [SMILES: COC(C)(C)C(=O)Nc1ncnn2c([C@]3(C#N)O[C@H](COC(=O)CC4CCCC4)[C@@H](OC(=O)[C@@H](N)C(C)(C)C)[C@H]3O)ccc12] | (2R,3S,4R,5R)-5-cyano-2-((2- cyclopentylacetoxy)methyl)-4-hydroxy- 5-(4-(2-methoxy-2- methylpropanamido)pyrrolo[2,1- f][1,2,4]triazin-7-yl)tetrahydrofuran-3- yl (S)-2-amino-3,3-dimethylbutanoate | 615.05 |
| 58 | 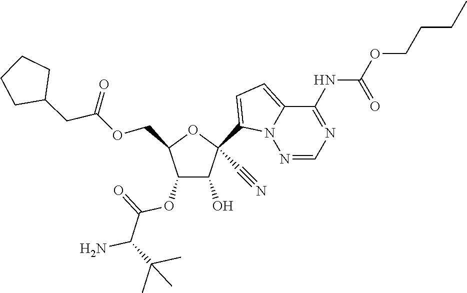 [SMILES: CCCCOC(=O)Nc1ncnn2c([C@]3(C#N)O[C@H](COC(=O)CC4CCCC4)[C@@H](OC(=O)[C@@H](N)C(C)(C)C)[C@H]3O)ccc12] | (2R,3S,4R,5R)-5-(4- ((butoxycarbonyl)amino)pyrrolo[2,1- f][1,2,4]triazin-7-yl)-5-cyano-2-((2- cyclopentylacetoxy)methyl)-4- hydroxytetrahydrofuran-3-yl (S)-2- amino-3,3-dimethylbutanoate | |
| 59 | 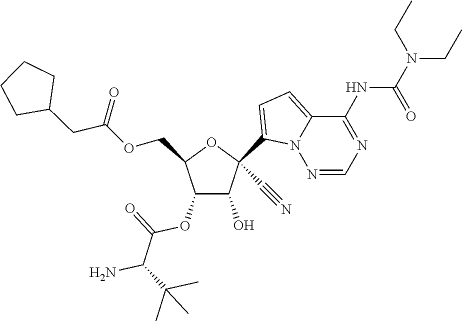 [SMILES: CCN(CC)C(=O)Nc1ncnn2c([C@]3(C#N)O[C@H](COC(=O)CC4CCCC4)[C@@H](OC(=O)[C@@H](N)C(C)(C)C)[C@H]3O)ccc12] | (2R,3S,4R,5R)-5-cyano-2-((2- cyclopentylacetoxy)methyl)-5-(4-(3,3- diethylureido)pyrrolo[2,1- f][1,2,4]triazin-7-yl)-4- hydroxytetrahydrofuran-3-yl (S)-2- amino-3,3-dimethylbutanoate | |
| 60 | 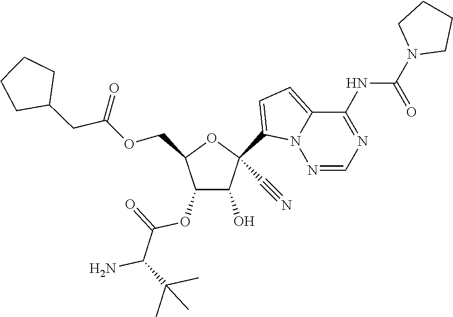 [SMILES: CC(C)(C)[C@H](N)C(=O)O[C@H]1[C@@H](O)[C@](C#N)(c2ccc3c(NC(=O)N4CCCC4)ncnn23)O[C@@H]1COC(=O)CC1CCCC1] | (2R,3S,4R,5R)-5-cyano-2-((2- cyclopentylacetoxy)methyl)-4-hydroxy- 5-(4-(pyrrolidine-1- carboxamido)pyrrolo[2,1- f][1,2,4]triazin-7-yl)tetrahydrofuran-3- yl (S)-2-amino-3,3-dimethylbutanoate | |
| 61 | 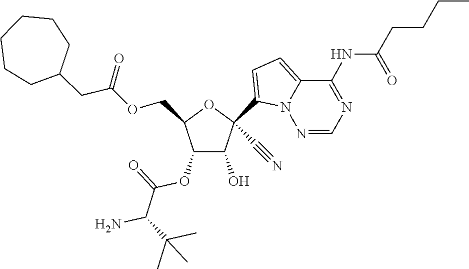 [SMILES: CCCCC(=O)Nc1ncnn2c([C@]3(C#N)O[C@H](COC(=O)CC4CCCCCC4)[C@@H](OC(=O)[C@@H](N)C(C)(C)C)[C@H]3O)ccc12] | (2R,3S,4R,5R)-5-cyano-2-((2- cycloheptylacetoxy)methyl)-4-hydroxy- 5-(4-pentanamidopyrrolo[2,1- f][1,2,4]triazin-7-yl)tetrahydrofuran-3- yl (S)-2-amino-3,3-dimethylbutanoate | 627.15 |
| 62 | 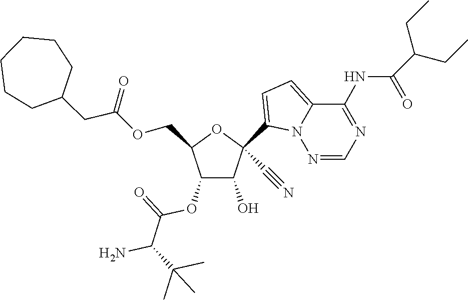 [SMILES: CCC(CC)C(=O)Nc1ncnn2c([C@]3(C#N)O[C@H](COC(=O)CC4CCCCCC4)[C@@H](OC(=O)[C@@H](N)C(C)(C)C)[C@H]3O)ccc12] | (2R,3S,4R,5R)-5-cyano-2-((2- cycloheptylacetoxy)methyl)-5-(4-(2- ethylbutanamido)pyrrolo[2,1- f][1,2,4]triazin-7-yl)-4- hydroxytetrahydrofuran-3-yl (S)-2- amino-3,3-dimethylbutanoate | 641.25 |
| 63 | 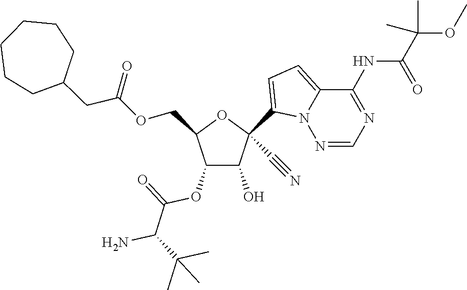 [SMILES: COC(C)(C)C(=O)Nc1ncnn2c([C@]3(C#N)O[C@H](COC(=O)CC4CCCCCC4)[C@@H](OC(=O)[C@@H](N)C(C)(C)C)[C@H]3O)ccc12] | (2R,3S,4R,5R)-5-cyano-2-((2- cycloheptylacetoxy)methyl)-4-hydroxy- 5-(4-(2-methoxy-2- methylpropanamido)pyrrolo[2,1- f][1,2,4]triazin-7-yl)tetrahydrofuran-3- yl (S)-2-amino-3,3-dimethylbutanoate | 643.25 |
| 64 | 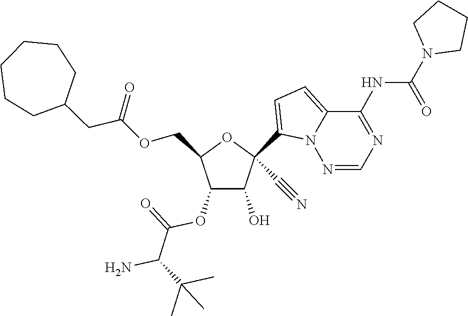 [SMILES: CC(C)(C)[C@H](N)C(=O)O[C@H]1[C@@H](O)[C@](C#N)(c2ccc3c(NC(=O)N4CCCC4)ncnn23)O[C@@H]1COC(=O)CC1CCCCCC1] | (2R,3S,4R,5R)-5-cyano-2-((2- cycloheptylacetoxy)methyl)-4-hydroxy- 5-(4-(pyrrolidine-1- carboxamido)pyrrolo[2,1- f][1,2,4]triazin-7-yl)tetrahydrofuran-3- yl (S)-2-amino-3,3-dimethylbutanoate | |
| 65 | 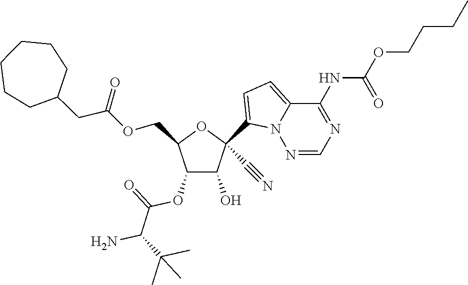 [SMILES: CCCCOC(=O)Nc1ncnn2c([C@]3(C#N)O[C@H](COC(=O)CC4CCCCCC4)[C@@H](OC(=O)[C@@H](N)C(C)(C)C)[C@H]3O)ccc12] | (2R,3S,4R,5R)-5-(4- ((butoxycarbonyl)amino)pyrrolo[2,1- f][1,2,4]triazin-7-yl)-5-cyano-2-((2- cycloheptylacetoxy)methyl)-4- hydroxytetrahydrofuran-3-yl (S)-2- amino-3,3-dimethylbutanoate | 643.20 |
| 66 | 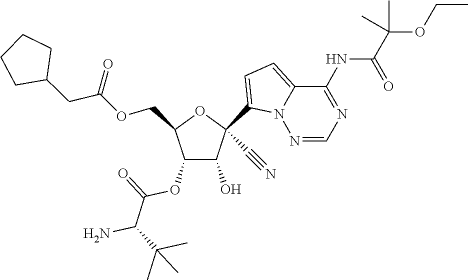 [SMILES: CCOC(C)(C)C(=O)Nc1ncnn2c([C@]3(C#N)O[C@H](COC(=O)CC4CCCC4)[C@@H](OC(=O)[C@@H](N)C(C)(C)C)[C@H]3O)ccc12] | (2R,3S,4R,5R)-5-cyano-2-((2- cyclopentylacetoxy)methyl)-5-(4-(2- ethoxy-2- methylpropanamido)pyrrolo[2,1- f][1,2,4]triazin-7-yl)-4- hydroxytetrahydrofuran-3-yl (S)-2- amino-3,3-dimethylbutanoate | 629.10 |
| 67 | 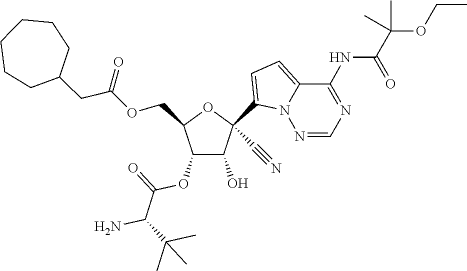 [SMILES: CCOC(C)(C)C(=O)Nc1ncnn2c([C@]3(C#N)O[C@H](COC(=O)CC4CCCCCC4)[C@@H](OC(=O)[C@@H](N)C(C)(C)C)[C@H]3O)ccc12] | (2R,3S,4R,5R)-5-cyano-2-((2- cycloheptylacetoxy)methyl)-5-(4-(2- ethoxy-2- methylpropanamido)pyrrolo[2,1- f][1,2,4]triazin-7-yl)-4- hydroxytetrahydrofuran-3-yl (S)-2- amino-3,3-dimethylbutanoate | 657.20 |
| 68 | 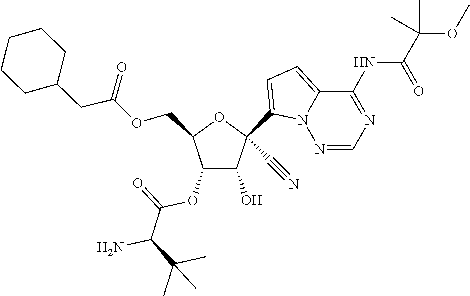 [SMILES: COC(C)(C)C(=O)Nc1ncnn2c([C@]3(C#N)O[C@H](COC(=O)CC4CCCCC4)[C@@H](OC(=O)[C@H](N)C(C)(C)C)[C@H]3O)ccc12] | (2R,3S,4R,5R)-5-cyano-2-((2- cyclohexylacetoxy)methyl)-4-hydroxy- 5-(4-(2-methoxy-2- methylpropanamido)pyrrolo[2,1- f][1,2,4]triazin-7-yl)tetrahydrofuran-3- yl (R)-2-amino-3,3-dimethylbutanoate | 629.15 |
| 69 | 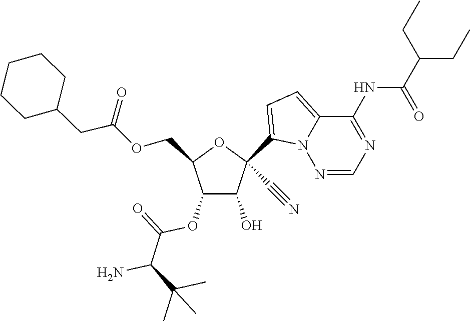 [SMILES: CCC(CC)C(=O)Nc1ncnn2c([C@]3(C#N)O[C@H](COC(=O)CC4CCCCC4)[C@@H](OC(=O)[C@H](N)C(C)(C)C)[C@H]3O)ccc12] | (2R,3S,4R,5R)-5-cyano-2-((2- cyclohexylacetoxy)methyl)-5-(4-(2- ethylbutanamido)pyrrolo[2,1- f][1,2,4]triazin-7-yl)-4- hydroxytetrahydrofuran-3-yl (R)-2- amino-3,3-dimethylbutanoate | 627.15 |
| 70 | 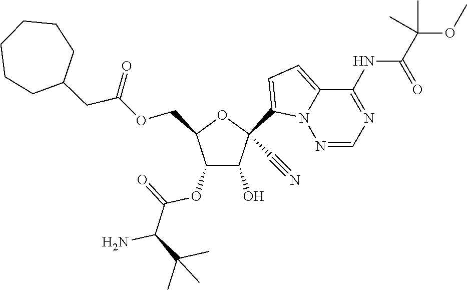 [SMILES: COC(C)(C)C(=O)Nc1ncnn2c([C@]3(C#N)O[C@H](COC(=O)CC4CCCCCC4)[C@@H](OC(=O)[C@H](N)C(C)(C)C)[C@H]3O)ccc12] | (2R,3S,4R,5R)-5-cyano-2-((2- cycloheptylacetoxy)methyl)-4-hydroxy- 5-(4-(2-methoxy-2- methylpropanamido)pyrrolo[2,1- f][1,2,4]triazin-7-yl)tetrahydrofuran-3- yl (R)-2-amino-3,3-dimethylbutanoate | 643.15 |
| 71 | 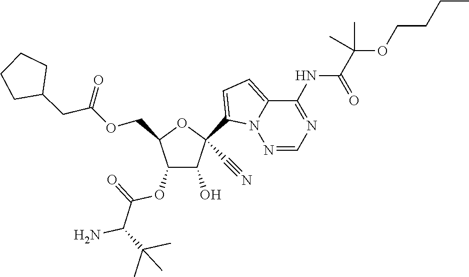 [SMILES: CCCCOC(C)(C)C(=O)Nc1ncnn2c([C@]3(C#N)O[C@H](COC(=O)CC4CCCC4)[C@@H](OC(=O)[C@@H](N)C(C)(C)C)[C@H]3O)ccc12] | (2R,3S,4R,5R)-5-(4-(2-butoxy-2- methylpropanamido)pyrrolo[2,1- f][1,2,4]triazin-7-yl)-5-cyano-2-((2- cyclopentylacetoxy)methyl)-4- hydroxytetrahydrofuran-3-yl (S)-2- amino-3,3-dimethylbutanoate | 657.20 |
| 72 | 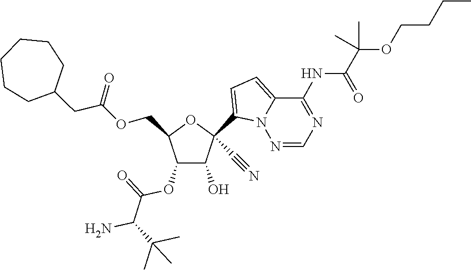 [SMILES: CCCCOC(C)(C)C(=O)Nc1ncnn2c([C@]3(C#N)O[C@H](COC(=O)CC4CCCCCC4)[C@@H](OC(=O)[C@@H](N)C(C)(C)C)[C@H]3O)ccc12] | (2R,3S,4R,5R)-5-(4-(2-butoxy-2- methylpropanamido)pyrrolo[2,1- f][1,2,4]triazin-7-yl)-5-cyano-2-((2- cycloheptylacetoxy)methyl)-4- hydroxytetrahydrofuran-3-yl (S)-2- amino-3,3-dimethylbutanoate | 685.30 |
| 73 | 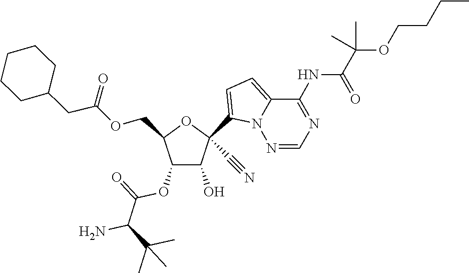 [SMILES: CCCCOC(C)(C)C(=O)Nc1ncnn2c([C@]3(C#N)O[C@H](COC(=O)CC4CCCCC4)[C@@H](OC(=O)[C@H](N)C(C)(C)C)[C@H]3O)ccc12] | (2R,3S,4R,5R)-5-(4-(2-butoxy-2- methylpropanamido)pyrrolo[2,1- f][1,2,4]triazin-7-yl)-5-cyano-2-((2- cyclohexylacetoxy)methyl)-4- hydroxytetrahydrofuran-3-yl (R)-2- amino-3,3-dimethylbutanoate | 671.20 |
| 74 | 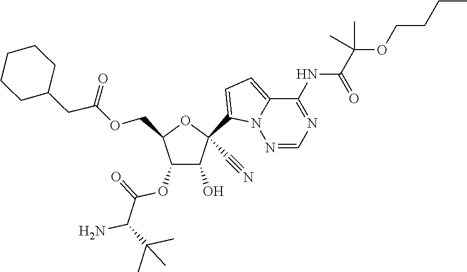 [SMILES: CCCCOC(C)(C)C(=O)Nc1ncnn2c([C@]3(C#N)O[C@H](COC(=O)CC4CCCCC4)[C@@H](OC(=O)[C@@H](N)C(C)(C)C)[C@H]3O)ccc12] | (2R,3S,4R,5R)-5-(4-(2-butoxy-2- methylpropanamido)pyrrolo[2,1- f][1,2,4]triazin-7-yl)-5-cyano-2-((2- cyclohexylacetoxy)methyl)-4- hydroxytetrahydrofuran-3-yl (S)-2- amino-3,3-dimethylbutanoate | 671.25 |
| 75 | 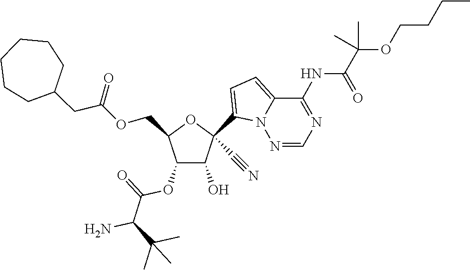 [SMILES: CCCCOC(C)(C)C(=O)Nc1ncnn2c([C@]3(C#N)O[C@H](COC(=O)CC4CCCCCC4)[C@@H](OC(=O)[C@H](N)C(C)(C)C)[C@H]3O)ccc12] | (2R,3S,4R,5R)-5-(4-(2-butoxy-2- methylpropanamido)pyrrolo[2,1- f][1,2,4]triazin-7-yl)-5-cyano-2-((2- cycloheptylacetoxy)methyl)-4- hydroxytetrahydrofuran-3-yl (R)-2- amino-3,3-dimethylbutanoate | 685.30 |
| 76 | 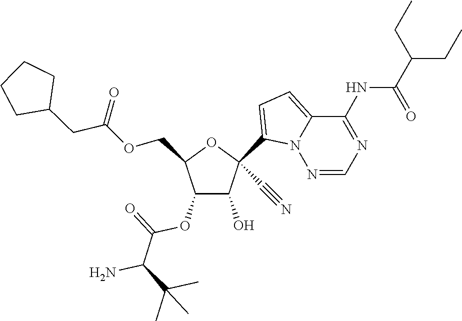 [SMILES: CCC(CC)C(=O)Nc1ncnn2c([C@]3(C#N)O[C@H](COC(=O)CC4CCCC4)[C@@H](OC(=O)[C@H](N)C(C)(C)C)[C@H]3O)ccc12] | (2R,3S,4R,5R)-5-cyano-2-((2- cyclopentylacetoxy)methyl)-5-(4-(2- ethylbutanamido)pyrrolo[2,1- f][1,2,4]triazin-7-yl)-4- hydroxytetrahydrofuran-3-yl (R)-2- amino-3,3-dimethylbutanoate | 613.10 |
| 77 | 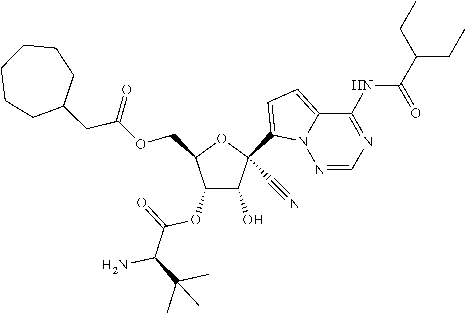 [SMILES: CCC(CC)C(=O)Nc1ncnn2c([C@]3(C#N)O[C@H](COC(=O)CC4CCCCCC4)[C@@H](OC(=O)[C@H](N)C(C)(C)C)[C@H]3O)ccc12] | (2R,3S,4R,5R)-5-cyano-2-((2- cycloheptylacetoxy)methyl)-5-(4-(2- ethylbutanamido)pyrrolo[2,1- f][1,2,4]triazin-7-yl)-4- hydroxytetrahydrofuran-3-yl (R)-2- amino-3,3-dimethylbutanoate | 641.15 |
| 78 | 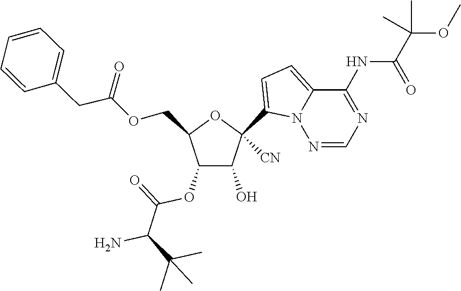 [SMILES: COC(C)(C)C(=O)Nc1ncnn2c([C@]3(C#N)O[C@H](COC(=O)Cc4ccccc4)[C@@H](OC(=O)[C@H](N)C(C)(C)C)[C@H]3O)ccc12] | (2R,3S,4R,5R)-5-cyano-4-hydroxy-5-(4-(2- methoxy-2- methylpropanamido)pyrrolo[2,1- f][1,2,4]triazin-7-yl)-2-((2- phenylacetoxy)methyl)tetrahydrofuran-3-yl (R)-2-amino-3,3-dimethylbutanoate | 623.05 |
| 79 | 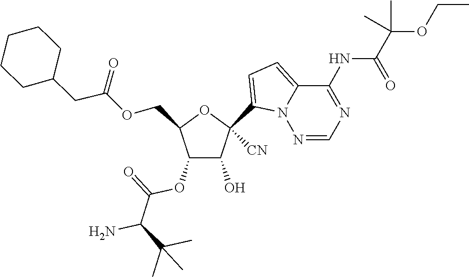 [SMILES: CCOC(C)(C)C(=O)Nc1ncnn2c([C@]3(C#N)O[C@H](COC(=O)CC4CCCCC4)[C@@H](OC(=O)[C@H](N)C(C)(C)C)[C@H]3O)ccc12] | (2R,3S,4R,5R)-5-cyano-2-((2- cyclohexylacetoxy)methyl)-5-(4-(2-ethoxy- 2-methylpropanamido)pyrrolo[2,1- f][1,2,4]triazin-7-yl)-4- hydroxytetrahydrofuran-3-yl (R)-2-amino- 3,3-dimethylbutanoate | 643.20 |
| 80 | 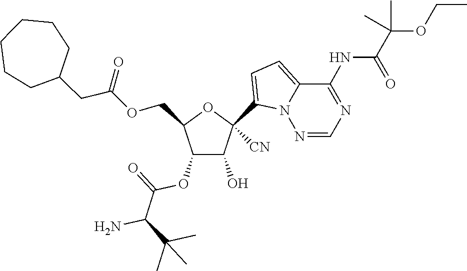 [SMILES: CCOC(C)(C)C(=O)Nc1ncnn2c([C@]3(C#N)O[C@H](COC(=O)CC4CCCCCC4)[C@@H](OC(=O)[C@H](N)C(C)(C)C)[C@H]3O)ccc12] | (2R,3S,4R,5R)-5-cyano-2-((2- cycloheptylacetoxy)methyl)-5-(4-(2-ethoxy- 2-methylpropanamido)pyrrolo[2,1- f][1,2,4]triazin-7-yl)-4- hydroxytetrahydrofuran-3-yl (R)-2-amino- 3,3-dimethylbutanoate | 657.15 |
| 81 | 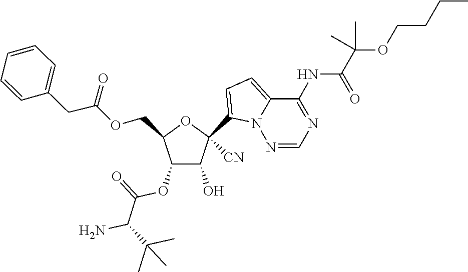 [SMILES: CCCCOC(C)(C)C(=O)Nc1ncnn2c([C@]3(C#N)O[C@H](COC(=O)Cc4ccccc4)[C@@H](OC(=O)[C@@H](N)C(C)(C)C)[C@H]3O)ccc12] | (2R,3S,4R,5R)-5-(4-(2-butoxy-2- methylpropanamido)pyrrolo[2,1- f][1,2,4]triazin-7-yl)-5-cyano-4-hydroxy-2- ((2-phenylacetoxy)methyl)tetrahydrofuran- 3-yl (S)-2-amino-3,3-dimethylbutanoate | 665.15 |
| 82 | 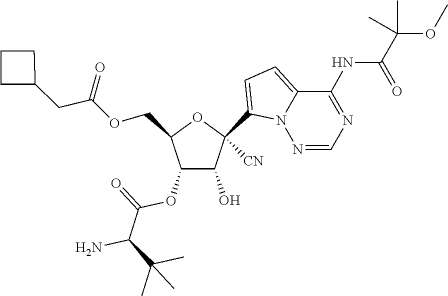 [SMILES: COC(C)(C)C(=O)Nc1ncnn2c([C@]3(C#N)O[C@H](COC(=O)CC4CCC4)[C@@H](OC(=O)[C@H](N)C(C)(C)C)[C@H]3O)ccc12] | (2R,3S,4R,5R)-5-cyano-2-((2- cyclobutylacetoxy)methyl)-4-hydroxy-5-(4- (2-methoxy-2- methylpropanamido)pyrrolo[2,1- f][1,2,4]triazin-7-yl)tetrahydrofuran-3-yl (R)-2-amino-3,3-dimethylbutanoate | |
| 83 | 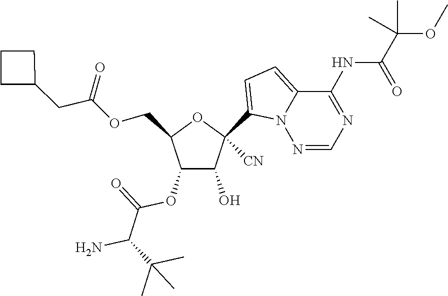 [SMILES: COC(C)(C)C(=O)Nc1ncnn2c([C@]3(C#N)O[C@H](COC(=O)CC4CCC4)[C@@H](OC(=O)[C@@H](N)C(C)(C)C)[C@H]3O)ccc12] | (2R,3S,4R,5R)-5-cyano-2-((2- cyclobutylacetoxy)methyl)-4-hydroxy-5-(4- (2-methoxy-2- methylpropanamido)pyrrolo[2,1- f][1,2,4]triazin-7-yl)tetrahydrofuran-3-yl (S)-2-amino-3,3-dimethylbutanoate | |
| 84 | 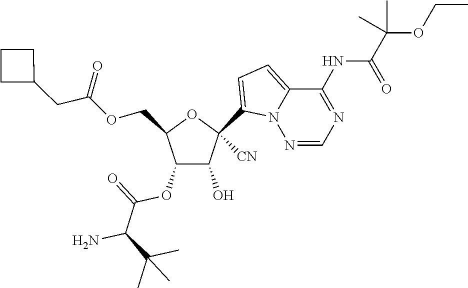 [SMILES: CCOC(C)(C)C(=O)Nc1ncnn2c([C@]3(C#N)O[C@H](COC(=O)CC4CCC4)[C@@H](OC(=O)[C@H](N)C(C)(C)C)[C@H]3O)ccc12] | (2R,3S,4R,5R)-5-cyano-2-((2- cyclobutylacetoxy)methyl)-5-(4-(2-ethoxy- 2-methylpropanamido)pyrrolo[2,1- f][1,2,4]triazin-7-yl)-4- hydroxytetrahydrofuran-3-yl(R)-2-amino- 3,3-dimethylbutanoate | |
| 85 | 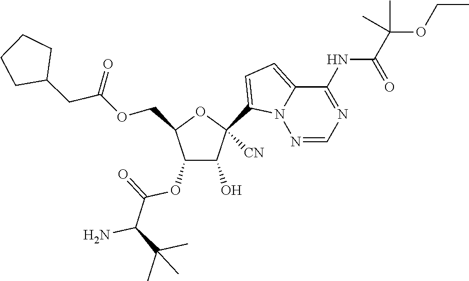 [SMILES: CCOC(C)(C)C(=O)Nc1ncnn2c([C@]3(C#N)O[C@H](COC(=O)CC4CCCC4)[C@@H](OC(=O)[C@H](N)C(C)(C)C)[C@H]3O)ccc12] | (2R,3S,4R,5R)-5-cyano-2-((2- cyclopentylacetoxy)methyl)-5-(4-(2- ethoxy-2-methylpropanamido)pyrrolo[2,1- f][1,2,4]triazin-7-yl)-4- hydroxytetrahydrofuran-3-yl(R)-2-amino- 3,3-dimethylbutanoate | |
| 86 | 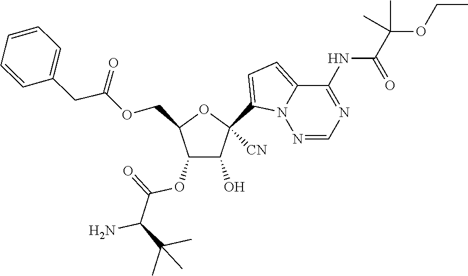 [SMILES: CCOC(C)(C)C(=O)Nc1ncnn2c([C@]3(C#N)O[C@H](COC(=O)Cc4ccccc4)[C@@H](OC(=O)[C@H](N)C(C)(C)C)[C@H]3O)ccc12] | (2R,3S,4R,5R)-5-cyano-5-(4-(2-ethoxy-2- methylpropanamido)pyrrolo[2,1- f][1,2,4]triazin-7-yl)-4-hydroxy-2-((2- phenylacetoxy)methyl)tetrahydrofuran-3-yl (R)-2-amino-3,3-dimethylbutanoate | |
| 87 | 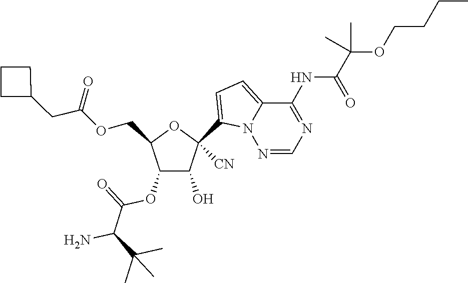 [SMILES: CCCCOC(C)(C)C(=O)Nc1ncnn2c([C@]3(C#N)O[C@H](COC(=O)CC4CCC4)[C@@H](OC(=O)[C@H](N)C(C)(C)C)[C@H]3O)ccc12] | (2R,3S,4R,5R)-5-(4-(2-butoxy-2- methylpropanamido)pyrrolo[2,1- f][1,2,4]triazin-7-yl)-5-cyano-2-((2- cyclobutylacetoxy)methyl)-4- hydroxytetrahydrofuran-3-yl(R)-2-amino- 3,3-dimethylbutanoate | |
| 88 | 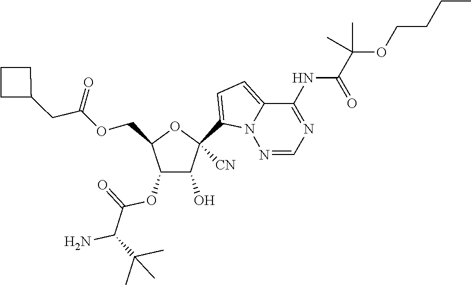 [SMILES: CCCCOC(C)(C)C(=O)Nc1ncnn2c([C@]3(C#N)O[C@H](COC(=O)CC4CCC4)[C@@H](OC(=O)[C@@H](N)C(C)(C)C)[C@H]3O)ccc12] | (2R,3S,4R,5R)-5-(4-(2-butoxy-2- methylpropanamido)pyrrolo[2,1- f][1,2,4]triazin-7-yl)-5-cyano-2-((2- cyclobutylacetoxy)methyl)-4- hydroxytetrahydrofuran-3-yl (S)-2-amino- 3,3-dimethylbutanoate | |
| 89 | 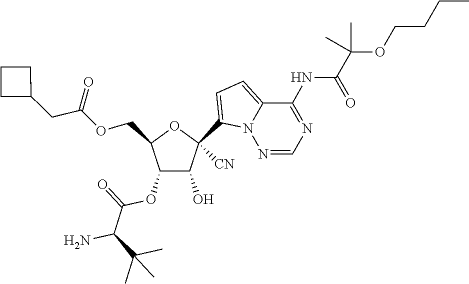 [SMILES: CCCCOC(C)(C)C(=O)Nc1ncnn2c([C@]3(C#N)O[C@H](COC(=O)CC4CCC4)[C@@H](OC(=O)[C@H](N)C(C)(C)C)[C@H]3O)ccc12] | (2R,3S,4R,5R)-5-(4-(2-butoxy-2- methylpropanamido)pyrrolo[2,1- f][1,2,4]triazin-7-yl)-5-cyano-2-((2- cyclobutylacetoxy)methyl)-4- hydroxytetrahydrofuran-3-yl (R)-2-amino- 3,3-dimethylbutanoate | |
| 90 | 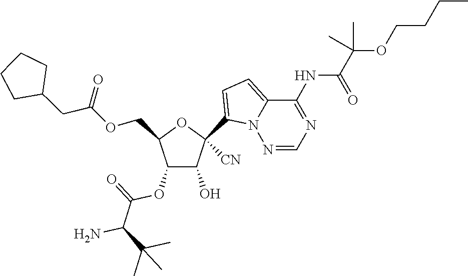 [SMILES: CCCCOC(C)(C)C(=O)Nc1ncnn2c([C@]3(C#N)O[C@H](COC(=O)CC4CCCC4)[C@@H](OC(=O)[C@H](N)C(C)(C)C)[C@H]3O)ccc12] | (2R,3S,4R,5R)-5-(4-(2-butoxy-2- methylpropanamido)pyrrolo[2,1- f][1,2,4]triazin-7-yl)-5-cyano-2-((2- cyclopentylacetoxy)methyl)-4- hydroxytetrahydrofuran-3-yl(R)-2-amino- 3,3-dimethylbutanoate | |
| 91 | 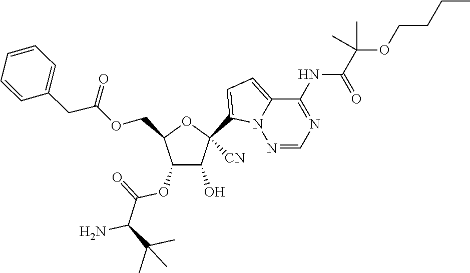 [SMILES: CCCCOC(C)(C)C(=O)Nc1ncnn2c([C@]3(C#N)O[C@H](COC(=O)Cc4ccccc4)[C@@H](OC(=O)[C@H](N)C(C)(C)C)[C@H]3O)ccc12] | (2R,3S,4R,5R)-5-(4-(2-butoxy-2- methylpropanamido)pyrrolo[2,1- f][1,2,4]triazin-7-yl)-5-cyano-4-hydroxy-2- ((2-phenylacetoxy)methyl)tetrahydrofuran- 3-yl(R)-2-amino-3,3-dimethylbutanoate | |
| 92 | 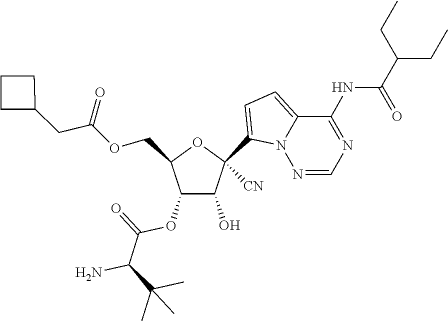 [SMILES: CCC(CC)C(=O)Nc1ncnn2c([C@]3(C#N)O[C@H](COC(=O)CC4CCC4)[C@@H](OC(=O)[C@H](N)C(C)(C)C)[C@H]3O)ccc12] | (2R,3S,4R,5R)-5-cyano-2-((2- cyclobutylacetoxy)methyl)-5-(4-(2- ethylbutanamido)pyrrolo[2,1- f][1,2,4]triazin-7-yl)-4- hydroxytetrahydrofuran-3-yl(R)-2-amino- 3,3-dimethylbutanoate | |
| 93 | 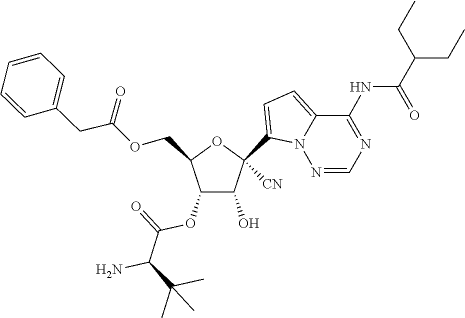 [SMILES: CCC(CC)C(=O)Nc1ncnn2c([C@]3(C#N)O[C@H](COC(=O)Cc4ccccc4)[C@@H](OC(=O)[C@H](N)C(C)(C)C)[C@H]3O)ccc12] | (2R,3S,4R,5R)-5-cyano-5-(4-(2- ethylbutanamido)pyrrolo[2,1- f][1,2,4]triazin-7-yl)-4-hydroxy-2-((2- phenylacetoxy)methyl)tetrahydrofuran-3-yl (R)-2-amino-3,3-dimethylbutanoate | |
| 94 | 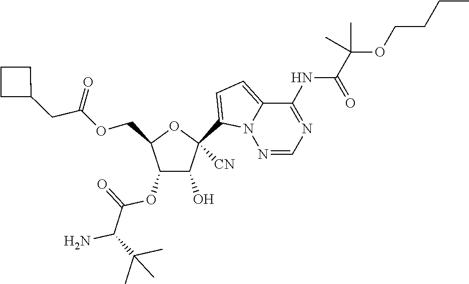 [SMILES: CCCCOC(C)(C)C(=O)Nc1ncnn2c([C@]3(C#N)O[C@H](COC(=O)CC4CCC4)[C@@H](OC(=O)[C@@H](N)C(C)(C)C)[C@H]3O)ccc12] | (2R,3S,4R,5R)-5-cyano-2-((2- cyclobutylacetoxy)methyl)-4-hydroxy-5-(4- ((R)-2-methylbutanamido)pyrrolo[2,1- f][1,2,4]triazin-7-yl)tetrahydrofuran-3-yl (R)-2-amino-3,3-dimethylbutanoate | |
| 95 | 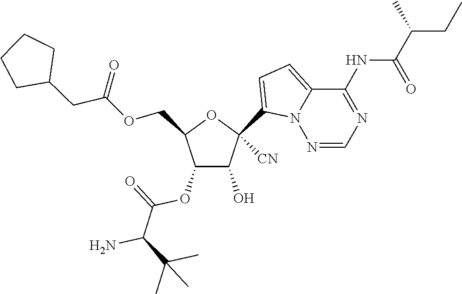 [SMILES: CC[C@@H](C)C(=O)Nc1ncnn2c([C@]3(C#N)O[C@H](COC(=O)CC4CCCC4)[C@@H](OC(=O)[C@H](N)C(C)(C)C)[C@H]3O)ccc12] | (2R,3S,4R,5R)-5-cyano-2-((2- cyclopentylacetoxy)methyl)-4-hydroxy-5- (4-((R)-2-methylbutanamido)pyrrolo[2,1- f][1,2,4]triazin-7-yl)tetrahydrofuran-3-yl (R)-2-amino-3,3-dimethylbutanoate | |
| 96 | 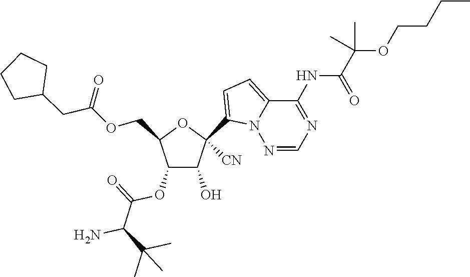 [SMILES: CCCCOC(C)(C)C(=O)Nc1ncnn2c([C@]3(C#N)O[C@H](COC(=O)CC4CCCC4)[C@@H](OC(=O)[C@H](N)C(C)(C)C)[C@H]3O)ccc12] | (2R,3S,4R,5R)-5-cyano-4-hydroxy-5-(4- ((R)-2-methylbutanamido)pyrrolo[2,1- f][1,2,4]triazin-7-yl)-2-((2- phenylacetoxy)methyl)tetrahydrofuran-3-yl (R)-2-amino-3,3-dimethylbutanoate | |
| 97 | 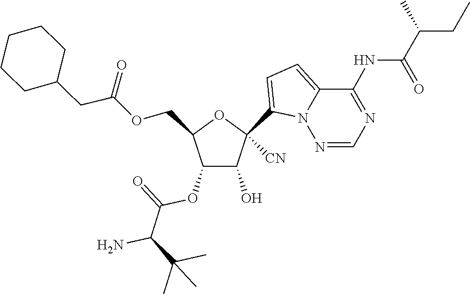 [SMILES: CC[C@@H](C)C(=O)Nc1ncnn2c([C@]3(C#N)O[C@H](COC(=O)CC4CCCCC4)[C@@H](OC(=O)[C@H](N)C(C)(C)C)[C@H]3O)ccc12] | (2R,3S,4R,5R)-5-cyano-2-((2- cyclohexylacetoxy)methyl)-4-hydroxy-5- (4-((R)-2-methylbutanamido)pyrrolo[2,1- f][1,2,4]triazin-7-yl)tetrahydrofuran-3-yl (R)-2-amino-3,3-dimethylbutanoate | |
| 98 | 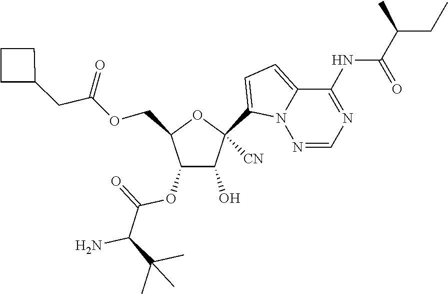 [SMILES: CC[C@H](C)C(=O)Nc1ncnn2c([C@]3(C#N)O[C@H](COC(=O)CC4CCC4)[C@@H](OC(=O)[C@H](N)C(C)(C)C)[C@H]3O)ccc12] | (2R,3S,4R,5R)-5-cyano-2-((2- cyclobutylacetoxy)methyl)-4-hydroxy-5-(4- ((S)-2-methylbutanamido)pyrrolo[2,1- f][1,2,4]triazin-7-yl)tetrahydrofuran-3-yl (R)-2-amino-3,3-dimethylbutanoate | |
| 99 | 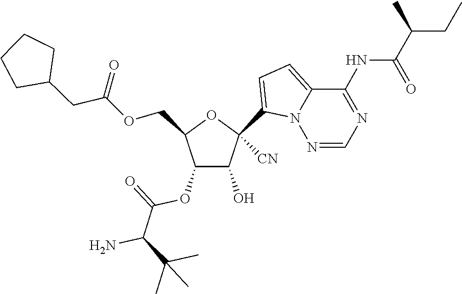 [SMILES: CC[C@H](C)C(=O)Nc1ncnn2c([C@]3(C#N)O[C@H](COC(=O)CC4CCCC4)[C@@H](OC(=O)[C@H](N)C(C)(C)C)[C@H]3O)ccc12] | (2R,3S,4R,5R)-5-cyano-2-((2- cyclopentylacetoxy)methyl)-4-hydroxy-5- (4-((S)-2-methylbutanamido)pyrrolo[2,1- f][1,2,4]triazin-7-yl)tetrahydrofuran-3-yl (R)-2-amino-3,3-dimethylbutanoate | |
| 100 | 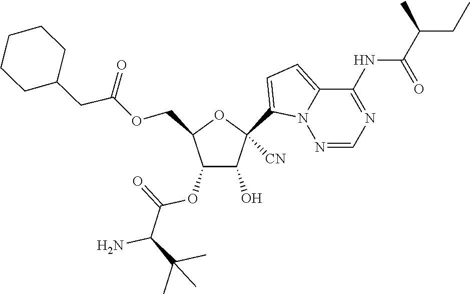 [SMILES: CC[C@H](C)C(=O)Nc1ncnn2c([C@]3(C#N)O[C@H](COC(=O)CC4CCCCC4)[C@@H](OC(=O)[C@H](N)C(C)(C)C)[C@H]3O)ccc12] | (2R,3S,4R,5R)-5-cyano-2-((2- cyclohexylacetoxy)methyl)-4-hydroxy-5- (4-((S)-2-methylbutanamido)pyrrolo[2,1- f][1,2,4]triazin-7-yl)tetrahydrofuran-3-yl (R)-2-amino-3,3-dimethylbutanoate | |
| 101 | 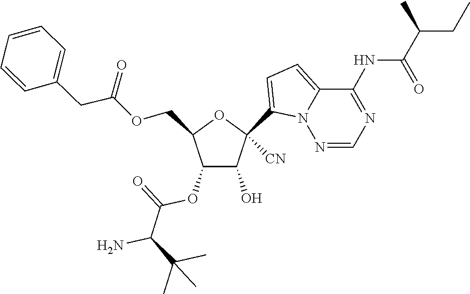 [SMILES: CC[C@H](C)C(=O)Nc1ncnn2c([C@]3(C#N)O[C@H](COC(=O)Cc4ccccc4)[C@@H](OC(=O)[C@H](N)C(C)(C)C)[C@H]3O)ccc12] | (2R,3S,4R,5R)-5-cyano-4-hydroxy-5-(4- ((S)-2-methylbutanamido)pyrrolo[2,1- f][1,2,4]triazin-7-yl)-2-((2- phenylacetoxy)methyl)tetrahydrofuran-3-yl (R)-2-amino-3,3-dimethylbutanoate | |
| 102 | 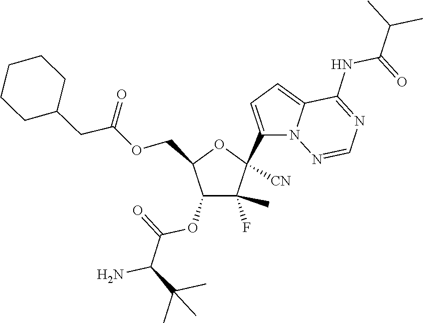 [SMILES: CC(C)C(=O)Nc1ncnn2c([C@]3(C#N)O[C@H](COC(=O)CC4CCCCC4)[C@@H](OC(=O)[C@H](N)C(C)(C)C)[C@@]3(C)F)ccc12] | (2R,3R,4R,5R)-5-cyano-2-((2- cyclohexylacetoxy)methyl)-4-fluoro-5-(4- isobutyramidopyrrolo[2,1-f][1,2,4]triazin-7- yl)-4-methyltetrahydrofuran-3-yl(R)-2- amino-3,3-dimethylbutanoate | |
| 103 | 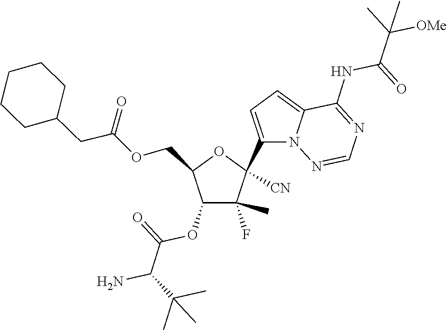 [SMILES: COC(C)(C)C(=O)Nc1ncnn2c([C@]3(C#N)O[C@H](COC(=O)CC4CCCCC4)[C@@H](OC(=O)[C@@H](N)C(C)(C)C)[C@@]3(C)F)ccc12] | (2R,3R,4R,5R)-5-cyano-2-((2- cyclohexylacetoxy)methyl)-4-fluoro-5-(4- (2-methoxy-2- methylpropanamido)pyrrolo[2,1- f][1,2,4]triazin-7-yl)-4- methyltetrahydrofuran-3-yl(S)-2-amino- 3,3-dimethylbutanoate | |
| 104 | 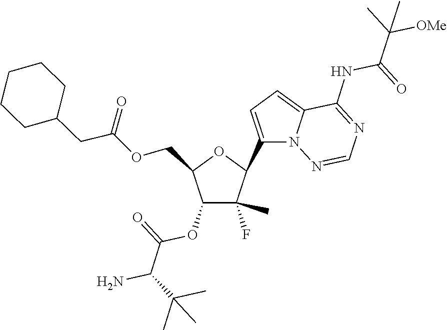 [SMILES: COC(C)(C)C(=O)Nc1ncnn2c([C@@H]3O[C@H](COC(=O)CC4CCCCC4)[C@@H](OC(=O)[C@@H](N)C(C)(C)C)[C@@]3(C)F)ccc12] | (2R,3R,4S,5S)-2-((2- cyclohexylacetoxy)methyl)-4-fluoro-5-(4- (2-methoxy-2- methylpropanamido)pyrrolo[2,1- f][1,2,4]triazin-7-yl)-4- methyltetrahydrofuran-3-yl(S)-2-amino- 3,3-dimethylbutanoate | |
| 105 | 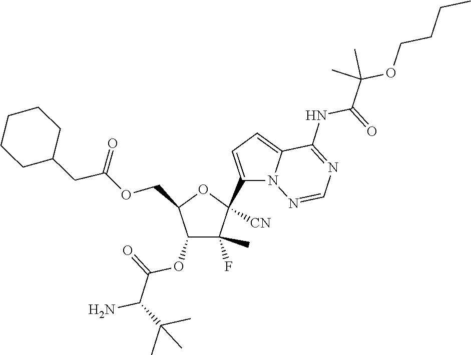 [SMILES: CCCCOC(C)(C)C(=O)Nc1ncnn2c([C@]3(C#N)O[C@H](COC(=O)CC4CCCCC4)[C@@H](OC(=O)[C@@H](N)C(C)(C)C)[C@@]3(C)F)ccc12] | (2R,3R,4R,5R)-5-(4-(2-butoxy-2- methylpropanamido)pyrrolo[2,1- f][1,2,4]triazin-7-yl)-5-cyano-2-((2- cyclohexylacetoxy)methyl)-4-fluoro-4- methyltetrahydrofuran-3-yl (S)-2-amino-3,3-dimethylbutanoate | |
| 106 | 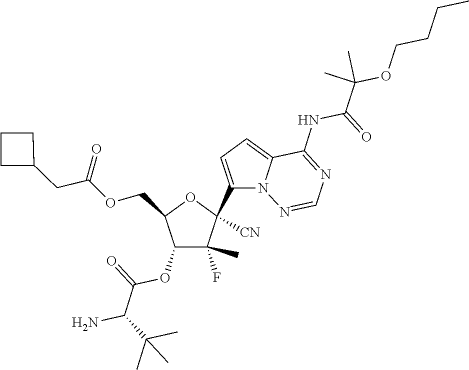 [SMILES: CCCCOC(C)(C)C(=O)Nc1ncnn2c([C@]3(C#N)O[C@H](COC(=O)CC4CCC4)[C@@H](OC(=O)[C@@H](N)C(C)(C)C)[C@@]3(C)F)ccc12] | (2R,3R,4R,5R)-5-(4-(2-butoxy-2- methylpropanamido)pyrrolo[2,1- f][1,2,4]triazin-7-yl)-5-cyano-2-((2- cyclobutylacetoxy)methyl)-4-fluoro-4- methyltetrahydrofuran-3-yl (S)-2-amino-3,3-dimethylbutanoate | |
| 107 | 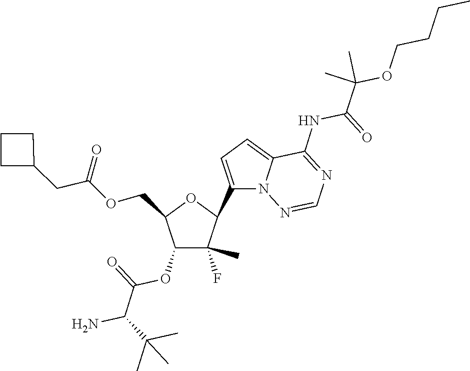 [SMILES: CCCCOC(C)(C)C(=O)Nc1ncnn2c([C@@H]3O[C@H](COC(=O)CC4CCC4)[C@@H](OC(=O)[C@@H](N)C(C)(C)C)[C@@]3(C)F)ccc12] | (2R,3R,4S,5S)-5-(4-(2-butoxy-2- methylpropanamido)pyrrolo[2,1- f][1,2,4]triazin-7-yl)-2-((2- cyclobutylacetoxy)methyl)-4-fluoro-4- methyltetrahydrofuran-3-yl(S)-2-amino- 3,3-dimethylbutanoate | |
| 108 | 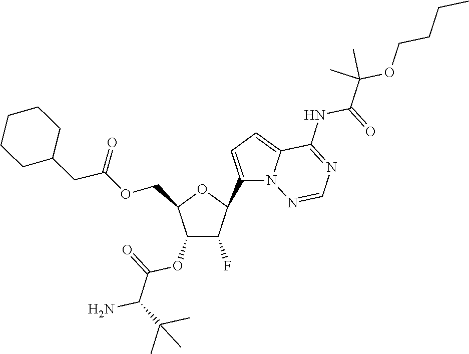 [SMILES: CCCCOC(C)(C)C(=O)Nc1ncnn2c([C@@H]3O[C@H](COC(=O)CC4CCCCC4)[C@@H](OC(=O)[C@@H](N)C(C)(C)C)[C@H]3F)ccc12] | (2R,3R,4S,5S)-5-(4-(2-butoxy-2- methylpropanamido)pyrrolo[2,1- f][1,2,4]triazin-7-yl)-2-((2- cyclohexylacetoxy)methyl)-4- fluorotetrahydrofuran-3-yl (S)-2-amino-3,3-dimethylbutanoate | |
| 109 | 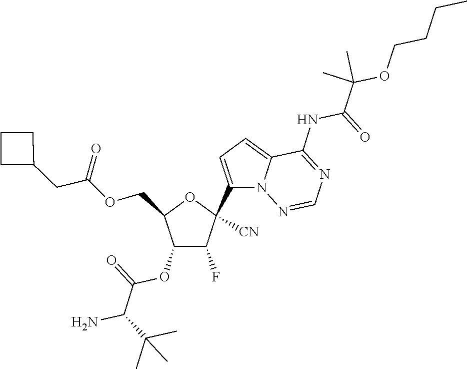 [SMILES: CCCCOC(C)(C)C(=O)Nc1ncnn2c([C@]3(C#N)O[C@H](COC(=O)CC4CCC4)[C@@H](OC(=O)[C@@H](N)C(C)(C)C)[C@H]3F)ccc12] | (2R,3R,4R,5R)-5-(4-(2-butoxy-2- methylpropanamido)pyrrolo[2,1- f][1,2,4]triazin-7-yl)-5-cyano-2-((2- cyclobutylacetoxy)methyl)-4- fluorotetrahydrofuran-3-yl (S)-2-amino-3,3-dimethylbutanoate | |
| 110 | 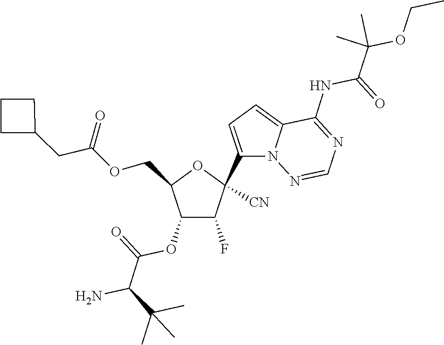 [SMILES: CCOC(C)(C)C(=O)Nc1ncnn2c([C@]3(C#N)O[C@H](COC(=O)CC4CCC4)[C@@H](OC(=O)[C@H](N)C(C)(C)C)[C@H]3F)ccc12] | (2R,3R,4R,5R)-5-cyano-2-((2- cyclobutylacetoxy)methyl)-5-(4-(2-ethoxy- 2-methylpropanamido)pyrrolo[2,1- f][1,2,4]triazin-7-yl)-4- fluorotetrahydrofuran-3-yl (R)-2-amino-3,3-dimethylbutanoate | |

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H. produces certain therapeutic advantages resulting from greater metabolic stability, for example increased in vivo half-life or reduced dosage requirements.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, solvate, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Prodrugs

“Prodrug” is meant to indicate a compound that is, in some embodiments, converted under physiological conditions or by solvolysis to a biologically active compound described herein. Thus, the term “prodrug” refers to a precursor of a biologically active compound that is pharmaceutically acceptable. A prodrug is typically inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). Prodrugs are delivered through any known methods described herein, including but not limited to orally, intravenously, intraperitoneal, or other method of administration known by those skilled in the art.

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987.

The term “prodrug” is also meant to include any covalently bonded carriers, which release the active compound in vivo when such prodrug is administered to a mammalian subject. Prodrugs of an active compound, as described herein, are prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. In some embodiments, prodrugs include any group bound to a heteroatom, such as the nitrogen of a pyridine which is cleaved in vivo to form the active compound or metabolite thereof. Examples of prodrugs include, but are not limited to, acetate, formate phosphate, and benzoate derivatives of alcohol or amine functional groups in the active compounds and the like.

In some embodiments, a prodrug is a salt. In some embodiments, a prodrug is a phosphate salt. In some embodiments, a prodrug is an alkyl phosphate salt. In some embodiments, a prodrug is an alkylated heteroaromatic salt. In some embodiments, a prodrug is a pyridinium salt. In some embodiments, a prodrug is a pyridinium alkylphosphate salt. In some embodiments, a prodrug is a pyridinium methylphosphate salt. In some embodiments, a prodrug comprises an alkyl phosphate bound to a heteroatom. In some embodiments, a prodrug comprises an alkyl phosphate bound to a heteroatom of a heterocycle.

In some embodiments, a prodrug is any compound that when administered to a biological system generates a biologically active compound as a result of spontaneous chemical reaction(s), enzyme catalyzed chemical reaction(s), and/or metabolic chemical reaction(s), or a combination of each. In other embodiments, prodrugs are formed using groups attached to functionality, e.g., HO—, HS—, HOOC—, NHR—, associated with the drug or active compounds, that cleave in vivo. In some embodiments, prodrugs include but are not limited to carboxylate esters where the group is alkyl, aryl, aralkyl, acyloxyalkyl, alkoxycarbonyloxyalkyl as well as esters of hydroxyl, thiol, and amines where the group attached is an acyl group, an alkoxycarbonyl, aminocarbonyl, phosphate or sulfate. The groups illustrated above are exemplary, not exhaustive, and other varieties of prodrugs are possible. Such prodrugs of disclosed compounds fall within this scope. In some embodiments, the compounds of the present application are prodrugs themselves and are converted into other forms, including the biologically active compound forms, when administered to a biological system.

In some embodiments, prodrugs undergo some form of a chemical transformation to produce the compound that is biologically active or is a precursor of the biologically active compound. In some cases, the prodrug is biologically active, more, or less than the intended active drug itself, and serves to improve drug efficacy or safety through improved oral bioavailability, and/or pharmacodynamic half-life, etc. Prodrug forms of compounds are utilized, for example, to improve bioavailability, improve subject acceptability such as by masking or reducing unpleasant characteristics such as bitter taste or gastrointestinal irritability, alter solubility such as for intravenous use, provide for prolonged or sustained release or delivery, improve ease of formulation, or provide site-specific delivery of the compound. Prodrugs are described in The Organic Chemistry of Drug Design and Drug Action, by Richard B. Silverman, Academic Press, San Diego, 1992, Chapter 8: “Prodrugs and Drug delivery Systems” pp. 352-401; Design of Prodrugs, edited by H. Bundgaard, Elsevier Science, Amsterdam, 1985; Design of Biopharmaceutical Properties through Prodrugs and Analogs, Ed. by E. B. Roche, American Pharmaceutical Association, Washington, 1977; and Drug Delivery Systems, ed. by R. L. Juliano, Oxford Univ. Press, Oxford, 1980.

In some embodiments, prodrugs comprise phosphorus moieties including phosphates or derivatives thereof. One such class of prodrugs is the aryl amidate (McGuigan) type. One report disclosed pharmacokinetic evaluation in the cynomolgus monkey of an aryl amidate prodrug of abacavir. (C. McGuigan et al., “Application of phosphoramidate pronucleotide technology to abacavir leads to a significant enhancement of antiviral potency,” J. Med. Chem. 2005, 48, 3504-3515).

In some embodiments, the compounds of the present application comprise a prodrug moiety that is carbamate, thiocarbamate, or urea to mask an amino or hydroxy group on the active compound. In some embodiments, the prodrug moiety of carbamate, thiocarbamate, or urea is metabolized in vivo to afford the free amino or hydroxy moiety on the active compound.

### Method of Treatment

In one aspect, the disclosure provides a method of treating, preventing and/or reducing the severity or extent of viral infections by administering to a subject in need thereof a therapeutically effective amount of a compound disclosed herein, or a pharmaceutically acceptable salt, solvate, or stereoisomer thereof, or a pharmaceutical composition comprising the compound.

The compounds described herein find use in a variety of applications for human and animal health. In some embodiments, the compounds described herein are inhibitors of coronavirus.

In some embodiments, the efficacy of treatment is determined using quantification of viral load or other evidence of infection.

In some embodiments, the compounds described herein reduce viral load in an individual suffering from a coronavirus infection.

As used herein, the term “administering” or “administration” refers to any route of introducing or delivering the composition or formulation to perform the intended function or treatment. Administration can be carried out by any route suitable for the delivery of composition or formulation. Thus, delivery routes can include intravenous, intramuscular, intraperitoneal, or subcutaneous delivery. Administration includes self-administration and the administration by another.

As used herein, he terms “patient” “subject” “individual” and the like are used interchangeably, and refer to any animal, or cells thereof whether in vitro or in situ, amenable to the methods described herein. In certain non-limiting embodiments, the patient, subject, or individual is a human.

As used herein, the terms “prevent” and “prevention” refer to acting prior to overt disease or disorder onset, to prevent the disease or disorder from developing, or to minimize the extent of the disease or disorder, or to slow its course of development. As used herein, the term “cure” refers to heal, to make well, or to restore to good health or to allow a time without recurrence of disease so that the risk of recurrence is small.

The subjects receiving the therapy described herein (e.g. a therapeutically effective amount of a compound disclosed herein, or a pharmaceutically acceptable salt, solvate, or stereoisomer thereof, or a pharmaceutical composition comprising the compound) may experience as a result of the therapy a reduction of virus count or improvement of at least one symptom associated with the virus infection, including, for example, fever, coughing, fatigue, pain, etc.

In one aspect, the disclosure provides a method for treating, preventing and/or reducing the severity or extent of viral infection, including, for example, a single-stranded positive sense RNA virus, coronavirus, severe acute respiratory syndrome coronavirus (SARS-CoV), Zika, dengue, yellow fever, West Nile, Hendra, Newcastle, Venezuelan equine encephalitis, chikungunya, Semliki Forest, Sindbis, Avian influenza A, Porcine Reproductive and Respiratory Syndrome, and Human immunodeficiency virus type 1. In another aspect, the disclosure provides a method for treating, preventing and/or reducing the severity or extent of viral infection, including, for example, a DNA virus, equine herpesvirus type 1, pseudorabies virus, BK polyomavirus, and porcine circovirus 2. COVID-19 pandemic, SARS-CoV-2, is a single stranded positive sense RNA virus that is closely related to severe acute respiratory syndrome coronavirus (SARS-CoV).

Also disclosed herein is a method of treating an infection in a subject, comprising administering to the subject a compound disclosed herein, or a pharmaceutically acceptable salt, solvate, or stereoisomer thereof.

Also disclosed herein is a method of treating an infection in a subject, comprising administering to the subject a pharmaceutical composition disclosed herein. In some embodiments, the infection is a viral infection. In some embodiments, the infection is caused by the SARS-CoV or SARS-CoV-2 virus. In some embodiments, the infection is COVID, or COVID-19. In some embodiments, the infection is caused by the Middle East respiratory syndrome coronavirus (MERS-CoV)/

In some embodiments, the viral infection is caused by a virus selected from the group consisting of coronavirus disease 2019 (SARS-CoV-2), YellowFever, Eastern Equine Encephalitis virus, Human Immunodeficiency virus (HIV), “African Swine Fever Viruses,” Arbovirus, Adenoviridae, Arenaviridae, Arterivirus, Astroviridae, Baculoviridae, Bimaviridae, Bimaviridae, Bunyaviridae, Caliciviridae, Caulimoviridae, Circoviridae, Coronaviridae, Cystoviridae, Ebolavirus, Deltaviridae, Filviridae, Filoviridae, Flaviviridae, Iridoviridae, Mononegavirus, Myoviridae, Papiloma virus, Papovaviridae, Paramyxoviridae, Prions, Parvoviridae, Phycodnaviridae, Poxviridae, Potyviridae, Reoviridae, Retroviridae, Rhabdoviridae, Tectiviridae, Togaviridae, pox, papilloma, corona, influenza, sendai virus (SeV), sindbis virus (SINV), vaccinia viruses, West Nile, Hanta, viruses which cause the common cold, and any combination thereof.

Zaire ebolavirus, more commonly known as Ebola virus (EBOV), is one of six known species within the genus Ebolavirus. Four of the six known ebolaviruses, including EBOV, cause a severe and often fatal hemorrhagic fever in humans and other mammals, known as Ebola virus disease (EVD). In some embodiments the viral infection is caused by one ebolavirus. In some embodiments the viral infection is caused by the Ebola virus. In some embodiments the viral infection is caused by the Marburg virus.

### Compositions/Formulations

The compounds described herein are administered to a subject in need thereof, either alone or in combination with pharmaceutically acceptable carriers, excipients, or diluents, in a pharmaceutical composition, according to standard pharmaceutical practice. In one embodiment, the compounds of this invention may be administered to animals. The compounds can be administered orally or parenterally, including the intravenous, intramuscular, intraperitoneal, subcutaneous, rectal, and topical routes of administration.

In another aspect, provided herein are pharmaceutical compositions comprising a compound describe herein, or a pharmaceutically acceptable salt, solvate, or stereoisomer thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical compositions described herein are administered to a subject by appropriate administration routes, including, but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

Pharmaceutical compositions including compounds described herein, or a pharmaceutically acceptable salt, solvate, or stereoisomer thereof are manufactured in a conventional manner, such as, by way of example only, by means of conventional mixing, dissolving, granulating, dragee-making, levigating, emulsifying, encapsulating, entrapping, or compression processes.

Pharmaceutical compositions for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

Pharmaceutical compositions that are administered orally include push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added.

Pharmaceutical compositions for parental use are formulated as infusions or injections. In some embodiments, the pharmaceutical composition suitable for injection or infusion includes sterile aqueous solutions, or dispersions, or sterile powders comprising a compound described herein, or a pharmaceutically acceptable salt, solvate, or stereoisomer thereof. In some embodiments, the pharmaceutical composition comprises a liquid carrier. In some embodiments, the liquid carrier is a solvent or liquid dispersion medium comprising, for example, water, saline, ethanol, a polyol (for example, glycerol, propylene glycol, liquid polyethylene glycols, and the like), vegetable oils, nontoxic glyceryl esters, and any combinations thereof. In some embodiments, the pharmaceutical compositions further comprise a preservative to prevent growth of microorganisms.

As used herein, the term “composition” or “pharmaceutical composition” refers to a composition comprising a therapeutically effective compound and a pharmaceutically acceptable carrier and optionally, other materials, e.g., one or more inert components (for example, a detectable agent or label) or one or more active components. The pharmaceutical composition facilitates administration of the therapeutically effective compound to a subject.

As used herein, the term “carrier” refers to a diluent, adjuvant, excipient, or vehicle in which the pharmaceutical composition is administered. Pharmaceutically acceptable carriers may include one or more solvents, dispersion media, coatings, isotonic and absorption delaying agents, and the like that are physiologically compatible. Compositions can include components such as diluents, binders, stabilizers, buffers, salts, lipophilic solvents, preservatives, or mixtures thereof. Examples of pharmaceutically acceptable carriers include but are not limited to water, saline, phosphate buffered saline, aqueous dextrose solutions, glycerol solutions. Suitable pharmaceutical excipients include starch, glucose, lactose, sucrose, dextrose, gelatin, mannitol, cellulose malt, rice, flour, chalk, silica gel, sodium stearate, glycerol monostearate, talc, sodium chloride, milk powder, glycerol, propylene, glycol, water, ethanol, and the like.

Carriers may also encompass a buffer or pH adjusting agent such as a salt prepared from an organic acid or base optionally mixed with a nontoxic surfactant. Examples of buffers include but are not limited to organic acid salts such as salts of citric acid, ascorbic acid, gluconic acid, carbonic acid, tartaric acid, succinic acid, acetic acid, or phthalic acid, Tris, tromethamine hydrochloride, and phosphate buffers. Additional carriers may include polymeric excipients or additives such as polyvinylpyrrolidones, ficolls (a polymeric sugar), dextrates (e.g., cyclodextrins, such as 2-hydroxypropyl-.quadrature.-cyclodextrin), polyethylene glycols, flavoring agents such as cherry or wintergreen flavor, antimicrobial agents, sweeteners, antioxidants, antistatic agents. The compositions may include a pharmaceutical carrier or excipient and a compound disclosed herein, and, in addition, may include other medicinal agents, pharmaceutical agents, carriers, adjuvants, binding agents etc. A pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylalted hydroxytoluene, etc.

The compositions can take the form of solid, semi-solid, lyophilized powder, or liquid dosage forms, such as for example, solutions, suspensions, emulsion, aerosols, gels, implants, microneedles, tablets, pills, capsules, soft elastic or hard gelatin capsules, dermal patch, gummy bears, powders, suspensions, extended-release formulations, and the like, for example, in unit dosage forms suitable for simple administration of precise dosages. In one embodiment the composition takes the forms of tablets, capsules, liquid caps, sublingual dissolving tablets, sublingual spray, nasal spray, gummy bear and/or dermal patch.

In one aspect, the composition is a liquid-based formulation including but not limited to an emulsion, suspension, solution, elixirs, or syrup in which the disclosed compound is dissolved and/or suspended, or in the form of a liquid-containing capsule in which the disclosed compound is dissolved and/or suspended in the liquid portion of the capsule core. The composition may be a capsule filled with an effective therapeutic amount of the liquid pharmaceutical formulation.

### Combination

Disclosed herein are methods of treating coronavirus infection using a compound disclosed herein in combination with additional therapeutic agents useful for treating coronavirus infection.

In some embodiments, the compound disclosed herein in combination with additional therapeutic agents useful for treating a coronavirus infection are administered simultaneously. In some embodiments, the compound disclosed herein in combination with additional therapeutic agents useful for treating a coronavirus infection are administered sequentially.

For the treatment of Arenaviridae virus infections or coronavirus infection, preferably, the other active therapeutic agent is active against Arenaviridae virus infections, particularly Lassa virus, coronavirus infection and Junin virus infections. Non-limiting examples of these other active therapeutic agents are ribavirin, favipiravir (also known as T-705 or Avigan), T-705 monophosphate, T-705 diphosphate, T-705 triphosphate, ST-193, and mixtures thereof. The compounds and compositions of the present disclosure are also intended for use with general care provided to patients with Arenaviridae viral infections, including parenteral fluids (including dextrose saline and Ringer's lactate) and nutrition, antibiotic (including metronidazole and cephalosporin antibiotics, such as ceftriaxone and cefuroxime) and/or antifungal prophylaxis, fever and pain medication, antiemetic (such as metoclopramide) and/or antidiarrheal agents, vitamin and mineral supplements (including Vitamin K and zinc sulfate), anti-inflammatory agents (such as ibuprofen), pain medications, and medications for other common diseases in the patient population, such anti-malarial agents (including artemether and artesunate-lumefantrine combination therapy), typhoid (including quinolone antibiotics, such as ciprofloxacin, macrolide antibiotics, such as azithromycin, cephalosporin antibiotics, such as ceftriaxone, or aminopenicillins, such as ampicillin), or shigellosis.

It is also possible to combine any compound of the disclosure with one or more additional active therapeutic agents in a unitary dosage form for simultaneous or sequential administration to a patient. The combination therapy may be administered as a simultaneous or sequential regimen. When administered sequentially, the combination may be administered in two or more administrations.

Co-administration of a compound of the disclosure with one or more other active therapeutic agents generally refers to simultaneous or sequential administration of a compound of the disclosure and one or more other active therapeutic agents, such that therapeutically effective amounts of the compound of the disclosure and one or more other active therapeutic agents are both present in the body of the patient.

Co-administration includes administration of unit dosages of the compounds of the disclosure before or after administration of unit dosages of one or more other active therapeutic agents, for example, administration of the compounds of the disclosure within seconds, minutes, or hours of the administration of one or more other active therapeutic agents. For example, a unit dose of a compound of the disclosure can be administered first, followed within seconds or minutes by administration of a unit dose of one or more other active therapeutic agents. Alternatively, a unit dose of one or more other therapeutic agents can be administered first, followed by administration of a unit dose of a compound of the disclosure within seconds or minutes. In some cases, it may be desirable to administer a unit dose of a compound of the disclosure first, followed, after a period of hours (e.g., 1-12 hours), by administration of a unit dose of one or more other active therapeutic agents. In other cases, it may be desirable to administer a unit dose of one or more other active therapeutic agents first, followed, after a period of hours (e.g., 1-12 hours), by administration of a unit dose of a compound of the disclosure.

The combination therapy may provide “synergy” and “synergistic”, i.e. the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g. in separate tablets, pills, or capsules, or by different injections in separate syringes. In general, during alternation therapy, an effective dosage of each active ingredient is administered sequentially, i.e. serially, whereas in combination therapy, effective dosages of two or more active ingredients are administered together. A synergistic anti-viral effect denotes an antiviral effect which is greater than the predicted purely additive effects of the individual compounds of the combination.

Additional non-limiting examples of additional therapeutic agents useful for treating coronavirus infections include:

### TLR Agonists

In some embodiments, the compound described herein is used in combination with a TLR agonist (TLR7, 8 and/or 9). In some embodiments, the TLR agonist is RG7795, GS-9620, SM360320, or AZD 8848.

### RIG-I Agonists

In some embodiments, the compound described herein is used in combination with a RIG-I agonist. In some embodiments, the RIG-I agonist is inarigivir.

### Interferons

In some embodiments, the compound described herein is used in combination with an interferon. In some embodiments, the interferon is interferon alpha (IFN-α), interferon alpha-2a, recombinant interferon alpha-2a, peginterferon alpha-2a, interferon alpha-2b, recombinant interferon alpha-2b, interferon alpha-2b XL, peginterferon alpha-2b, glycosylated interferon alpha-2b, interferon alpha-2c, recombinant interferon alpha-2c, interferon beta, interferon beta-1a, peginterferon beta-1a, interferon delta, interferon lambda (IFN-1), peginterferon lambda-1, interferon omega, interferon tau, interferon gamma (IFN-g), interferon alfacon-1, interferon alpha-nl, interferon alpha-n3, albinterferon alpha-2b, BLX-883, DA-3021, PI 101 (also known as AOP2014), PEG-infergen, belerofon, INTEFEN-IFN, albumin/interferon alpha 2a fusion protein, rHSA-IFN alpha2a, rHSA-IFN alpha 2b, PEG-IFN-SA, interferon alpha biobetter; in particular, peginterferon alpha-2a, peginterferon alpha-2b, glycosylated interferon alpha-2b, peginterferon beta-1a, or peginterferon lambda-1.

### Antiviral Agents

In some embodiments, the compound described herein is used in combination with an antiviral agent. In some embodiments, the antiviral agent is abacavir, acyclovir, adefovir, brivudine, cidofovir, didanosine, edoxudine, emtricitabine, entecavir, famciclovir, favipiravir, ganciclovir, idoxuridine, lamivudine, molnupiravir, penciclovir, remdesivir, ribavirin, sofosbuvir, ST-193, stavudine, T-705 diphosphate,T-705 monophosphate, T-705 triphosphate, telbivudine, tenofovir alafenamide, tenofovir disoproxil, trifluridine, valaciclovir, valganciclovir, vidarabine, zalcitabine, zidovudine, or any combinations thereof. In some embodiments, the antiviral agent is molnupiravir.

### Monoclonal Antibodies

In some embodiments, the compound described herein is used in combination with a monoclonal antibody. In some embodiments, the monoclonal antibody is bamlanivimab, REGEN-COV (casirivimab and imdevimab, administered together), bamlanivimab and etesevimab (administered together), sotrovimab, or tocilizumab.

### Guanosine Nucleotide Analog

In some embodiments, the compound described herein is used in combination with a Guanosine Nucleotide Analog. In some embodiments, the Guanosine Nucleotide Analog is AT-527.

### Protease Inhibitors

In some embodiments, the compound described herein is used in combination with a protease inhibitor. In some embodiments, the protease inhibitor is nirmatrelvir, ritonavir, or a combination thereof.

### Preparation of Compounds

The size and scale of the synthetic methods will vary depending on the desired amount of end product. It is understood that while specific reactants and amounts are provided in the Examples, one of skill in the art knows other alternative and equally feasible sets of reactants that will also yield the same compounds. Thus, where general oxidizers, reducers, solvents of various nature (aprotic, apolar, polar, etc.) are utilized, equivalents will be known in the art and are herein contemplated for use in the present methods.

For instance, in all instances, where a drying agent is used, contemplated drying agents include all those reported in the literature and known to one of skill, such as, but not limited to, magnesium sulfate, sodium sulfate, calcium sulfate, calcium chloride, potassium chloride, potassium hydroxide, sulfuric acid, quicklime, phosphorous pentoxide, potassium carbonate, sodium, silica gel, aluminum oxide, calcium hydride, lithium aluminum hydride (LAH), potassium hydroxide, and the like. (See, Burfield et al., “Desiccant Efficiency in Solvent Drying. A Reappraisal by Application of aNovel Method for Solvent Water Assay,”. J Org. Chem., 42(18):3060-3065, 1977). The amount of drying agent to add in each work up may be optimized by one of skill in the art and is not particularly limited. Further, although general guidance is provided for work-up of the intermediates in each step, it is generally understood by one of skill that other optional solvents and reagents may be equally substituted during the work-up steps. However, in some exceptional instances, it was found the very specific work-up conditions are required to maintain an unstable intermediate. Those instances are indicated below in the steps in which they occur.

Many of the steps below indicate various work-ups following termination of the reaction. A work-up involves generally quenching of a reaction to terminate any remaining catalytic activity and starting reagents. This is generally followed by addition of an organic solvent and separation of the aqueous layer from the organic layer. The product is typically obtained from the organic layer and unused reactants and other spurious side products and unwanted chemicals are generally trapped in the aqueous layer and discarded. The work-up in standard organic synthetic procedures found throughout the literature is generally followed by drying the product by exposure to a drying agent to remove any excess water or aqueous byproducts remaining partially dissolved in the organic layer and concentration of the remaining organic layer. Concentration of product dissolved in solvent may be achieved by any known means, such as evaporation under pressure, evaporation under increased temperature and pressure, and the like. Such concentrating may be achieved by use of standard laboratory equipment such as rotary-evaporator distillation, and the like. This is optionally followed by one or more purification steps which may include, but is not limited to, flash column chromatography, filtration through various media and/or other preparative methods known in the art. (See, for instance, Addison Ault, “Techniques and Experiments for Organic Chemistry,” 6^{th}Ed., University Science Books, Sausalito, Calif., 1998, Ann B. McGuire, Ed., pp. 45-59). Though certain organic co-solvents and quenching agents may be indicated in the steps described below, other equivalent organic solvents and quenching agents known to one of skill may be employed equally as well and are fully contemplated herein. Further, most of the work-ups in most steps may be further altered according to preference and desired end use or end product. Drying and evaporation, routine steps at the organic synthetic chemist bench, need not be employed and may be considered in all steps to be optional. The number of extractions with organic solvent may be as many as one, two, three, four, five, or ten or more, depending on the desired result and scale of reaction. Except where specifically noted, the volume, amount of quenching agent, and volume of organic solvents used in the work-up may be varied depending on specific reaction conditions and optimized to yield the best results.

Additionally, where inert gas or noble gas is indicated, any inert gas commonly used in the art may be substituted for the indicated inert gas, such as argon, nitrogen, helium, neon, etc.

### EXAMPLES

Examples below are intended to illustrate the general procedures used for preparing the compounds of the present invention.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1. Synthesis of (2R,3S,4R,5R)-5-(4-butyramidopyrrolo[2,1-f][1,2,4]triazin-7-yl)-5-cyano-2-((2-cyclohexylacetoxy)methyl)-4-hydroxytetrahydrofuran-3-yl (S)-2-amino-3,3-dimethylbutanoate (1)

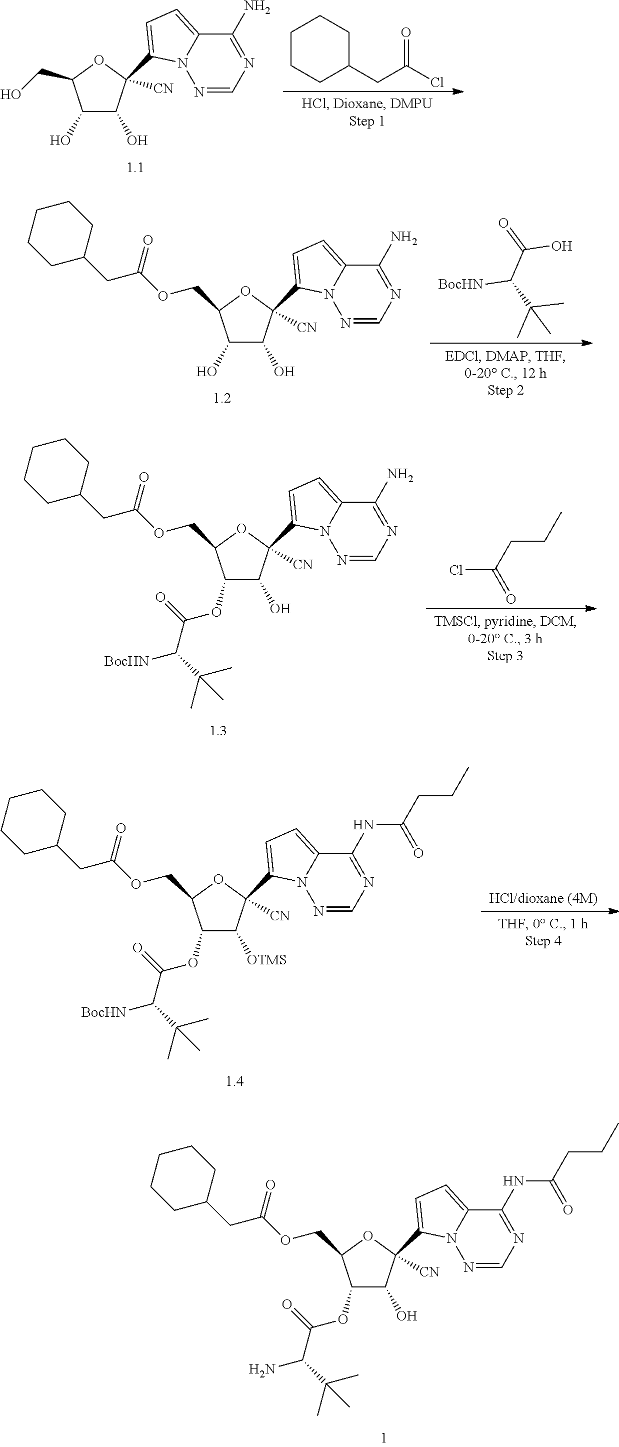

### Step 1. Synthesis of ((2R,3S,4R,5R)-5-(4-aminopyrrolo[2,1-f][1,2,4]triazin-7-yl)-5-cyano-3,4-dihydroxytetrahydrofuran-2-yl)methyl 2-cyclohexylacetate (1.2)

To a solution of compound 1.1 (10.0 g, 34.3 mmol) in DMPU (20 mL) was added HCl/1,4-dioxane (17.2 mL) at 0° C. The mixture solution was stirred for 10 min, and cyclohexylacetyl chloride (10.5 mL, 68.6 mmol) was added into the flask vessel dropwise. The reaction mixture was stirred at 0° C. for 2 hours. The mixture was adjusted to pH 8 with aqueous NaHCO₃. The mixture was diluted with water (150 mL) and extracted with EtOAc (200 mL×3). The organic phase was washed with brine water (50 mL×3), then dried over anhydrous sodium sulfate, filtered, and concentrated to remove the solvent. The crude product was purified by chromatography (SiO₂, Dichloromethane/Methanol=0% to 6%) to obtain compound 1.2 (11.5 g, 73% yield) as a white solid. MS (ESI): mass calcd. for C₂₀H₂₅N₅O₅, 415.19, m/z found 416.2 [M+H]⁺. ¹H NMR (400 MHz, DMSO-d₆) δ 7.92 (br s, 3H), 6.91 (d, J=4.4 Hz, 1H), 6.80 (d, J=4.4 Hz, 1H), 6.31 (d, J=6 Hz, 1H), 5.37 (d, J=6 Hz, 1H), 4.68 (d, J=5.2 Hz, 1H), 4.22 (d, J=6 Hz, 1H), 3.9-4.15 (m, 2H), 3.92 (m, 1H), 2.07 (m, 2H), 1.5 (br m, 6H), 1.0-1.2 (m, 3H), 0.86-1.1 (m, 2H).

### Step 2. Synthesis of (2R,3S,4R,5R)-5-(4-aminopyrrolo[2,1-f][1,2,4]triazin-7-yl)-5-cyano-2-((2-cyclohexylacetoxy)methyl)-4-hydroxytetrahydrofuran-3-yl (S)-2-((tert-butoxycarbonyl)amino)-3,3-dimethylbutanoate (1.3)

To a solution of compound 1.2 (580 mg, 1.40 mmol) in THF (25 mL) was added (2S)-2-{[(tert-butoxy)carbonyl]amino}-3,3-dimethylbutanoic acid (323 mg, 1.40 mmol), EDCI (803 mg, 4.19 mmol) and DMAP (512 mg, 4.19 mmol), the mixture was stirred at 25° C. for 16 h. After completion, the mixture was concentrated in vacuo to afford a residue. Then the reaction residue was diluted with water (50 mL) and extracted with EtOAc (50 mL×3). The organic layer was dried over sodium sulphate and concentrated under reduced pressure to afford a crude. The residue was purified by prep-HPLC [Gradient: 5-65% ACN in water (0.1% FA)] to obtain compound 1.3 (150 mg, 16% yield) as a white solid. MS (ESI): mass calcd. for C₃₁H₄₄N₆O₈, 628.32, found 629.25 [M+H]⁺. ¹H NMR (400 MHz, DMSO) δ 7.93 (br s, 3H), 7.05 (d, J=8.0 Hz, 1H), 6.94 (d, J=4.8 Hz, 1H), 6.86 (d, J=4.8 Hz, 1H), 6.70-6.63 (m, 1H), 5.13-5.07 (m, 2H), 4.43 (d, J=3.2 Hz, 1H), 4.34-4.25 (m, 1H), 4.17 (dd, J=12.4, 5.2 Hz, 1H), 3.94 (d, J=8.0 Hz, 1H), 2.18-2.09 (m, 2H), 1.65-1.53 (m, 6H), 1.39 (s, 9H), 1.20-1.15 (m, 3H), 0.98 (s, 9H), 0.91-0.82 (m, 2H).

### Step 3. Synthesis of (2R,3R,4R,5R)-5-(4-butyramidopyrrolo[2,1-f][1,2,4]triazin-7-yl)-5-cyano-2-((2-cyclohexylacetoxy)methyl)-4-((trimethylsilyl)oxy)tetrahydrofuran-3-yl (S)-2-((tert-butoxycarbonyl)amino)-3,3-dimethylbutanoate (1.4)

To a solution of compound 1.3 (150 mg, 0.239 mmol) and pyridine (0.23 mL, 2.86 mmol) in DCM (1.0 mL) was added TMSCI (77.8 mg, 0.72 mmol) at 0° C., the mixture was stirred at 20° C. for 1 h. To the reaction was added butanoyl chloride (50.9 mg, 0.48 mmol) at 0° C., the mixture was stirred at 0° C. for 1 h. Then the reaction was diluted with water (50 mL) and extracted with DCM (50 mL×3). The organic phase was washed with brine water (50 mL×3), then dried over anhydrous sodium sulfate, filtered, and concentrated to remove the solvent. The crude product was purified by chromatography (SiO₂, Petroleum ether/Ethyl acetate from 0% to 30%) to obtain compound 1.4 (120 mg, 62% yield) as a white solid. MS (ESI): m/z calcd. for C₃₈H₅₈N₆O₉Si, 770.40, found 771.35 [M+H]⁺. H NMR (400 MHz, DMSO) δ 10.95 (s, 1H), 8.39 (s, 1H), 7.35 (d, J=4.8 Hz, 1H), 7.13-7.03 (m, 2H), 5.26 (d, J=5.2 Hz, 1H), 5.14 (t, J=5.6 Hz, 1H), 4.58-4.51 (m, 1H), 4.35 (dd, J=12.4, 3.2 Hz, 1H), 4.26 (dd, J=12.4, 5.2 Hz, 1H), 4.02 (d, J=9.2 Hz, 1H), 2.70 (t J=7.2 Hz, 2H), 2.23-2.13 (m, 2H), 1.68-1.54 (m, 8H), 1.38 (s, 9H), 1.17-1.03 (m, 3H), 0.98-0.91 (m, 12H), 0.90-0.81 (m, 2H), 0.02 (s, 9H).

### Step 4. Synthesis of (2R,3S,4R,5R)-5-(4-butyramidopyrrolo[2,1-f][1,2,4]triazin-7-yl)-5-cyano-2-((2-cyclohexylacetoxy)methyl)-4-hydroxytetrahydrofuran-3-yl (S)-2-amino-3,3-dimethylbutanoate (1)

To a solution of compound 1.4 (120 mg, 0.156 mmol) in THF (1.0 mL) was added HCl in dioxane (1.0 mL, 4 M) at 0° C., then the reaction was stirred at 0° C. for 1 h. After completion, the mixture was concentrated in vacuo to afford a residue, the residue was diluted with ACN (2 mL) and purified by prep-HPLC (mobile phase: ACN-H₂O (0.1% FA), 5%-40%) to obtain compound 1 (28.8 mg, 29.6% yield) as a white solid. MS (ESI): mass calcd. for C₃₀H₄₂N₆O₇, 598.31, m/z found 599.25 [M+H]⁺. ^{t}H NMR (400 MHz, DMSO) δ 10.94 (s, 1H), 8.40 (s, 1H), 7.32 (d, J=4.8 Hz, 1H), 7.11 (d, J=4.8 Hz, 1H), 6.78-6.68 (m, 1H), 5.22-4.92 (m, 2H), 4.54-4.49 (m, 1H), 4.35-4.18 (m, 2H), 3.09 (s, 1H), 2.70 (t J=7.2 Hz, 2H), 2.18-2.06 (m, 2H), 1.69-1.51 (m, 8H), 1.20-1.04 (m, 3H), 0.98-0.89 (m, 12H), 0.88-0.76 (m, 2H).

### Example 2. Synthesis of (2R,3S,4R,5R)-5-cyano-2-((2-cyclohexylacetoxy)methyl)-5-(4-(2-ethylbutanamido)pyrrolo[2,1-f][1,2,4]triazin-7-yl)-4-hydroxytetrahydrofuran-3-yl (S)-2-amino-3,3-dimethylbutanoate (2)

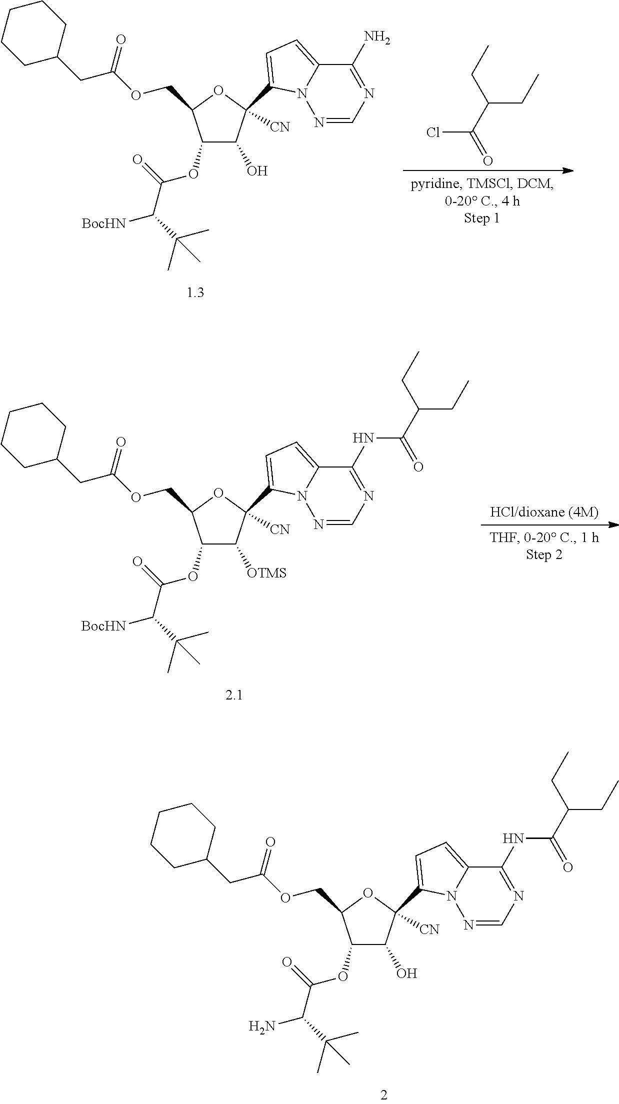

### Step 1: Synthesis of (2R,3R,4R,5R)-5-cyano-2-((2-cyclohexylacetoxy)methyl)-5-(4-(2-ethylbutanamido)pyrrolo[2,1-f][1,2,4]triazin-7-yl)-4-((trimethylsilyl)oxy)tetrahydrofuran-3-yl (S)-2-((tert-butoxycarbonyl)amino)-3,3-dimethylbutanoate (2.1)

The title compound was prepared from compound 1.3 according to the procedure of Example 1, Step 3, using 2-ethylbutanoyl chloride. MS (ESI): m/z calcd. for C₄₀H₆₂N₆O₉Si, 798.43, found 799.45 [M+H]⁺. ¹H NMR (400 MHz, DMSO) δ 11.00 (s, 1H), 8.42 (s, 1H), 7.28 (d, J=4.8 Hz, 1H), 7.12 (d, J=4.8 Hz, 1H), 7.07 (d, J=8.8 Hz, 1H), 5.27 (d, J=5.2 Hz, 1H), 5.18-5.08 (m, 1H), 4.53 (dd, J=8.8, 5.2 Hz, 1H), 4.35 (dd, J=12.4, 3.2 Hz, 1H), 4.25 (dd, J=12.4, 4.8 Hz, 1H), 4.02 (d, J=9.2 Hz, 1H), 2.83-2.71 (m, 1H), 2.22-2.05 (m, 2H), 1.65-1.50 (m, 10H), 1.37 (s, 9H), 1.28-1.20 (m, 3H), 0.93 (s, 9H), 0.90-0.86 (m, 8H), 0.05 (s, 9H).

### Step 2: Synthesis of (2R,3S,4R,5R)-5-cyano-2-((2-cyclohexylacetoxy)methyl)-5-(4-(2-ethylbutanamido)pyrrolo[2,1-f][1,2,4]triazin-7-yl)-4-hydroxytetrahydrofuran-3-yl (S)-2-amino-3,3-dimethylbutanoate (2)

The title compound was prepared according to the procedure of Example 1, Step 4, using 2.1. MS (ESI): mass calcd. for C₃₂H₄₆N₆O₇, 626.34, found 627.15 [M+H]⁺. ¹H NMR (400 MHz, DMSO) δ 10.99 (s, 1H), 8.42 (s, 1H), 7.25 (d, J=4.8 Hz, 1H), 7.13 (d, J=4.8 Hz, 1H), 6.80-6.68 (m, 1H), 5.17-5.04 (m, 2H), 4.52 (dd, J=8.0, 4.0 Hz, 1H), 4.34-4.25 (m, 1H), 4.24-4.16 (m, 1H), 3.10 (s, 1H), 2.82-2.73 (m, 1H), 2.18-2.06 (m, 2H), 1.69-1.46 (m, 10H), 1.15-1.00 (m, 3H), 0.97-0.77 (m, 17H).

### Example 3. Synthesis of (2R,3S,4R,5R)-5-cyano-2-((2-cyclohexylacetoxy)methyl)-4-hydroxy-5-(4-(2-methoxy-2-methylpropanamido)pyrrolo[2,1-f][1,2,4]triazin-7-yl)tetrahydrofuran-3-yl (S)-2-amino-3,3-dimethylbutanoate (3)

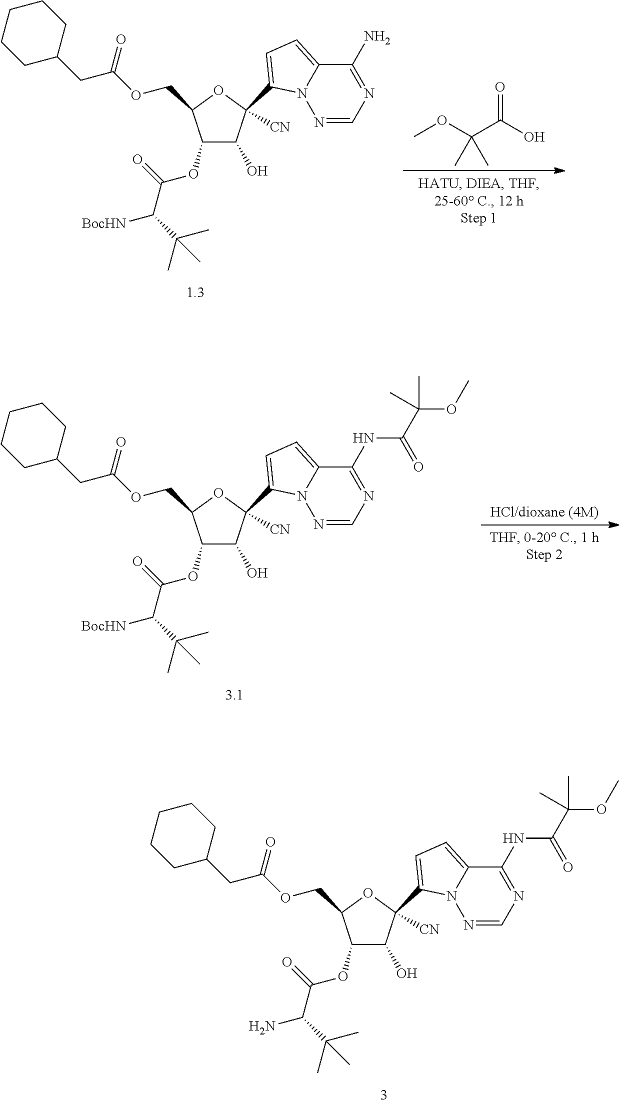

### Step 1: Synthesis of (2R,3S,4R,5R)-5-cyano-2-((2-cyclohexylacetoxy)methyl)-4-hydroxy-5-(4-(2-methoxy-2-methylpropanamido)pyrrolo[2,1-f][1,2,4]triazin-7-yl)tetrahydrofuran-3-yl (S)-2-((tert-butoxycarbonyl)amino)-3,3-dimethylbutanoate (3.1)

To a solution of 2-methoxy-2-methylpropanoic acid (51.0 mg, 0.432 mmol) in THF (40 mL) was added HATU (87.5 mg, 0.648 mmol), the solution was stirred at 25° C. for 1 h. To the above solution was added compound 1.3 (149 mg, 0.237 mmol) and DIEA (167 mg, 1.30 mmol), the mixture was stirred at 60° C. for 16 h. The mixture was diluted with water (50 mL) and extracted with EtOAc (50 mL×3). The organic phase was washed with brine water (50 mL×3), then dried over anhydrous sodium sulfate, filtered, and concentrated to remove the solvent. The crude product was purified by chromatography (SiO₂, Petroleum ether/Ethyl acetate from 0% to 30%) to obtain compound 3.1 (80 mg, 22.9%) as a white solid. MS (ESI): mass calcd. for C₃₆H₅₂N₆O₁₀, 728.37, found 729.30 [M+H]⁺. ¹H NMR (400 MHz, DMSO) δ 10.32 (s, 1H), 8.49 (s, 1H), 7.22 (d, J=4.8 Hz, 1H), 7.17 (d, J=4.8 Hz, 1H), 7.06 (d, J=8.0 Hz, 1H), 6.78 (d, J=6.4 Hz, 1H), 5.15-5.08 (m, 2H), 4.52-4.45 (m, 1H), 4.35-4.25 (m, 1H), 4.23-4.15 (m, 1H), 3.95 (d, J=8.4 Hz, 1H), 3.29 (s, 3H), 2.13 (t, J=6.8 Hz, 2H), 1.60-1.50 (m, 6H), 1.44 (s, 6H), 1.40 (s, 9H), 1.15-1.02 (m, 3H), 0.99 (s, 9H), 0.87-0.79 (m, 2H).

### Step 2: Synthesis of (2R,3S,4R,5R)-5-cyano-2-((2-cyclohexylacetoxy)methyl)-4-hydroxy-5-(4-(2-methoxy-2-methylpropanamido)pyrrolo[2,1-f][1,2,4]triazin-7-yl)tetrahydrofuran-3-yl (S)-2-amino-3,3-dimethylbutanoate (3)

The title compound was prepared according to the procedure of Example 1, Step 4, using 3.1. MS (ESI): mass calcd. for C₃₁H₄₄N₆O₈, 628.32, found 629.15 [M+H]⁺. ¹H NMR (400 MHz, DMSO) δ 8.47 (s, 1H), 7.21 (d, J=4.8 Hz, 1H), 7.17 (d, J=4.8 Hz, 1H), 6.85-6.65 (m, 1H), 5.19-5.00 (m, 2H), 4.52 (dd, J=8.0, 4.0 Hz, 1H), 4.39-4.26 (m, 1H), 4.25-4.14 (m, 1H), 3.29 (s, 3H), 3.10 (s, 1H), 2.21-2.05 (m, 2H), 1.60-1.51 (m, 6H), 1.44 (s, 6H), 1.19-1.03 (m, 3H), 0.93 (s, 9H), 0.88-0.76 (m, 2H).

### Example 4. Synthesis of (2R,3S,4R,5R)-5-cyano-2-((2-cyclohexylacetoxy)methyl)-4-hydroxy-5-(4-(3-isopropylureido)pyrrolo[2,1-f][1,2,4]triazin-7-yl)tetrahydrofuran-3-yl (S)-2-amino-3,3-dimethylbutanoate (4)

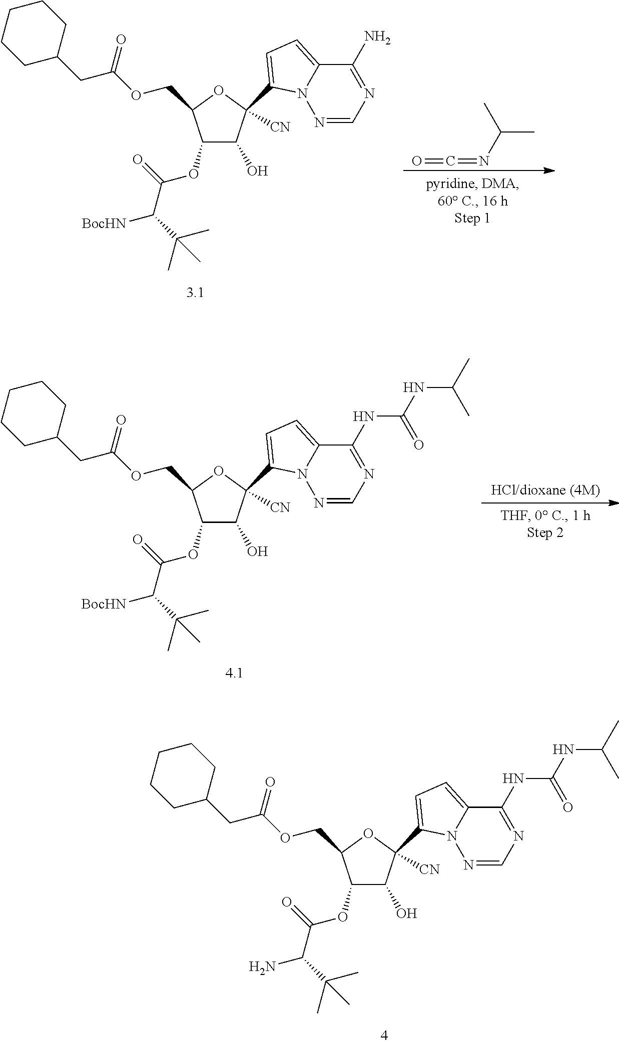

### Step 1: Synthesis of (2R,3S,4R,5R)-5-cyano-2-((2-cyclohexylacetoxy)methyl)-4-hydroxy-5-(4-(3-isopropylureido)pyrrolo[2,1-f][1,2,4]triazin-7-yl)tetrahydrofuran-3-yl (S)-2-((tert-butoxycarbonyl)amino)-3,3-dimethylbutanoate (4.1)

To a solution of compound 3.1 (110 mg, 0.175 mmol) and pyridine (41.5 mg, 0.525 mmol) in DMA (1 mL) was added 2-isocyanatopropane (22.3 mg, 0.263 mmol) at 20° C., the mixture was stirred at 60° C. for 16 h. After completion, the mixture was concentrated in vacuo to afford a residue, the residue was diluted with ACN (2 mL) and purified by prep-HPLC (mobile phase: ACN-H₂O (0.1% FA), 20%-70%) to obtain compound 4.1 (50.0 mg, 38% yield) as a white solid. MS (ESI): mass calcd. for C₃₅H₅₁N₇O₉, 713.37, found 714.25 [M+H]⁺.

### Step 2: Synthesis of (2R,3S,4R,5R)-5-cyano-2-((2-cyclohexylacetoxy)methyl)-4-hydroxy-5-(4-(3-isopropylureido)pyrrolo[2,1-f][1,2,4]triazin-7-yl)tetrahydrofuran-3-yl (S)-2-amino-3,3-dimethylbutanoate (4)

The title compound was prepared according to the procedure of Example 1, Step 4, using compound 4.1. MS (ESI): mass calcd. for C₃₀H₄₃N₇O₇, 613.32, found 614.15 [M+H]⁺. ¹H NMR (400 MHz, DMSO) δ 10.53 (s, 1H), 9.32 (d, J=7.2 Hz, 1H), 8.30 (s, 1H), 7.50 (d, J=4.8 Hz, 1H), 7.02 (d, J=4.8 Hz, 1H), 6.76-6.65 (m, 1H), 5.13-5.03 (m, 2H), 4.50 (dd, J=8.4, 4.4 Hz, 1H), 4.37-4.25 (m, 1H), 4.23-4.10 (m, 1H), 3.98-3.87 (m, 1H), 3.11 (s, 1H), 2.18-2.02 (m, 2H), 1.62-1.49 (m, 6H), 1.21 (d, J=6.4 Hz, 6H), 1.17-1.02 (m, 3H), 0.93 (s, 9H), 0.89-0.73 (m, 2H).

### Example 5. Synthesis of (2R,3S,4R,5R)-5-cyano-4-hydroxy-5-(4-(2-methoxy-2-methylpropanamido)pyrrolo[2,1-f][1,2,4]triazin-7-yl)-2-((2-phenylacetoxy)methyl)tetrahydrofuran-3-yl (S)-2-amino-3,3-dimethylbutanoate (5)

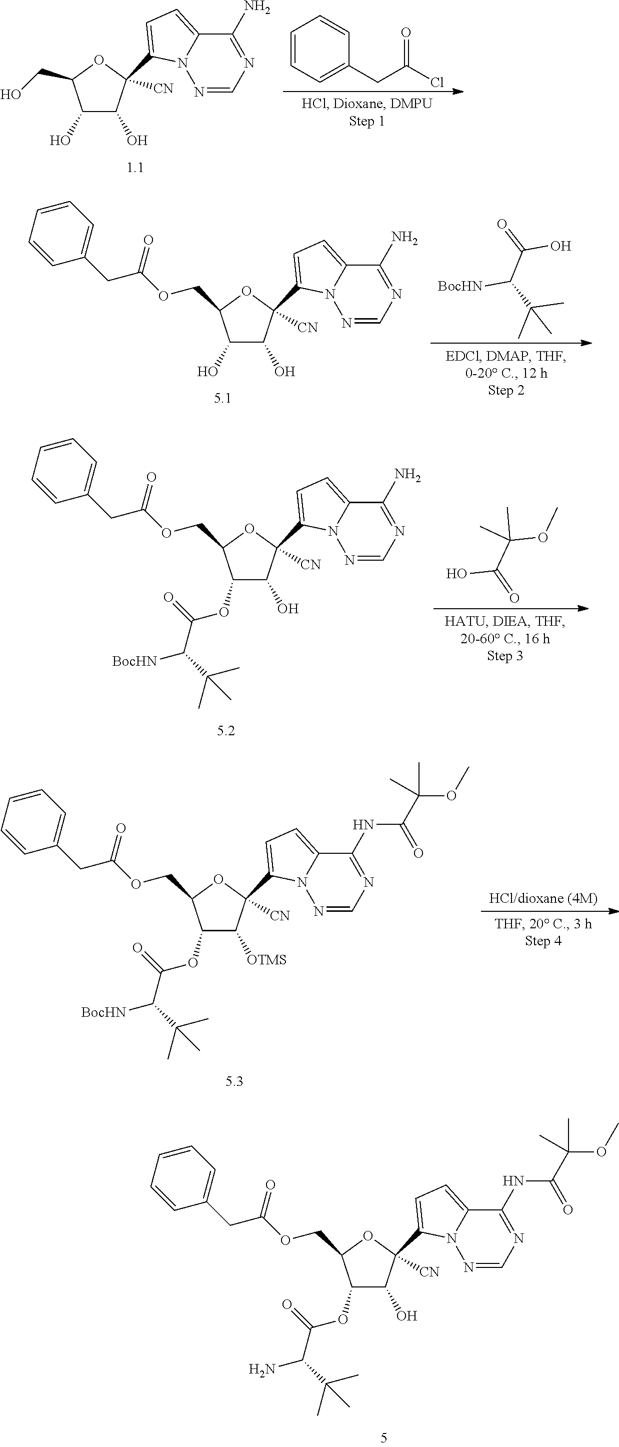

### Step 1. Synthesis of ((2R,3S,4R,5R)-5-(4-aminopyrrolo[2,1-f][1,2,4]triazin-7-yl)-5-cyano-3,4-dihydroxytetrahydrofuran-2-yl)methyl 2-phenylacetate (5.1)

The title compound was prepared according to the procedure of Example 1, Step 1, using compound 1.1 and phenylacetyl chloride. MS (ESI): mass calcd. for C₂₀H₁₉N₅O₅, 409.14, m/z found 410.0 [M+H]⁺. ¹HNMR (400 MHz, DMSO-d₆) δ 8.13 (br s, 2H), 7.95 (s, 1H), 7.21-7.28 (m, 5H), 6.91 (d, J=4.4 Hz, 1H), 6.79 (d, J=4.4 Hz, 1H), 6.3 (br s, 1H), 5.39 (br s, 1H), 4.65 (d, J=4.8 Hz, 1H), 4.35 (m, 1H), 3.9-4.2 (m, 2H), 3.67 (m, 2H).

### Step 2. Synthesis of (2R,3S,4R,5R)-5-(4-aminopyrrolo[2,1-f][1,2,4]triazin-7-yl)-5-cyano-4-hydroxy-2-((2-phenylacetoxy)methyl)tetrahydrofuran-3-yl (S)-2-((tert-butoxycarbonyl)amino)-3,3-dimethylbutanoate (5.2)

The title compound was prepared from compound 5.1 according to the procedure of Example 1, Step 2, using (R)-Boc-tert-leucine.

### Step 3. Synthesis of (2R,3S,4R,5R)-5-cyano-4-hydroxy-5-(4-(2-methoxy-2-methylpropanamido)pyrrolo[2,1-f][1,2,4]triazin-7-yl)-2-((2-phenylacetoxy)methyl)tetrahydrofuran-3-yl (S)-2-((tert-butoxycarbonyl)amino)-3,3-dimethylbutanoate (5.3)

Title compound 5.3 was prepared according to the procedure of Example 3, Step 1, using 5.2 and 2-methoxy-2-methylpropanoic acid. MS (ESI): mass calcd. for C₃₆H₄₆N₆O₁₀, 722.33, m/z found 723.2 [M+H]⁺.

### Step 4. Synthesis of (2R,3S,4R,5R)-5-cyano-4-hydroxy-5-(4-(2-methoxy-2-methylpropanamido)pyrrolo[2,1-f][1,2,4]triazin-7-yl)-2-((2-phenylacetoxy)methyl)tetrahydrofuran-3-yl (S)-2-amino-3,3-dimethylbutanoate (5)

The title compound was prepared from carbamate 5.3 according to the procedure of Example 1, Step 4. MS (ESI): mass calcd. for C₃₁H₃₈N₆O₈, 622.28, m/z found 623.15 [M+H]⁺. ¹H NMR (400 MHz, DMSO) δ 8.48 (s, 1H), 7.29-7.18 (m, 6H), 7.14 (d, J=4.8 Hz, 1H), 6.72-6.70 (m, 1H), 5.20-5.10 (m, 1H), 5.06-5.02 (m, 1H), 4.53 (dd, J=8.4, 4.4 Hz, 1H), 4.29-4.20 (m, 2H), 3.66 (s, 2H), 3.30 (s, 3H), 3.10 (s, 1H), 1.45 (s, 6H), 0.93 (s, 9H).

### Example 6. Synthesis of (2R,3S,4R,5R)-5-cyano-5-(4-(2-ethoxy-2-methylpropanamido)pyrrolo[2,1-f][1,2,4]triazin-7-yl)-4-hydroxy-2-((2-phenylacetoxy)methyl)tetrahydrofuran-3-yl (S)-2-amino-3,3-dimethylbutanoate (6)

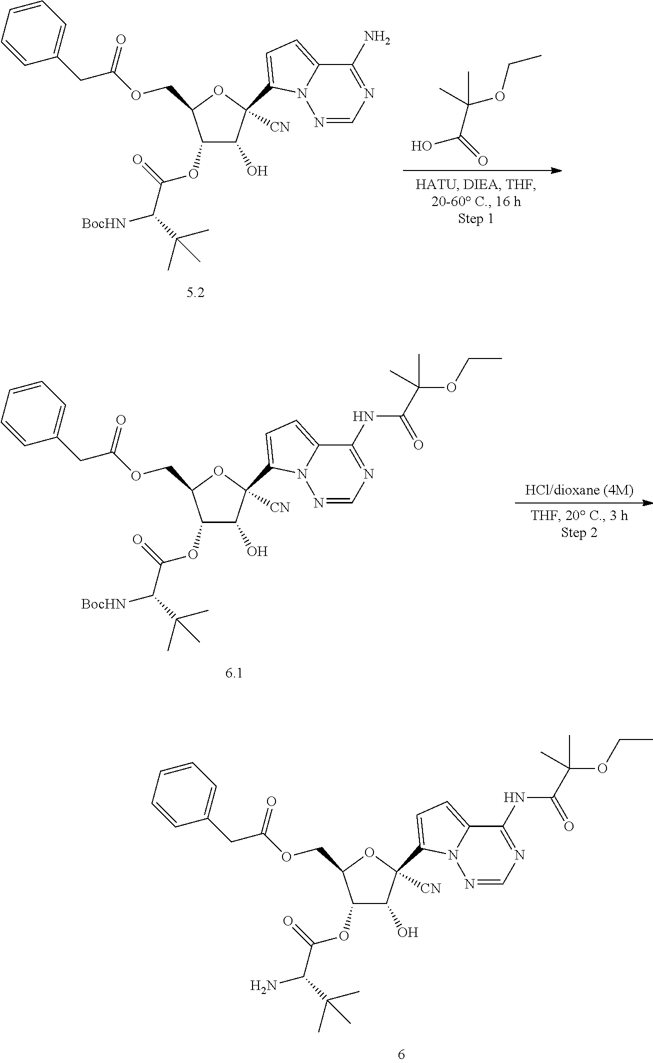

### Step 1. Synthesis of (2R,3S,4R,5R)-5-cyano-5-(4-(2-ethoxy-2-methylpropanamido)pyrrolo[2,1-f][1,2,4]triazin-7-yl)-4-hydroxy-2-((2-phenylacetoxy)methyl)tetrahydrofuran-3-yl (S)-2-((tert-butoxycarbonyl)amino)-3,3-dimethylbutanoate (6.1)

Title compound 6.1 was prepared according to the procedure of Example 3, Step 1, using 5.2 and 2-ethoxy-2-methylpropanoic acid. MS (ESI): mass calcd. for C₃₇H₄₈N₆O₁₀, 736.34, m/z found 737.2 [M+H]⁺.

### Step 2. Synthesis of (2R,3S,4R,5R)-5-cyano-5-(4-(2-ethoxy-2-methylpropanamido)pyrrolo[2,1-f][1,2,4]triazin-7-yl)-4-hydroxy-2-((2-phenylacetoxy)methyl)tetrahydrofuran-3-yl (S)-2-amino-3,3-dimethylbutanoate (6)

The title compound was prepared from carbamate 6.1 according to the procedure of Example 1, Step 4. MS (ESI): mass calcd. for C₃₂H₄₀N₆O₈, 636.29, m/z found 637.15 [M+H]⁺. ¹H NMR (400 MHz, DMSO) δ 8.47 (s, 1H), 7.29-7.18 (m, 6H), 7.14 (d, J=4.8 Hz, 1H) 6.73 (d, J=5.6 Hz, 1H), 5.15-5.09 (m, 1H), 5.06-5.02 (m, 1H), 4.53 (dd, J=8.0, 4.4 Hz, 1H), 4.29-4.20 (m, 2H), 3.66 (s, 2H), 3.50 (q, J=6.8 Hz, 2H), 3.09 (s, 1H), 1.45 (s, 6H), 1.22 (t J=6.8 Hz, 3H), 0.94 (s, 9H).

### Example 7. Synthesis of (2R,3S,4R,5R)-5-cyano-2-((2-cyclohexylacetoxy)methyl)-5-(4-(2-ethoxy-2-methylpropanamido)pyrrolo[2,1-f][1,2,4]triazin-7-yl)-4-hydroxytetrahydrofuran-3-yl (S)-2-amino-3,3-dimethylbutanoate-(7)

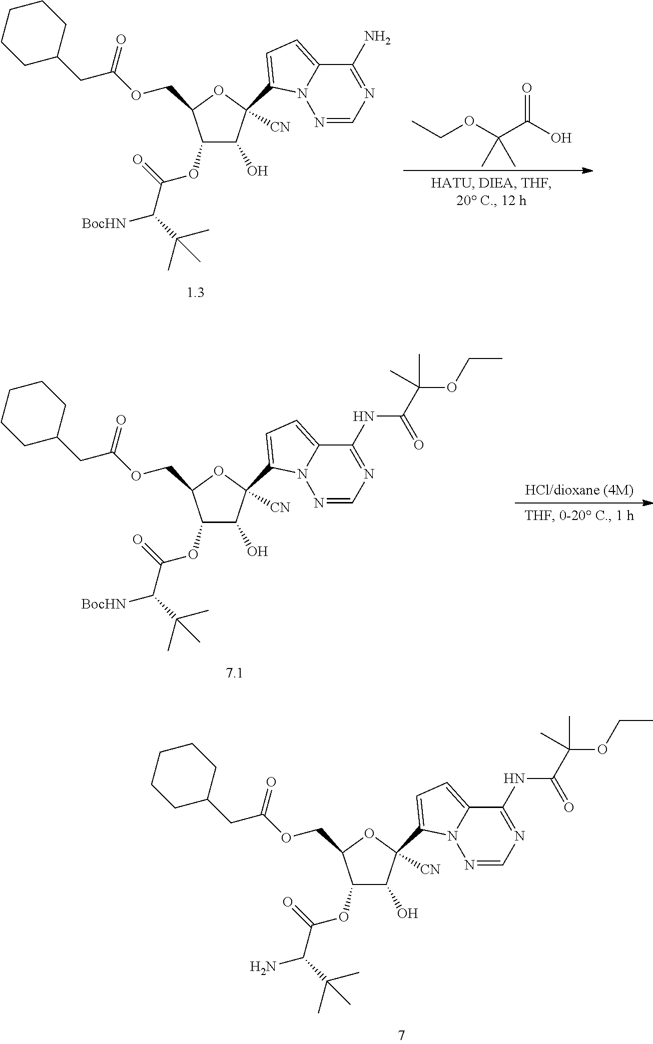

Title compound 7 was prepared from compound 1.3 according to the procedure of Example 3, Steps 1-2, using 2-ethoxy-2-methylpropanoic acid. MS (ESI): mass calcd. for C₃₂H₄₆N₆O₈, 642.34, m/z found 643.25 [M+H]⁺. ¹H NMR (400 MHz, DMSO) δ 10.17 (s, 1H), 8.47 (s, 1H), 7.21 (d, J=4.8 Hz, 1H), 7.16 (d, J=4.8 Hz, 1H), 6.78-6.70 (m, 1H), 5.16-5.03 (m, 2H), 4.52 (dd, J=8.0, 4.0 Hz, 1H), 4.36-4.27 (m, 1H), 4.24-4.16 (m, 1H), 3.49 (q, J=6.8 Hz, 2H), 3.10 (s, 1H), 2.20-2.07 (m, 2H), 1.63-1.52 (m, 6H), 1.45 (s, 6H), 1.22 (t, J=7.2 Hz, 3H), 1.18-1.05 (m, 3H), 0.93 (s, 9H), 0.87-0.76 (m, 2H).

### Example 8. Synthesis of (2R,3S,4R,5R)-5-cyano-5-(4-(2-ethylbutanamido)pyrrolo[2,1-f][1,2,4]triazin-7-yl)-4-hydroxy-2-((2-phenylacetoxy)methyl)tetrahydrofuran-3-yl (S)-2-amino-3,3-dimethylbutanoate (8)

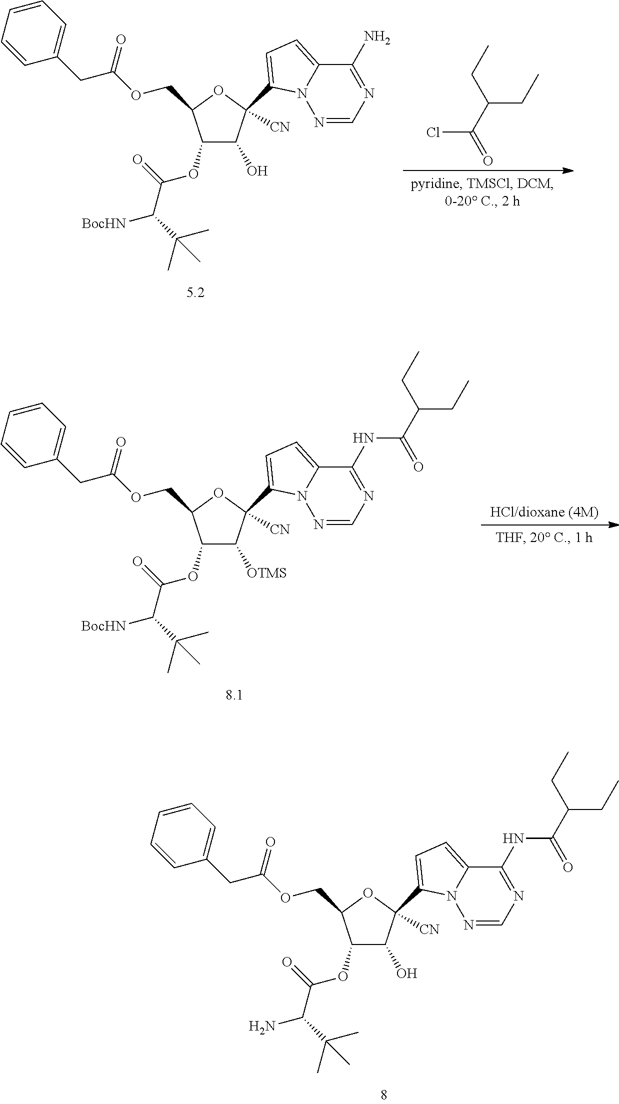

Title compound was prepared from 5.2 according to the procedure of Example 2, Steps 1-2, using 2-ethylbutanoyl chloride. MS (ESI): mass calcd. for C₃₂H₄₀N₆O₇, 620.30, m/z found 621.10 [M+H]⁺. ¹H NMR (400 MHz, DMSO) δ 11.00 (s, 1H), 8.43 (s, 1H), 7.39-7.15 (m, 6H), 7.10 (d, J=4.8 Hz, 1H), 6.73-6.70 (m, 1H), 5.16-5.09 (m, 1H), 5.06 (d, J=5.6 Hz, 1H), 4.52 (dd, J=8.0, 4.4 Hz, 1H), 4.34 (dd, J=12.4, 3.6 Hz, 1H), 4.25 (dd, J=12.4, 4.8 Hz, 1H), 3.72-3.60 (m, 2H), 3.09 (s, 1H), 2.78-2.70 (m, 1H), 1.69-1.46 (m, 4H), 1.01-0.86 (m, 15H).

### Example 9. Synthesis of (2R,3S,4R,5R)-5-cyano-4-hydroxy-5-(4-((R)-2-methylbutanamido)pyrrolo[2,1-f][1,2,4]triazin-7-yl)-2-((2-phenylacetoxy)methyl)tetrahydrofuran-3-yl (S)-2-amino-3,3-dimethylbutanoate (9)

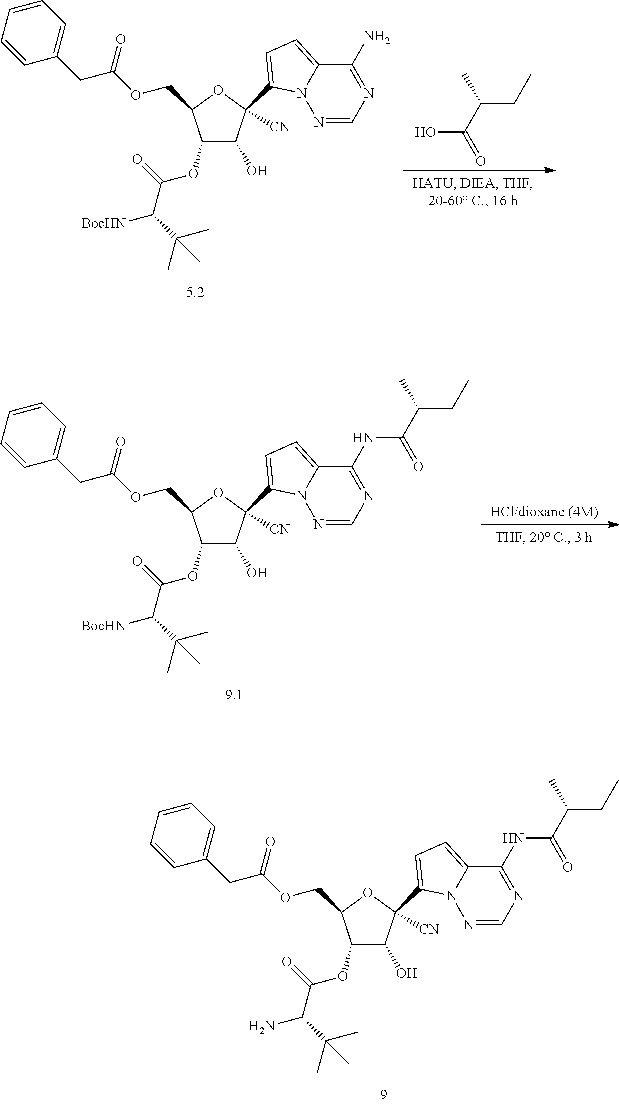

Title compound 9 was prepared from 5.2 according to the procedure of Example 3, Steps 1-2, using (R)-2-methylbutanoic acid. MS (ESI): mass calcd. for C₃₁H₃₈N₆O₇, 606.28, m/z found 607.10 [M+H]⁺. ¹H NMR (400 MHz, DMSO) δ 10.91 (s, 1H), 8.41 (s, 1H), 7.27-7.18 (m, 6H), 7.09 (d, J=4.8 Hz, 1H), 6.68 (d, J=5.6 Hz, 1H), 5.18-5.09 (m, 1H), 5.06 (d, J=5.2 Hz, 1H), 4.52 (dd, J=8.0, 4.4 Hz, 1H), 4.36-4.24 (m, 2H), 3.66 (s, 2H), 3.10 (s, 1H), 2.94-2.90 (m, 1H), 1.73-1.65 (m, 1H), 1.47-1.40 (m, 1H), 1.14 (d, J=6.8 Hz, 3H), 0.97-0.90 (m, 12H).

### Example 10. Synthesis of (2R,3S,4R,5R)-5-cyano-4-hydroxy-5-(4-((S)-2-methylbutanamido)pyrrolo[2,1-f][1,2,4]triazin-7-yl)-2-((2-phenylacetoxy)methyl)tetrahydrofuran-3-yl (S)-2-amino-3,3-dimethylbutanoate (10)

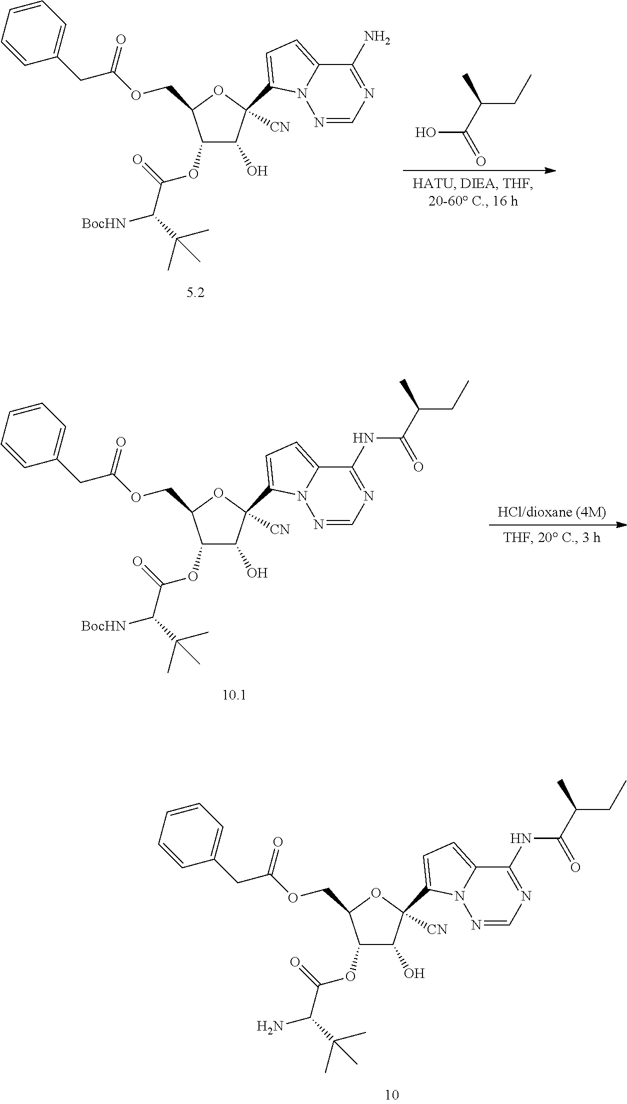

Title compound 10 was prepared from 5.2 according to the procedure of Example 3, Steps 1-2, using (S)-2-methylbutanoic acid. MS (ESI): mass calcd. for C₃₁H₃₈N₆O₇, 606.28, m/z found 607.10 [M+H]⁺. ¹H NMR (400 MHz, DMSO) δ 10.94 (s, 1H), 8.42 (s, 1H), 7.23-7.17 (m, 6H), 7.09 (d, J=4.8 Hz, 1H), 6.70-6.65 (m, 1H), 5.11 (t, J=5.2 Hz, 1H), 5.05-5.02 (m, 1H), 4.52 (dd, J=8.4, 4.0 Hz, 1H), 4.35-4.23 (m, 2H), 3.66 (s, 2H), 3.10 (s, 1H), 2.98-2.92 (m, 1H), 1.69-1.60 (m, 1H), 1.47-1.41 (m, 1H), 1.14 (d, J=6.8 Hz, 3H), 0.96-0.85 (m, 12H).

### Example 11. Synthesis of (2R,3S,4R,5R)-5-(4-(((acetoxymethoxy)carbonyl)amino)pyrrolo[2,1-f][1,2,4]triazin-7-yl)-5-cyano-4-hydroxy-2-(hydroxymethyl)tetrahydrofuran-3-yl (S)-2-amino-3,3-dimethylbutanoate (11)

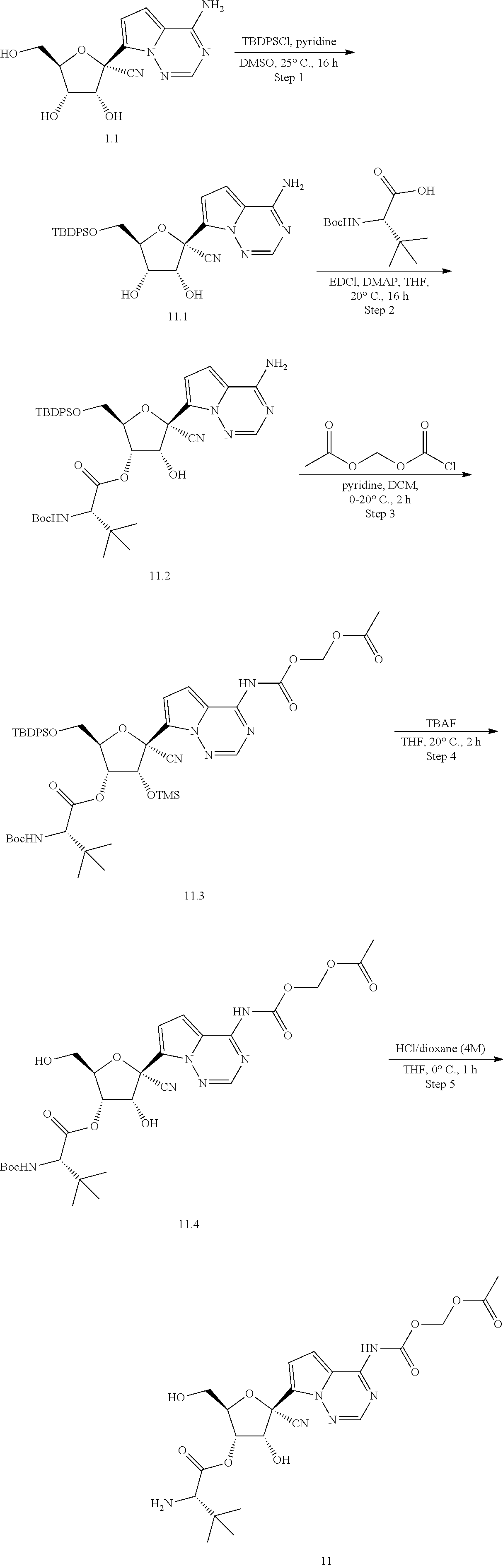

### Step 1: Synthesis of (2R,3R,4S,5R)-2-(4-aminopyrrolo[2,1-f][1,2,4]triazin-7-yl)-5-(((tert-butyldiphenylsilyl)oxy)methyl)-3,4-dihydroxytetrahydrofuran-2-carbonitrile (11.1)

To a solution of compound 1.1 (10.0 g, 34.3 mmol) and pyridine (5.6 mL, 68.6 mmol) in dry DMSO (100 mL) was added TBDPSCI (9.8 mL, 37.7 mmol) dropwise. The reaction mixture was stirred at 25° C. for 16 h. The reaction mixture was poured into cold water (250 mL) and white solid precipitated. The mixture was filtered, and the filter cake was dried to give compound 11.1 (17.9 g, 94% yield) as a white solid. MS (ESI): mass calcd. for C₂₈H₃₁N₅O₄Si, 529.21, found 530.20 [M+H]⁺.

### Step 2: Synthesis of (2R,3S,4R,5R)-5-(4-aminopyrrolo[2,1-f][1,2,4]triazin-7-yl)-2-(((tert-butyldiphenylsilyl)oxy)methyl)-5-cyano-4-hydroxytetrahydrofuran-3-yl (S)-2-((tert-butoxycarbonyl)amino)-3,3-dimethylbutanoate (11.2)

To a solution of compound 11.1 (1.00 g, 1.89 mmol) in THF (50 mL) was added (2S)-2-{[(tert-butoxy)carbonyl]amino}-3,3-dimethylbutanoic acid (524 mg, 2.26 mmol), EDCI (1086 mg, 5.66 mmol) and DMAP (692 mg, 5.66 mmol), the mixture was stirred at 25° C. for 16 h. After completion, the mixture was concentrated in vacuo to afford a residue. Then the reaction residue was diluted with water (100 mL) and extracted with EtOAc (100 mL×3). The organic layer was dried over sodium sulphate and concentrated under reduce pressure to afford a crude product. The residue was purified by prep-HPLC [Gradient: 5-75% ACN in water (0.1% FA)] to obtain compound 11.2 (300 mg, 20% yield) as a white solid. MS (ESI): mass calcd. for C₃₉H₅₀N₆O₇Si, 742.35, found 743.35 [M+H]⁺. ¹HNMR (400 MHz, DMSO) δ 7.88 (br s, 3H), 7.53 (d, J=6.8 Hz, 2H), 7.48-7.31 (m, 6H), 7.24 (t, J=7.6 Hz, 2H), 6.99 (d, J=8.4 Hz, 1H), 6.85 (d, J=4.8 Hz, 1H), 6.80 (d, J=4.4 Hz, 1H), 6.61 (d, J=6.4 Hz, 1H), 5.44-5.32 (m, 1H), 5.10 (t, J=6.0 Hz, 1H), 4.36 (d, J=4.0 Hz, 1H), 4.00 (d, J=8.8 Hz, 1H), 3.87-3.64 (m, 2H), 1.39 (s, 9H), 1.00 (s, 9H), 0.91 (s, 9H).

### Step 3: Synthesis (2R,3R,4R,5R)-5-(4-(((acetoxymethoxy)carbonyl)amino)pyrrolo[2,1-f][1,2,4]triazin-7-yl)-2-(((tert-butyldiphenylsilyl)oxy)methyl)-5-cyano-4-((trimethylsilyl)oxy)tetrahydrofuran-3-yl (S)-2-((tert-butoxycarbonyl)amino)-3,3-dimethylbutanoate (11.3)

To a solution of compound 11.2 (100 mg, 0.135 mmol) and pyridine (0.13 mL, 1.62 mmol) in DCM (1 mL) was added TMSCI (43.9 mg, 0.404 mmol) at 0° C., the mixture was stirred at 20° C. for 1 h. And then the reaction was added [(chlorocarbonyl)oxy]methyl acetate (30.8 mg, 0.202 mmol) at 0° C., the mixture was stirred at 0° C. for 1 h. The reaction was quenched by water (50 mL) and extracted with DCM (50 mL×3). The organic phase was washed with brine water (50 mL×3), then dried over anhydrous sodium sulfate, filtered, and concentrated to remove the solvent. The crude product was purified by chromatography (SiO₂, Petroleumether/Ethyl acetate from 0% to 20%) to obtain compound 11.3 (100 mg, 75.8% yield) as a white solid. MS (ESI): mass calcd. for C₄₆H₆₂N₆O₁₁Si₂, 930.40, found 931.25 [M+H]⁺. ¹H NMR (400 MHz, DMSO) δ 11.29 (s, 1H), 8.24 (s, 1H), 7.58-7.53 (m, 4H), 7.48-7.41 (m, 2H), 7.38-7.32 (m, 4H), 7.30 (d, J=4.4 Hz, 1H), 7.08 (d, J=9.2 Hz, 1H), 7.03 (d, J=4.4 Hz, 1H), 5.81 (s, 2H), 5.41 (t, J=5.2 Hz, 1H), 5.25 (d, J=5.2 Hz, 1H), 4.45 (d, J=4.8 Hz, 1H), 4.08-4.03 (m, 1H), 3.98-3.89 (m, 1H), 3.79 (dd, J=12.0, 3.6 Hz, 1H), 2.12 (s, 3H), 1.39 (s, 9H), 0.96-0.91 (m, 18H), −0.00 (s, 9H).

### Step 4: Synthesis of (2R,3S,4R,5R)-5-(4-(((acetoxymethoxy)carbonyl)amino)pyrrolo[2,1-f][1,2,4]triazin-7-yl)-5-cyano-4-hydroxy-2-(hydroxymethyl)tetrahydrofuran-3-yl (S)-2-((tert-butoxycarbonyl)amino)-3,3-dimethylbutanoate (11.4)

To a solution of compound 11.3 (100 mg, 0.107 mmol) in THF (1 mL) was added TBAF (0.2 mL, 1 M in THF) at 0° C., then the reaction was stirred at 20° C. for 1 h. The reaction was diluted with water (50 mL) and extracted with EtOAc (50 mL×3). The organic layer was dried over sodium sulphate and concentrated under reduced pressure to afford a crude. The crude product was purified by chromatography (SiO₂, Dichloromethane/Methanol from 0% to 4%) to obtain compound 11.4 (50.0 mg, 57% yield) as a white solid. MS (ESI): mass calcd. for C₂₇H₃₆N₆O₁₁, 620.24, found 621.15 [M+H]⁺. ¹H NMR (400 MHz, DMSO) δ 11.28 (s, 1H), 8.44 (s, 1H), 7.33 (s, 1H), 7.14 (d, J=4.4 Hz, 1H), 6.99 (d, J=8.4 Hz, 1H), 6.61 (d, J=6.8 Hz, 1H), 5.82 (s, 2H), 5.19-5.12 (m, 1H), 5.10-5.03 (m, 1H), 5.00-4.90 (m, 1H), 4.27 (d, J=3.6 Hz, 1H), 3.96 (d, J=8.4 Hz, 1H), 3.65-3.50 (m, 1H), 2.12 (s, 3H), 1.40 (s, 9H), 0.99 (s, 9H).

### Step 5. Synthesis of (2R,3S,4R,5R)-5-(4-(((acetoxymethoxy)carbonyl)amino)pyrrolo[2,1-f][1,2,4]triazin-7-yl)-5-cyano-4-hydroxy-2-(hydroxymethyl)tetrahydrofuran-3-yl (S)-2-amino-3,3-dimethylbutanoate (11)

The title compound was prepared from carbamate 11.4 according to the procedure of Example 1, Step 4. MS (ESI): mass calcd. for C₂₂H₂₈N₆O₉, 520.19, found 521.10 [M+H]⁺. ¹H NMR (400 MHz, DMSO) δ 8.39 (s, 1H), 7.29 (d, J=4.4 Hz, 1H), 7.12 (d, J=4.4 Hz, 1H), 6.58 (d, J=6.0 Hz, 1H), 5.81 (s, 2H), 5.20-5.12 (m, 1H), 5.06 (t, J=5.6 Hz, 1H), 5.03-4.93 (m, 1H), 4.35-4.26 (m, 1H), 3.67-3.51 (m, 2H), 3.12 (s, 1H), 2.12 (s, 3H), 0.95 (s, 9H).

### Example 12. Synthesis of (2R,3S,4R,5R)-5-cyano-2-((2-cyclohexylacetoxy)methyl)-4-hydroxy-5-(4-((R)-2-methylbutanamido)pyrrolo[2,1-f][1,2,4]triazin-7-yl)tetrahydrofuran-3-yl (S)-2-amino-3,3-dimethylbutanoate (12)

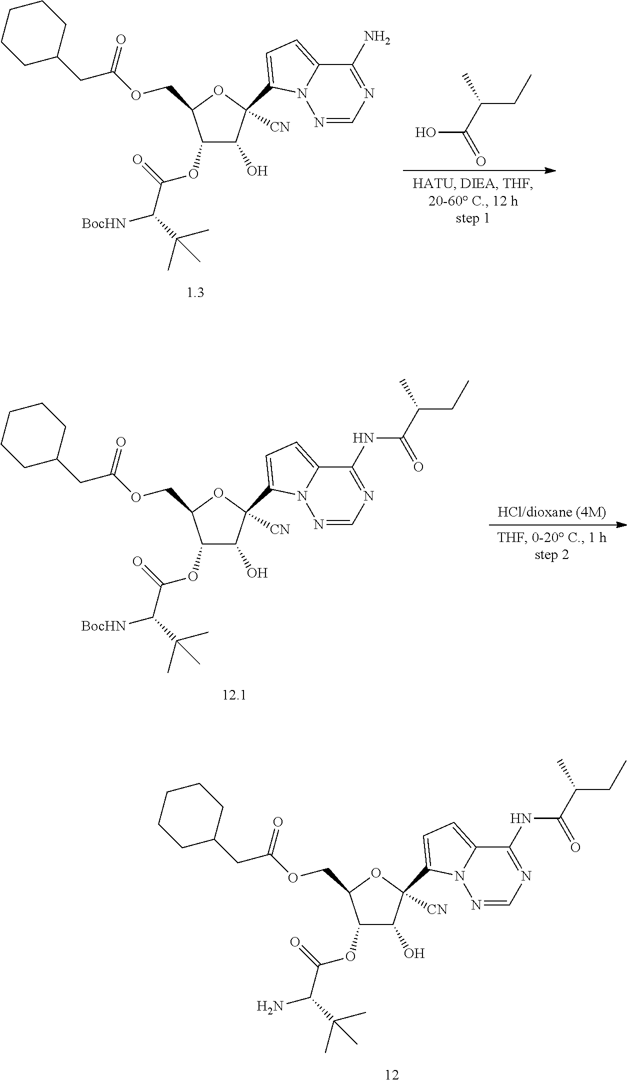

### Step 1. Synthesis of (2R,3S,4R,5R)-5-cyano-2-((2-cyclohexylacetoxy)methyl)-4-hydroxy-5-(4-((R)-2-methylbutanamido)pyrrolo[2,1-f][1,2,4]triazin-7-yl)tetrahydrofuran-3-yl (S)-2-((tert-butoxycarbonyl)amino)-3,3-dimethylbutanoate (12.1)

Title compound 12.1 was prepared according to the procedure of Example 3, Step 1, using 1.3 and (R)-2-methylbutanoic acid. MS (ESI): mass calcd. for C₃₆H₅₂N₆O₉, 712.38, m/z found 713.25 [M+H]⁺. ¹H NMR (400 MHz, DMSO) δ 10.94 (s, 1H), 8.42 (s, 1H), 7.27 (d, J=4.8 Hz, 1H), 7.12 (d, J=4.8 Hz, 1H), 7.07 (d, J=8.4 Hz, 1H), 6.77 (d, J=6.4 Hz, 1H), 5.15-5.05 (m, 2H), 4.53-4.45 (m, 1H), 4.40-4.27 (m, 1H), 4.17 (dd, J=12.4, 5.2 Hz, 1H), 3.95 (d, J=8.4 Hz, 1H), 2.94 (dd, J=13.6, 6.8 Hz, 1H), 2.20-2.05 (m, 2H), 1.75-1.62 (m, 1H), 1.60-1.50 (m, 6H), 1.52-1.42 (m, 1H), 1.39 (s, 9H), 1.15-1.05 (m, 6H), 0.98 (s, 9H), 0.90 (t, J=7.2 Hz, 3H), 0.86-0.78 (m, 2H).

### Step 2. Synthesis of (2R,3S,4R,5R)-5-cyano-2-((2-cyclohexylacetoxy)methyl)-4-hydroxy-5-(4-((R)-2-methylbutanamido)pyrrolo[2,1-f][1,2,4]triazin-7-yl)tetrahydrofuran-3-yl (S)-2-amino-3,3-dimethylbutanoate (12)

The title compound was prepared from carbamate 12.1 according to the procedure of Example 1, Step 4. MS (ESI): mass calcd. for C₃₁H₄₄N₆O₇, 612.33, m/z found 613.25 [M+H]⁺. ¹H NMR (400 MHz, DMSO) δ 10.93 (s, 1H), 8.42 (s, 1H), 7.27 (d, J=4.8 Hz, 1H), 7.12 (d, J=4.8 Hz, 1H), 6.78-6.67 (m, 1H), 5.16-5.02 (m, 2H), 4.55-4.48 (m, 1H), 4.37-4.26 (m, 1H), 4.25-4.14 (m, 1H), 3.10 (s, 1H), 3.00-2.88 (m, 1H), 2.22-2.04 (m, 2H), 1.74-1.63 (m, 1H), 1.62-1.50 (m, 6H), 1.48-1.41 (m, 1H), 1.20-1.03 (m, 6H), 0.97-0.81 (m, 14H).

### Example 13. Synthesis of (2R,3S,4R,5R)-5-cyano-2-((2-cyclohexylacetoxy)methyl)-4-hydroxy-5-(4-((S)-2-methylbutanamido)pyrrolo[2,1-f][1,2,4]triazin-7-yl)tetrahydrofuran-3-yl (S)-2-amino-3,3-dimethylbutanoate (13)

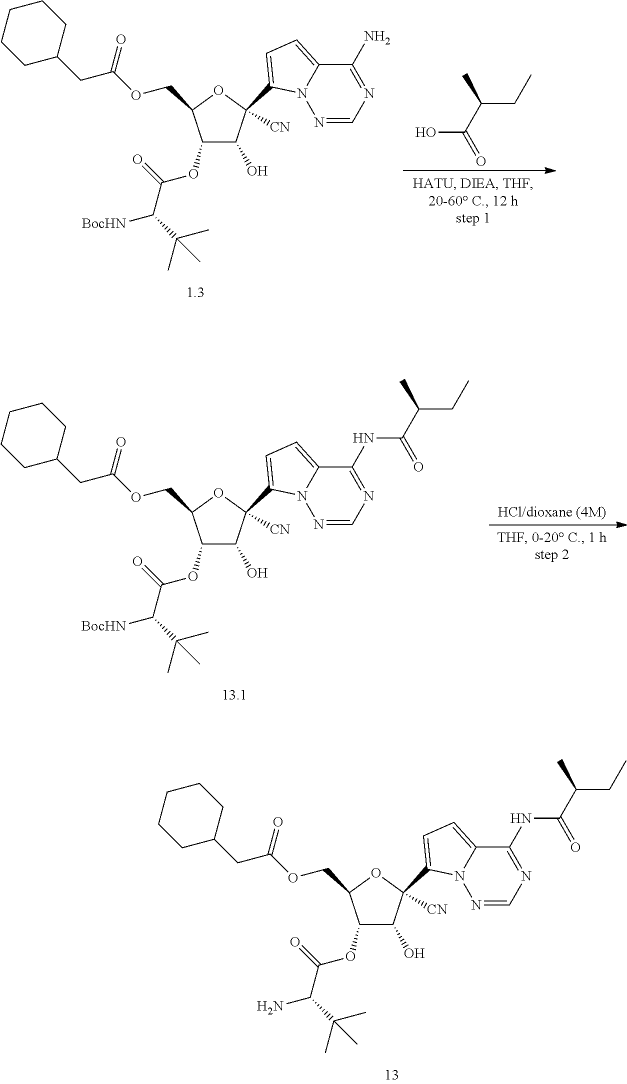

### Step 1. Synthesis of (2R,3S,4R,5R)-5-cyano-2-((2-cyclohexylacetoxy)methyl)-4-hydroxy-5-(4-((S)-2-methylbutanamido)pyrrolo[2,1-f][1,2,4]triazin-7-yl)tetrahydrofuran-3-yl (S)-2-((tert-butoxycarbonyl)amino)-3,3-dimethylbutanoate (13.1)

Title compound 13.1 was prepared according to the procedure of Example 3, Step 1, using 1.3 and (S)-2-methylbutanoic acid. MS (ESI): mass calcd. for C₃₆H₅₂N₆O₉, 712.38, m/z found 713.25 [M+H]⁺.

### Step 2. Synthesis of (2R,3S,4R,5R)-5-cyano-2-((2-cyclohexylacetoxy)methyl)-4-hydroxy-5-(4-((S)-2-methylbutanamido)pyrrolo[2,1-f][1,2,4]triazin-7-yl)tetrahydrofuran-3-yl (S)-2-amino-3,3-dimethylbutanoate (13)

The title compound was prepared from carbamate 13.1 according to the procedure of Example 1, Step 4. MS (ESI): mass calcd. for C₃₁H₄₄N₆O₇, 612.33, m/z found 613.15 [M+H]⁺. ¹H NMR (400 MHz, DMSO) δ 10.94 (s, 1H), 8.42 (s, 1H), 7.27 (d, J=4.8 Hz, 1H), 7.12 (d, J=4.8 Hz, 1H), 6.78-6.65 (m, 1H), 5.17-5.01 (m, 2H), 4.55-4.48 (m, 1H), 4.40-4.28 (m, 1H), 4.26-4.17 (m, 1H), 3.11 (s, 1H), 2.98-2.90 (m, 1H), 2.19-2.07 (m, 2H), 1.75-1.66 (m, 1H), 1.64-1.50 (m, 6H), 1.49-1.40 (m, 1H), 1.19-1.03 (m, 6H), 0.98-0.77 (m, 14H).

### Example 14. Synthesis of (2R,3S,4R,5R)-5-cyano-2-((2-cyclohexylacetoxy)methyl)-5-(4-((R)-2,3-dimethylbutanamido)pyrrolo[2,1-f][1,2,4]triazin-7-yl)-4-hydroxytetrahydrofuran-3-yl (S)-2-amino-3,3-dimethylbutanoate (14)

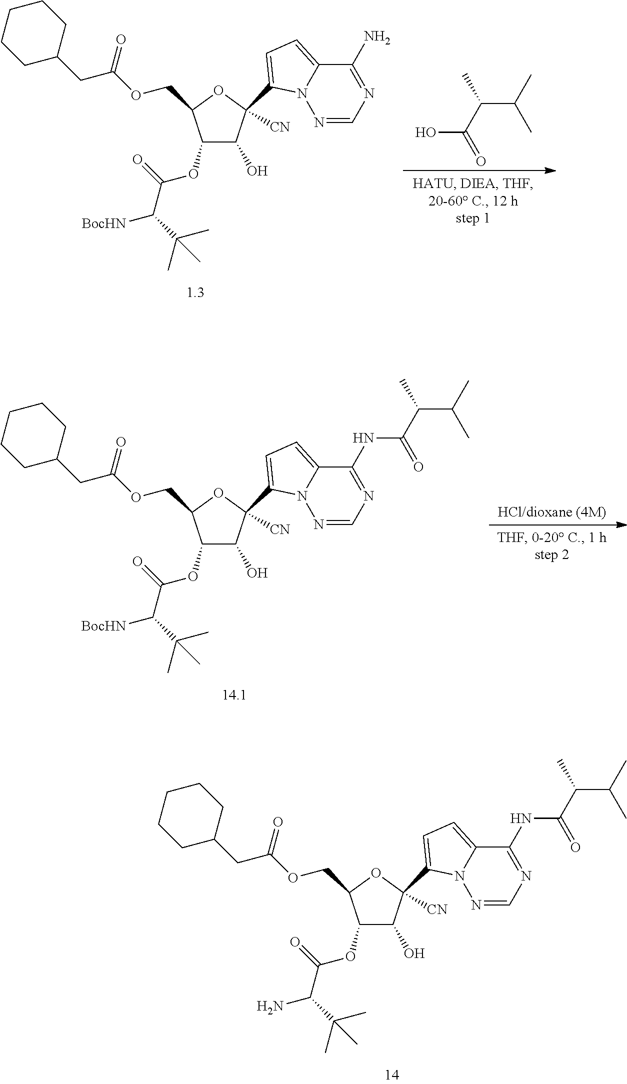

### Step 1. Synthesis of (2R,3S,4R,5R)-5-cyano-2-((2-cyclohexylacetoxy)methyl)-5-(4-((R)-2,3-dimethylbutanamido)pyrrolo[2,1-f][1,2,4]triazin-7-yl)-4-hydroxytetrahydrofuran-3-yl (S)-2-((tert-butoxycarbonyl)amino)-3,3-dimethylbutanoate (14.1)

Title compound 14.1 was prepared according to the procedure of Example 3, Step 1, using 1.3 and (R)-2,3-dimethylbutanoic acid. MS (ESI): mass calcd. for C₃₇H₅₄N₆O₉, 726.40, m/z found 727.25 [M+H]⁺. H NMR (400 MHz, DMSO) δ 10.91 (s, 1H), 8.42 (s, 1H), 7.25 (d, J=4.8 Hz, 1H), 7.12 (d, J=4.8 Hz, 1H), 7.05 (d, J=8.4 Hz, 1H), 6.76 (d, J=6.4 Hz, 1H), 5.15-5.05 (m, 2H), 4.60-4.45 (m, 1H), 4.38-4.25 (m, 1H), 4.19-4.16 (m, 1H), 3.95 (d, J=8.4 Hz, 1H), 2.83-2.73 (m, 1H), 2.18-2.06 (m, 2H), 1.98-1.86 (m, 1H), 1.64-1.50 (m, 6H), 1.40 (s, 9H), 1.15-1.04 (m, 6H), 1.03-0.90 (m, 15H), 0.88-0.78 (m, 2H).

### Step 2. Synthesis of (2R,3S,4R,5R)-5-cyano-2-((2-cyclohexylacetoxy)methyl)-5-(4-((R)-2,3-dimethylbutanamido)pyrrolo[2,1-f][1,2,4]triazin-7-yl)-4-hydroxytetrahydrofuran-3-yl (S)-2-amino-3,3-dimethylbutanoate (14)

The title compound was prepared from carbamate 14.1 according to the procedure of Example 1, Step 4. MS (ESI): mass calcd. for C₃₂H₄₆N₆O₇, 626.34, m/z found 627.15 [M+H]⁺. H NMR (400 MHz, DMSO) 10.91 (s, 1H), 8.42 (s, 1H), 7.25 (d, J=4.8 Hz, 1H), 7.13 (d, J=4.8 Hz, 1H), 6.82-6.68 (m, 1H), 5.15-5.05 (m, 2H), 4.55-4.52 (m, 1H), 4.33-4.26 (m, 1H), 4.25-4.16 (m, 1H), 3.10 (s, 1H), 2.83-2.73 (m, 1H), 2.18-2.06 (m, 2H), 1.97-1.86 (m, 1H), 1.64-1.47 (m, 6H), 1.18-1.03 (m, 6H), 0.96-0.77 (m, 17H).

### Example 15. Synthesis of (2R,3S,4R,5R)-5-cyano-2-((2-cyclohexylacetoxy)methyl)-5-(4-((S)-2,3-dimethylbutanamido)pyrrolo[2,1-f][1,2,4]triazin-7-yl)-4-hydroxytetrahydrofuran-3-yl (S)-2-amino-3,3-dimethylbutanoate (15)

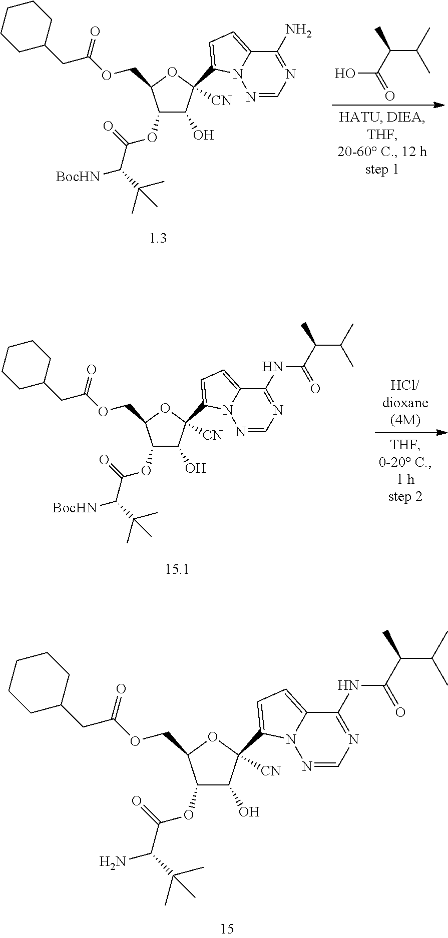

### Step 1. Synthesis of (2R,3S,4R,5R)-5-cyano-2-((2-cyclohexylacetoxy)methyl)-5-(4-((S)-2,3-dimethylbutanamido)pyrrolo[2,1-f][1,2,4]triazin-7-yl)-4-hydroxytetrahydrofuran-3-yl (S)-2-((tert-butoxycarbonyl)amino)-3,3-dimethylbutanoate (15.1)

Title compound 15.1 was prepared according to the procedure of Example 3, Step 1, using 1.3 and (S)-2,3-dimethylbutanoic acid. MS (ESI): mass calcd. for C₃₇H₅₄N₆O₉, 726.40, m/z found 727.30 [M+H]⁺. H NMR (400 MHz, DMSO) δ 10.91 (s, 1H), 8.42 (s, 1H), 7.25 (d, J=4.8 Hz, 1H), 7.12 (d, J=4.8 Hz, 1H), 7.05 (d, J=8.4 Hz, 1H), 6.76 (d, J=6.4 Hz, 1H), 5.15-5.05 (m, 2H), 4.60-4.45 (m, 1H), 4.38-4.27 (m, 1H), 4.22-4.16 (m, 1H), 3.95 (d, J=8.4 Hz, 1H), 2.83-2.73 (m, 1H), 2.18-2.06 (m, 2H), 1.98-1.86 (m, 1H), 1.62-1.50 (m, 6H), 1.39 (s, 9H), 1.13-1.04 (m, 6H), 1.01-0.89 (m, 15H), 0.88-0.80 (m, 2H).

### Step 2. Synthesis of (2R,3S,4R,5R)-5-cyano-2-((2-cyclohexylacetoxy)methyl)-5-(4-((S)-2,3-dimethylbutanamido)pyrrolo[2,1-f][1,2,4]triazin-7-yl)-4-hydroxytetrahydrofuran-3-yl (S)-2-amino-3,3-dimethylbutanoate (15)

The title compound was prepared from carbamate 15.1 according to the procedure of Example 1, Step 4. MS (ESI): mass calcd. for C₃₂H₄₆N₆O₇, 626.34, m/z found 627.20 [M+H]⁺. H NMR (400 MHz, DMSO) 10.92 (s, 1H), 8.42 (s, 1H), 7.25 (d, J=4.8 Hz, 1H), 7.13 (d, J=4.8 Hz, 1H), 6.78-6.68 (m, 1H), 5.13-5.07 (m, 2H), 4.56-4.47 (m, 1H), 4.35-4.16 (m, 2H), 3.10 (s, 1H), 2.84-2.73 (m, 1H), 2.20-2.06 (m, 2H), 1.98-1.86 (m, 1H), 1.64-1.47 (m, 6H), 1.18-1.02 (m, 6H), 0.97-0.78 (m, 17H).

### Example 21. Synthesis of (2R,3S,4R,5R)-5-cyano-5-(4-((R)-2,3-dimethylbutanamido)pyrrolo[2,1-f][1,2,4]triazin-7-yl)-4-hydroxy-2-((2-phenylacetoxy)methyl)tetrahydrofuran-3-yl (S)-2-amino-3,3-dimethylbutanoate (21)

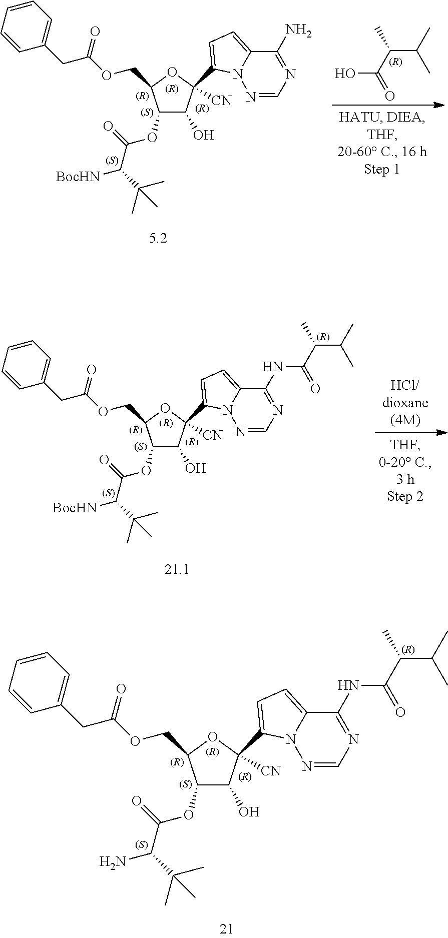

### Step 1. Synthesis of (2R,3S,4R,5R)-5-cyano-5-(4-((R)-2,3-dimethylbutanamido)pyrrolo[2,1-f][1,2,4]triazin-7-yl)-4-hydroxy-2-((2-phenylacetoxy)methyl)tetrahydrofuran-3-yl (S)-2-((tert-butoxycarbonyl)amino)-3,3-dimethylbutanoate (21.1)

The title compound was prepared according to the procedure of Example 3, Step 1, using Compound 5.2 and (R)-2,3-dimethylbutanoic acid. MS (ESI): mass calcd. for C₃₇H₄₈N₆O₉, 720.35, m/z found 721.2 [M+H]⁺.

### Step 2. Synthesis of (2R,3S,4R,5R)-5-cyano-5-(4-((R)-2,3-dimethylbutanamido)pyrrolo[2,1-f][1,2,4]triazin-7-yl)-4-hydroxy-2-((2-phenylacetoxy)methyl)tetrahydrofuran-3-yl (S)-2-amino-3,3-dimethylbutanoate (21)

The title compound was prepared according to the procedure of Example 1, Step 4, using Compound 21.1. MS (ESI): mass calcd. for C₃₂H₄₀N₆O₇, 620.30, m/z found 621.15 [M+H]⁺. H NMR (400 MHz, DMSO) δ 10.93 (s, 1H), 8.42 (s, 1H), 7.33-7.14 (m, 6H), 7.09 (d, J=4.8 Hz, 1H), 6.70 (d, J=6.0 Hz, 1H), 5.17-5.09 (m, 1H), 5.05 (t, J=5.6 Hz, 1H), 4.52-4.50 (m, 1H), 4.34 (dd, J=12.4, 3.6 Hz, 1H), 4.25 (dd, J=12.4, 4.8 Hz, 1H), 3.66 (s, 2H), 3.10 (s, 1H), 2.86-2.73 (m, 1H), 1.92-1.85 (m, 1H), 1.10 (d, J=6.8 Hz, 3H), 1.03-0.84 (m, 15H).

### Example 22. Synthesis of (2R,3S,4R,5R)-5-cyano-5-(4-((S)-2,3-dimethylbutanamido)pyrrolo[2,1-f][1,2,4]triazin-7-yl)-4-hydroxy-2-((2-phenylacetoxy)methyl)tetrahydrofuran-3-yl (S)-2-amino-3,3-dimethylbutanoate (22)

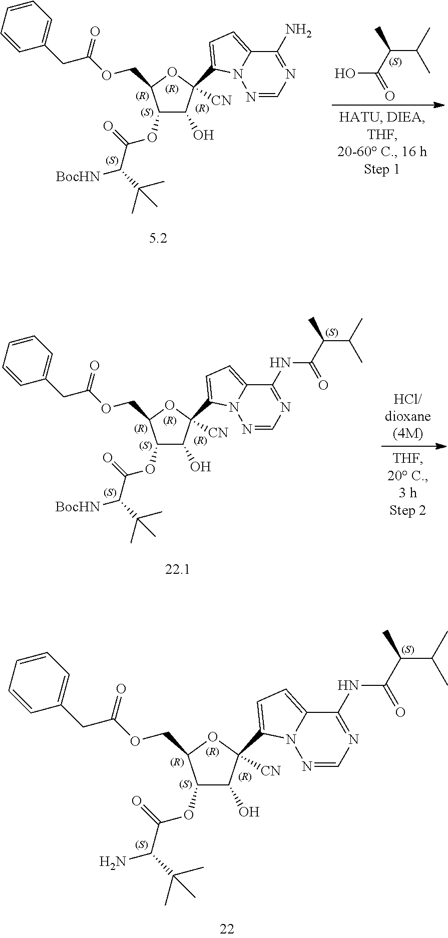

### Step 1. Synthesis of (2R,3S,4R,5R)-5-cyano-5-(4-((S)-2,3-dimethylbutanamido)pyrrolo[2,1-f][1,2,4]triazin-7-yl)-4-hydroxy-2-((2-phenylacetoxy)methyl)tetrahydrofuran-3-yl (S)-2-((tert-butoxycarbonyl)amino)-3,3-dimethylbutanoate (22.1)

The title compound was prepared according to the procedure of Example 3, Step 1, using Compound 5.2 and (S)-2,3-dimethylbutanoic acid. MS (ESI): mass calcd. for C₃₇H₄₈N₆O₉, 720.35, m/z found 721.2 [M+H]⁺.

### Step 2. Synthesis of (2R,3S,4R,5R)-5-cyano-5-(4-((S)-2,3-dimethylbutanamido)pyrrolo[2,1-f][1,2,4]triazin-7-yl)-4-hydroxy-2-((2-phenylacetoxy)methyl)tetrahydrofuran-3-yl (S)-2-amino-3,3-dimethylbutanoate (22)

The title compound was prepared according to the procedure of Example 1, Step 4, using Compound 22.1. MS (ESI): mass calcd. for C₃₂H₄₀N₆O₇, 620.30, m/z found 621.15 [M+H]⁺. ¹HNMR (400 MHz, DMSO) δ 10.92 (s, 1H), 8.42 (s, 1H), 7.26-7.24 (m, 4H), 7.21-7.16 (m, 2H), 7.09 (d, J=4.8 Hz, 1H), 6.70 (d, J=6.0 Hz, 1H), 5.15-5.09 (m, 1H), 5.05 (t, J=5.6 Hz, 1H), 4.52-4.50 (m, 1H), 4.34 (dd, J=12.4, 3.6 Hz, 1H), 4.25 (dd, J=12.4, 4.8 Hz, 1H), 3.66 (s, 2H), 3.12 (s, 1H), 2.78-2.75 (m, 1H), 1.92-1.85 (m, 1H), 1.10 (d, J=6.8 Hz, 3H), 0.97-0.91 (m, 15H).

### Example 29. Synthesis of (2R,3S,4R,5R)-5-cyano-4-hydroxy-5-(4-(3-isopropylureido)pyrrolo[2,1-f][1,2,4]triazin-7-yl)-2-((2-phenylacetoxy)methyl)tetrahydrofuran-3-yl (S)-2-amino-3,3-dimethylbutanoate (29)

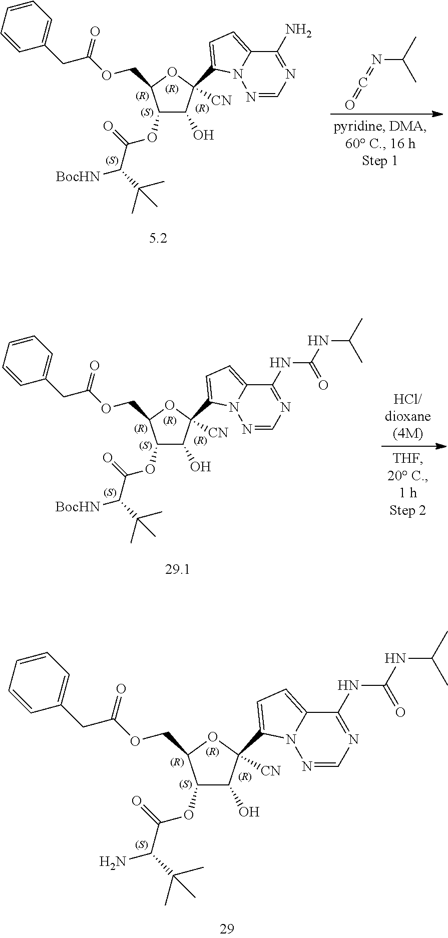

### Step 1: Synthesis of (2R,3S,4R,5R)-5-cyano-4-hydroxy-5-(4-(3-isopropylureido)pyrrolo[2,1-f][1,2,4]triazin-7-yl)-2-((2-phenylacetoxy)methyl)tetrahydrofuran-3-yl (S)-2-((tert-butoxycarbonyl)amino)-3,3-dimethylbutanoate (29.1)

The title compound was prepared according to the procedure of Example 4, Step 1, using Compound 5.2 and 2-isocyanatopropane. MS (ESI): mass calcd. for C₃₅H₄₅N₇O₉, 707.33, m/z found 708.25 [M+H]⁺.

### Step 2: Synthesis of (2R,3S,4R,5R)-5-cyano-4-hydroxy-5-(4-(3-isopropylureido)pyrrolo[2,1-f][1,2,4]triazin-7-yl)-2-((2-phenylacetoxy)methyl)tetrahydrofuran-3-yl (S)-2-amino-3,3-dimethylbutanoate (29)

The title compound was prepared according to the procedure of Example 1, Step 4, using compound 29.1. MS (ESI): mass calcd. for C₃₀H₃₇N₇O₇, 607.28, found 608.15 [M+H]⁺. H NMR (400 MHz, DMSO) δ 10.53 (s, 1H), 9.32 (d, J=7.2 Hz, 1H), 8.29 (s, 1H), 7.49 (d, J=4.8 Hz, 1H), 7.30-7.16 (m, 5H), 6.99 (d, J=4.8 Hz, 1H), 6.69 (d, J=6.0 Hz, 1H), 5.17-5.08 (m, 1H), 5.03 (t, J=5.6 Hz, 1H), 4.51 (dd, J=8.0, 4.4 Hz, 1H), 4.28-4.20 (m, 2H), 3.93-3.85 (m, 1H), 3.66 (d, J=2.8 Hz, 2H), 3.10 (s, 1H), 1.22 (d, J=6.4 Hz, 6H), 0.94 (s, 9H).

### Example 30. Synthesis of (2R,3S,4R,5R)-5-(4-(((acetoxymethoxy)carbonyl)amino)pyrrolo[2,1-f][1,2,4]triazin-7-yl)-5-cyano-4-hydroxy-2-((2-phenylacetoxy)methyl)tetrahydrofuran-3-yl (S)-2-amino-3,3-dimethylbutanoate (30)

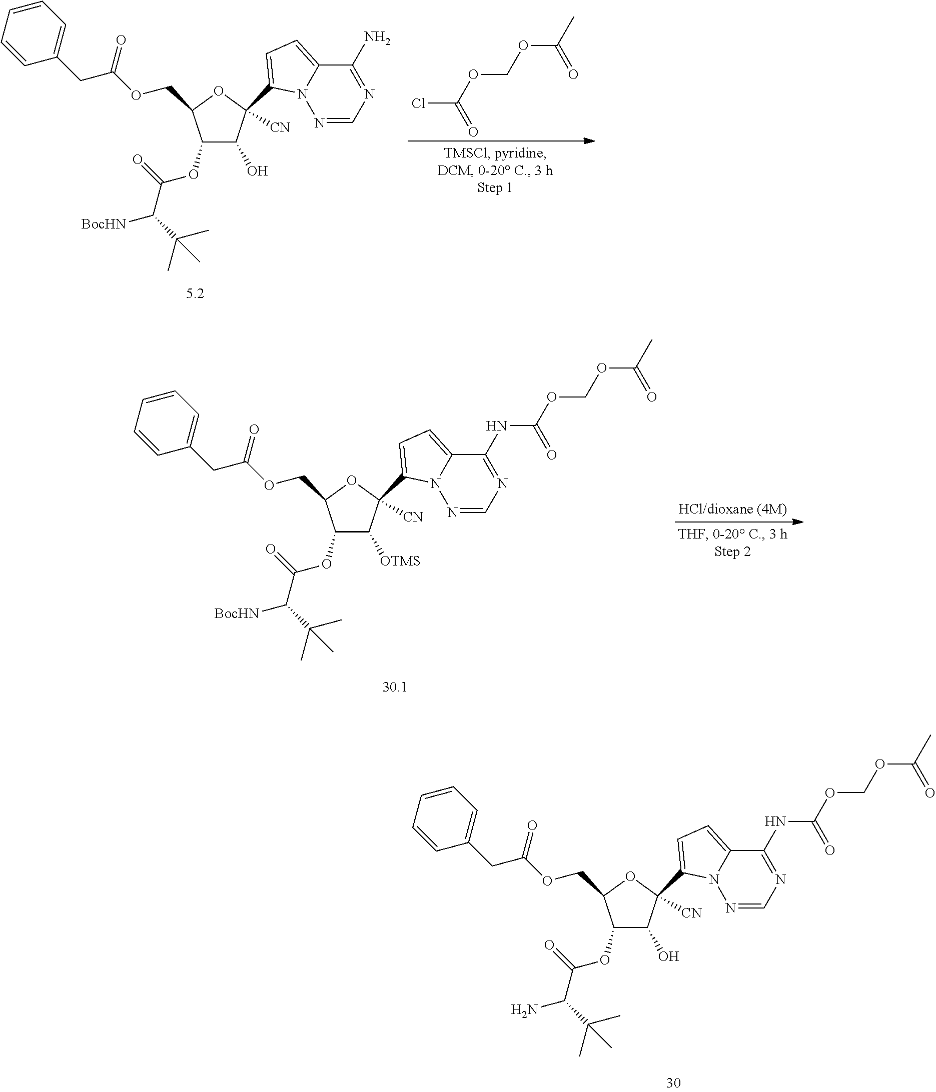

### Step 1. Synthesis of (2R,3S,4R,5R)-5-(4-(((acetoxymethoxy)carbonyl)amino)pyrrolo[2,1-f][1,2,4]triazin-7-yl)-5-cyano-4-hydroxy-2-((2-phenylacetoxy)methyl)tetrahydrofuran-3-yl (S)-2-((tert-butoxycarbonyl)amino)-3,3-dimethylbutanoate (30.1)

The title compound was prepared according to the procedure of Example 1, Step 3, using Compound 30.2 and ((chlorocarbonyl)oxy)methyl acetate. MS (ESI): mass calcd. for C₃₈H₅₀N₆O₁₂Si, 810.33, m/z found 811.2 [M+H]⁺.

### Step 2. Synthesis of (2R,3S,4R,5R)-5-(4-(((acetoxymethoxy)carbonyl)amino)pyrrolo[2,1-f][1,2,4]triazin-7-yl)-5-cyano-4-hydroxy-2-((2-phenylacetoxy)methyl)tetrahydrofuran-3-yl (S)-2-amino-3,3-dimethylbutanoate (Compound 30)

The title compound was prepared according to the procedure of Example 1, Step 4, using compound 30.3. MS (ESI): mass calcd. for C₃₀H₃₄N₆O₁₀, 638.23, m/z found 639.3 [M+H]⁺. H NMR (400 MHz, DMSO) δ 8.38 (s, 1H), 7.42-7.14 (m, 6H), 7.04 (d, J=4.4 Hz, 1H), 6.70 (d, J=6.4 Hz, 1H), 5.81 (s, 2H), 5.20-5.10 (m, 1H), 5.04 (t, J=6.0 Hz, 1H), 4.51 (dd, J=8.0, 4.4 Hz, 1H), 4.33 (dd, J=12.4, 3.6 Hz, 1H), 4.25 (dd, J=12.4, 5.2 Hz, 1H), 3.66 (s, 2H), 3.11 (s, 1H), 2.12 (s, 3H), 0.93 (s, 9H).

### Example 31. Synthesis of (2R,3S,4R,5R)-5-cyano-4-hydroxy-5-(4-isobutyramidopyrrolo[2,1-f][1,2,4]triazin-7-yl)-2-((2-phenylacetoxy)methyl)tetrahydrofuran-3-yl (S)-2-amino-3,3-dimethylbutanoate (31)

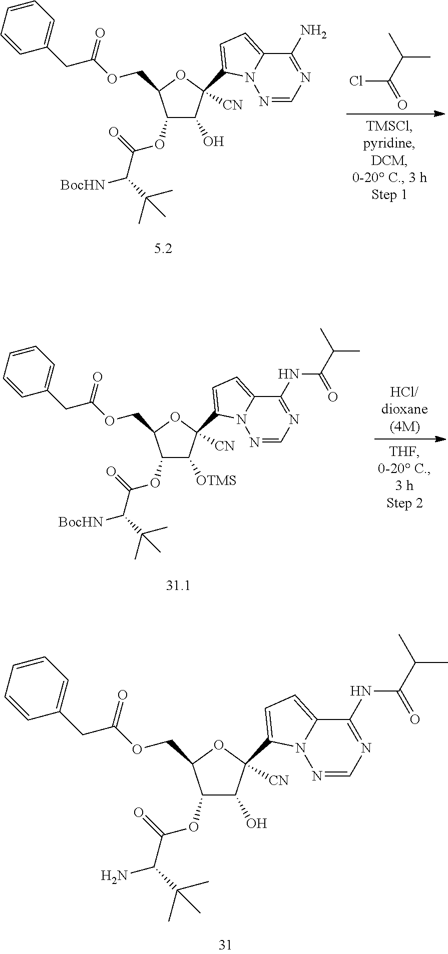

### Step 1. Synthesis of (2R,3R,4R,5R)-5-cyano-5-(4-isobutyramidopyrrolo[2,1-f][1,2,4]triazin-7-yl)-2-((2-phenylacetoxy)methyl)-4-((trimethylsilyl)oxy)tetrahydrofuran-3-yl (S)-2-((tert-butoxycarbonyl)amino)-3,3-dimethylbutanoate (31.1)

The title compound was prepared according to the procedure of Example 1, Step 3, using compound 5.2 and isobutyryl chloride. MS (ESI): mass calcd. for C₃₈H₅₂N₆O₉Si, 764.36, m/z found 765.3.

### Step 2. Synthesis of (2R,3S,4R,5R)-5-cyano-4-hydroxy-5-(4-isobutyramidopyrrolo[2,1-f][1,2,4]triazin-7-yl)-2-((2-phenylacetoxy)methyl)tetrahydrofuran-3-yl (S)-2-amino-3,3-dimethylbutanoate (31)

The title compound was prepared according to the procedure of Example 1, Step 4, using compound 31.1. MS (ESI): mass calcd. for C₃₀H₃₆N₆O₇, 592.26, m/z found 593.3 [M+H]⁺. ¹H NMR (400 MHz, DMSO) δ 10.92 (s, 1H), 8.41 (s, 1H), 7.32-7.15 (m, 6H), 7.08 (d, J=4.8 Hz, 1H), 6.71 (d, J=6.4 Hz, 1H), 5.17-5.08 (m, 1H), 5.05 (t, J=6.0 Hz, 1H), 4.52 (dd, J=8.0, 4.4 Hz, 1H), 4.33 (dd, J=12.0, 3.6 Hz, 1H), 4.25 (dd, J=12.4, 4.8 Hz, 1H), 3.66 (s, 2H), 3.18-3.11 (m, 1H), 3.09 (s, 1H), 1.16 (d, J=6.8 Hz, 6H), 0.93 (s, 9H).

### Example 32. Synthesis of ((2R,3S,4R,5R)-5-(4-aminopyrrolo[2,1-f][1,2,4]triazin-7-yl)-5-cyano-3-(2-cyclohexylacetoxy)-4-hydroxytetrahydrofuran-2-yl)methyl (S)-2-amino-3,3-dimethylbutanoate (32)

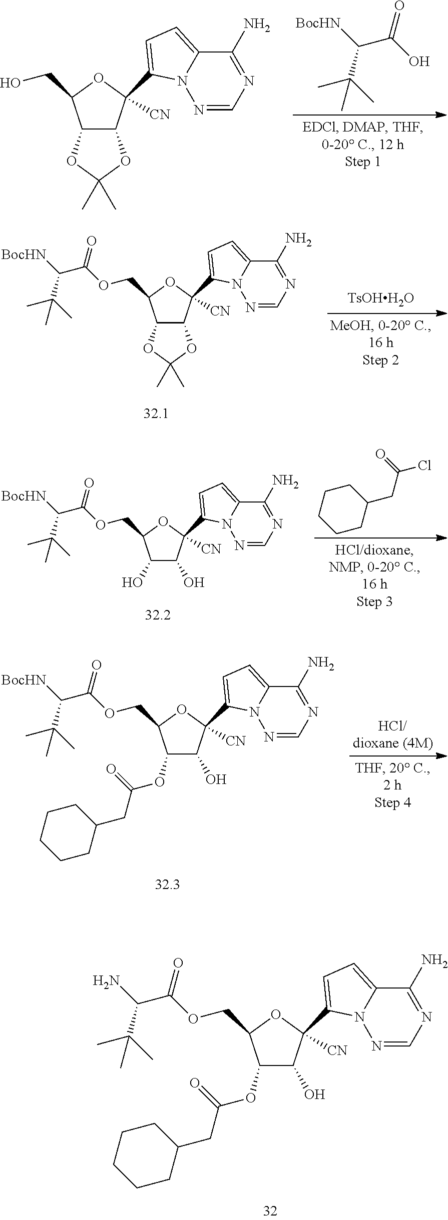

### Step 1. Synthesis of ((3aR,4R,6R,6aR)-6-(4-aminopyrrolo[2,1-f][1,2,4]triazin-7-yl)-6-cyano-2,2-dimethyltetrahydrofuro[3,4-d][1,3]dioxol-4-yl)methyl (S)-2-((tert-butoxycarbonyl)amino)-3,3-dimethylbutanoate (32.1)

To a solution of (3aR,4R,6R,6aR)-4-(4-aminopyrrolo[2,1-f][1,2,4]triazin-7-yl)-6-(hydroxymethyl)-2,2-dimethyltetrahydrofuro[3,4-d][1,3]dioxole-4-carbonitrile (1.50 g, 4.53 mmol) in THF (80 mL) was added (2S)-2-{[(tert-butoxy)carbonyl]amino}-3,3-dimethylbutanoic acid (1.35 g, 5.85 mmol), EDCI (2.59 g, 13.5 mmol) and DMAP (1.65 g, 13.5 mmol), the mixture was stirred at 25° C. for 16 h. After completion, the mixture was concentrated in vacuo to afford a residue. Then the reaction residue was diluted with water (100 mL) and extracted with EtOAc (100 mL×3). The organic layer was dried over sodium sulphate and concentrated under reduce pressure to afford a crude. The residue was purified by prep-HPLC [Gradient: 5-40% ACN in water (0.1% FA)] to obtain 32.1 (1.80 g, 69% yield) as a white solid. MS (ESI): mass calcd. for C₂₋₆H₃₆N₆O₇, 544.26 m/z found 545.3 [M+H]⁺. ¹H NMR (400 MHz, DMSO) δ 7.95 (br s, 3H), 7.09 (d, J=8.4 Hz, 1H), 6.90 (d, J=4.4 Hz, 1H), 6.85 (d, J=4.4 Hz, 1H), 5.44 (d, J=6.4 Hz, 1H), 4.94 (dd, J=6.4, 3.2 Hz, 1H), 4.52 (s, 1H), 4.27-4.20 (m, 1H), 4.17-4.11 (m, 1H), 3.82 (d, J=8.4 Hz, 1H), 1.64 (s, 3H), 1.40-1.26 (m, 1H), 0.87 (s, 9H).

### Step 2. ((2R,3S,4R,5R)-5-(4-aminopyrrolo[2,1-f][1,2,4]triazin-7-yl)-5-cyano-3,4-dihydroxytetrahydrofuran-2-yl)methyl (S)-2-((tert-butoxycarbonyl)amino)-3,3-dimethylbutanoate (32.2)

To a solution of compound 32.1 (600 mg, 1.10 mmol) in MeOH (12 mL) was added TsOH·H₂O (419 mg, 2.20 mmol) at 0° C. The mixture was stirred at 0° C. for 30 min and then warmed up to 20° C. and stirred for 16 h. The mixture was adjusted to pH 8 with aqueous NaHCO₃. The reaction mixture was concentrated under reduced pressure to remove MeOH. Then the reaction residue was diluted with water (50 mL) and extracted with EtOAc (50 mL×3). The organic phase was washed with brine water (50 mL×3), then dried over anhydrous sodium sulfate, filtered, and concentrated to remove the solvent. The crude product was purified by chromatography (SiO₂, Dichloromethane/Methanol from 0% to 6%) to obtain 32.2 (250 mg, 42.7% yield) as a white solid. MS (ESI): mass calcd. for C₂₃H₃₂N₆O₇, 504.23 m/z found 505.2 [M+H]⁺. ¹H NMR (400 MHz, DMSO) δ 7.92 (br s, 3H), 7.05 (d, J=8.4 Hz, 1H), 6.92 (d, J=4.4 Hz, 1H), 6.83 (d, J=4.4 Hz, 1H), 6.33 (d, J=6.0 Hz, 1H), 5.39 (d, J=6.0 Hz, 1H), 4.7 0 (t, J=5.6 Hz, 1H), 4.40-4.14 (m, 3H), 3.96-3.80 (m, 2H), 1.43-1.29 (m, 9H), 0.89 (s, 9H).

### Step 3. Synthesis of ((2R,3S,4R,5R)-5-(4-aminopyrrolo[2,1-f][1,2,4]triazin-7-yl)-5-cyano-3-(2-cyclohexylacetoxy)-4-hydroxytetrahydrofuran-2-yl)methyl (S)-2-((tert-butoxycarbonyl)amino)-3,3-dimethylbutanoate (32.3)

To a solution of 32.2 (250 mg, 0.496 mmol) in NMP (1 mL), 4 M HCl solution in 1-4 dioxane (0.2 mL, 4 M) was added and stirred at 20° C. for 15 min. The reaction mixture was cooled at 0° C. and 2-cyclohexylacetyl chloride (0.61 mL, 3.96 mmol) was added at once. The reaction was stirred at 20° C. for 16 h. The reaction was diluted with ACN (1 mL) and purified by prep-HPLC (column: Gemini—C₁₈150×21.2 mm, 5 μm; mobile phase: ACN—H₂O (0.1% FA); gradient: 35%-70%) to obtain 32.3 (140 mg, 42.7% yield) as a white solid. MS (ESI): mass calcd. for C₃₁H₄₄N₆O₈, 628.32 m/z found 629.3 [M+H]⁺. ¹H NMR (400 MHz, DMSO) δ 7.93 (br s, 3H), 7.07 (d, J=8.4 Hz, 1H), 6.93 (d, J=4.8 Hz, 1H), 6.88 (d, J=4.8 Hz, 1H), 6.66-6.59 (m, 1H), 5.09 (d, J=5.2 Hz, 2H), 4.46 (d, J=4.0 Hz, 1H), 4.33-4.25 (m, 2H), 3.84 (d, J=8.4 Hz, 1H), 2.25 (d, J=6.4 Hz, 2H), 1.80-1.68 (m, 3H), 1.66-1.55 (m, 3H), 1.37 (s, 7H), 1.29-1.04 (m, 5H), 1.02-0.91 (m, 2H), 0.85 (s, 9H).

### Step 4. Synthesis of ((2R,3S,4R,5R)-5-(4-aminopyrrolo[2,1-f][1,2,4]triazin-7-yl)-5-cyano-3-(2-cyclohexylacetoxy)-4-hydroxytetrahydrofuran-2-yl)methyl (S)-2-amino-3,3-dimethylbutanoate (32)

The title compound 32 was prepared according to the procedure of Example 1, Step 4, using 32.3. MS (ESI): mass calcd. for C₂₈H₃₈N₆O₆, 528.27 m/z found 529.3 [M+H]⁺. ¹H NMR (400 MHz, DMSO) δ 7.92 (br s, 3H), 6.93 (d, J=4.4 Hz, 1H), 6.87 (d, J=4.8 Hz, 1H), 6.62 (d, J=6.0 Hz, 1H), 5.13-5.07 (m, 2H), 4.49-4.44 (m, 1H), 4.37-4.17 (m, 2H), 2.96 (s, 1H), 2.25 (d, J=6.4 Hz, 2H), 1.81-1.55 (m, 6H), 1.31-1.07 (m, 3H), 1.00-0.89 (m, 2H), 0.86-0.78 (m, 9H).

### Example 33. Synthesis of ((2R,3S,4R,5R)-5-(4-acetamidopyrrolo[2,1-f][1,2,4]triazin-7-yl)-5-cyano-3-(2-cyclohexylacetoxy)-4-hydroxytetrahydrofuran-2-yl)methyl (S)-2-amino-3,3-dimethylbutanoate (33)

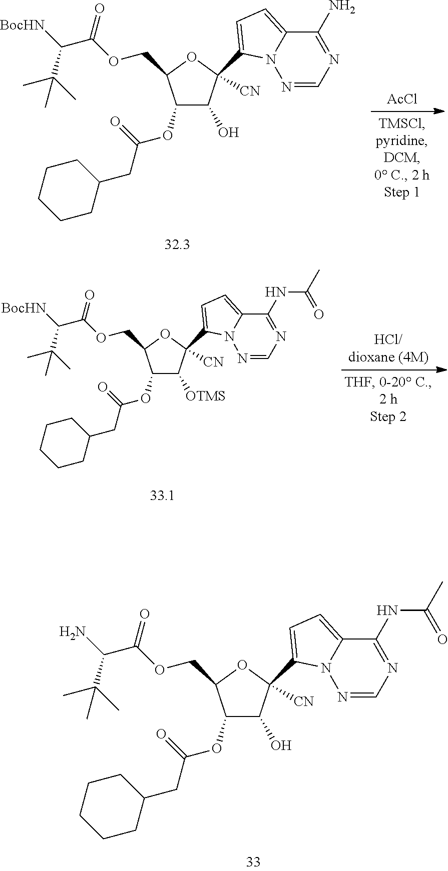

### Step 1. Synthesis of ((2R,3R,4R,5R)-5-(4-acetamidopyrrolo[2,1-f][1,2,4]triazin-7-yl)-5-cyano-3-(2-cyclohexylacetoxy)-4-((trimethylsilyl)oxy)tetrahydrofuran-2-yl)methyl (S)-2-((tert-butoxycarbonyl)amino)-3,3-dimethylbutanoate (33.1)

The title compound 33.1 was prepared according to the procedure of Example 1, Step 3, using 32.3 and acetyl chloride. MS (ESI): mass calcd. for C₃₆H₅₄N₆O₉Si, 742.37 m/z found 743.3 [M+H]⁺. ¹H NMR (400 MHz, DMSO) δ 10.99 (s, 1H), 8.38 (s, 1H), 7.35 (d, J=4.8 Hz, 1H), 7.19-7.06 ((m, 2H), 5.31 (d, J=5.2 Hz, 1H), 5.18 (t, J=5.2 Hz, 1H), 4.54 (dd, J=9.2, 4.8 Hz, 1H), 4.36 (s, 2H), 3.88 (d, J=8.4 Hz, 1H), 2.39 (s, 3H), 2.29-2.21 (m, 2H), 1.80-1.67 (m, 3H), 1.70-1.54 (m, 3H), 1.45-1.30 (m, 9H), 1.23-1.05 (m, 3H), 1.04-0.92 (m, 2H), 0.87 (s, 9H), −0.03 (s, 9H).

### Step 2. Synthesis of ((2R,3S,4R,5R)-5-(4-acetamidopyrrolo[2,1-f][1,2,4]triazin-7-yl)-5-cyano-3-(2-cyclohexylacetoxy)-4-hydroxytetrahydrofuran-2-yl)methyl (S)-2-amino-3,3-dimethylbutanoate (33)

The title compound 33 was prepared according to the procedure of Example 1, Step 4, using 33.1. MS (ESI): mass calcd. for C₂₈H₃₈N₆O₇, 570.28 m/z found 571.3 [M+H]⁺. ¹H NMR (400 MHz, DMSO) δ 10.99 (s, 1H), 8.40 (s, 1H), 7.32 (d, J=4.8 Hz, 1H), 7.11 (d, J=4.8 Hz, 1H), 6.80-6.68 (m, 1H), 5.17-5.11 (m, 1H), 5.09-5.04 (m, 1H), 4.54-4.48 (m, 1H), 4.33-4.22 (m, 2H), 2.98-2.96 (m, 1H), 2.40 (s, 3H), 2.28-2.25 (m, 2H), 1.80-1.69 (m, 3H), 1.67-1.56 (m, 3H), 1.27-1.08 (m, 3H), 1.00-0.89 (m, 2H), 0.85-0.79 (m, 9H).

### Example 34. Synthesis of (2R,3S,4R,5R)-5-(4-aminopyrrolo[2,1-f][1,2,4]triazin-7-yl)-5-cyano-2-((2-cyclohexylacetoxy)methyl)-4-hydroxytetrahydrofuran-3-yl (S)-2-amino-3,3-dimethylbutanoate (34)

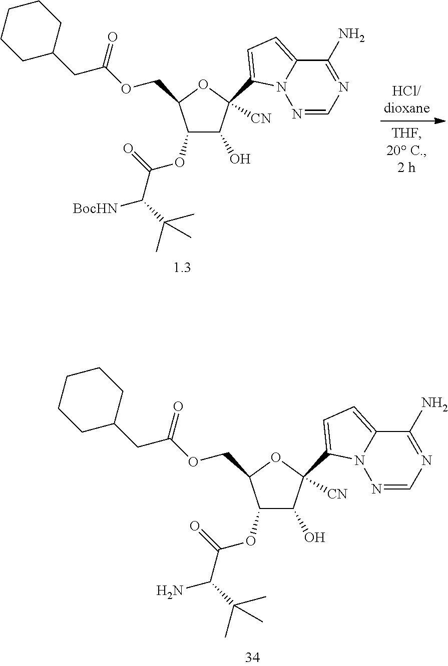 [SMILES: CC(C)(C)OC(=O)N[C@H](C(=O)O[C@H]1[C@@H](O)[C@](C#N)(c2ccc3c(N)ncnn23)O[C@@H]1COC(=O)CC1CCCCC1)C(C)(C)C ; CC(C)(C)[C@H](N)C(=O)O[C@H]1[C@@H](O)[C@](C#N)(c2ccc3c(N)ncnn23)O[C@@H]1COC(=O)CC1CCCCC1]

The title compound 34 was prepared from compound 1.3 according to the procedure of Example 1, Step 4. MS (ESI): mass calcd. for C₂₋₆H₃₆N₆O₆, 528.27, m/z found 529.2 [M+H]⁺. ¹H NMR (400 MHz, DMSO) δ 7.98-7.93 (m, 3H), 6.94 (d, J=4.4 Hz, 1H), 6.86 (d, J=4.4 Hz, 1H), 6.63-6.60 (m, 1H), 5.22-4.98 (m, 2H), 4.46 (dd, J=8.4, 4.4 Hz, 1H), 4.30 (dd, J=12.4, 3.6 Hz, 1H), 4.25-4.15 (m, 1H), 3.08 (s, 1H), 2.15 (dd, J=6.8, 3.6 Hz, 2H), 1.58-1.57 (m, 6H), 1.11-1.08 (m, 3H), 1.00-0.77 (m, 11H).

### Example 35. Synthesis of ((2R,3S,4R,5R)-5-cyano-3,4-dihydroxy-5-(4-((((pivaloyloxy)methoxy)carbonyl)amino)pyrrolo[2,1-f][1,2,4]triazin-7-yl)tetrahydrofuran-2-yl)methyl (S)-2-amino-3,3-dimethylbutanoate (35)

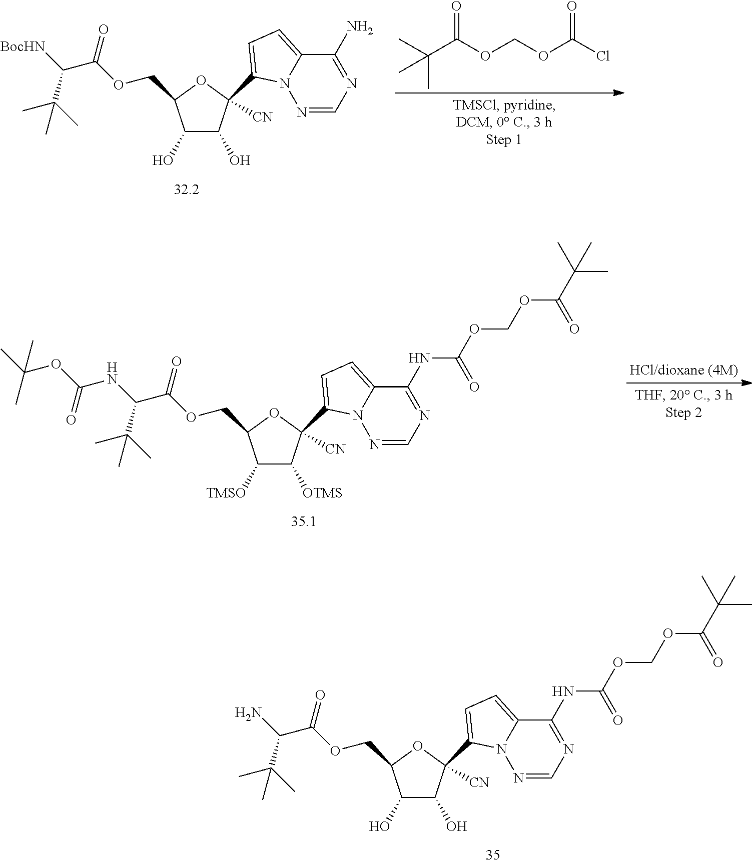

### Step 1. Synthesis of ((2R,3R,4R,5R)-5-cyano-5-(4-((((pivaloyloxy)methoxy)carbonyl)amino)pyrrolo[2,1-f][1,2,4]triazin-7-yl)-3,4-bis((trimethylsilyl)oxy)tetrahydrofuran-2-yl)methyl (S)-2-((tert-butoxycarbonyl)amino)-3,3-dimethylbutanoate (35.1)

The title compound 35.1 was prepared according to the procedure of Example 1, Step 3, using compound 32.2 and ((chlorocarbonyl)oxy)methyl pivalate. MS (ESI): mass calcd. for C₃₆H₅₈N₆O₁Si₂, 806.37, m/z found 807.3 [M+H]⁺.

### Step 2. Synthesis of ((2R,3S,4R,5R)-5-cyano-3,4-dihydroxy-5-(4-((((pivaloyloxy)methoxy)carbonyl)amino)pyrrolo[2,1-f][1,2,4]triazin-7-yl)tetrahydrofuran-2-yl)methyl (S)-2-amino-3,3-dimethylbutanoate (35)

The title compound 35 was prepared according to the procedure of Example 1, Step 4, using Compound 35.1. MS (ESI): mass calcd. for C₂₅H₃₄N₆O₉, 562.24, m/z found 563.2 [M+H]⁺. ¹H NMR (400 MHz, DMSO) δ 8.37 (s, 1H), 7.29 (d, J=4.4 Hz, 1H), 7.07 (d, J=4.8 Hz, 1H), 6.45 (d, J=6.0 Hz, 1H), 5.84 (s, 2H), 5.46 (d, J=5.6 Hz, 1H), 4.72-4.67 (m, 1H), 4.31-4.23 (m, 3H), 3.94 (d, J=4.4 Hz, 1H), 3.07 (s, 1H), 1.17 (s, 9H), 0.84 (s, 9H).

### Example 36. Synthesis of ((2R,3S,4R,5R)-5-(4-(((acetoxymethoxy)carbonyl)amino)pyrrolo[2,1-f][1,2,4]triazin-7-yl)-5-cyano-3-(2-cyclohexylacetoxy)-4-hydroxytetrahydrofuran-2-yl)methyl (S)-2-amino-3,3-dimethylbutanoate (36)

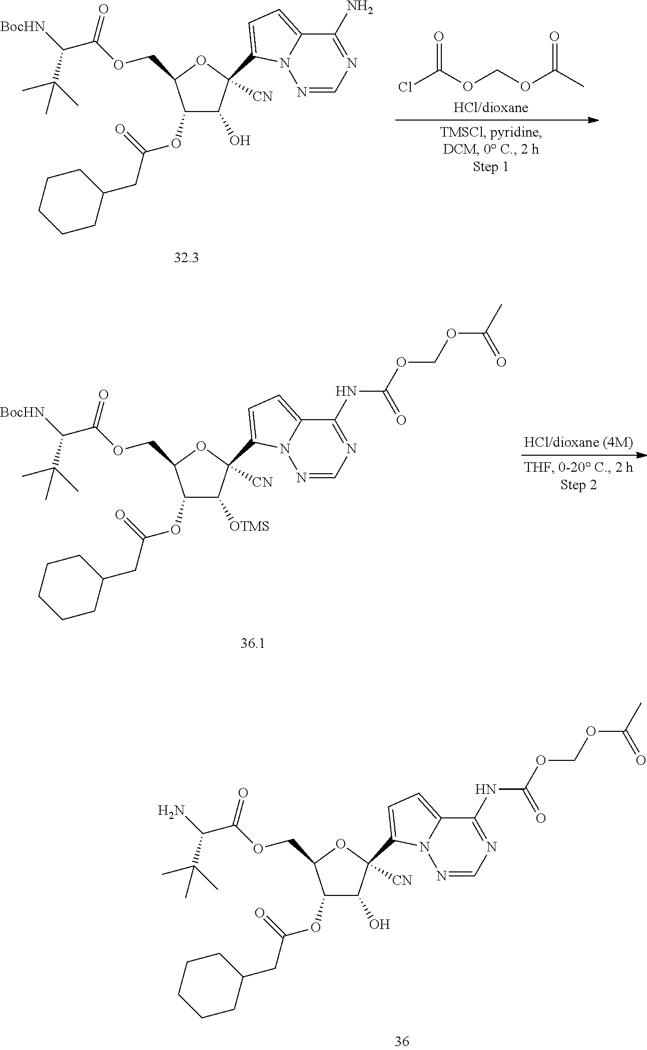

The title compound was prepared according to the procedure of Example 33, utilizing ((chlorocarbonyl)oxy)methyl acetate in place of acetyl chloride in Step 1. MS (ESI): mass calcd. for C₃₀H₄₀N₆O₁₀, 644.28, m/z found 645.3 [M+H]⁺. ¹H NMR (400 MHz, DMSO) δ 8.37 (s, 1H), 7.29 (d, J=4.8 Hz, 1H), 7.09 (d, J=4.8 Hz, 1H), 6.73 (d, J=6.4 Hz, 1H), 5.81 (s, 2H), 5.22-5.09 (m, 1H), 5.06 (t, J=6.0 Hz, 1H), 4.51 (dd, J=8.4, 4.4 Hz, 1H), 4.33-4.22 (m, 2H), 3.00-2.94 (m, 1H), 2.35-2.24 (m, 2H), 2.12 (s, 3H), 1.83-1.68 (m, 3H), 1.67-1.56 (m, 3H), 1.27-1.08 (m, 3H), 1.01-0.92 (m, 2H), 0.86-0.75 (m, 9H).

### Example 37. Synthesis of ((2R,3S,4R,5R)-5-(4-aminopyrrolo[2,1-f][1,2,4]triazin-7-yl)-5-cyano-3-(2-cyclohexylacetoxy)-4-hydroxytetrahydrofuran-2-yl)methyl (R)-2-amino-3,3-dimethylbutanoate (37)

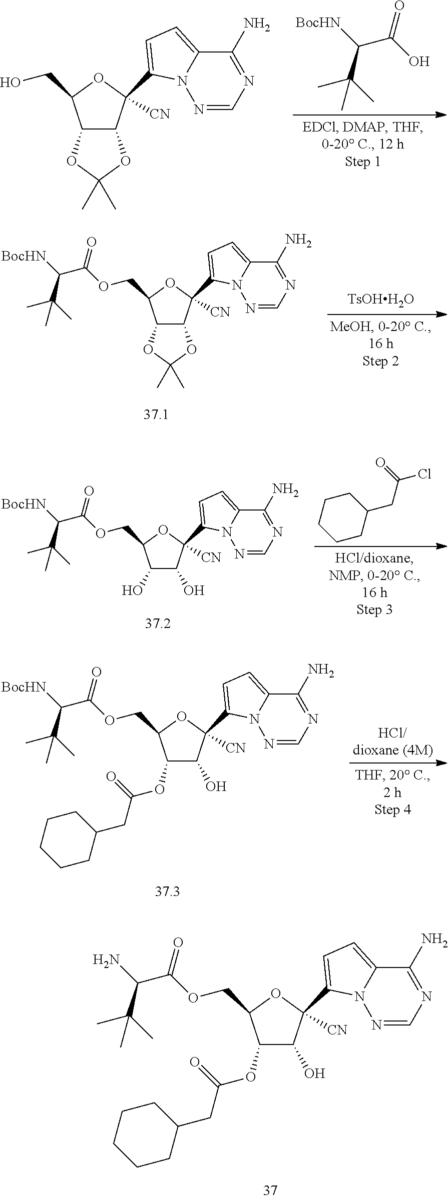

The title compound 37 was prepared according to the procedure of Example 32, using (2R)-2-{[(tert-butoxy)carbonyl]amino}-3,3-dimethylbutanoic acid in Step 1. MS (ESI): mass calcd. for C₂₋₆H₃₆N₆O₆, 528.27, m/z found 529.3 [M+H]⁺. ¹H NMR (400 MHz, DMSO) δ 7.93 (br s, 3H), 6.92 (d, J=4.4 Hz, 1H), 6.86 (d, J=4.8 Hz, 1H), 6.58 (d, J=6.0 Hz, 1H), 5.19-5.14 (m, 1H), 5.11 (t, J=6.0 Hz, 1H), 4.45 (dd, J=8.8, 4.4 Hz, 1H), 4.32-4.18 (m, 2H), 2.96 (s, 1H), 2.26 (d, J=6.8 Hz, 2H), 1.78-1.70 (m, 3H), 1.69-1.54 (m, 3H), 1.29-1.08 (m, 3H), 1.28-1.10 (m, 2H), 0.85-0.74 (m, 9H).

### Example 38. Synthesis of ((2R,3S,4R,5R)-5-(4-aminopyrrolo[2,1-f][1,2,4]triazin-7-yl)-5-cyano-3-(2-cyclohexylacetoxy)-4-hydroxytetrahydrofuran-2-yl)methyl (S)-2-acetamido-3,3-dimethylbutanoate (38)

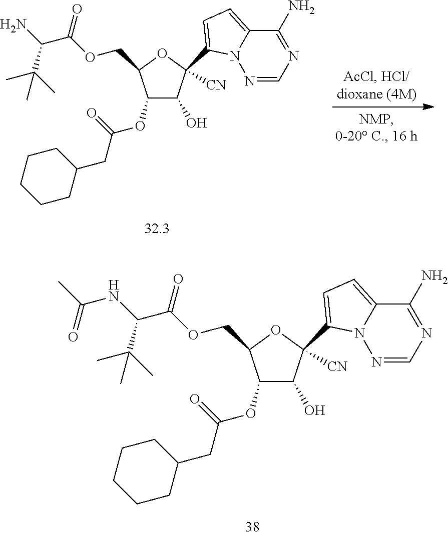 [SMILES: CC(C)(C)[C@H](N)C(=O)OC[C@H]1O[C@@](C#N)(c2ccc3c(N)ncnn23)[C@H](O)[C@@H]1OC(=O)CC1CCCCC1 ; CC(=O)N[C@H](C(=O)OC[C@H]1O[C@@](C#N)(c2ccc3c(N)ncnn23)[C@H](O)[C@@H]1OC(=O)CC1CCCCC1)C(C)(C)C]

To a solution of 32.3 (60.0 mg, 0.113 mmol) in NMP (2 mL) was added HCl in dioxane (4 M, 0.1 mL). The reaction was stirred at 25° C. for 15 min, then AcCl (266 mg, 3.41 mmol) was added to the solution at 0° C., the resulting mixture was stirred at 25° C. for 16 h. The mixture was diluted with ACN (1 mL) and purified by prep-HPLC (mobile phase: ACN-H₂O (0.1% FA), 5%-70%) to obtain the total compound 38 (16.48 mg, 25% yield) as a white solid. MS (ESI): mass calcd. for C₂₈H₃₈N₆O₇, 570.28, m/z found 571.2 [M+H]⁺. ¹H NMR (400 MHz, DMSO) δ 8.07 (d, J=8.4 Hz, 1H), 7.92 (br s, 3H), 6.92 (d, J=4.4 Hz, 1H), 6.88 (d, J=4.8 Hz, 1H), 6.65-6.60 (m, 1H), 5.13-5.01 (m, 2H), 4.48-4.45 (m, 1H), 4.30 (d, J=4.4 Hz, 2H), 4.15 (d, J=8.4 Hz, 1H), 2.25 (d, J=6.8 Hz, 2H), 1.89 (s, 3H), 1.83-1.57 (m, 6H), 1.27-1.09 (m, 3H), 0.98-0.93 (m, 2H), 0.86 (s, 9H)

### Example 39. Synthesis of ((2R,3S,4R,5R)-5-cyano-5-(4-(2-cyclohexylacetamido)pyrrolo[2,1-f][1,2,4]triazin-7-yl)-3,4-dihydroxytetrahydrofuran-2-yl)methyl (S)-2-amino-3,3-dimethylbutanoate (39)

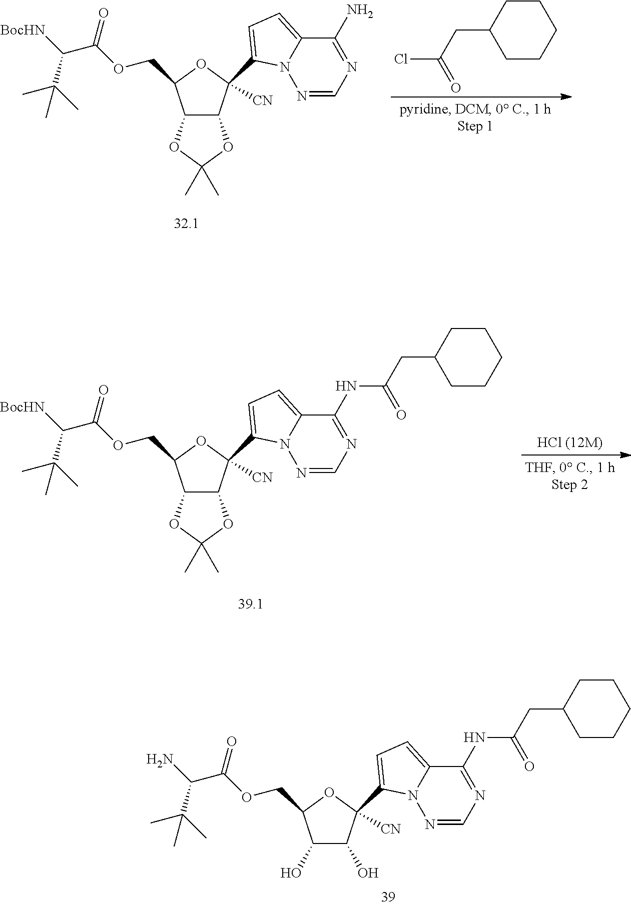 [SMILES: CC(C)(C)OC(=O)N[C@H](C(=O)OC[C@H]1O[C@@](C#N)(c2ccc3c(N)ncnn23)[C@@H]2OC(C)(C)O[C@@H]21)C(C)(C)C ; O=C(Cl)CC1CCCCC1 ; CC(C)(C)OC(=O)N[C@H](C(=O)OC[C@H]1O[C@@](C#N)(c2ccc3c(NC(=O)CC4CCCCC4)ncnn23)[C@@H]2OC(C)(C)O[C@@H]21)C(C)(C)C ; CC(C)(C)[C@H](N)C(=O)OC[C@H]1O[C@@](C#N)(c2ccc3c(NC(=O)CC4CCCCC4)ncnn23)[C@H](O)[C@@H]1O]

### Step 1. Synthesis of ((3aR,4R,6R,6aR)-6-cyano-6-(4-(2-cyclohexylacetamido)pyrrolo[2,1-f][1,2,4]triazin-7-yl)-2,2-dimethyltetrahydrofuro[3,4-d][1,3]dioxol-4-yl)methyl (S)-2-((tert-butoxycarbonyl)amino)-3,3-dimethylbutanoate (39.1)

To a solution of compound 32.1 (150 mg, 0.275 mmol) and pyridine (0.27 mL, 3.30 mmol) in DCM (1.0 mL) was added 2-cyclohexylacetyl chloride (57.5 mg, 0.358 mmol) at 0° C., the mixture was stirred at 0° C. for 1 h. Then the reaction was diluted with water (50 mL) and extracted with DCM (50 mL×3). The organic phase was washed with brine water (50 mL×3), then dried over anhydrous sodium sulfate, filtered, and concentrated to remove the solvent. The crude product was purified by chromatography (SiO₂, Petroleum ether/Ethyl acetate from 0% to 22%) to obtain compound 39.1 (170 mg, 88% yield) as a pink solid. MS (ESI): mass calcd. for C₃₄H₄₈N₆O₈, 668.35 m/z found 669.4 [M+H]⁺. ¹H NMR (400 MHz, DMSO) δ 10.91 (s, 1H), 8.43 (s, 1H), 7.24 (d, J=4.8 Hz, 1H), 7.14-7.08 (m, 2H), 5.44 (d, J=6.4 Hz, 1H), 4.94 (dd, J=6.4, 2.8 Hz, 1H), 4.61-4.55 (m, 1H), 4.24 (dd, J=12.0, 4.4 Hz, 1H), 4.13 (dd, J=12.0, 6.8 Hz, 1H), 3.80 (d, J=8.4 Hz, 1H), 2.57 (d, J=6.8 Hz, 2H), 1.88-1.78 (m, 1H), 1.76-1.67 (m, 3H), 1.66-1.57 (m, 5H), 1.38-1.32 (m, 11H), 1.30-1.21 (m, 4H), 1.05-0.95 (m, 2H), 0.86 (s, 9H).

### Step 2. Synthesis of ((2R,3S,4R,5R)-5-cyano-5-(4-(2-cyclohexylacetamido)pyrrolo[2,1-f][1,2,4]triazin-7-yl)-3,4-dihydroxytetrahydrofuran-2-yl)methyl (S)-2-amino-3,3-dimethylbutanoate (39)

To a solution of compound 39.1 (150 mg, 0.224 mmol) in THF (1.0 mL) was added HCl (1.5 mL, 12 M) at 0° C., then the reaction was stirred at 0° C. for 1 h. After completion, the mixture was concentrated in vacuo to afford a residue, the residue was diluted with ACN (2 mL) and purified by prep-HPLC (mobile phase: ACN-H₂O (0.1% FA), 5%-40%) to obtain compound 39 (66.44 mg, 55% yield) as a white solid. MS (ESI): mass calcd. for C₂₆H₃₆N₆O₆, 528.27 m/z found 529.2 [M+H]⁺. H NMR (400 MHz, DMSO) δ 10.88 (s, 1H), 8.39 (s, 1H), 7.26 (d, J=4.8 Hz, 1H), 7.09 (d, J=4.4 Hz, 1H), 6.46 (d, J=5.6 Hz, 1H), 5.50-5.40 (m, 1H), 4.69 (t, J=4.8 Hz, 1H), 4.32-4.19 (m, 3H), 3.97-3.90 (m, 1H), 2.99 (s, 1H), 2.57 (d, J=6.8 Hz, 2H), 1.88-1.78 (m, 1H), 1.77-1.57 (m, 5H), 1.31-1.09 (m, 3H), 1.06-0.94 (m, 2H), 0.90-0.80 (m, 9H).

### Example 40. Synthesis of ((2R,3S,4R,5R)-5-cyano-4-hydroxy-5-(4-((((pivaloyloxy)methoxy)carbonyl)amino)pyrrolo[2,1-f][1,2,4]triazin-7-yl)-3-(propionyloxy)tetrahydrofuran-2-yl)methyl (S)-2-amino-3,3-dimethylbutanoate (40)

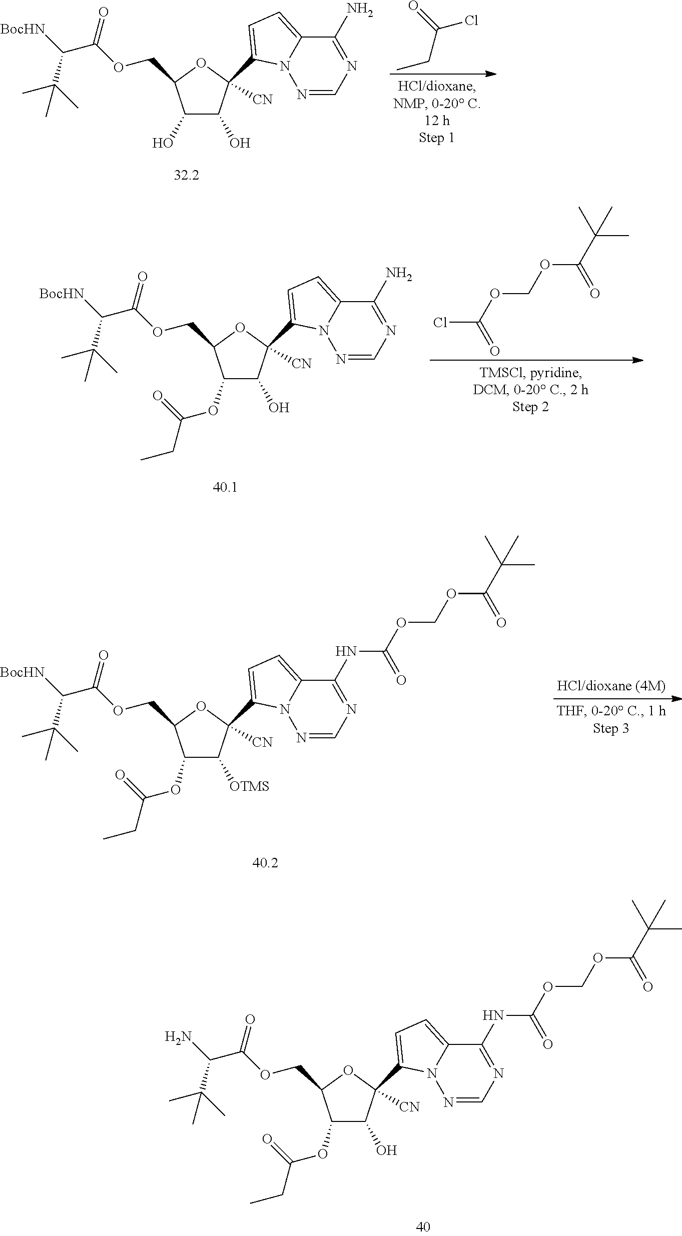

### Step 1. Synthesis of ((2R,3S,4R,5R)-5-(4-aminopyrrolo[2,1-f][1,2,4]triazin-7-yl)-5-cyano-4-hydroxy-3-(propionyloxy)tetrahydrofuran-2-yl)methyl (S)-2-((tert-butoxycarbonyl)amino)-3,3-dimethylbutanoate (40.1)

The title compound 40.1 was prepared according to the procedure of Example 32, Step 3 using 32.2 and propionyl chloride. MS (ESI): mass calcd. for C₂₆H₃₆N₆O₈, 560.26 m/z found 561.2 [M+H]⁺. H NMR (400 MHz, DMSO) δ 7.92 (br s, 3H), 7.07 (d, J=8.4 Hz, 1H), 6.92 (d, J=4.8 Hz, 1H), 6.87 (d, J=4.8 Hz, 1H), 6.60 (d, J=6.0 Hz, 1H), 5.13-5.08 (m, 2H), 4.50-4.42 (m, 1H), 4.30 (d, J=4.4 Hz, 2H), 3.84 (d, J=8.4 Hz, 1H), 2.40 (q, J=7.6 Hz, 2H), 1.42-1.20 (m, 9H), 1.07 (t, J=7.6 Hz, 3H), 0.86 (s, 9H).

### Step 2. ((2R,3R,4R,5R)-5-cyano-5-(4-((((pivaloyloxy)methoxy)carbonyl)amino)pyrrolo[2,1-f][1,2,4]triazin-7-yl)-3-(propionyloxy)-4-((trimethylsilyl)oxy)tetrahydrofuran-2-yl)methyl (S)-2-((tert-butoxycarbonyl)amino)-3,3-dimethylbutanoate (40.2)

The title compound 40.2 was prepared according to the procedure of Example 33, Step 1, using 40.1 and ((chlorocarbonyl)oxy)methyl pivalate. MS (ESI): mass calcd. for C₃₆H₅₄N₆O₁₂Si, 790.36 m/z found 791.3 [M+H]⁺.

### Step 3. Synthesis of ((2R,3S,4R,5R)-5-cyano-4-hydroxy-5-(4-((((pivaloyloxy)methoxy)carbonyl)amino)pyrrolo[2,1-f][1,2,4]triazin-7-yl)-3-(propionyloxy)tetrahydrofuran-2-yl)methyl (S)-2-amino-3,3-dimethylbutanoate (40)

The title compound 40 was prepared according to the procedure of Example 1, Step 4, using 40.2. MS (ESI): mass calcd. for C₂₈H₃₈N₆O₁₀, 618.26 m/z found 619.3 [M+H]⁺. H NMR (400 MHz, DMSO) δ 8.30 (s, 1H), 7.20-7.18 (m, 1H), 7.05-7.02 (m, 1H), 6.68 (d, J=6.4 Hz, 1H), 5.81 (s, 2H), 5.16 (t, J=5.2 Hz, 1H), 5.07 (t, J=5.6 Hz, 1H), 4.51-4.48 (m, 1H), 4.32-4.21 (m, 2H), 2.96 (s, 1H), 2.40 (q, J=7.6 Hz, 2H), 1.17 (s, 9H), 1.08 (t, J=7.6 Hz, 3H), 0.81 (s, 9H).

### Example 41. Synthesis of ((2R,3S,4R,5R)-5-cyano-5-(4-(((hexyloxy)carbonyl)amino)pyrrolo[2,1-f][1,2,4]triazin-7-yl)-3,4-dihydroxytetrahydrofuran-2-yl)methyl (S)-2-amino-3,3-dimethylbutanoate (41)

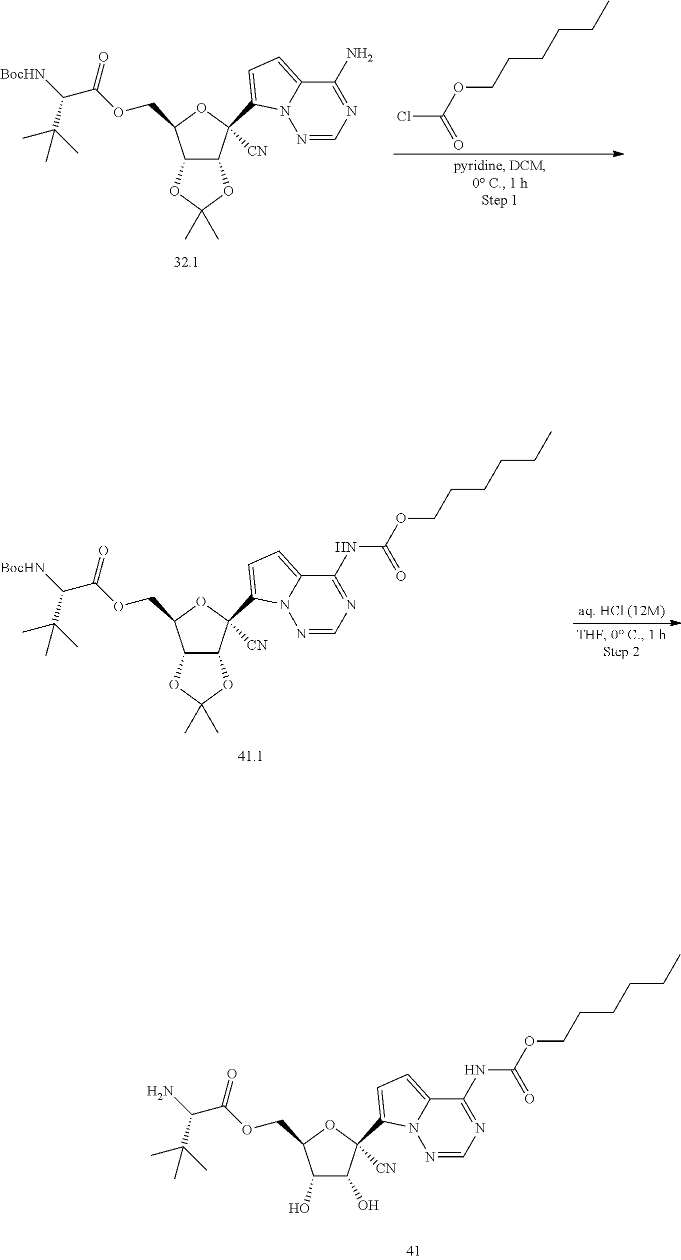

Title compound 41 was prepared from 32.1 according to the procedure of Example 39, Steps 1-2, using hexyl carbonochloridate. MS (ESI): mass calcd. for C₂₅H₃₆N₆O₇, 532.26, m/z found 533.3 [M+H]⁺. H NMR (400 MHz, DMSO) δ 8.35 (s, 1H), 7.30 (d, J=4.8 Hz, 1H), 7.05 (d, J=4.8 Hz, 1H), 6.44 (d, J=5.6 Hz, 1H), 5.58-5.35 (m, 1H), 4.69 (t, J=5.2 Hz, 1H), 4.32-4.21 (m, 3H), 4.17 (t, J=6.8 Hz, 2H), 3.97-3.91 (m, 1H), 3.01 (s, 1H), 1.72-1.62 (m, 2H), 1.43-1.26 (m, 6H), 0.88 (t, J=6.8 Hz, 3H), 0.84 (s, 9H).

### Example 42. Synthesis of ((2R,3S,4R,5R)-5-cyano-5-(4-(((hexyloxy)carbonyl)amino)pyrrolo[2,1-f][1,2,4]triazin-7-yl)-4-hydroxy-3-(propionyloxy)tetrahydrofuran-2-yl)methyl (S)-2-amino-3,3-dimethylbutanoate (42)

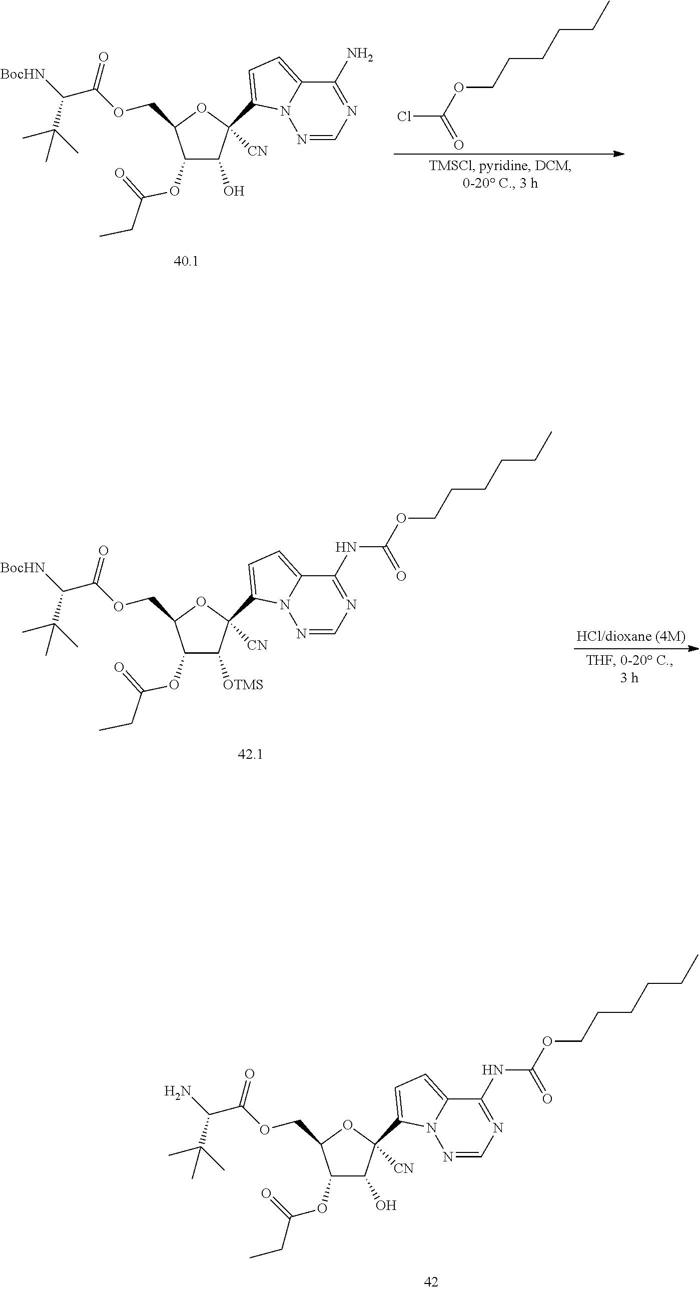

Title compound 42 was prepared from 40.1 according to the procedure of Example 1, Steps 3-4, using hexyl carbonochloridate. MS (ESI): mass calcd. for C₂₈H₄₀N₆O₈, 588.29, m/z found 589.05 [M+H]⁺. ¹H NMR (400 MHz, DMSO) δ 8.35 (s, 1H), 7.31 (d, J=4.8 Hz, 1H), 7.07 (d, J=4.8 Hz, 1H), 6.69 (d, J=6.4 Hz, 1H), 5.26-5.11 (m, 1H), 5.07 (t, J=6.0 Hz, 1H), 4.52-4.50 (m, 1H), 4.26-4.24 (m, 2H), 4.17 (t, J=6.8 Hz, 2H), 2.95 (s, 1H), 2.44-2.38 (m, 2H), 1.73-1.60 (m, 2H), 1.45-1.35 (m, 2H), 1.31-1.28 (m, 4H), 1.08 (t, J=7.6 Hz, 3H), 0.88 (t, J=6.8 Hz, 3H), 0.81 (s, 9H).

### Example 43. Synthesis of ((2R,3S,4R,5R)-5-cyano-5-(4-(((hexyloxy)carbonyl)amino)pyrrolo[2,1-f][1,2,4]triazin-7-yl)-4-hydroxy-3-(propionyloxy)tetrahydrofuran-2-yl)methyl (S)-2-amino-3,3-dimethylbutanoate (43)

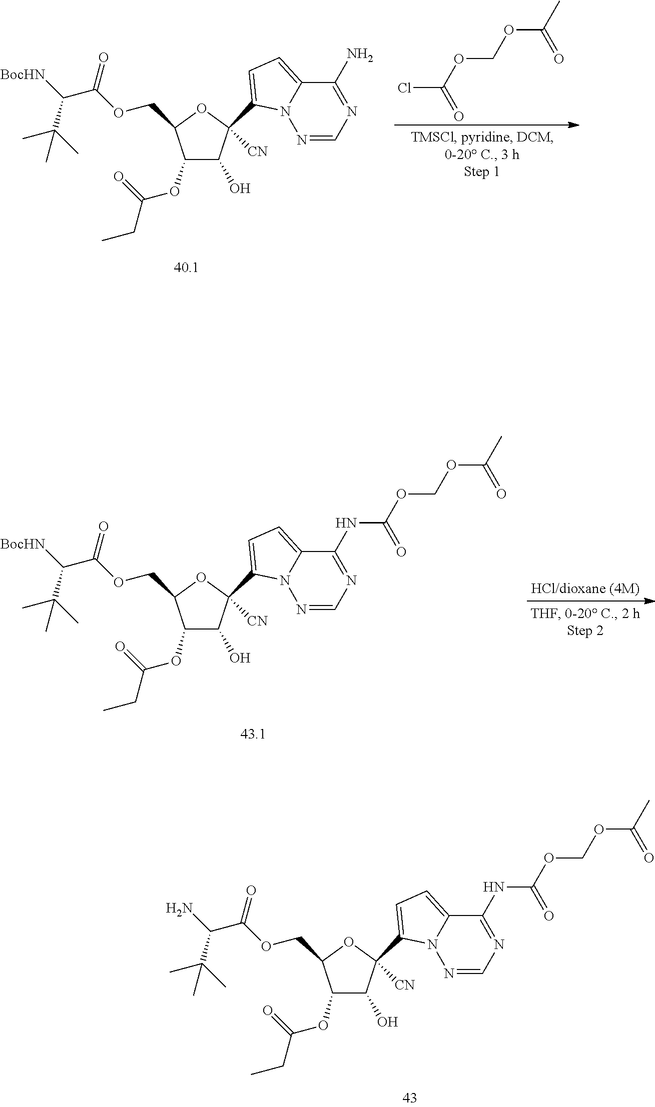

Title compound 43 was prepared from 40.1 according to the procedure of Example 1, Steps 3-4, using ((chlorocarbonyl)oxy)methyl acetate. MS (ESI): mass calcd. for C₂₅H₃₂N₆O₁₀, 576.22, m/z found 577.15 [M+H]⁺. ¹H NMR (400 MHz, DMSO) δ 8.28 (s, 1H), 7.18-7.12 (m, 1H), 7.03 (d, J=4.8 Hz, 1H), 6.70-6.63 (m, 1H), 5.79 (s, 2H), 5.21-5.13 (m, 1H), 5.11-5.03 (m, 1H), 4.51 (d, J=4.4 Hz, 1H), 4.33-4.23 (m, 2H), 2.97 (s, 1H), 2.41 (q, J=7.6 Hz, 2H), 2.11 (s, 3H), 1.08 (t J=7.6 Hz, 3H), 0.92-0.72 (m, 9H).

### Example 44. Synthesis of ((2R,3S,4R,5R)-5-(4-aminopyrrolo[2,1-f][1,2,4]triazin-7-yl)-5-cyano-3,4-dihydroxytetrahydrofuran-2-yl)methyl (S)-2-amino-3,3-dimethylbutanoate (44)

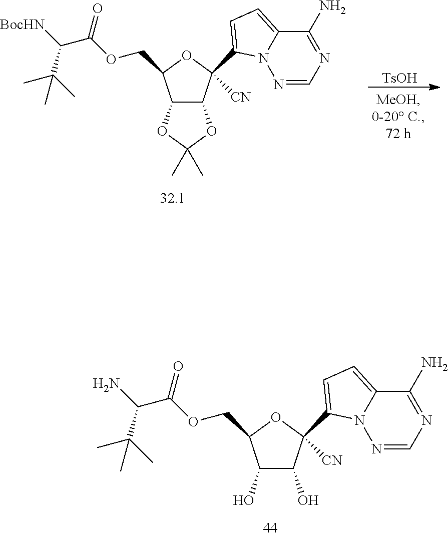

To a solution of compound 32.1 (1200 mg, 2.20 mmol) in MeOH (24 mL) was added TsOH·H₂O (837 mg, 4.40 mmol) at 0° C. The mixture was stirred at 0° C. for 30 min and then warmed up to 20° C. and stirred for 72 h. The mixture was adjusted to pH 8 with aqueous NaHCO₃. The reaction mixture was concentrated under reduced pressure to remove MeOH. Then the reaction residue was diluted with water (50 mL) and extracted with EtOAc (50 mL×3). The organic phase was washed with brine water (50 mL×3), then dried over anhydrous sodium sulfate, filtered, and concentrated to remove the solvent. The crude product was purified by chromatography (SiO₂, Dichloromethane/Methanol from 0% to 6%) to obtained crude product. And the mixture was diluted with ACN (2 mL) and purified by prep-HPLC (mobile phase: ACN-H₂O (0.1% FA), 5%-30%) to obtain compound 44 (120 mg, 12% yield) as a white solid. MS (ESI): mass calcd. for C₁₈H₂₄N₆O₅, 404.18, m/z found 405.2 [M+H]⁺. ¹H NMR (400 MHz, DMSO) δ 7.92 (br s, 3H), 6.91 (d, J=4.4 Hz, 1H), 6.83 (d, J=4.4 Hz, 1H), 6.40-6.30 (m, 1H), 5.45-5.35 (m, 1H), 4.71 (s, 1H), 4.30-4.19 (m, 3H), 3.96-3.92 (m, 1H), 3.01 (s, 1H), 0.84 (s, 9H).

### Example 45. Synthesis of ((2R,3S,4R,5R)-5-(4-aminopyrrolo[2,1-f][1,2,4]triazin-7-yl)-5-cyano-3,4-dihydroxytetrahydrofuran-2-yl)methyl (R)-2-amino-3,3-dimethylbutanoate (45)

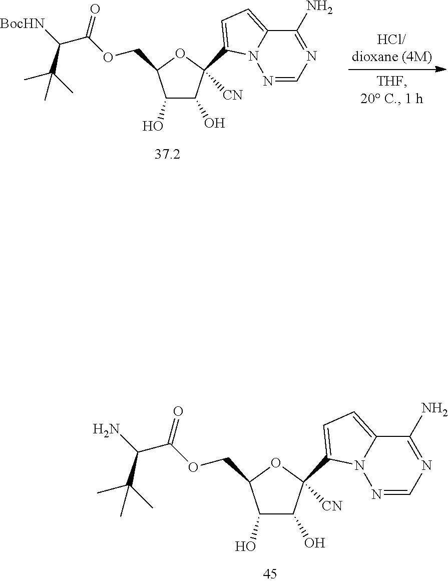 [SMILES: CC(C)(C)OC(=O)N[C@@H](C(=O)OC[C@H]1O[C@@](C#N)(c2ccc3c(N)ncnn23)[C@H](O)[C@@H]1O)C(C)(C)C ; CC(C)(C)[C@@H](N)C(=O)OC[C@H]1O[C@@](C#N)(c2ccc3c(N)ncnn23)[C@H](O)[C@@H]1O]

The title compound 45 was prepared from compound 37.2 according to the procedure of Example 1, Step 4. MS (ESI): mass calcd. for C₁₈H₂₄N₆O₅, 404.18, m/z found 405.2 [M+H]⁺. ¹H NMR (400 MHz, DMSO) δ 7.92 (br s, 3H), 6.91 (d, J=4.4 Hz, 1H), 6.82 (d, J=4.4 Hz, 1H), 6.50-6.20 (m, 1H), 5.50-5.25 (m, 1H), 4.72 (d, J=5.2 Hz, 1H), 4.30-4.18 (m, 1H), 3.99-3.94 (m, 1H), 3.07-3.00 (m, 1H), 0.85 (s, 9H).

### Example 46. Synthesis of ((2R,3S,4R,5R)-5-(4-(((acetoxymethoxy)carbonyl)amino)pyrrolo[2,1-f][1,2,4]triazin-7-yl)-5-cyano-4-hydroxy-3-(propionyloxy)tetrahydrofuran-2-yl)methyl (S)-2-amino-3,3-dimethylbutanoate (46)

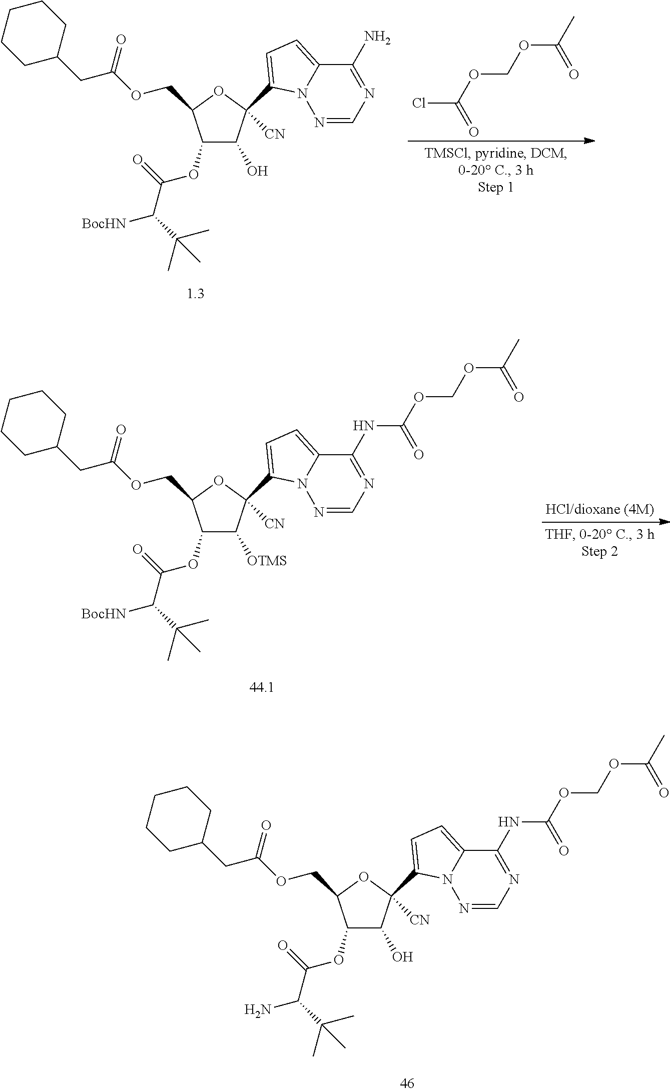

Title compound 46 was prepared from 1.3 according to the procedure of Example 1, Steps 3-4, using ((chlorocarbonyl)oxy)methyl acetate. MS (ESI): mass calcd. for C₃₀H₄N₆O₁₀, 644.28, m/z found 645.20 [M+H]⁺. ¹H NMR (400 MHz, DMSO) δ 8.32 (s, 1H), 7.25-7.18 (m, 1H), 7.04 (d, J=4.4 Hz, 1H), 6.78-6.63 (m, 1H), 5.79 (s, 2H), 5.15-5.04 (m, 2H), 4.49 (dd, J=8.4, 4.4 Hz, 1H), 4.29 (dd, J=12.4, 3.6 Hz, 1H), 4.21 (dd, J=12.0, 4.8 Hz, 1H), 3.10 (s, 1H), 2.18-2.08 (m, 5H), 1.64-1.52 (m, 6H), 1.20-1.02 (m, 3H), 0.99-0.74 (m, 12H).

### Example 47. Synthesis of (2R,3S,4R,5R)-5-cyano-2-((2-cyclobutylacetoxy)methyl)-4-hydroxy-5-(4-pentanamidopyrrolo[2,1-f][1,2,4]triazin-7-yl)tetrahydrofuran-3-yl (S)-2-amino-3,3-dimethylbutanoate (47)

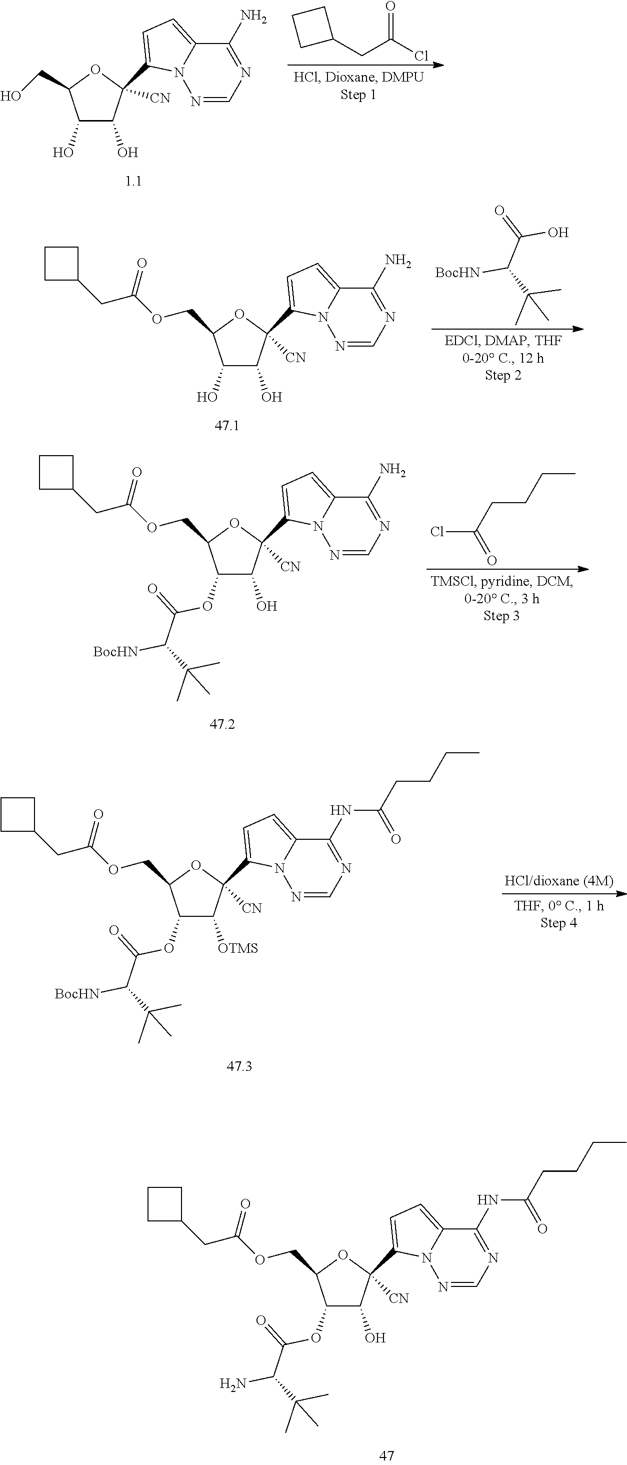

### Step 1. Synthesis of ((2R,3S,4R,5R)-5-(4-aminopyrrolo[2,1-f][1,2,4]triazin-7-yl)-5-cyano-3,4-dihydroxytetrahydrofuran-2-yl)methyl 2-cyclobutylacetate (47.1)

Title compound 47.1 was prepared according to the procedure of Example 1, Step 1, using 1.1 and 2-cyclobutylacetyl chloride. MS (ESI): mass calcd. for C₁₈H₂₁N₅O₅, 387.15, m/z found 387.95 [M+H]⁺. ¹H NMR (400 MHz, DMSO) δ 7.93 (br s, 3H), 6.93 (d, J=4.4 Hz, 1H), 6.82 (d, J=4.4 Hz, 1H), 6.32 (d, J=6.0 Hz, 1H), 5.38 (d, J=6.0 Hz, 1H), 4.76-4.64 (m, 1H), 4.35-4.28 (m, 1H), 4.25-4.18 (m, 1H), 4.17-4.10 (m, 1H), 3.97-3.91 (m, 1H), 2.60-2.51 (m, 1H), 2.42-2.36 (m, 2H), 2.06-1.93 (m, 2H), 1.86-1.70 (m, 2H), 1.68-1.55 (m, 2H).

### Step 2. Synthesis of (2R,3S,4R,5R)-5-(4-aminopyrrolo[2,1-f][1,2,4]triazin-7-yl)-5-cyano-2-((2-cyclobutylacetoxy)methyl)-4-hydroxytetrahydrofuran-3-yl (S)-2-((tert-butoxycarbonyl)amino)-3,3-dimethylbutanoate (47.2)

Title compound 47.2 was prepared according to the procedure of Example 1, Step 2, using 47.1 and (S)-2-((tert-butoxycarbonyl)amino)-3,3-dimethylbutanoic acid. MS (ESI): mass calcd. for C₂₉H₄₀N₆O₈, 600.29, m/z found 601.20 [M+H]⁺. ¹H NMR (400 MHz, DMSO) δ 7.93 (br s, 3H), 7.03 (d, J=8.4 Hz, 1H), 6.94 (d, J=4.4 Hz, 1H), 6.87 (d, J=4.4 Hz, 1H), 6.68-6.60 (m, 1H), 5.20-5.05 (m, 2H), 4.48-4.35 (m, 1H), 4.33-4.24 (m, 1H), 4.20-4.10 (m, 1H), 3.94 (d, J=8.0 Hz, 1H), 2.55-2.51 (m, 1H), 2.43-2.35 (m, 2H), 2.05-1.93 (m, 2H), 1.85-1.70 (m, 2H), 1.65-1.54 (m, 2H), 1.39 (s, 9H), 0.98 (s, 9H).

### Step 3. Synthesis of (2R,3R,4R,5R)-5-cyano-2-((2-cyclobutylacetoxy)methyl)-5-(4-pentanamidopyrrolo[2,1-f][1,2,4]triazin-7-yl)-4-((trimethylsilyl)oxy)tetrahydrofuran-3-yl (S)-2-((tert-butoxycarbonyl)amino)-3,3-dimethylbutanoate (47.3)

Title compound 47.3 was prepared according to the procedure of Example 1, Step 3, using 47.2 and pentanoyl chloride. MS (ESI): m/z calcd. for C₃₇H₅₆N₆O₉Si, 756.39, found 757.40 [M+H]⁺. ¹H NMR (400 MHz, DMSO) δ 10.94 (s, 1H), 8.39 (s, 1H), 7.34 (d, J=4.8 Hz, 1H), 7.10 (d, J=4.8 Hz, 1H), 7.04 (d, J=8.8 Hz, 1H), 5.27 (d, J=5.2 Hz, 1H), 5.15 (t, J=5.6 Hz, 1H), 4.56-4.46 (m, 1H), 4.40-4.32 (m, 1H), 4.28-4.20 (m, 1H), 4.05-4.02 (m, 1H), 2.72 (t, J=7.2 Hz, 2H), 2.58-2.52 (m, 1H), 2.44-2.39 (m, 2H), 1.82-1.71 (m, 2H), 1.67-1.56 (m, 4H), 1.42-1.23 (m, 13H), 0.98-0.88 (m, 12H), 0.01 (s, 9H).

### Step 4. Synthesis of (2R,3S,4R,5R)-5-cyano-2-((2-cyclobutylacetoxy)methyl)-4-hydroxy-5-(4-pentanamidopyrrolo[2,1-f][1,2,4]triazin-7-yl)tetrahydrofuran-3-yl (S)-2-amino-3,3-dimethylbutanoate (47)

The title compound was prepared from carbamate 47.3 according to the procedure of Example 1, Step 4. MS (ESI): mass calcd. for C₂₉H₄₀N₆O₇, 584.30, m/z found 585.00 [M+H]⁺. ¹H NMR (400 MHz, DMSO) δ 10.93 (s, 1H), 8.40 (s, 1H), 7.31 (d, J=4.8 Hz, 1H), 7.11 (d, J=4.8 Hz, 1H), 6.80-6.65 (m, 1H), 5.17-5.06 (m, 2H), 4.49 (dd, J=8.0, 4.0 Hz, 1H), 4.34-4.25 (m, 1H), 4.23-4.13 (m, 1H), 3.10 (s, 1H), 2.72 (t, J=7.2 Hz, 2H), 2.49-2.45 (m, 1H), 2.42-2.35 (m, 2H), 2.03-1.92 (m, 2H), 1.81-1.70 (m, 2H), 1.65-1.54 (m, 4H), 1.41-1.30 (m, 2H), 0.96-0.88 (m, 12H).

### Example 48. Synthesis of (2R,3S,4R,5R)-5-cyano-2-((2-cyclobutylacetoxy)methyl)-5-(4-hexanamidopyrrolo[2,1-f][1,2,4]triazin-7-yl)-4-hydroxytetrahydrofuran-3-yl (S)-2-amino-3,3-dimethylbutanoate (48)

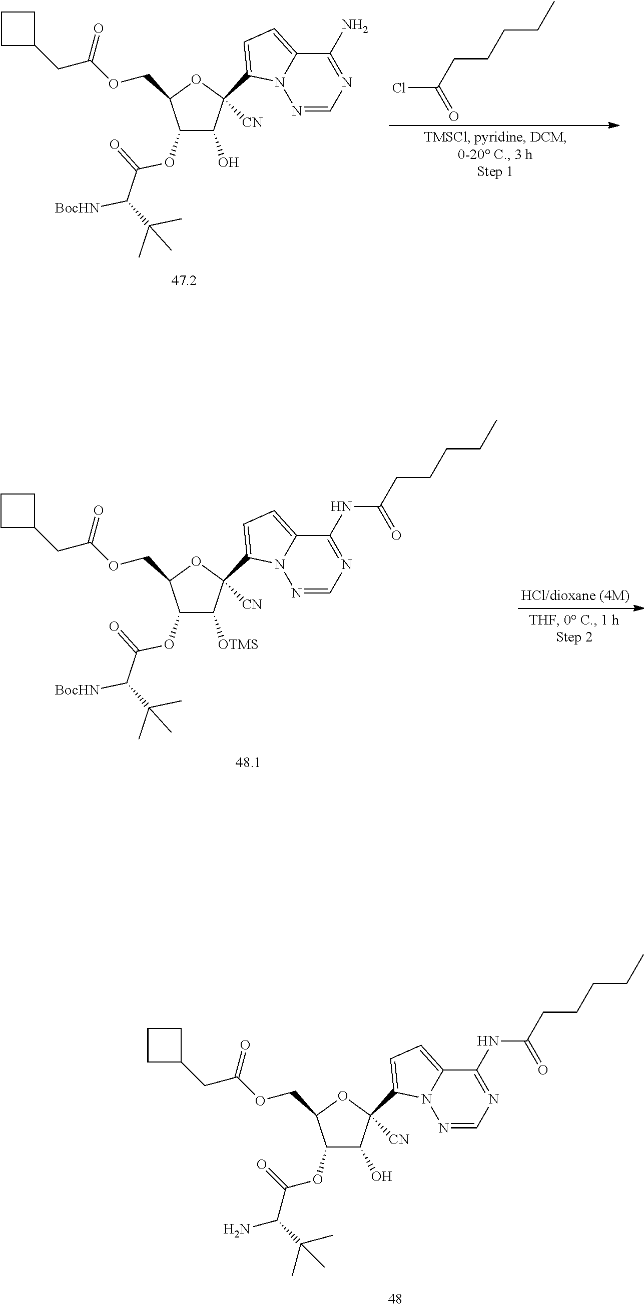

### Step 1. Synthesis of (2R,3R,4R,5R)-5-cyano-2-((2-cyclobutylacetoxy)methyl)-5-(4-hexanamidopyrrolo[2,1-f][1,2,4]triazin-7-yl)-4-((trimethylsilyl)oxy)tetrahydrofuran-3-yl (S)-2-((tert-butoxycarbonyl)amino)-3,3-dimethylbutanoate (48.1)

Title compound 48.1 was prepared according to the procedure of Example 1, Step 3, using 47.2 and hexanoyl chloride. MS (ESI): mass calcd. for C₃₈H₅₈N₆O₉Si, 770.40, m/z found 771.35 [M+H]⁺. ¹H NMR (400 MHz, DMSO) δ 10.94 (s, 1H), 8.39 (s, 1H), 7.34 (d, J=4.8 Hz, 1H), 7.10 (d, J=4.8 Hz, 1H), 7.04 (d, J=8.8 Hz, 1H), 5.27 (d, J=5.2 Hz, 1H), 5.15 (t, J=5.6 Hz, 1H), 4.55-4.47 (m, 1H), 4.39-4.33 (m, 1H), 4.28-4.20 (m, 1H), 4.04-4.02 (m, 1H), 2.70 (t, J=7.6 Hz, 2H), 2.57-2.53 (m, 1H), 2.43-2.40 (m, 2H), 2.03-1.97 (m, 2H), 1.84-1.70 (m, 2H), 1.65-1.57 (m, 4H), 1.38 (s, 9H), 1.33-1.29 (m, 4H), 0.95 (s, 9H), 0.90-0.87 (m, 3H), 0.01 (s, 9H).

### Step 2. Synthesis of (2R,3S,4R,5R)-5-cyano-2-((2-cyclobutylacetoxy)methyl)-5-(4-hexanamidopyrrolo[2,1-f][1,2,4]triazin-7-yl)-4-hydroxytetrahydrofuran-3-yl (S)-2-amino-3,3-dimethylbutanoate (48)

The title compound was prepared from carbamate 48.1 according to the procedure of Example 1, Step 4. MS (ESI): mass calcd. for C₃₀H₄₂N₆O₇, 598.31, m/z found 599.20 [M+H]⁺. ¹H NMR (400 MHz, DMSO) δ 10.93 (s, 1H), 8.40 (s, 1H), 7.32 (d, J=4.8 Hz, 1H), 7.11 (d, J=4.8 Hz, 1H), 6.80-6.65 (m, 1H), 5.20-5.04 (m, 2H), 4.50 (dd, J=8.0, 4.0 Hz, 1H), 4.35-4.27 (m, 1H), 4.23-4.14 (m, 1H), 3.12 (s, 1H), 2.75-2.65 (m, 2H), 2.49-2.41 (m, 1H), 2.40-2.35 (m, 2H), 2.03-1.91 (m, 2H), 1.82-1.70 (m, 2H), 1.68-1.53 (m, 4H), 1.38-1.28 (m, 4H), 0.99-0.92 (m, 9H), 0.91-0.85 (m, 3H).

### Example 49. Synthesis of (2R,3S,4R,5R)-5-cyano-2-((2-cyclobutylacetoxy)methyl)-5-(4-(2-ethoxy-2-methylpropanamido)pyrrolo[2,1-f][1,2,4]triazin-7-yl)-4-hydroxytetrahydrofuran-3-yl (S)-2-amino-3,3-dimethylbutanoate (49)

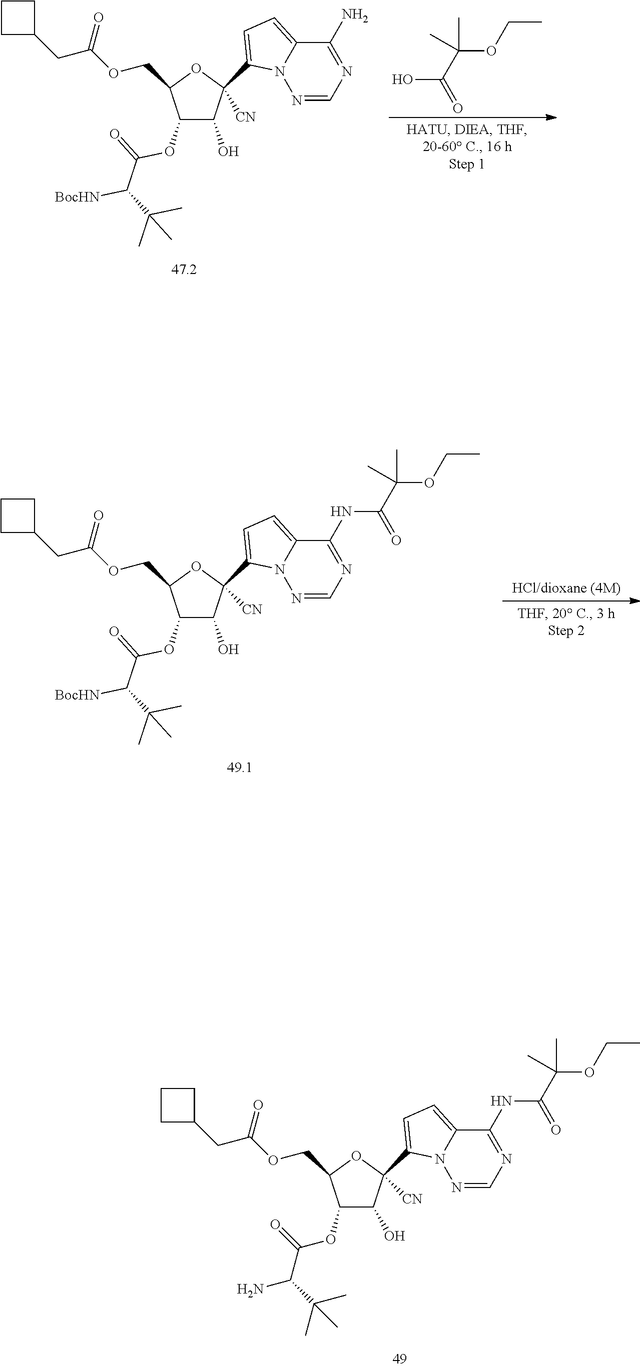

### Step 1. Synthesis of (2R,3S,4R,5R)-5-cyano-2-((2-cyclobutylacetoxy)methyl)-5-(4-(2-ethoxy-2-methylpropanamido)pyrrolo[2,1-f][1,2,4]triazin-7-yl)-4-hydroxytetrahydrofuran-3-yl (S)-2-((tert-butoxycarbonyl)amino)-3,3-dimethylbutanoate (49.1)

Title compound 49.1 was prepared according to the procedure of Example 3, Step 1, using 47.2 and 2-ethoxy-2-methylpropanoic acid. MS (ESI): mass calcd. for C₃₅H₅₀N₆O₁₀, 714.36, m/z found 715.20 [M+H]⁺.

### Step 2. Synthesis of (2R,3S,4R,5R)-5-cyano-2-((2-cyclobutylacetoxy)methyl)-5-(4-(2-ethoxy-2-methylpropanamido)pyrrolo[2,1-f][1,2,4]triazin-7-yl)-4-hydroxytetrahydrofuran-3-yl (S)-2-amino-3,3-dimethylbutanoate (49)

The title compound was prepared from carbamate 49.1 according to the procedure of Example 1, Step 4. MS (ESI): mass calcd. for C₃₀H₄₂N₆O₈, 614.31, m/z found 615.10 [M+H]⁺. ¹H NMR (400 MHz, DMSO) δ 8.48 (s, 1H), 7.22 (d, J=4.8 Hz, 1H), 7.18 (d, J=4.8 Hz, 1H), 6.85-6.65 (m, 1H), 5.18-5.05 (m, 2H), 4.55-4.48 (m, 1H), 4.29 (dd, J=12.4, 3.6 Hz, 1H), 4.20 (dd, J=12.4, 4.8 Hz, 1H), 3.49 (q, J=7.2 Hz, 2H), 3.10 (s, 1H), 2.48-2.43 (m, 1H), 2.40-2.33 (m, 2H), 2.03-1.91 (m, 2H), 1.84-1.68 (m, 2H), 1.64-1.53 (m, 2H), 1.45 (s, 6H), 1.22 (t, J=7.2 Hz, 3H), 0.94 (s, 9H).

### Example 50. Synthesis of (2R,3S,4R,5R)-5-cyano-2-((2-cyclobutylacetoxy)methyl)-5-(4-(2-ethylbutanamido)pyrrolo[2,1-f][1,2,4]triazin-7-yl)-4-hydroxytetrahydrofuran-3-yl (S)-2-amino-3,3-dimethylbutanoate (50)

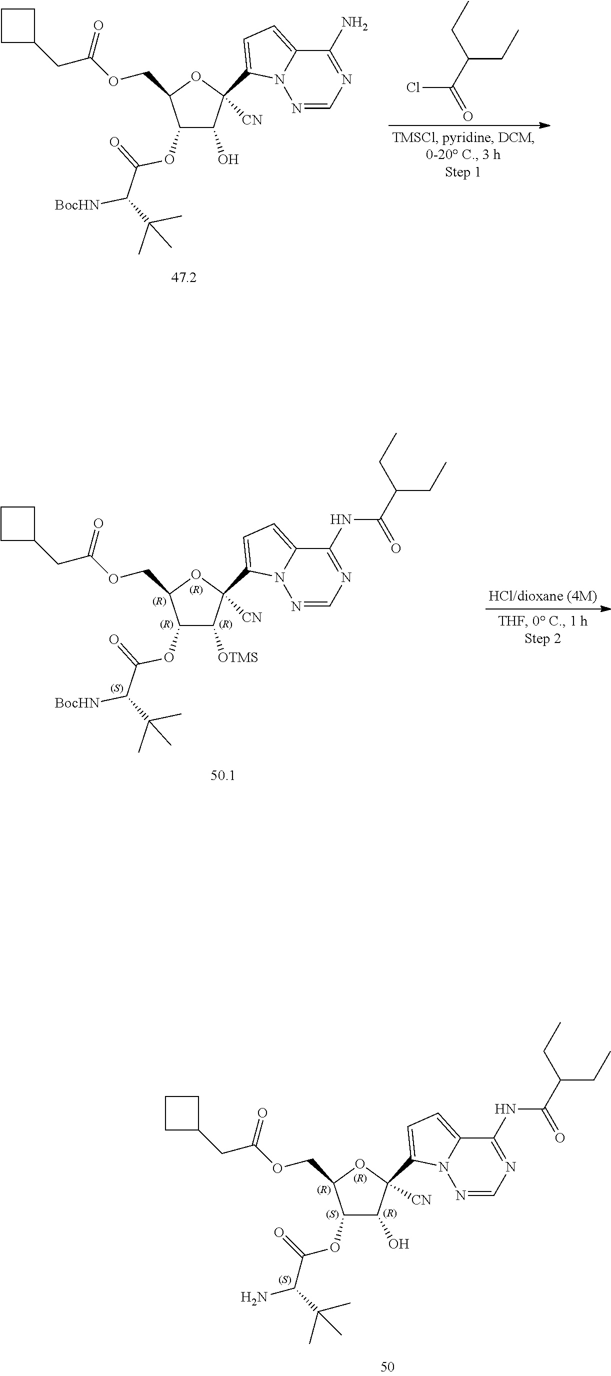

### Step 1. Synthesis of (2R,3R,4R,5R)-5-cyano-2-((2-cyclobutylacetoxy)methyl)-5-(4-(2-ethylbutanamido)pyrrolo[2,1-f][1,2,4]triazin-7-yl)-4-((trimethylsilyl)oxy)tetrahydrofuran-3-yl (S)-2-((tert-butoxycarbonyl)amino)-3,3-dimethylbutanoate (50.1)

Title compound 50.1 was prepared according to the procedure of Example 1, Step 3, using 47.2 and 2-ethylbutanoyl chloride. MS (ESI): mass calcd. for C₃₈H₅₈N₆O₉Si, 770.40, m/z found 771.40 [M+H]⁺. H NMR (400 MHz, DMSO) δ 11.00 (s, 1H), 8.42 (s, 1H), 7.28 (d, J=4.8 Hz, 1H), 7.12 (d, J=4.8 Hz, 1H), 7.06 (d, J=8.8 Hz, 1H), 5.26 (d, J=5.2 Hz, 1H), 5.13 (t J=5.6 Hz, 1H), 4.54-4.47 (m, 1H), 4.40-4.33 (m, 1H), 4.28-4.17 (m, 1H), 4.06-4.03 (m, 1H), 2.83-2.73 (m, 1H), 2.58-2.51 (m, 1H), 2.43-2.32 (m, 2H), 2.03-1.99 (m, 1H), 1.98-1.94 (m, 1H), 1.82-1.70 (m, 2H), 1.68-1.57 (m, 4H), 1.55-1.46 (m, 2H), 1.38 (s, 9H), 0.95 (s, 9H), 0.92-0.87 (m, 6H), 0.04 (s, 9H).

### Step 2. Synthesis of (2R,3S,4R,5R)-5-cyano-2-((2-cyclobutylacetoxy)methyl)-5-(4-(2-ethylbutanamido)pyrrolo[2,1-f][1,2,4]triazin-7-yl)-4-hydroxytetrahydrofuran-3-yl (S)-2-amino-3,3-dimethylbutanoate (50)

The title compound was prepared from carbamate 47.2 according to the procedure of Example 1, Step 4. MS (ESI): mass calcd. for C₃₀H₄₂N₆O₇, 598.31, m/z found 599.05 [M+H]⁺. (H NMR (400 MHz, DMSO) δ 10.99 (s, 1H), 8.43 (s, 1H), 7.26 (d, J=4.8 Hz, 1H), 7.14 (d, J=4.8 Hz, 1H), 6.80-6.65 (m, 1H), 5.17-5.07 (m, 2H), 4.50 (dd, J=8.0, 4.0 Hz, 1H), 4.33-4.26 (m, 1H), 4.23-4.15 (m, 1H), 3.11 (s, 1H), 2.83-2.71 (m, 1H), 2.48-2.42 (m, 1H), 2.40-2.07 (m, 2H), 2.03-1.90 (m, 2H), 1.84-1.68 (m, 2H), 1.66-1.46 (m, 6H), 0.98-0.87 (m, 15H).

### Example 54. Synthesis of (2R,3S,4R,5R)-5-cyano-2-((2-cyclopentylacetoxy)methyl)-4-hydroxy-5-(4-pentanamidopyrrolo[2,1-f][1,2,4]triazin-7-yl)tetrahydrofuran-3-yl (S)-2-amino-3,3-dimethylbutanoate (54)

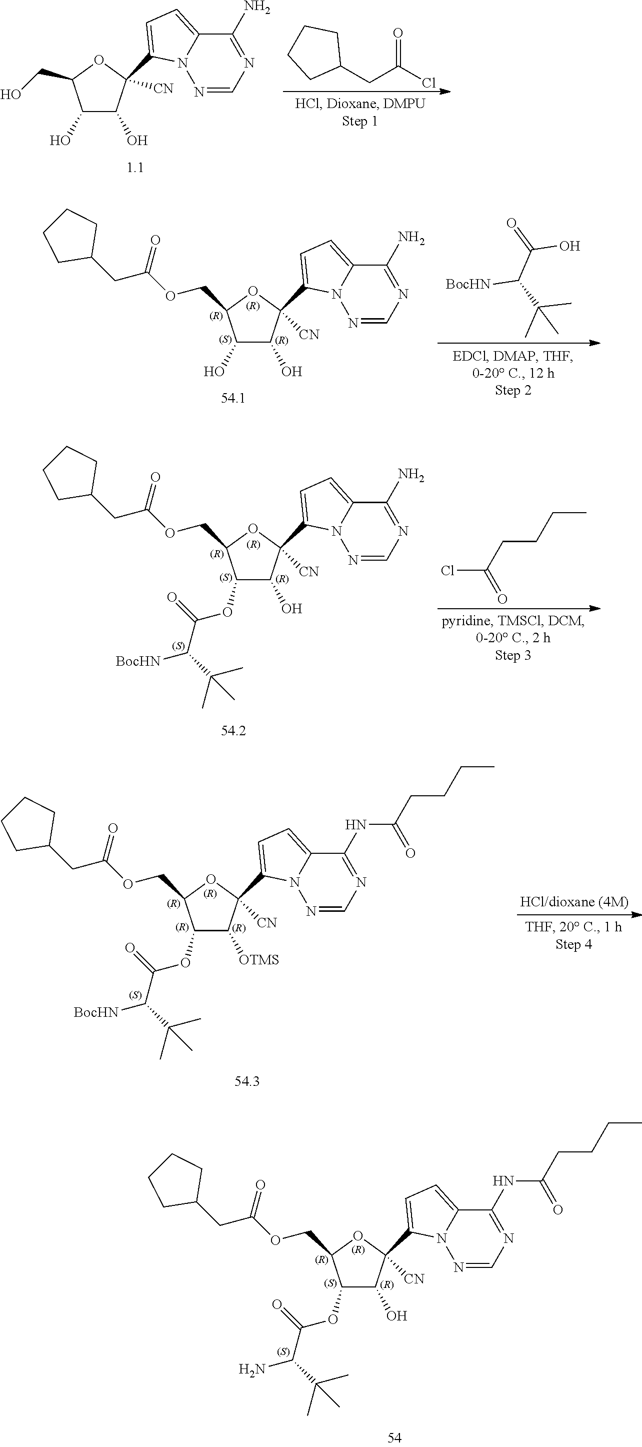

### Step 1: Synthesis of ((2R,3S,4R,5R)-5-(4-aminopyrrolo[2,1-f][1,2,4]triazin-7-yl)-5-cyano-3,4-dihydroxytetrahydrofuran-2-yl)methyl 2-cyclopentylacetate (54.1)

The title compound was prepared according to the procedure of Example 1, Step 1, using Compound 1.1 and 2-cyclopentylacetyl chloride. MS (ESI): mass calcd. for C₁₉H₂₃N₅O₅, 401.17, m/z found 402.2 [M+H]⁺.

### Step 2: Synthesis of (2R,3S,4R,5R)-5-(4-aminopyrrolo[2,1-f][1,2,4]triazin-7-yl)-5-cyano-2-((2-cyclopentylacetoxy)methyl)-4-hydroxytetrahydrofuran-3-yl (S)-2-((tert-butoxycarbonyl)amino)-3,3-dimethylbutanoate (54.2)

The title compound was prepared according to the procedure of Example 1, Step 2, using Compound 54.1 and (S)-2-((tert-butoxycarbonyl)amino)-3,3-dimethylbutanoic acid. MS (ESI): mass calcd. for C₃₀H₄₂N₆O₈, 614.31, m/z found 615.25 [M+H]⁺.

### Step 3: Synthesis (2R,3R,4R,5R)-5-cyano-2-((2-cyclopentylacetoxy)methyl)-5-(4-pentanamidopyrrolo[2,1-f][1,2,4]triazin-7-yl)-4-((trimethylsilyl)oxy)tetrahydrofuran-3-yl (S)-2-((tert-butoxycarbonyl)amino)-3,3-dimethylbutanoate (54.3)

The title compound was prepared according to the procedure of Example 1 Step 3, using Compound 54.2 and pentanoyl chloride. MS (ESI): mass calcd. for C₃H₅₈N₆O₉Si, 770.40, m/z found 771.2 [M+H]⁺.

### Step 4: Synthesis of (2R,3S,4R,5R)-5-cyano-2-((2-cyclopentylacetoxy)methyl)-4-hydroxy-5-(4-pentanamidopyrrolo[2,1-f][1,2,4]triazin-7-yl)tetrahydrofuran-3-yl (S)-2-amino-3,3-dimethylbutanoate (54)

The title compound 54 was prepared according to the procedure of Example 1, Step 4, using Compound 54.3. MS (ESI): mass calcd. for C₃₀H₄₂N₆O₇, 598.31, m/z found 599.05 [M+H]⁺. ¹H NMR (400 MHz, DMSO) δ 10.93 (s, 1H), 8.40 (s, 1H), 7.31 (d, J=4.8 Hz, 1H), 7.11 (d, J=4.8 Hz, 1H), 6.73 (d, J=6.0 Hz, 1H), 5.12-5.05 (m, 2H), 4.51-4.48 (m, 1H), 4.36-4.27 (dd, J=12.4, 3.6 Hz, 1H), 4.23 (dd, J=12.4, 4.8 Hz, 1H), 3.17 (s, 1H), 2.72 (t, J=7.6 Hz, 2H), 2.26 (dd, J=7.6, 2.8 Hz, 2H), 2.11-1.96 (m, 1H), 1.70-1.62 (m, 4H), 1.56-1.40 (m, 4H), 1.40-1.35 (m, 2H), 1.14-0.86 (m, 14H).

### Example 55. Synthesis of (2R,3S,4R,5R)-5-cyano-2-((2-cyclopentylacetoxy)methyl)-5-(4-hexanamidopyrrolo[2,1-f][1,2,4]triazin-7-yl)-4-hydroxytetrahydrofuran-3-yl (S)-2-amino-3,3-dimethylbutanoate (55)

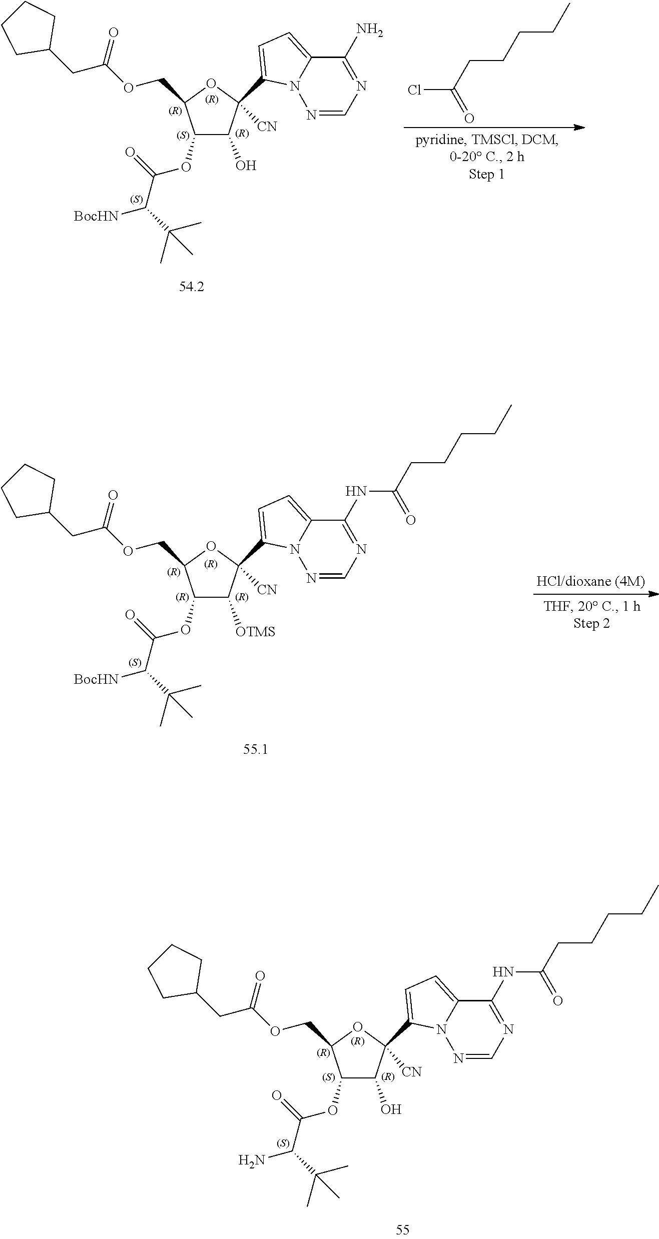

### Step 1: Synthesis of (2R,3R,4R,5R)-5-cyano-2-((2-cyclopentylacetoxy)methyl)-5-(4-hexanamidopyrrolo[2,1-f][1,2,4]triazin-7-yl)-4-((trimethylsilyl)oxy)tetrahydrofuran-3-yl (S)-2-((tert-butoxycarbonyl)amino)-3,3-dimethylbutanoate (55.1)

The title compound was prepared according to the procedure of Example 1, Step 3, using Compound 54.2 and hexanoyl chloride. MS (ESI): mass calcd. for C₃₉H₆₀N₆O₉Si, 784.42, m/z found 785.2 [M+H]⁺.

### Step 2: Synthesis of (2R,3S,4R,5R)-5-cyano-2-((2-cyclopentylacetoxy)methyl)-5-(4-hexanamidopyrrolo[2,1-f][1,2,4]triazin-7-yl)-4-hydroxytetrahydrofuran-3-yl (S)-2-amino-3,3-dimethylbutanoate (55)

The title compound 55 was prepared according to the procedure of Example 1, Step 4, using Compound 55.1. MS (ESI): mass calcd. for C₃₁H₄N₆O₇, 612.33, m/z found 613.05 [M+H]⁺. H NMR (400 MHz, DMSO) δ 10.92 (s, 1H), 8.40 (s, 1H), 7.31 (d, J=4.8 Hz, 1H), 7.11 (d, J=4.8 Hz, 1H), 6.73 (d, J=5.6 Hz, 1H), 5.17-5.03 (m, 2H), 4.51-4.45 (m, 1H), 4.30 (dd, J=12.4, 3.6 Hz, 1H), 4.21 (dd, J=12.4, 4.8 Hz, 1H), 3.10 (s, 1H), 2.71 (t J=7.6 Hz, 2H), 2.26 (dd, J=7.6, 3.2 Hz, 2H), 2.01-1.95 (m, 1H), 1.63-1.60 (m, 4H), 1.55-1.40 (m, 4H), 1.31-1.25 (m, 4H), 1.03-0.98 (m, 2H), 0.96-0.92 (m, 9H), 0.88 (t, J=7.2 Hz, 3H).

### Example 56. Synthesis of (2R,3S,4R,5R)-5-cyano-2-((2-cyclopentylacetoxy)methyl)-5-(4-(2-ethylbutanamido)pyrrolo[2,1-f][1,2,4]triazin-7-yl)-4-hydroxytetrahydrofuran-3-yl (S)-2-amino-3,3-dimethylbutanoate (56)

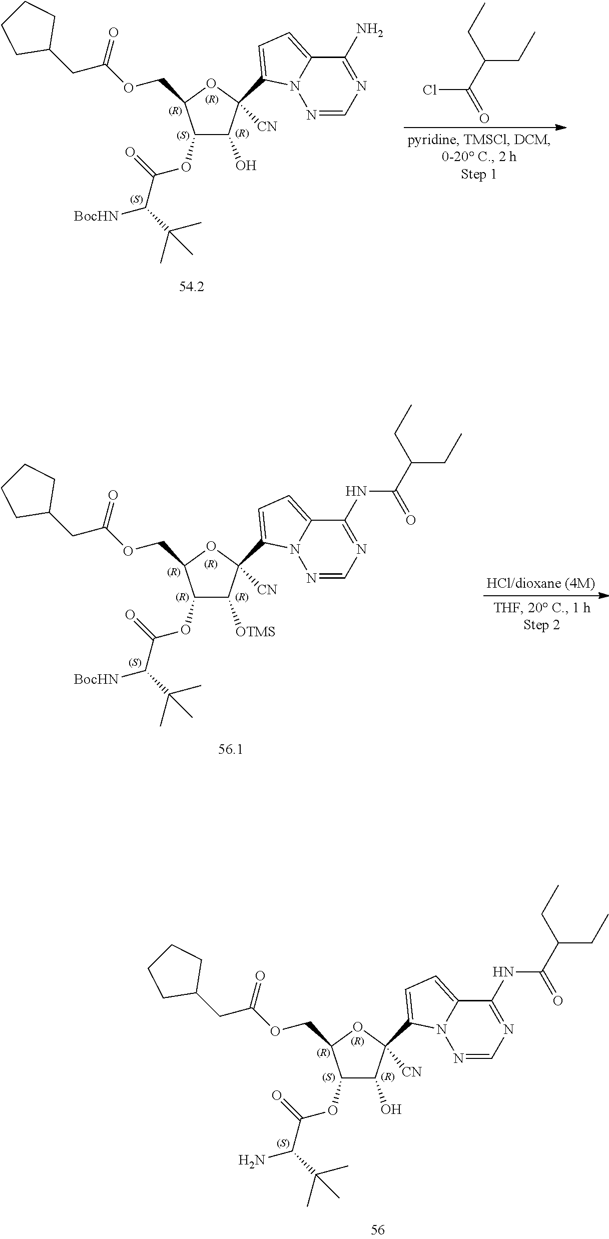

### Step 1: Synthesis of (2R,3R,4R,5R)-5-cyano-2-((2-cyclopentylacetoxy)methyl)-5-(4-(2-ethylbutanamido)pyrrolo[2,1-f][1,2,4]triazin-7-yl)-4-((trimethylsilyl)oxy)tetrahydrofuran-3-yl (S)-2-((tert-butoxycarbonyl)amino)-3,3-dimethylbutanoate (56.1)

The title compound was prepared according to the procedure of Example 1, Step 3, using Compound 54.2 and 2-ethylbutanoyl chloride. MS (ESI): mass calcd. for C₃₉H₆₀N₆O₉Si, 784.42, m/z found 785.2 [M+H]⁺.

### Step 2: Synthesis of (2R,3S,4R,5R)-5-cyano-2-((2-cyclopentylacetoxy)methyl)-5-(4-(2-ethylbutanamido)pyrrolo[2,1-f][1,2,4]triazin-7-yl)-4-hydroxytetrahydrofuran-3-yl (S)-2-amino-3,3-dimethylbutanoate (56)

The title compound 56 was prepared according to the procedure of Example 1, Step 4, using Compound 56.1. MS (ESI): mass calcd. for C₃₁H₄₄N₆O₇, 612.33, m/z found 613.10 [M+H]⁺. H NMR (400 MHz, DMSO) δ 10.99 (s, 1H), 8.43 (s, 1H), 7.25 (d, J=4.8 Hz, 1H), 7.12 (d, J=4.8 Hz, 1H), 6.74 (d, J=5.2 Hz, 1H), 5.27-5.00 (m, 2H), 4.52-4.50 (m, 1H), 4.31 (dd, J=12.4, 3.6 Hz, 1H), 4.21 (dd, J=12.4, 4.8 Hz, 1H), 3.19 (s, 1H), 2.83-2.71 (m, 1H), 2.25 (dd, J=7.6, 4.0 Hz, 2H), 2.09-1.95 (m, 1H), 1.63-1.60 (m, 4H), 1.58-1.47 (m, 4H), 1.42-1.35 (m, 2H), 1.02-1.00 (m, 2H), 0.95 (s, 9H), 0.89 (t, J=7.6 Hz, 6H).

### Example 57. Synthesis of (2R,3S,4R,5R)-5-cyano-2-((2-cyclopentylacetoxy)methyl)-4-hydroxy-5-(4-(2-methoxy-2-methylpropanamido)pyrrolo[2,1-f][1,2,4]triazin-7-yl)tetrahydrofuran-3-yl (S)-2-amino-3,3-dimethylbutanoate (57)

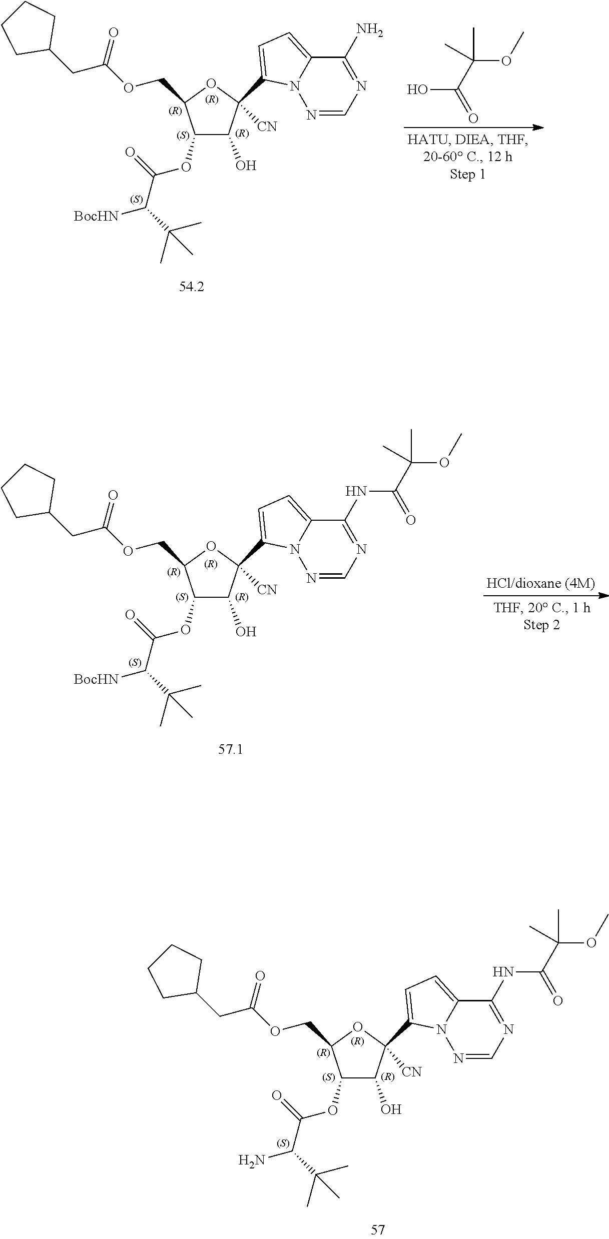

### Step 1: Synthesis of (2R,3S,4R,5R)-5-cyano-2-((2-cyclopentylacetoxy)methyl)-4-hydroxy-5-(4-(2-methoxy-2-methylpropanamido)pyrrolo[2,1-f][1,2,4]triazin-7-yl)tetrahydrofuran-3-yl (S)-2-((tert-butoxycarbonyl)amino)-3,3-dimethylbutanoate (57.1)

The title compound was prepared according to the procedure of Example 3, Step 1, using Compound 54.2 and 2-methoxy-2-methylpropanoic acid. MS (ESI): mass calcd. for C₃₅H₅₀N₆O₁₀, 714.36, m/z found 715.25 [M+H]⁺.

### Step 2: Synthesis of (2R,3S,4R,5R)-5-cyano-2-((2-cyclopentylacetoxy)methyl)-4-hydroxy-5-(4-(2-methoxy-2-methylpropanamido)pyrrolo[2,1-f][1,2,4]triazin-7-yl)tetrahydrofuran-3-yl (S)-2-amino-3,3-dimethylbutanoate (57)

The title compound 57 was prepared according to the procedure of Example 1, Step 4, using Compound 57.1. MS (ESI): mass calcd. for C₃₀H₄₂N₆O₈, 614.31, m/z found 615.05 [M+H]⁺. H NMR (400 MHz, DMSO) δ 8.48 (s, 1H), 7.21 (d, J=4.8 Hz, 1H), 7.17 (d, J=4.8 Hz, 1H), 6.74 (d, J=5.2 Hz, 1H), 5.14-5.10 (m, 2H), 4.52-4.50 (m, 1H), 4.30 (dd, J=12.4, 3.6 Hz, 1H), 4.22 (dd, J=12.4, 4.8 Hz, 1H), 3.29 (s, 3H), 3.15 (s, 1H), 2.30-2.23 (m, 2H), 2.03-1.98 (m, 1H), 1.65-1.60 (m, 2H), 1.58-1.49 (m, 2H), 1.47-1.40 (m, 8H), 1.05-1.00 (m, 2H), 0.95 (s, 9H).

### Example 61. Synthesis of (2R,3S,4R,5R)-5-cyano-2-((2-cycloheptylacetoxy)methyl)-4-hydroxy-5-(4-pentanamidopyrrolo[2,1-f][1,2,4]triazin-7-yl)tetrahydrofuran-3-yl (S)-2-amino-3,3-dimethylbutanoate (61)

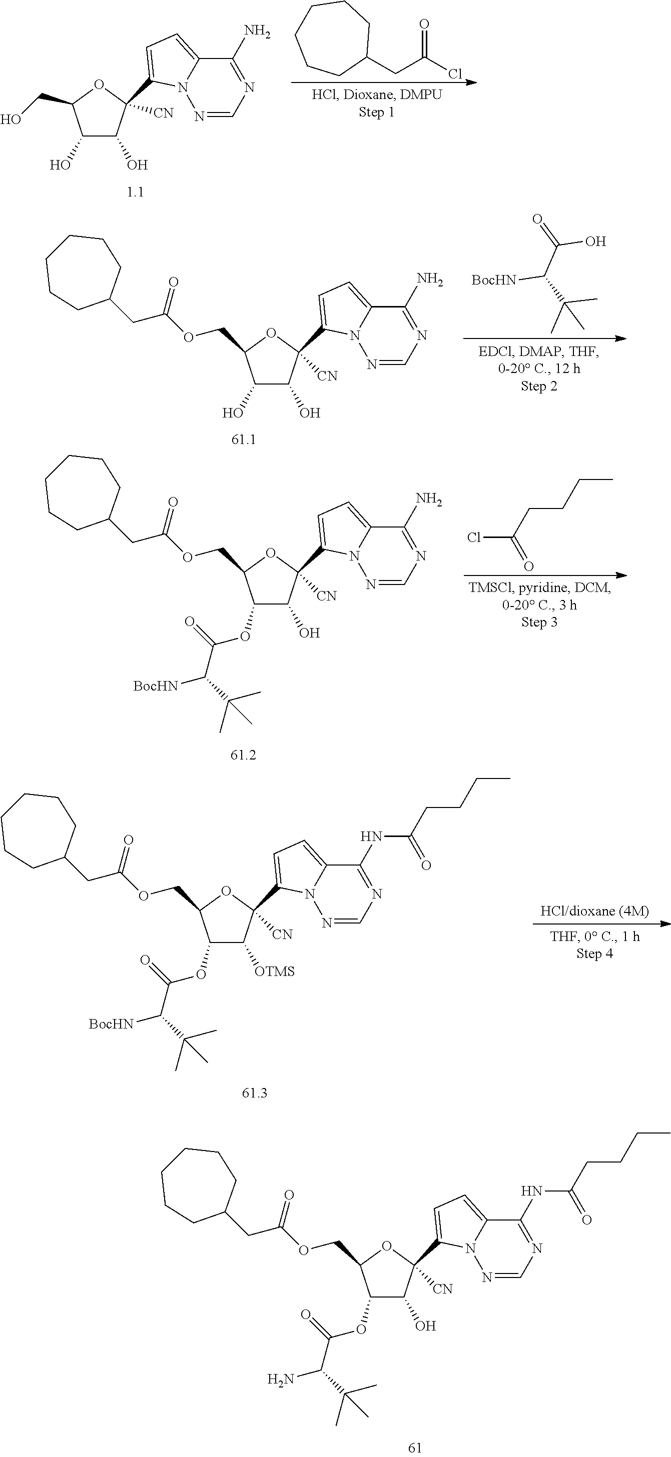

### Step 1. Synthesis of ((2R,3S,4R,5R)-5-(4-aminopyrrolo[2,1-f][1,2,4]triazin-7-yl)-5-cyano-3,4-dihydroxytetrahydrofuran-2-yl)methyl 2-cycloheptylacetate (61.1)

Title compound 61.1 was prepared according to the procedure of Example 1, Step 1, using 1.1 and 2-cycloheptylacetyl chloride. MS (ESI): mass calcd. for C₂₁H₂₇N₅O₅, 429.20, m/z found 430.15 [M+H]⁺. H NMR (400 MHz, DMSO) δ 7.92 (s, 3H), 6.92 (d, J=4.4 Hz, 1H), 6.81 (d, J=4.4 Hz, 1H), 6.32 (d, J=6.0 Hz, 1H), 5.37 (d, J=6.0 Hz, 1H), 4.74-4.64 (m, 1H), 4.35-4.28 (m, 1H), 4.25-4.12 (m, 2H), 3.98-3.91 (m, 1H), 2.24-2.14 (m, 2H), 1.90-1.79 (m, 1H), 1.66-1.48 (m, 6H), 1.46-1.27 (m, 4H), 1.17-1.07 (m, 2H).

### Step 2. Synthesis of (2R,3S,4R,5R)-5-(4-aminopyrrolo[2,1-f][1,2,4]triazin-7-yl)-5-cyano-2-((2-cycloheptylacetoxy)methyl)-4-hydroxytetrahydrofuran-3-yl (S)-2-((tert-butoxycarbonyl)amino)-3,3-dimethylbutanoate (61.2)

Title compound 47.2 was prepared according to the procedure of Example 1, Step 2, using 61.1 and (S)-2-((tert-butoxycarbonyl)amino)-3,3-dimethylbutanoic acid. MS (ESI): mass calcd. for C₃₂H₄₆N₆O₈, 642.34, m/z found 643.25 [M+H]⁺. H NMR (400 MHz, DMSO) δ 7.93 (br s, 3H), 7.02 (d, J=8.0 Hz, 1H), 6.94 (d, J=4.8 Hz, 1H), 6.86 (d, J=4.8 Hz, 1H), 6.69-6.61 (m, 1H), 5.14-5.06 (m, 2H), 4.48-4.40 (m, 1H), 4.35-4.28 (m, 1H), 4.23-4.15 (m, 1H), 3.94 (d, J=8.4 Hz, 1H), 2.25-2.09 (m, 2H), 1.89-1.72 (m, 1H), 1.62-1.46 (m, 6H), 1.44-1.28 (m, 13H), 1.17-1.06 (m, 2H), 0.98 (s, 9H).

### Step 3. Synthesis of (2R,3R,4R,5R)-5-cyano-2-((2-cycloheptylacetoxy)methyl)-5-(4-pentanamidopyrrolo[2,1-f][1,2,4]triazin-7-yl)-4-((trimethylsilyl)oxy)tetrahydrofuran-3-yl (S)-2-((tert-butoxycarbonyl)amino)-3,3-dimethylbutanoate (61.3)

Title compound 61.3 was prepared according to the procedure of Example 1, Step 3, using 61.2 and pentanoyl chloride. MS (ESI): m/z calcd. for C₄₀H₆₂N₆O₉Si, 798.43, found 799.55 [M+H]⁺. ¹H NMR (400 MHz, DMSO) δ 10.94 (s, 1H), 8.38 (s, 1H), 7.33 (d, J=4.8 Hz, 1H), 7.10 (d, J=4.8 Hz, 1H), 7.04 (d, J=8.8 Hz, 1H), 5.27 (d, J=5.2 Hz, 1H), 5.13 (t, J=5.6 Hz, 1H), 4.60-4.48 (m, 1H), 4.40-4.31 (m, 1H), 4.27 (dd, J=12.4, 5.2 Hz, 1H), 4.02 (d, J=8.8 Hz, 1H), 2.72 (t, J=7.2 Hz, 2H), 2.26-2.16 (m, 2H), 1.90-1.75 (m, 1H), 1.62-1.46 (m, 8H), 1.39-1.28 (m, 15H), 1.15-1.10 (m, 2H), 0.98-0.88 (m, 12H), 0.02 (s, 9H).

### Step 4. Synthesis of (2R,3S,4R,5R)-5-cyano-2-((2-cycloheptylacetoxy)methyl)-4-hydroxy-5-(4-pentanamidopyrrolo[2,1-f][1,2,4]triazin-7-yl)tetrahydrofuran-3-yl (S)-2-amino-3,3-dimethylbutanoate (61)

The title compound was prepared from carbamate 61.3 according to the procedure of Example 1, Step 4. MS (ESI): mass calcd. for C₃₂H₄₆N₆O₇, 626.34, m/z found 627.15 [M+H]⁺. ¹H NMR (400 MHz, DMSO) δ 10.93 (s, 1H), 8.40 (s, 1H), 7.32 (d, J=4.8 Hz, 1H), 7.11 (d, J=4.8 Hz, 1H), 6.78-6.68 (m, 1H), 5.24-4.98 (m, 2H), 4.60-4.46 (m, 1H), 4.35-4.15 (m, 2H), 3.11 (s, 1H), 2.72 (t, J=7.2 Hz, 2H), 2.22-2.08 (m, 2H), 1.85-1.73 (m, 1H), 1.66-1.45 (m, 8H), 1.43-1.23 (m, 6H), 1.15-1.05 (m, 2H), 1.03-0.83 (m, 12H).

### Example 62. Synthesis of (2R,3S,4R,5R)-5-cyano-2-((2-cycloheptylacetoxy)methyl)-5-(4-(2-ethylbutanamido)pyrrolo[2,1-f][1,2,4]triazin-7-yl)-4-hydroxytetrahydrofuran-3-yl (S)-2-amino-3,3-dimethylbutanoate (62)

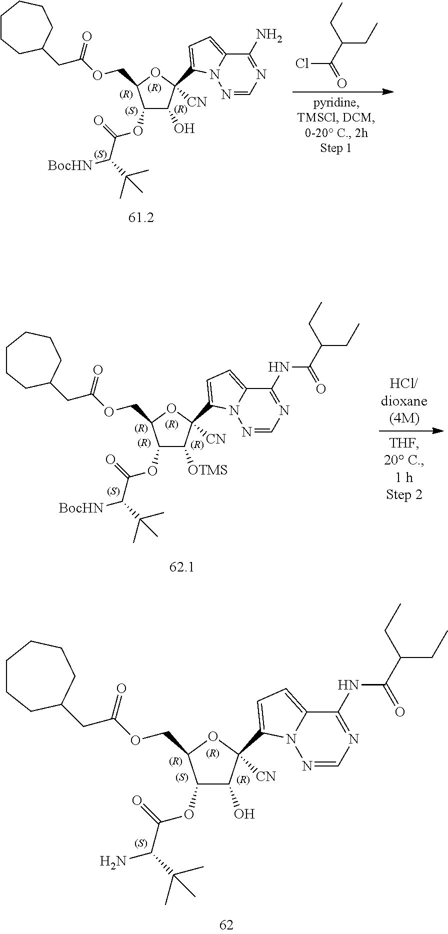

### Step 1: Synthesis of (2R,3R,4R,5R)-5-cyano-2-((2-cycloheptylacetoxy)methyl)-5-(4-(2-ethylbutanamido)pyrrolo[2,1-f][1,2,4]triazin-7-yl)-4-((trimethylsilyl)oxy)tetrahydrofuran-3-yl (S)-2-((tert-butoxycarbonyl)amino)-3,3-dimethylbutanoate (62.1)

The title compound was prepared according to the procedure of Example 1, Step 3, using Compound 61.2 and 2-ethylbutanoyl chloride. MS (ESI): mass calcd. for C₄₁H₆₄N₆O₉Si, 812.45, m/z found 813.2 [M+H]⁺.

### Step 2: Synthesis of (2R,3S,4R,5R)-5-cyano-2-((2-cycloheptylacetoxy)methyl)-5-(4-(2-ethylbutanamido)pyrrolo[2,1-f][1,2,4]triazin-7-yl)-4-hydroxytetrahydrofuran-3-yl (S)-2-amino-3,3-dimethylbutanoate (62)

The title compound 62 was prepared according to the procedure of Example 1, Step 4, using Compound 62.1. MS (ESI): mass calcd. for C₃₃H₄₈N₆O₇, 640.36, m/z found 641.25 [M+H]⁺. H NMR (400 MHz, DMSO) δ 10.99 (s, 1H), 8.43 (s, 1H), 7.26 (d, J=4.8 Hz, 1H), 7.13 (d, J=4.8 Hz, 1H), 6.73-6.70 (m, 1H), 5.10-5.05 (m, 2H), 4.52-4.50 (m, 1H), 4.29 (dd, J=12.4, 3.6 Hz, 1H), 4.22 (dd, J=12.4, 4.8 Hz, 1H), 3.10 (s, 1H), 2.82-2.72 (m, 1H), 2.14-2.10 (m, 2H), 1.82-1.72 (m, 1H), 1.70-1.43 (m, 10H), 1.42-1.20 (m, 4H), 1.16-0.99 (m, 2H), 0.99-0.84 (m, 15H).

### Example 63. Synthesis of (2R,3S,4R,5R)-5-cyano-2-((2-cycloheptylacetoxy)methyl)-4-hydroxy-5-(4-(2-methoxy-2-methylpropanamido)pyrrolo[2,1-f][1,2,4]triazin-7-yl)tetrahydrofuran-3-yl (S)-2-amino-3,3-dimethylbutanoate (63)

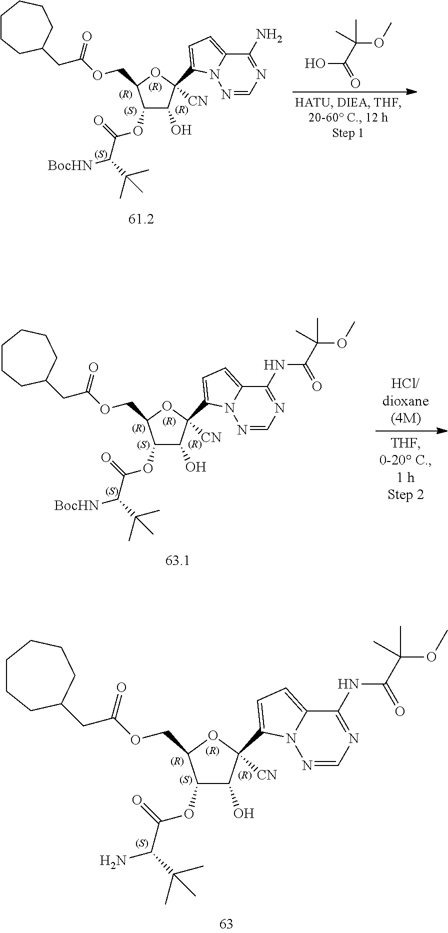

### Step 1: Synthesis of (2R,3S,4R,5R)-5-cyano-2-((2-cycloheptylacetoxy)methyl)-4-hydroxy-5-(4-(2-methoxy-2-methylpropanamido)pyrrolo[2,1-f][1,2,4]triazin-7-yl)tetrahydrofuran-3-yl (S)-2-((tert-butoxycarbonyl)amino)-3,3-dimethylbutanoate (63.1)

The title compound was prepared according to the procedure of Example 3, Step 1, using Compound 61.2 and 2-methoxy-2-methylpropanoic acid. MS (ESI): mass calcd. for C₃₇H₅₄N₆O₁₀, 742.39, m/z found 743.35 [M+H]⁺.

### Step 2: Synthesis of (2R,3S,4R,5R)-5-cyano-2-((2-cycloheptylacetoxy)methyl)-4-hydroxy-5-(4-(2-methoxy-2-methylpropanamido)pyrrolo[2,1-f][1,2,4]triazin-7-yl)tetrahydrofuran-3-yl (S)-2-amino-3,3-dimethylbutanoate (63)

The title compound 63 was prepared according to the procedure of Example 1, Step 4, using Compound 63.1. MS (ESI): mass calcd. for C₃₂H₄₆N₆O₈, 642.34, m/z found 643.25 [M+H]⁺. H NMR (400 MHz, DMSO) δ 8.48 (s, 1H), 7.22 (d, J=4.8 Hz, 1H), 7.17 (d, J=4.8 Hz, 1H), 6.74-6.70 (m, 1H), 5.12-5.10 (m, 2H), 4.52-4.50 (m, 1H), 4.29 (dd, J=12.4, 3.6 Hz, 1H), 4.22 (dd, J=12.4, 4.8 Hz, 1H), 3.29 (s, 3H), 3.11 (s, 1H), 2.17-2.10 (m, 2H), 1.92-1.71 (m, 1H), 1.54-1.43 (m, 12H), 1.42-1.25 (m, 4H), 1.17-1.05 (m, 2H), 0.94 (s, 9H).

### Example 65. Synthesis of (2R,3S,4R,5R)-5-(4-((butoxycarbonyl)amino)pyrrolo[2,1-f][1,2,4]triazin-7-yl)-5-cyano-2-((2-cycloheptylacetoxy)methyl)-4-hydroxytetrahydrofuran-3-yl (S)-2-amino-3,3-dimethylbutanoate (65)

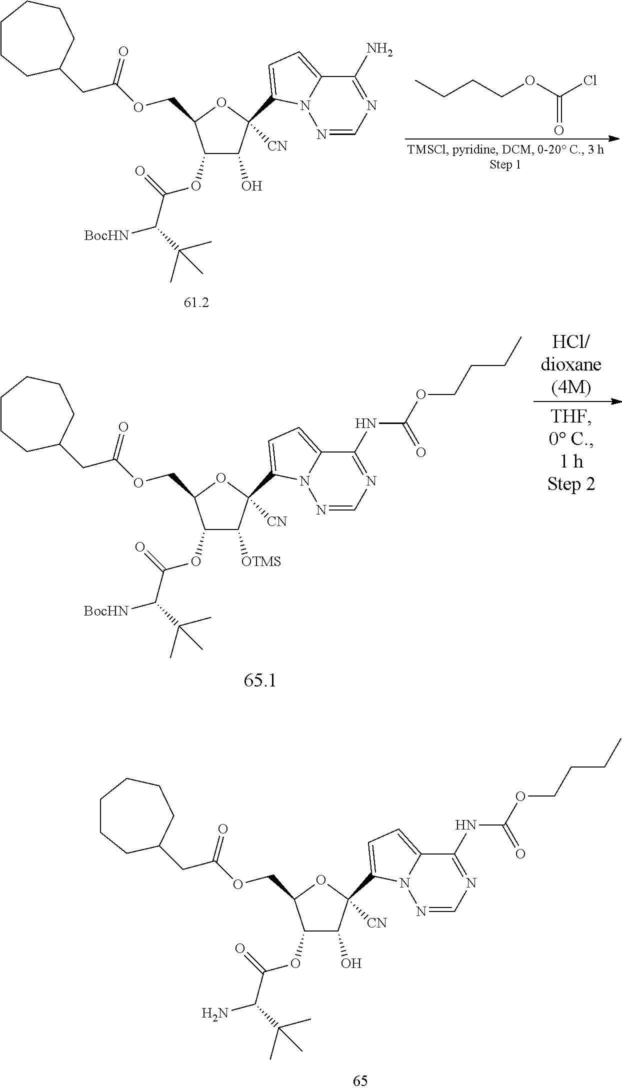

### Step 1. Synthesis of (2R,3R,4R,5R)-5-(4-((butoxycarbonyl)amino)pyrrolo[2,1-f][1,2,4]triazin-7-yl)-5-cyano-2-((2-cycloheptylacetoxy)methyl)-4-((trimethylsilyl)oxy)tetrahydrofuran-3-yl(S)-2-((tert-butoxycarbonyl)amino)-3,3-dimethylbutanoate (65.1)

Title compound 65.1 was prepared according to the procedure of Example 1, Step 3, using 61.2 and butyl carbonochloridate. MS (ESI): m/z calcd. for C₄₀H₆₂N₆O₁₀Si, 814.43, found 815.35 [M+H]⁺. ¹H NMR (400 MHz, DMSO) δ 10.97 (s, 1H), 8.36 (s, 1H), 7.37 (d, J=4.8 Hz, 1H), 7.10-7.03 (m, 2H), 5.27 (d, J=5.2 Hz, 1H), 5.15 (t, J=5.6 Hz, 1H), 4.54-4.47 (m, 1H), 4.39-4.32 (m, 1H), 4.30-4.24 (m, 1H), 4.20 (t, J=6.4 Hz, 2H), 2.26-2.15 (m, 2H), 1.91-1.77 (m, 1H), 1.70-1.62 (m, 2H), 1.61-1.47 (m, 6H), 1.43-1.29 (m, 15H), 1.16-1.07 (m, 2H), 0.98-0.87 (m, 12H), 0.01 (s, 9H).

### Step 2. Synthesis of (2R,3S,4R,5R)-5-(4-((butoxycarbonyl)amino)pyrrolo[2,1-f][1,2,4]triazin-7-yl)-5-cyano-2-((2-cycloheptylacetoxy)methyl)-4-hydroxytetrahydrofuran-3-yl (S)-2-amino-3,3-dimethylbutanoate (65)

The title compound was prepared from carbamate 65.1 according to the procedure of Example 1, Step 4. MS (ESI): mass calcd. for C₃₂H₄₆N₆O₈, 642.34, m/z found 643.20 [M+H]⁺. ¹H NMR (400 MHz, DMSO) δ 8.36 (s, 1H), 7.33 (d, J=4.8 Hz, 1H), 7.07 (d, J=4.8 Hz, 1H), 6.71 (d, J=4.8 Hz, 1H), 5.15-5.05 (m, 2H), 4.50 (dd, J=8.0, 4.0 Hz, 1H), 4.34-4.26 (m, 1H), 4.25-4.15 (m, 3H), 3.11 (s, 1H), 2.23-2.09 (m, 2H), 1.86-1.74 (m, 1H), 1.72-1.61 (m, 2H), 1.59-1.45 (m, 6H), 1.44-1.36 (m, 4H), 1.33-1.23 (m, 2H), 1.17-1.03 (m, 2H), 0.98-0.86 (m, 12H).

### Example 66. Synthesis of (2R,3S,4R,5R)-5-cyano-2-((2-cyclopentylacetoxy)methyl)-5-(4-(2-ethoxy-2-methylpropanamido)pyrrolo[2,1-f][1,2,4]triazin-7-yl)-4-hydroxytetrahydrofuran-3-yl (S)-2-amino-3,3-dimethylbutanoate (66)

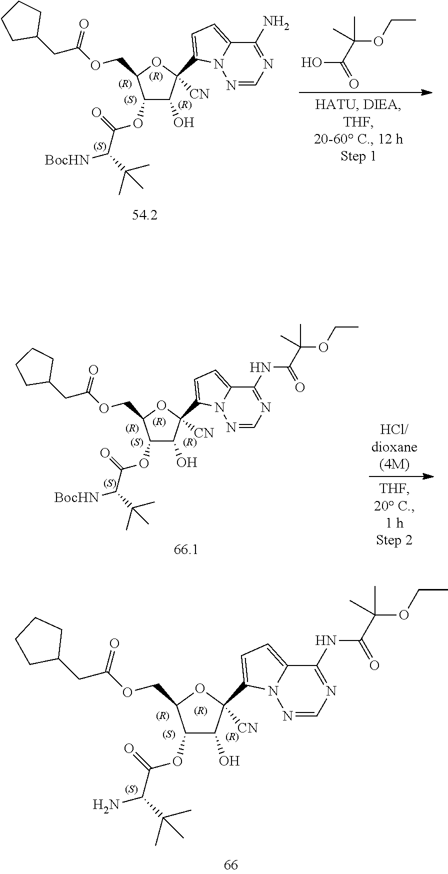

### Step 1: Synthesis of (2R,3S,4R,5R)-5-cyano-2-((2-cyclopentylacetoxy)methyl)-5-(4-(2-ethoxy-2-methylpropanamido)pyrrolo[2,1-f][1,2,4]triazin-7-yl)-4-hydroxytetrahydrofuran-3-yl (S)-2-((tert-butoxycarbonyl)amino)-3,3-dimethylbutanoate (66.1)

The title compound was prepared according to the procedure of Example 3, Step 1, using Compound 54.2 and 2-ethoxy-2-methylpropanoic acid. MS (ESI): mass calcd. for C₃₆H₅₂N₆O₁₀, 728.37, m/z found 729.20 [M+H]⁺.

### Step 2: Synthesis of (2R,3S,4R,5R)-5-cyano-2-((2-cyclopentylacetoxy)methyl)-5-(4-(2-ethoxy-2-methylpropanamido)pyrrolo[2,1-f][1,2,4]triazin-7-yl)-4-hydroxytetrahydrofuran-3-yl (S)-2-amino-3,3-dimethylbutanoate (66)

The title compound 66 was prepared according to the procedure of Example 1, Step 4, using Compound 66.1. MS (ESI): mass calcd. for C₃₁H₄₄N₆O₈, 628.32, m/z found 629.10 [M+H]⁺. H NMR (400 MHz, DMSO) δ 8.47 (s, 1H), 7.21 (d, J=4.8 Hz, 1H), 7.17 (d, J=4.8 Hz, 1H), 6.74 (d, J=6.0 Hz, 1H), 5.13-5.08 (m, 2H), 4.52-4.48 (m, 1H), 4.30 (dd, J=12.4, 3.6 Hz, 1H), 4.22 (dd, J=12.0, 4.4 Hz, 1H), 3.50 (q, J=7.2 Hz, 2H), 3.10 (s, 1H), 2.26 (dd, J=7.2, 3.2 Hz, 2H), 2.05-1.99 (m, 1H), 1.65-1.60 (m, 2H), 1.55-1.42 (m, 10H), 1.22 (t J=7.2 Hz, 3H), 1.08-1.00 (m, 2H), 0.94 (s, 9H).

### Example 67. Synthesis of (2R,3S,4R,5R)-5-cyano-2-((2-cycloheptylacetoxy)methyl)-5-(4-(2-ethoxy-2-methylpropanamido)pyrrolo[2,1-f][1,2,4]triazin-7-yl)-4-hydroxytetrahydrofuran-3-yl (S)-2-amino-3,3-dimethylbutanoate (67)

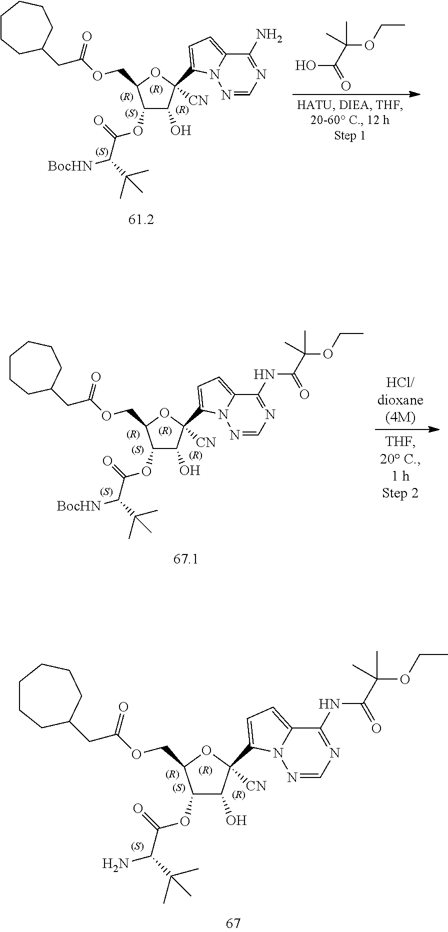

### Step 1: Synthesis of (2R,3S,4R,5R)-5-cyano-2-((2-cycloheptylacetoxy)methyl)-5-(4-(2-ethoxy-2-methylpropanamido)pyrrolo[2,1-f][1,2,4]triazin-7-yl)-4-hydroxytetrahydrofuran-3-yl (S)-2-((tert-butoxycarbonyl)amino)-3,3-dimethylbutanoate (67.1)

The title compound was prepared according to the procedure of Example 3, Step 1, using Compound 61.2 and 2-ethoxy-2-methylpropanoic acid. MS (ESI): mass calcd. for C₃₈H₅₆N₆O₁₀, 756.41, m/z found 757.30 [M+H]⁺.

### Step 2: Synthesis of (2R,3S,4R,5R)-5-cyano-2-((2-cycloheptylacetoxy)methyl)-5-(4-(2-ethoxy-2-methylpropanamido)pyrrolo[2,1-f][1,2,4]triazin-7-yl)-4-hydroxytetrahydrofuran-3-yl (S)-2-amino-3,3-dimethylbutanoate (67)

The title compound 67 was prepared according to the procedure of Example 1, Step 4, using Compound 67.1. MS (ESI): mass calcd. for C₃₃H₄₈N₆O₈, 656.35, m/z found 657.20 [M+H]⁺. H NMR (400 MHz, DMSO) δ 8.47 (s, 1H), 7.22 (d, J=4.8 Hz, 1H), 7.17 (d, J=4.8 Hz, 1H), 6.75-6.70 (m, 1H), 5.17-5.03 (m, 2H), 4.58-4.47 (m, 1H), 4.29 (dd, J=12.4, 3.6 Hz, 1H), 4.22 (dd, J=12.4, 4.8 Hz, 1H), 3.49 (q, J=7.2 Hz, 2H), 3.10 (s, 1H), 2.18 (dd, J=14.0, 7.2 Hz, 2H), 1.84-1.75 (m, 1H), 1.54-1.43 (m, 12H), 1.42-1.25 (m, 4H), 1.22 (t, J=7.2 Hz, 3H), 1.14-1.05 (m, 2H), 0.93 (s, 9H).

### Example 68. Synthesis of (2R,3S,4R,5R)-5-cyano-2-((2-cyclohexylacetoxy)methyl)-4-hydroxy-5-(4-(2-methoxy-2-methylpropanamido)pyrrolo[2,1-f][1,2,4]triazin-7-yl)tetrahydrofuran-3-yl (R)-2-amino-3,3-dimethylbutanoate (68)

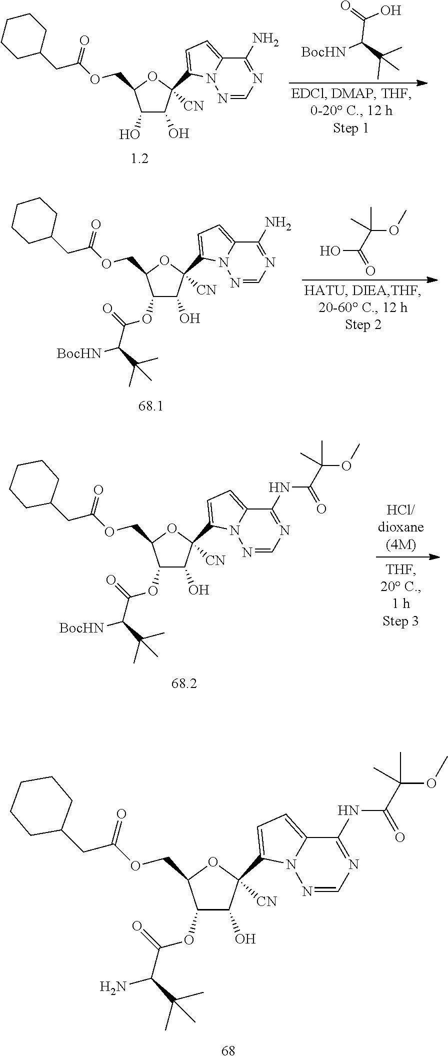

### Step 1. Synthesis of (2R,3S,4R,5R)-5-(4-aminopyrrolo[2,1-f][1,2,4]triazin-7-yl)-5-cyano-2-((2-cyclohexylacetoxy)methyl)-4-hydroxytetrahydrofuran-3-yl (R)-2-((tert-butoxycarbonyl)amino)-3,3-dimethylbutanoate (68.1)

Title compound 68.1 was prepared according to the procedure of Example 1, Step 2, using 1.2 and (R)-2-((tert-butoxycarbonyl)amino)-3,3-dimethylbutanoic acid. MS (ESI): mass calcd. for C₃₁H₄₄N₆O₈, 628.32, m/z found 629.20 [M+H]⁺. ¹H NMR (400 MHz, DMSO) δ 7.93 (m, 3H), 7.03 (d, J=8.8 Hz, 1H), 6.93 (d, J=4.8 Hz, 1H), 6.82 (d, J=4.4 Hz, 1H), 6.70 (d, J=6.0 Hz, 1H), 5.20-5.03 (m, 2H), 4.50-4.43 (m, 1H), 4.38-4.26 (m, 1H), 4.21 (dd, J=12.0, 5.2 Hz, 1H), 3.94 (d, J=8.8 Hz, 1H), 2.22-2.06 (m, 2H), 1.67-1.53 (m, 6H), 1.39 (s, 9H), 1.25-1.05 (m, 3H), 0.98 (s, 9H), 0.90-0.79 (m, 2H).

### Step 2. Synthesis of (2R,3S,4R,5R)-5-cyano-2-((2-cyclohexylacetoxy)methyl)-4-hydroxy-5-(4-(2-methoxy-2-methylpropanamido)pyrrolo[2,1-f][1,2,4]triazin-7-yl)tetrahydrofuran-3-yl (R)-2-((tert-butoxycarbonyl)amino)-3,3-dimethylbutanoate (68.2)

Title compound 68.2 was prepared according to the procedure of Example 3, Step 1, using 68.1 and 2-methoxy-2-methylpropanoic acid. MS (ESI): m/z calcd. for C₃₆H₅₂N₆O₈, 728.37, found 729.25 [M+H]⁺. ¹H NMR (400 MHz, DMSO) δ 10.31 (s, 1H), 8.49 (s, 1H), 7.24-7.20 (m, 1H), 7.18-7.13 (m, 1H), 7.05 (d, J=8.4 Hz, 1H), 6.83 (d, J=6.0 Hz, 1H), 5.20-5.14 (m, 1H), 5.13-5.05 (m, 1H), 4.56-4.48 (m, 1H), 4.40-4.28 (m, 1H), 4.27-4.16 (m, 1H), 3.95 (d, J=8.4 Hz, 1H), 3.29 (s, 3H), 2.20-2.08 (m, 2H), 1.64-1.53 (m, 6H), 1.44 (s, 6H), 1.39 (s, 9H), 1.15-1.06 (m, 3H), 0.99 (s, 9H), 0.90-0.78 (m, 2H).

### Step 3. Synthesis of (2R,3S,4R,5R)-5-cyano-2-((2-cyclohexylacetoxy)methyl)-4-hydroxy-5-(4-(2-methoxy-2-methylpropanamido)pyrrolo[2,1-f][1,2,4]triazin-7-yl)tetrahydrofuran-3-yl (R)-2-amino-3,3-dimethylbutanoate (68)

The title compound was prepared from carbamate 68.2 according to the procedure of Example 1, Step 4. MS (ESI): mass calcd. for C₃₁H₄₄N₆O₈, 628.32, m/z found 629.15 [M+H]⁺. ¹H NMR (400 MHz, DMSO) δ 8.48 (s, 1H), 7.21 (d, J=4.8 Hz, 1H), 7.16 (d, J=4.8 Hz, 1H), 6.90-6.55 (m, 1H), 5.25-5.16 (m, 1H), 5.10 (d, J=5.6 Hz, 1H), 4.55-4.50 (m, 1H), 4.37-4.27 (m, 1H), 4.28-4.16 (m, 1H), 3.29 (s, 3H), 3.11 (s, 1H), 2.20-2.07 (m, 2H), 1.62-1.51 (m, 6H), 1.44 (s, 6H), 1.19-1.03 (m, 3H), 0.96 (s, 9H), 0.89-0.76 (m, 2H).

### Example 69. Synthesis of (2R,3S,4R,5R)-5-cyano-2-((2-cyclohexylacetoxy)methyl)-5-(4-(2-ethylbutanamido)pyrrolo[2,1-f][1,2,4]triazin-7-yl)-4-hydroxytetrahydrofuran-3-yl (R)-2-amino-3,3-dimethylbutanoate (69)

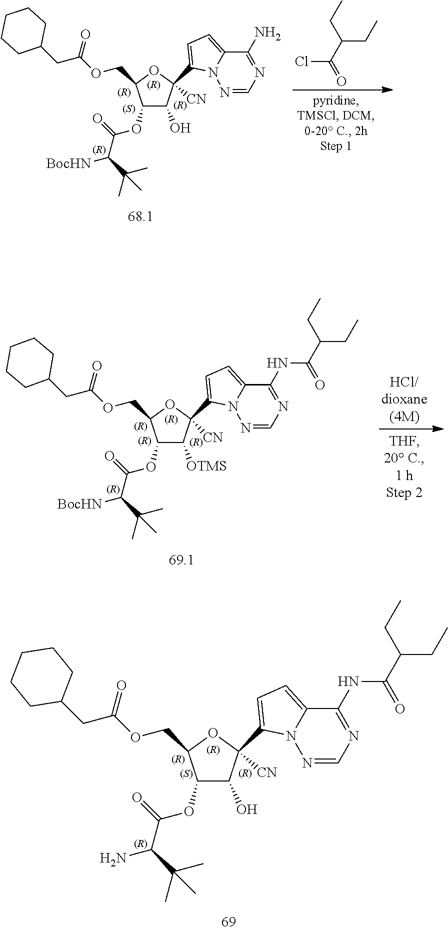

### Step 1: Synthesis of (2R,3R,4R,5R)-5-cyano-2-((2-cyclohexylacetoxy)methyl)-5-(4-(2-ethylbutanamido)pyrrolo[2,1-f][1,2,4]triazin-7-yl)-4-((trimethylsilyl)oxy)tetrahydrofuran-3-yl (R)-2-((tert-butoxycarbonyl)amino)-3,3-dimethylbutanoate (69.1)

The title compound was prepared according to the procedure of Example 1, Step 3, using Compound 68.1 and 2-ethylbutanoyl chloride. MS (ESI): mass calcd. for C₄₀H₆₂N₆O₉Si, 798.43, m/z found 799.2 [M+H]⁺.

### Step 2: Synthesis of (2R,3S,4R,5R)-5-cyano-2-((2-cyclohexylacetoxy)methyl)-5-(4-(2-ethylbutanamido)pyrrolo[2,1-f][1,2,4]triazin-7-yl)-4-hydroxytetrahydrofuran-3-yl (R)-2-amino-3,3-dimethylbutanoate (69)

The title compound 69 was prepared according to the procedure of Example 1, Step 4, using Compound 69.1. MS (ESI): mass calcd. for C₃₂H₄₆N₆O₇, 626.34, m/z found 627.15 [M+H]⁺. ¹H NMR (400 MHz, DMSO) δ 10.99 (s, 1H), 8.43 (s, 1H), 7.25 (d, J=4.8 Hz, 1H), 7.13 (d, J=4.8 Hz, 1H), 6.75-6.60 (m, 1H), 5.23-5.16 (m, 1H), 5.09 (d, J=5.6 Hz, 1H), 4.52-4.50 (m, 1H), 4.30 (dd, J=12.4, 3.6 Hz, 1H), 4.21 (dd, J=12.4, 4.8 Hz, 1H), 3.08 (s, 1H), 2.78-2.70 (m, 1H), 2.12-2.05 (m, 2H), 1.70-1.50 (m, 10H), 1.28-1.00 (m, 4H), 0.95 (s, 9H), 0.88 (t, J=7.6 Hz, 6H), 0.80-0.75 (m, 2H).

### Example 70. Synthesis of (2R,3S,4R,5R)-5-cyano-2-((2-cycloheptylacetoxy)methyl)-4-hydroxy-5-(4-(2-methoxy-2-methylpropanamido)pyrrolo[2,1-f][1,2,4]triazin-7-yl)tetrahydrofuran-3-yl (R)-2-amino-3,3-dimethylbutanoate (70)

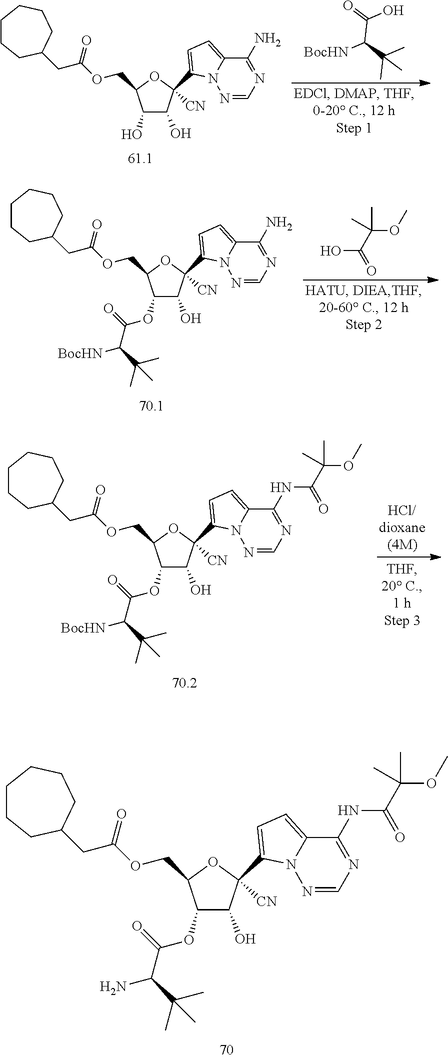

### Step 1. Synthesis of (2R,3S,4R,5R)-5-(4-aminopyrrolo[2,1-f][1,2,4]triazin-7-yl)-5-cyano-2-((2-cycloheptylacetoxy)methyl)-4-hydroxytetrahydrofuran-3-yl (R)-2-((tert-butoxycarbonyl)amino)-3,3-dimethylbutanoate (70.1)

Title compound 68.1 was prepared according to the procedure of Example 1, Step 2, using 61.1 and (R)-2-((tert-butoxycarbonyl)amino)-3,3-dimethylbutanoic acid. MS (ESI): mass calcd. for C₃₂H₄₆N₆O₈, 642.34, m/z found 643.25 [M+H]⁺. ¹H NMR (400 MHz, DMSO) δ 7.92 (br s, 3H), 7.03 (d, J=8.8 Hz, 1H), 6.94 (d, J=4.8 Hz, 1H), 6.86 (d, J=4.8 Hz, 1H), 6.70 (d, J=5.6 Hz, 1H), 5.20-5.07 (m, 2H), 4.47 (dd, J=8.8, 4.4 Hz, 1H), 4.37-4.27 (m, 1H), 4.21 (dd, J=12.0, 4.8 Hz, 1H), 3.94 (d, J=8.8 Hz, 1H), 2.26-2.10 (m, 2H), 1.92-1.75 (m, 1H), 1.63-1.46 (m, 6H), 1.44-1.27 (m, 13H), 1.18-1.06 (m, 2H), 0.98 (s, 9H).

### Step 2. Synthesis of (2R,3S,4R,5R)-5-cyano-2-((2-cycloheptylacetoxy)methyl)-4-hydroxy-5-(4-(2-methoxy-2-methylpropanamido)pyrrolo[2,1-f][1,2,4]triazin-7-yl)tetrahydrofuran-3-yl (R)-2-((tert-butoxycarbonyl)amino)-3,3-dimethylbutanoate (70.2)

Title compound 70.2 was prepared according to the procedure of Example 3, Step 1, using 70.1 and 2-methoxy-2-methylpropanoic acid. MS (ESI): m/z calcd. for C₃₇H₅₄N₆O₁₀, 742.39, found 743.25 [M+H]⁺. ¹H NMR (400 MHz, DMSO) δ 10.30 (s, 1H), 8.49 (s, 1H), 7.22 (d, J=4.8 Hz, 1H), 7.17 (d, J=4.8 Hz, 1H), 7.05 (d, J=8.4 Hz, 1H), 6.82 (d, J=6.4 Hz, 1H), 5.15 (t J=5.6 Hz, 1H), 5.09 (t, J=6.0 Hz, 1H), 4.53 (dd, J=8.8, 4.8 Hz, 1H), 4.35-4.28 (m, 1H), 4.25-4.18 (m, 1H), 3.95 (d, J=8.8 Hz, 1H), 3.29 (s, 3H), 2.19-2.13 (m, 2H), 1.85-1.75 (m, 1H), 1.61-1.47 (m, 6H), 1.44 (s, 6H), 1.39 (s, 9H), 1.36-1.23 (m, 4H), 1.14-1.06 (m, 2H), 0.99 (s, 9H).

### Step 3. Synthesis of (2R,3S,4R,5R)-5-cyano-2-((2-cycloheptylacetoxy)methyl)-4-hydroxy-5-(4-(2-methoxy-2-methylpropanamido)pyrrolo[2,1-f][1,2,4]triazin-7-yl)tetrahydrofuran-3-yl (R)-2-amino-3,3-dimethylbutanoate (70)

The title compound was prepared from carbamate 70.2 according to the procedure of Example 1, Step 4. MS (ESI): mass calcd. for C₃₂H₄₆N₆O₈, 642.34, m/z found 643.15 [M+H]⁺. ¹H NMR (400 MHz, DMSO) δ 8.47 (s, 1H), 7.21 (d, J=4.8 Hz, 1H), 7.16 (d, J=4.8 Hz, 1H), 6.82-6.70 (m, 1H), 5.24-5.17 (m, 1H), 5.14-5.08 (m, 1H), 4.55-4.50 (m, 1H), 4.35-4.26 (m, 1H), 4.22 (dd, J=12.4, 4.8 Hz, 1H), 3.29 (s, 3H), 3.12 (s, 1H), 2.24-2.09 (m, 2H), 1.87-1.73 (m, 1H), 1.60-1.46 (m, 6H), 1.44 (s, 6H), 1.42-1.23 (m, 4H), 1.15-1.03 (m, 2H), 0.96 (s, 9H).

### Example 71. Synthesis of (2R,3S,4R,5R)-5-(4-(2-butoxy-2-methylpropanamido)pyrrolo[2,1-f][1,2,4]triazin-7-yl)-5-cyano-2-((2-cyclopentylacetoxy)methyl)-4-hydroxytetrahydrofuran-3-yl(S)-2-amino-3,3-dimethylbutanoate (71)

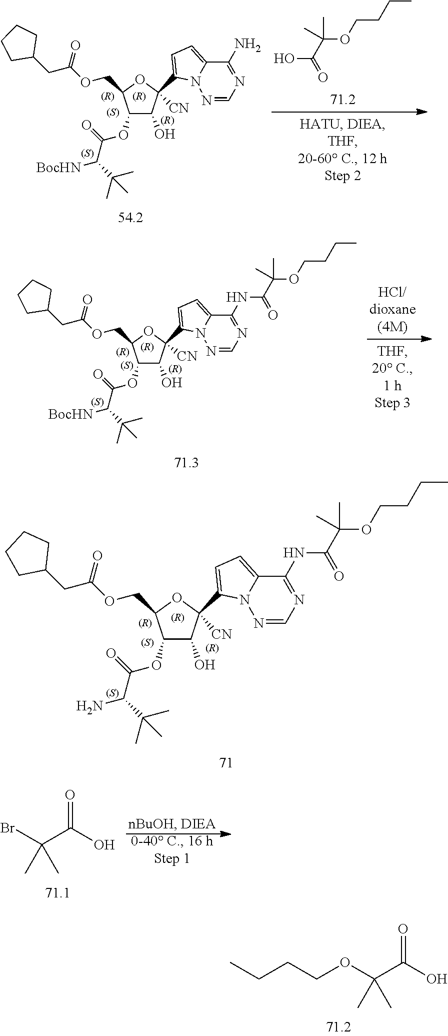

### Step 1: Synthesis of 2-butoxy-2-methylpropanoic acid (71.2)

To a solution of compound 71.1 (5.00 g, 0.0290 mol) in n-BuOH (40 mL) was added DIEA (8.11 g, 0.0620 mol) dropwise at −5° C., the reaction was then warmed to 40° C. for 16 h. The reaction mixture was cooled to 20° C. and concentrated in vacuo to obtain the slurry. MTBE (20 mL) and water (10 mL) was added to the slurry and cooled to 0° C. The mixture was acidified to pH=4 with 10% HCl (20 mL). Then the mixture was extracted with MTBE (10 mL×3). The organic phase was washed with brine (10 mL×3), then dried over anhydrous sodium sulfate, filtered, and concentrated in vacuo to obtain the compound 71.2 (3.50 g, 62% yield) as a light-yellow oil. MS (ESI): mass calcd. for C₈H₁₆O₃, 160.21, m/z found 161.2 [M+H]⁺. ¹H NMR (400 MHz, DMSO-d₆) δ 12.48 (s, 1H), 3.31 (t, J=6.4 Hz, 2H), 1.45-1.40 (m, 2H), 1.38-1.25 (m, 8H), 0.86 (t, J=7.2 Hz, 3H).

### Step 2: Synthesis of (2R,3S,4R,5R)-5-(4-(2-butoxy-2-methylpropanamido)pyrrolo[2,1-f][1,2,4]triazin-7-yl)-5-cyano-2-((2-cyclopentylacetoxy)methyl)-4-hydroxytetrahydrofuran-3-yl(S)-2-((tert-butoxycarbonyl)amino)-3,3-dimethylbutanoate (71.3)

The title compound was prepared according to the procedure of Example 3, Step 1, using Compound 54.2 and 2-butoxy-2-methylpropanoic acid. MS (ESI): mass calcd. for C₃₈H₅₆N₆O₁₀, 756.41, m/z found 757.30 [M+H]⁺.

### Step 3: Synthesis of (2R,3S,4R,5R)-5-(4-(2-butoxy-2-methylpropanamido)pyrrolo[2,1-f][1,2,4]triazin-7-yl)-5-cyano-2-((2-cyclopentylacetoxy)methyl)-4-hydroxytetrahydrofuran-3-yl(S)-2-amino-3,3-dimethylbutanoate (71)

The title compound 71 was prepared according to the procedure of Example 1, Step 4, using Compound 71.3. MS (ESI): mass calcd. for C₃₃H₄₈N₆O₈, 656.35, m/z found 657.20 [M+H]⁺. ¹H NMR (400 MHz, DMSO) δ 8.48 (s, 1H), 7.22 (d, J=4.8 Hz, 1H), 7.17 (d, J=4.8 Hz, 1H), 6.75-6.70 (m, 1H), 5.12-5.05 (m, 2H), 4.52-4.48 (m, 1H), 4.30 (dd, J=12.4, 3.6 Hz, 1H), 4.21 (dd, J=12.4, 4.8 Hz, 1H), 3.44 (t, J=6.8 Hz, 2H), 3.10 (s, 1H), 2.30-2.23 (m, 2H), 2.02-1.95 (m, 1H), 1.74-1.51 (m, 6H), 1.49-1.36 (m, 10H), 1.09-0.99 (m, 2H), 0.94 (s, 9H), 0.89 (t, J=7.6 Hz, 3H).

### Example 72. Synthesis of (2R,3S,4R,5R)-5-(4-(2-butoxy-2-methylpropanamido)pyrrolo[2,1-f][1,2,4]triazin-7-yl)-5-cyano-2-((2-cycloheptylacetoxy)methyl)-4-hydroxytetrahydrofuran-3-yl(S)-2-amino-3,3-dimethylbutanoate (72)

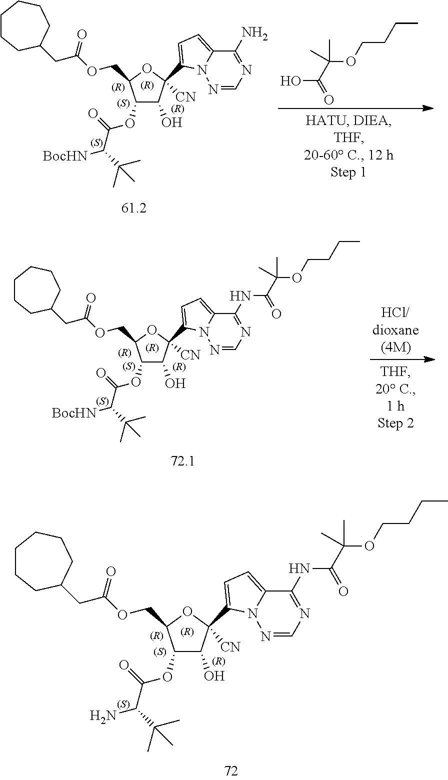

### Step 1: Synthesis of (2R,3S,4R,5R)-5-(4-(2-butoxy-2-methylpropanamido)pyrrolo[2,1-f][1,2,4]triazin-7-yl)-5-cyano-2-((2-cycloheptylacetoxy)methyl)-4-hydroxytetrahydrofuran-3-yl(S)-2-((tert-butoxycarbonyl)amino)-3,3-dimethylbutanoate (72.1)

The title compound was prepared according to the procedure of Example 3, Step 1, using Compound 61.2 and 2-butoxy-2-methylpropanoic acid. MS (ESI): mass calcd. for C₄₀H₆₀N₆O₁₀, 784.44, m/z found 785.35 [M+H]⁺.

### Step 2: Synthesis of (2R,3S,4R,5R)-5-(4-(2-butoxy-2-methylpropanamido)pyrrolo[2,1-f][1,2,4]triazin-7-yl)-5-cyano-2-((2-cycloheptylacetoxy)methyl)-4-hydroxytetrahydrofuran-3-yl(S)-2-amino-3,3-dimethylbutanoate (72)

The title compound 72 was prepared according to the procedure of Example 1, Step 4, using Compound 72.1. MS (ESI): mass calcd. for C₃₅H₅₂N₆O₈, 684.38, m/z found 685.30 [M+H]⁺. H NMR (400 MHz, DMSO) δ 8.48 (s, 1H), 7.23 (d, J=4.8 Hz, 1H), 7.17 (d, J=4.8 Hz, 1H), 6.75-6.70 (m, 1H), 5.19-5.03 (m, 2H), 4.53-4.50 (m, 1H), 4.29 (dd, J=12.4, 3.6 Hz, 1H), 4.22 (dd, J=12.4, 4.8 Hz, 1H), 3.44 (t, J=6.8 Hz, 2H), 3.12 (s, 1H), 2.22-2.12 (m, 2H), 1.77-1.70 (m, 1H), 1.65-1.35 (m, 14H), 1.39-1.35 (m, 4H), 1.28-1.24 (m, 2H), 1.15-1.02 (m, 2H), 0.94 (s, 9H), 0.90 (t, J=7.6 Hz, 3H).

### Example 73. Synthesis of (2R,3S,4R,5R)-5-(4-(2-butoxy-2-methylpropanamido)pyrrolo[2,1-f][1,2,4]triazin-7-yl)-5-cyano-2-((2-cyclohexylacetoxy)methyl)-4-hydroxytetrahydrofuran-3-yl (R)-2-amino-3,3-dimethylbutanoate (73)

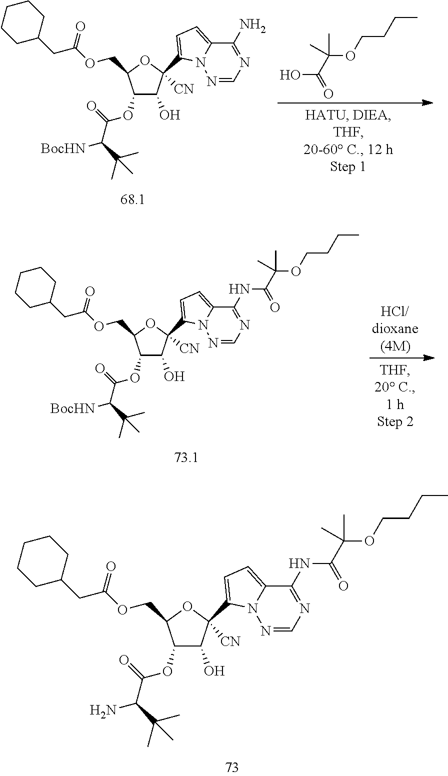 [SMILES: CC(C)(C)OC(=O)N[C@@H](C(=O)O[C@H]1[C@@H](O)[C@](C#N)(c2ccc3c(N)ncnn23)O[C@@H]1COC(=O)CC1CCCCC1)C(C)(C)C ; CCCCOC(C)(C)C(=O)O ; CCCCOC(C)(C)C(=O)Nc1ncnn2c([C@]3(C#N)O[C@H](COC(=O)CC4CCCCC4)[C@@H](OC(=O)[C@H](NC(=O)OC(C)(C)C)C(C)(C)C)[C@H]3O)ccc12 ; CCCCOC(C)(C)C(=O)Nc1ncnn2c([C@]3(C#N)O[C@H](COC(=O)CC4CCCCC4)[C@@H](OC(=O)[C@H](N)C(C)(C)C)[C@H]3O)ccc12]

### Step 1. Synthesis of (2R,3S,4R,5R)-5-(4-(2-butoxy-2-methylpropanamido)pyrrolo[2,1-f][1,2,4]triazin-7-yl)-5-cyano-2-((2-cyclohexylacetoxy)methyl)-4-hydroxytetrahydrofuran-3-yl (R)-2-((tert-butoxycarbonyl)amino)-3,3-dimethylbutanoate (73.1)

Title compound 73.1 was prepared according to the procedure of Example 3, Step 1, using 68.1 and 2-butoxy-2-methylpropanoic acid. MS (ESI): m/z calcd. for C₃₉H₅₈N₆O₁₀, 770.42, found 771.30 [M+H]⁺. H NMR (400 MHz, DMSO) δ 10.08 (s, 1H), 8.49 (s, 1H), 7.22 (t, J=4.8 Hz, 1H), 7.17 (d, J=4.8 Hz, 1H), 7.05 (d, J=8.8 Hz, 1H), 6.83 (d, J=6.8 Hz, 1H), 5.15 (t J=5.2 Hz, 1H), 5.12-5.03 (m, 1H), 4.53 (dd, J=8.8, 4.4 Hz, 1H), 4.42-4.28 (m, 1H), 4.21 (dd, J=12.4, 4.8 Hz, 1H), 3.95 (d, J=8.8 Hz, 1H), 3.44 (t, J=6.4 Hz, 2H), 2.21-2.06 (m, 2H), 1.68-1.52 (m, 8H), 1.45 (s, 6H), 1.42-1.32 (m, 11H), 1.16-1.04 (m, 3H), 0.98 (s, 9H), 0.90 (t, J=7.2 Hz, 3H), 0.87-0.75 (m, 2H).

### Step 2. Synthesis of (2R,3S,4R,5R)-5-(4-(2-butoxy-2-methylpropanamido)pyrrolo[2,1-f][1,2,4]triazin-7-yl)-5-cyano-2-((2-cyclohexylacetoxy)methyl)-4-hydroxytetrahydrofuran-3-yl (R)-2-amino-3,3-dimethylbutanoate (73)

The title compound was prepared from carbamate 73.1 according to the procedure of Example 1, Step 4. MS (ESI): mass calcd. for C₃₄H₅₀N₆O₈, 670.37, m/z found 671.20 [M+H]⁺. ¹H NMR (400 MHz, DMSO) δ 8.47 (s, 1H), 7.22 (d, J=4.8 Hz, 1H), 7.17 (d, J=4.8 Hz, 1H), 6.82-6.66 (m, 1H), 5.19 (t, J=5.2 Hz, 1H), 5.14-5.06 (m, 1H), 4.53 (dd, J=8.4, 4.4 Hz, 1H), 4.38-4.27 (m, 1H), 4.22 (dd, J=12.4, 4.8 Hz, 1H), 3.44 (t, J=6.4 Hz, 2H), 3.09 (s, 1H), 2.20-2.04 (m, 2H), 1.65-1.49 (m, 8H), 1.44 (s, 6H), 1.42-1.33 (m, 2H), 1.20-1.03 (m, 3H), 1.01-0.74 (m, 14H).

### Example 74. Synthesis of (2R,3S,4R,5R)-5-(4-(2-butoxy-2-methylpropanamido)pyrrolo[2,1-f][1,2,4]triazin-7-yl)-5-cyano-2-((2-cyclohexylacetoxy)methyl)-4-hydroxytetrahydrofuran-3-yl (S)-2-amino-3,3-dimethylbutanoate (74)

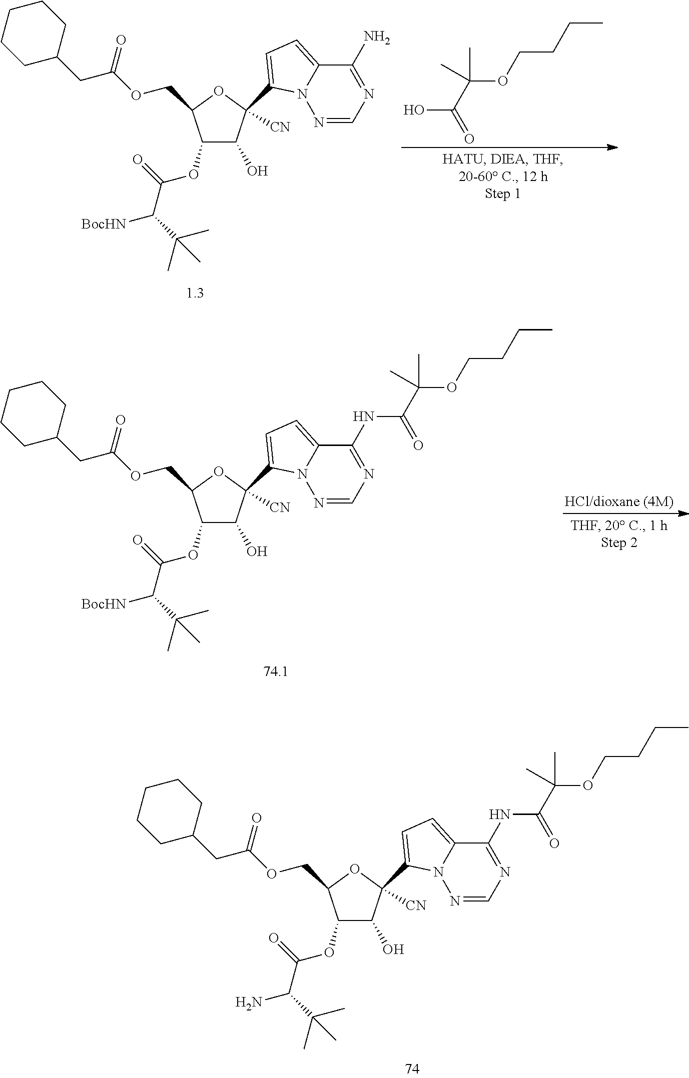 [SMILES: CC(C)(C)OC(=O)N[C@H](C(=O)O[C@H]1[C@@H](O)[C@](C#N)(c2ccc3c(N)ncnn23)O[C@@H]1COC(=O)CC1CCCCC1)C(C)(C)C ; CCCCOC(C)(C)C(=O)O ; CCCCOC(C)(C)C(=O)Nc1ncnn2c([C@]3(C#N)O[C@H](COC(=O)CC4CCCCC4)[C@@H](OC(=O)[C@@H](NC(=O)OC(C)(C)C)C(C)(C)C)[C@H]3O)ccc12 ; CCCCOC(C)(C)C(=O)Nc1ncnn2c([C@]3(C#N)O[C@H](COC(=O)CC4CCCCC4)[C@@H](OC(=O)[C@@H](N)C(C)(C)C)[C@H]3O)ccc12]

### Step 1. Synthesis of (2R,3S,4R,5R)-5-(4-(2-butoxy-2-methylpropanamido)pyrrolo[2,1-f][1,2,4]triazin-7-yl)-5-cyano-2-((2-cyclohexylacetoxy)methyl)-4-hydroxytetrahydrofuran-3-yl (S)-2-((tert-butoxycarbonyl)amino)-3,3-dimethylbutanoate (74.1)

Title compound 74.1 was prepared according to the procedure of Example 3, Step 1, using 1.3 and 2-butoxy-2-methylpropanoic acid. MS (ESI): m/z calcd. for C₃₉H₅₈N₆O₁₀, 770.42, found 771.45 [M+H]⁺. ¹H NMR (400 MHz, DMSO) δ 10.08 (s, 1H), 8.49 (s, 1H), 7.23 (t, J=4.8 Hz, 1H), 7.17 (d, J=4.8 Hz, 1H), 7.05 (d, J=8.4 Hz, 1H), 6.78 (d, J=6.4 Hz, 1H), 5.39-5.35 (m, 1H), 5.15-5.11 (m, 1H), 4.53-4.43 (m, 1H), 4.34-4.27 (m, 1H), 4.19 (dd, J=12.4, 4.8 Hz, 1H), 3.98-3.93 (m, 1H), 3.44 (t, J=6.4 Hz, 2H), 2.20-2.07 (m, 2H), 1.63-1.51 (m, 8H), 1.45 (s, 6H), 1.40 (s, 9H), 1.38-1.30 (m, 2H), 1.15-1.04 (m, 3H), 0.99 (s, 9H), 0.92-0.83 (m, 5H).

### Step 2. Synthesis of (2R,3S,4R,5R)-5-(4-(2-butoxy-2-methylpropanamido)pyrrolo[2,1-f][1,2,4]triazin-7-yl)-5-cyano-2-((2-cyclohexylacetoxy)methyl)-4-hydroxytetrahydrofuran-3-yl (S)-2-amino-3,3-dimethylbutanoate (74.1)

The title compound was prepared from carbamate 74.1 according to the procedure of Example 1, Step 4. MS (ESI): mass calcd. for C₃₄H₅₀N₆O₈, 670.37, m/z found 671.25 [M+H]⁺. ¹H NMR (400 MHz, DMSO) δ 10.07 (s, 1H), 8.48 (s, 1H), 7.23 (d, J=4.8 Hz, 1H), 7.17 (d, J=4.8 Hz, 1H), 6.80-6.70 (m, 1H), 5.17-5.05 (m, 2H), 4.56-4.49 (m, 1H), 4.30 (dd, J=12.4, 3.2 Hz, 1H), 4.21 (dd, J=12.4, 4.8 Hz, 1H), 3.44 (t, J=6.4 Hz, 2H), 3.13 (s, 1H), 2.22-2.03 (m, 2H), 1.62-1.51 (m, 8H), 1.45 (s, 6H), 1.43-1.34 (m, 2H), 1.18-1.03 (m, 3H), 0.94 (s, 9H), 0.92-0.80 (m, 5H).

### Example 75. Synthesis of (2R,3S,4R,5R)-5-(4-(2-butoxy-2-methylpropanamido)pyrrolo[2,1-f][1,2,4]triazin-7-yl)-5-cyano-2-((2-cycloheptylacetoxy)methyl)-4-hydroxytetrahydrofuran-3-yl(R)-2-amino-3,3-dimethylbutanoate (75)

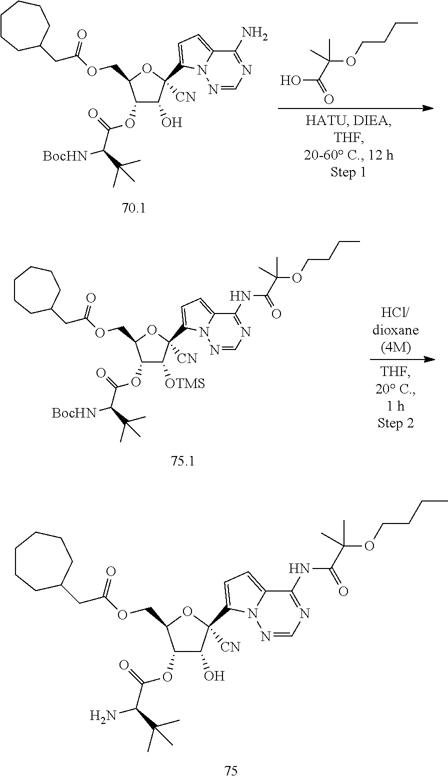

### Step 1. Synthesis of (2R,3S,4R,5R)-5-(4-(2-butoxy-2-methylpropanamido)pyrrolo[2,1-f][1,2,4]triazin-7-yl)-5-cyano-2-((2-cycloheptylacetoxy)methyl)-4-hydroxytetrahydrofuran-3-yl(R)-2-((tert-butoxycarbonyl)amino)-3,3-dimethylbutanoate (75.1)

Title compound 75.1 was prepared according to the procedure of Example 3, Step 1, using 70.1 and 2-butoxy-2-methylpropanoic acid. MS (ESI): m/z calcd. for C₄₀H₆₀N₆O₁₀, 784.44, found 785.35 [M+H]⁺. H NMR (400 MHz, DMSO) δ 10.08 (s, 1H), 8.49 (s, 1H), 7.23 (d, J=4.8 Hz, 1H), 7.17 (d, J=4.8 Hz, 1H), 7.05 (d, J=8.4 Hz, 1H), 6.83 (d, J=5.6 Hz, 1H), 5.14 (t J=5.6 Hz, 1H), 5.11-5.04 (m, 1H), 4.53 (dd, J=8.8, 4.8 Hz, 1H), 4.35-4.28 (m, 1H), 4.22 (dd, J=12.4, 4.8 Hz, 1H), 3.95 (d, J=8.8 Hz, 1H), 3.44 (t, J=6.4 Hz, 2H), 2.22-2.09 (m, 2H), 1.85-1.72 (m, 1H), 1.64-1.47 (m, 8H), 1.45 (s, 6H), 1.43-1.30 (m, 15H), 1.13-1.05 (m, 2H), 0.98 (s, 9H), 0.90 (t, J=7.2 Hz, 3H).

### Step 2. Synthesis of (2R,3S,4R,5R)-5-(4-(2-butoxy-2-methylpropanamido)pyrrolo[2,1-f][1,2,4]triazin-7-yl)-5-cyano-2-((2-cycloheptylacetoxy)methyl)-4-hydroxytetrahydrofuran-3-yl(R)-2-amino-3,3-dimethylbutanoate (75)

The title compound was prepared from carbamate 75.1 according to the procedure of Example 1, Step 4. MS (ESI): mass calcd. for C₃₅H₅₂N₆O₈, 684.38, m/z found 685.30 [M+H]⁺. ¹H NMR (400 MHz, DMSO) δ 10.08 (s, 1H), 8.47 (s, 1H), 7.22 (d, J=4.8 Hz, 1H), 7.18 (d, J=4.8 Hz, 1H), 6.86-6.72 (m, 1H), 5.20 (t, J=5.2 Hz, 1H), 5.15-5.07 (m, 1H), 4.58-4.48 (m, 1H), 4.31 (dd, J=12.4, 3.6 Hz, 1H), 4.22 (dd, J=12.4, 4.8 Hz, 1H), 3.44 (t, J=6.4 Hz, 2H), 3.19 (s, 1H), 2.24-2.09 (m, 2H), 1.87-1.72 (m, 1H), 1.64-1.46 (m, 8H), 1.45 (s, 6H), 1.42-1.33 (m, 4H), 1.32-1.22 (m, 2H), 1.17-1.02 (m, 2H), 1.01-0.81 (m, 12H).

### Example 76. Synthesis of (2R,3S,4R,5R)-5-cyano-2-((2-cyclopentylacetoxy)methyl)-5-(4-(2-ethylbutanamido)pyrrolo[2,1-f][1,2,4]triazin-7-yl)-4-hydroxytetrahydrofuran-3-yl (R)-2-amino-3,3-dimethylbutanoate (76)

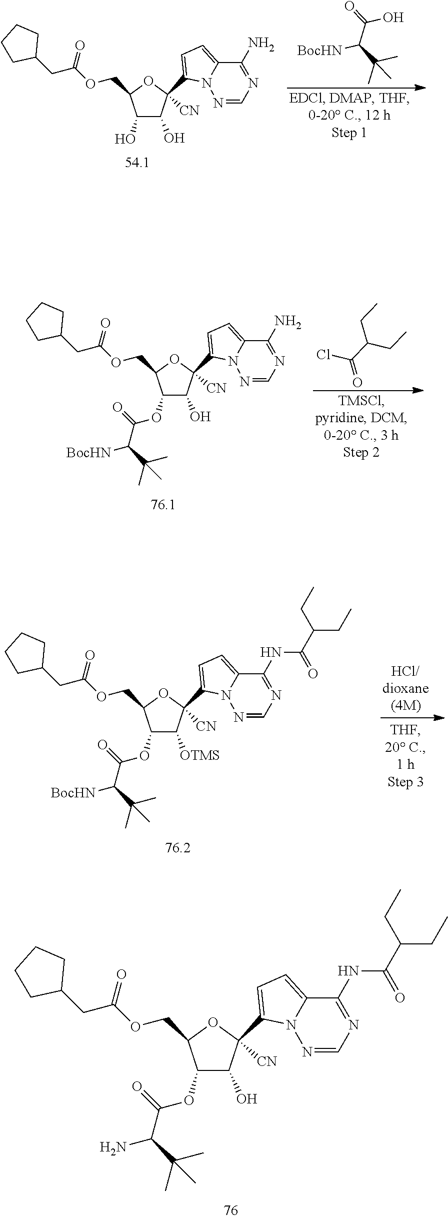

### Step 1. Synthesis of (2R,3S,4R,5R)-5-(4-aminopyrrolo[2,1-f][1,2,4]triazin-7-yl)-5-cyano-2-((2-cyclopentylacetoxy)methyl)-4-hydroxytetrahydrofuran-3-yl (R)-2-((tert-butoxycarbonyl)amino)-3,3-dimethylbutanoate (76.1)

Title compound 76.1 was prepared according to the procedure of Example 1, Step 2, using 54.1 and (R)-2-((tert-butoxycarbonyl)amino)-3,3-dimethylbutanoic acid. MS (ESI): mass calcd. for C₃₀H₄₂N₆O₈, 614.31, m/z found 615.25 [M+H]⁺. H NMR (400 MHz, DMSO) δ 7.92 (br s, 3H), 7.03 (d, J=8.8 Hz, 1H), 6.93 (d, J=4.8 Hz, 1H), 6.86 (d, J=4.8 Hz, 1H), 6.70 (d, J=6.4 Hz, 1H), 5.17-5.04 (m, 2H), 4.46 (dd, J=8.8, 4.8 Hz, 1H), 4.38-4.28 (m, 1H), 4.20 (dd, J=12.0, 4.8 Hz, 1H), 3.94 (d, J=8.8 Hz, 1H), 2.34-2.20 (m, 2H), 2.10-1.99 (m, 1H), 1.73-1.63 (m, 2H), 1.59-1.41 (m, 4H), 1.38 (s, 9H), 1.12-1.01 (m, 2H), 0.98 (s, 9H).

### Step 2. Synthesis of (2R,3R,4R,5R)-5-cyano-2-((2-cyclopentylacetoxy)methyl)-5-(4-(2-ethylbutanamido)pyrrolo[2,1-f][1,2,4]triazin-7-yl)-4-((trimethylsilyl)oxy)tetrahydrofuran-3-yl (R)-2-((tert-butoxycarbonyl)amino)-3,3-dimethylbutanoate (76.2)

Title compound 76.2 was prepared according to the procedure of Example 1, Step 3, using 76.1 and 2-ethylbutanoyl chloride. MS (ESI): m/z calcd. for C₃₀H₆₀N₆O₉Si, 784.42, found 785.40 [M+H]⁺.

### Step 3. Synthesis of (2R,3S,4R,5R)-5-cyano-2-((2-cyclopentylacetoxy)methyl)-5-(4-(2-ethylbutanamido)pyrrolo[2,1-f][1,2,4]triazin-7-yl)-4-hydroxytetrahydrofuran-3-yl (R)-2-amino-3,3-dimethylbutanoate (76)

The title compound was prepared from carbamate 76.2 according to the procedure of Example 1, Step 4. MS (ESI): mass calcd. for C₃₁H₄₄N₆O₇, 612.33, m/z found 613.10 [M+H]⁺. ¹H NMR (400 MHz, DMSO) δ 10.98 (s, 1H), 8.43 (s, 1H), 7.24 (d, J=4.8 Hz, 1H), 7.13 (d, J=4.8 Hz, 1H), 6.76-6.70 (m, 1H), 5.21 (t, J=5.2 Hz, 1H), 5.11 (d, J=5.6 Hz, 1H), 4.53-4.50 (m, 1H), 4.31 (dd, J=12.0, 3.2 Hz, 1H), 4.21 (dd, J=12.4, 4.8 Hz, 1H), 3.17 (s, 1H), 2.77-2.70 (m, 1H), 2.28-2.19 (m, 2H), 2.01-1.95 (m, 1H), 1.74-1.59 (m, 4H), 1.57-1.47 (m, 4H), 1.42-1.38 (m, 2H), 1.13-0.99 (m, 2H), 0.95 (s, 9H), 0.89 (t, J=7.6 Hz, 6H).

### Example 77. Synthesis of (2R,3S,4R,5R)-5-cyano-2-((2-cycloheptylacetoxy)methyl)-5-(4-(2-ethylbutanamido)pyrrolo[2,1-f][1,2,4]triazin-7-yl)-4-hydroxytetrahydrofuran-3-yl (R)-2-amino-3,3-dimethylbutanoate (77)

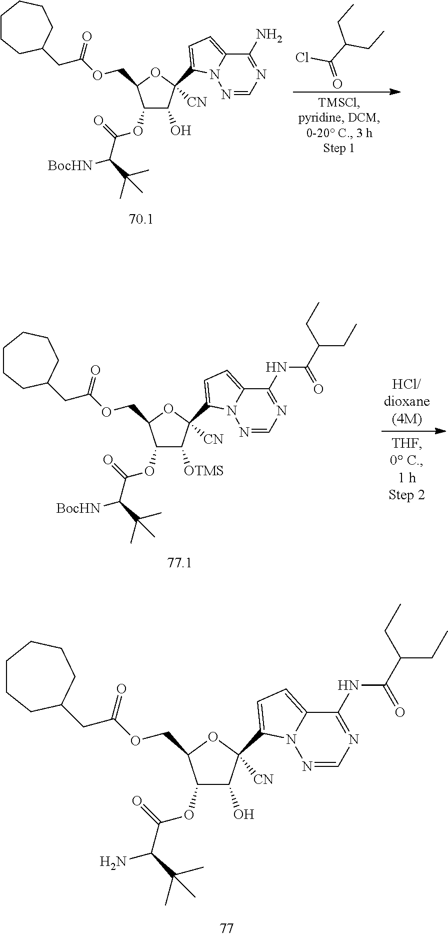

### Step 1. Synthesis of (2R,3R,4R,5R)-5-cyano-2-((2-cycloheptylacetoxy)methyl)-5-(4-(2-ethylbutanamido)pyrrolo[2,1-f][1,2,4]triazin-7-yl)-4-((trimethylsilyl)oxy)tetrahydrofuran-3-yl (R)-2-((tert-butoxycarbonyl)amino)-3,3-dimethylbutanoate (77.1)

Title compound 77.1 was prepared according to the procedure of Example 1, Step 3, using 70.1 and 2-ethylbutanoyl chloride. MS (ESI): m/z calcd. for C₄₁H₆₄N₆O₉Si, 812.45, found 813.45 [M+H]⁺. ¹H NMR (400 MHz, DMSO) δ 10.99 (s, 1H), 8.41 (s, 1H), 7.27 (d, J=4.8 Hz, 1H), 7.14 (d, J=4.8 Hz, 1H), 7.11 (d, J=8.8 Hz, 1H), 5.34 (d, J=5.2 Hz, 1H), 5.16 (t J=5.2 Hz, 1H), 4.59-4.48 (m, 1H), 4.43-4.28 (m, 2H), 3.96 (d, J=8.8 Hz, 1H), 2.83-2.73 (m, 1H), 2.28-2.15 (m, 2H), 1.89-1.78 (m, 1H), 1.67-1.46 (m, 11H), 1.42-1.28 (m, 13H), 1.15-1.06 (m, 2H), 0.95 (s, 9H), 0.89 (t, J=7.2 Hz, 6H), −0.01 (s, 9H).

### Step 2. Synthesis of (2R,3S,4R,5R)-5-cyano-2-((2-cycloheptylacetoxy)methyl)-5-(4-(2-ethylbutanamido)pyrrolo[2,1-f][1,2,4]triazin-7-yl)-4-hydroxytetrahydrofuran-3-yl (R)-2-amino-3,3-dimethylbutanoate (77)

The title compound was prepared from carbamate 77.1 according to the procedure of Example 1, Step 4. MS (ESI): mass calcd. for C₃₃H₄₈N₆O₇, 640.36, m/z found 641.15 [M+H]⁺. ¹H NMR (400 MHz, DMSO) δ 10.98 (s, 1H), 8.42 (s, 1H), 7.25 (d, J=4.8 Hz, 1H), 7.13 (d, J=4.8 Hz, 1H), 6.78-6.68 (m, 1H), 5.22-5.13 (m, 1H), 5.12-5.07 (m, 1H), 4.55-4.49 (m, 1H), 4.30 (dd, J=12.4, 3.6 Hz, 1H), 4.22 (dd, J=12.4, 4.8 Hz, 1H), 3.08 (s, 1H), 2.82-2.72 (m, 1H), 2.23-2.09 (m, 2H), 1.77 (s, 1H), 1.68-1.44 (m, 10H), 1.40-1.23 (m, 4H), 1.14-1.02 (m, 2H), 0.97-0.87 (m, 15H).

### Example I: Oral Composition of a Compounds of Formula (I), or a Pharmaceutically Acceptable Salt, Solvate, or Stereoisomer Thereof

To prepare a pharmaceutical composition for oral delivery, 400 mg of compound described herein, or a pharmaceutically acceptable salt, solvate, or stereoisomer thereof and the following ingredients are mixed intimately and pressed into single scored tablets.

### Tablet Formulation

| Ingredient | Quantity per tablet (mg) |
|---|---|
| compound | 400 |
| cornstarch | 50 |
| croscarmellose sodium | 25 |
| lactose | 120 |
| magnesium stearate | 5 |

The following ingredients are mixed intimately and loaded into a hard-shell gelatin capsule.

### Capsule Formulation

| Ingredient | Quantity per capsule (mg) |
|---|---|
| compound | 200 |
| lactose spray dried | 148 |
| magnesium stearate | 2 |

### Example II: Evaluation of Cellular Permeability in Caco-2 Cell Monolayers

The bidirectional permeability of compounds across the Caco-2 cell monolayer was assessed. The Caco-2 model is a widely used in vitro model for small intestinal absorption and potential for efflux. Cell culture. Caco-2 cells were grown in DMEM supplemented with 10% fetal bovine serum (FBS), 1% Penicillin-Streptomycin and 1% MEMNEAA. The cells were incubated at 37° C., 5% CO₂/95% air and saturated humidity. After reaching 80-90% confluency, the cells were gently detached with trypsin. Cells at passage 39 were seeded on the 24-well BD insert system at the density of 8×104 cells/cm² and cultured for 19 days with medium changed every 2-3 days. Measure the transepithelial electrical resistance (TEER) value of each well. The wells can be used only when their TEER values are greater than 600 Ohms/cm².

Transport assay. After removing the cell culture medium from the 24-well insert plate, the cells were rinsed with warm transport buffer. Appropriate dosing and receiving solutions were applied to the donor and receiver chambers to initiate the transport assay in apical to basolateral (A to B) or basolateral to apical (B to A) directions (500 and 1300 μL for apical and basolateral wells, respectively). Duplicate wells in each direction were used for the test compound and control compound. The plate was incubated in CO₂ incubator at 37° C., with 5% CO₂/95% air and saturated humidity without shaking. The sample after 10-minute incubation was used as the To sample, and the sample after 90-minute incubation was used as the T₉₀ sample. To and T₉₀ samples were collected from the donor and receiver side of each well at the designed timepoint, mixed with the transport buffer and acetonitrile/MeOH (1:1, v/v) and the internal standard for LC/MS/MS analysis. All samples were vortexed and centrifuged at 4000 rpm at 4° C. for 15 minutes, diluted with pure water and stored at 4° C. before bioanalysis by LC/MS/MS.

Sample analysis. The concentrations of test compounds and control compounds in Caco-2 cells were quantitatively determined by LC/MS/MS method after protein precipitation.

Calculations. The apparent permeability (Pₐₚₚ, cm/s), efflux ratio (ER) and recovery parameters were calculated for Caco-2 drug transport assay using the following equations:

$${P}_{app} = \frac{{V}_{R}}{Area \times Time} \times \frac{{[ drug ]}_{recevier}}{{[ drug ]}_{initial , donor}} = \frac{{V}_{R}}{Area \times Time} \times \frac{{C}_{R}}{{C}_{0}}$$

$$ER = \frac{{P}_{app} ( B to A )}{{P}_{app} ( A to B )}$$

where V_{R} is the solution volume in the receiver chamber (0.4 mL on the apical side, 1.2 mL on the basolateral side); Area is the surface area for the insert membrane, i.e., 0.3 cm² for the area of the monolayer; Time is incubation time, expressed in seconds, i. e., 5400 s (90 min) for the current experiment; Co is the initial concentration of test compound or peak area ratio of control compounds in the donor chamber (M); V_{D} is the volume in the donor chambers (0.4 mL on the apical side, 1.2 mL on the basolateral side); C_{D} and C_{R} are the final concentrations of test compounds or peak area ratio of control compounds in donor and receiver chambers, respectively.

Data for representative compounds is shown in Table 2. Compounds with Pₐₚₚ<1.0 are classified as low permeability, Pₐₚₚ between 1-10 are classified as medium permeability, and Pₐₚₚ>10 are classified as high permeability. An efflux ratio (ER)>2 indicates the compounds are potential substrates for intestinal cell efflux transporters.

TABLE 2
Bidirectional Caco-2 permeability assay results.

| | Pₐₚₚ, A-B | Papp, B-A, | |
|---|---|---|---|
| Compound No. | (×10⁻⁶ cm s⁻¹) | (×10⁻⁶ cm s⁻¹) | ER |
| 1 | 6.80 | 8.72 | 1.28 |
| 2 | 4.08 | 6.13 | 1.50 |
| 3 | 8.49 | 9.30 | 1.10 |
| 4 | 2.50 | 3.48 | 1.37 |
| 5 | 17.68 | 16.48 | 0.93 |
| 6 | 13.97 | 15.38 | 1.10 |
| 7 | 7.82 | 8.41 | 1.07 |
| 8 | 0.52 | 0.13 | 0.26 |
| 10 | 0.59 | 0.10 | 0.17 |
| 12 | 3.48 | 25.52 | 7.34 |
| 13 | 3.16 | 30.54 | 9.68 |
| 14 | 1.96 | 1.38 | 0.70 |
| 15 | 1.93 | 1.76 | 0.91 |
| 21 | 3.10 | 21.00 | 6.77 |
| 22 | 2.79 | 6.10 | 2.19 |
| 29 | 1.69 | 19.62 | 11.64 |
| 30 | 0.09 | 3.11 | 34.6 |
| 31 | 3.76 | 15.3 | 4.07 |
| 32 | 15.4 | 20.7 | 1.34 |
| 33 | 24.4 | 29.1 | 1.19 |
| 34 | 2.80 | 7.33 | 2.62 |
| 35 | 0.01 | 0.24 | 19.3 |
| 36 | 0.93 | 1.15 | 1.24 |
| 37 | 5.56 | 16.4 | 2.95 |
| 38 | 4.29 | 13.8 | 3.21 |
| 39 | 0.72 | 16.9 | 23.4 |
| 40 | 0.13 | 4.50 | 34.4 |
| 41 | 1.41 | 14.6 | 10.4 |
| 42 | 4.24 | 3.97 | 0.94 |
| 43 | 1.49 | 1.02 | 0.69 |
| 44 | 0.47 | 1.49 | 3.19 |
| 45 | 0.12 | 2.50 | 20.7 |
| 46 | 1.45 | 0.79 | 0.54 |
| 47 | 4.57 | 12.24 | 2.68 |
| 48 | 2.02 | 4.72 | 2.34 |
| 49 | 6.24 | 5.67 | 0.91 |
| 50 | 8.66 | 8.25 | 0.95 |
| 54 | 3.41 | 5.47 | 1.60 |
| 55 | 2.74 | 3.63 | 1.33 |
| 56 | 7.61 | 7.57 | 0.99 |
| 57 | 16.43 | 10.05 | 0.61 |
| 61 | 1.07 | 0.65 | 0.60 |
| 62 | 1.40 | 0.89 | 0.64 |
| 63 | 4.01 | 2.79 | 0.70 |
| 65 | 0.79 | 0.50 | 0.62 |
| 66 | 5.10 | 5.79 | 1.13 |
| 67 | 4.78 | 2.54 | 0.53 |
| 68 | 3.22 | 4.04 | 1.25 |
| 69 | 3.19 | 4.38 | 1.37 |
| 70 | 2.13 | 2.20 | 1.03 |
| 71 | 0.67 | 8.18 | 12.13 |
| 72 | 0.09 | 7.60 | 88.79 |
| 73 | 0.24 | 0.53 | 2.23 |
| 74 | 0.41 | 0.41 | 1.00 |
| 75 | 0.22 | 0.12 | 0.52 |
| 76 | 2.5 | 2.4 | 1.0 |
| 77 | 1.0 | 1.6 | 1.7 |

### Example III: Evaluation of Oral Bioavailability in Sprague-Dawley Rats

The single dose pharmacokinetics of Example Compounds is evaluated in fasted male Sprague-Dawley rats following oral gavage. Compounds are dosed as solutions or acceptable suspensions in vehicles generally-regarded as safe for in vivo studies. Groups of three rats per compound are utilized, and blood samples collected at 0.25, 0.5, 1, 2, 4, 8 and 24 h post-dose, stored on ice prior to plasma preparation by centrifugation at 4° C. at 6000 rpm for 5 min. Plasma samples are stored at −80° C. Concentrations of Example Compounds and corresponding nucleosides resulting from metabolic processing are determined using ultra-high performance liquid chromatography-triple-quadrupole mass spectrometry, with an internal standard. Pharmacokinetic parameters are derived from the resulting plasma concentration/time graphs, and include the following: plasma half-life (t_{1/2}), time to maximum plasma concentration (Tₘₐₓ), maximum plasma concentration (Cₘₐₓ), area under the curve of the plasma concentration/time graph (AUCₗₐₛₜ, through last timepoint obtained, and AUC_{inf}, including the extrapolated area), and mean residence time (MRT_{inf}). The resulting AUC_{inf} for the resulting nucleosides are compared with that resulting from intravenous bolus administration of the corresponding nucleosides in three rats, and an oral bioavailability (F, %) was be calculated as

$$F = \frac{AUCinf , PO}{AUCinf , IV} \times \frac{Dose ( IV )}{Dose ( PO )} \times \frac{FW ( prodrug )}{FW ( nucleoside )}$$

### Example II: Evaluation of Stability of Example Compounds in Human Fasted-State Simulated Gastric Fluid, and Fasted-State Simulated Intestinal Fluid

The chemical stability of example compounds was determined using human fasted-state simulated gastric fluid, and fasted-state simulated intestinal fluid, prepared as follows.

### FaSSGF:

Weigh 1.0 g NaCl and dissolved it in 0.49 L water
Adjust pH value to 1.2, and adjust volume to 0.5 L in water
Filter the NaCl/HCl buffer through a 0.22 μm filter
Weigh 3.0 mg FaSSGF powder (Vendor: Biorelevant, catalog #FFF01), dissolve it in 50 mL NaCl/HCl buffer
Dissolve the FaSSGF powder in NaCl/HCl buffer and make up the volume to 50 mL
Store at 4° C. for future use.

### FaSSIF:

Weigh 0.695 g NaOH, 1.115 g maleic acid and 2.005 g NaCl, and dissolve in 0.49 L water
Adjusted the pH value to 6.8 and adjust volume to 0.5 L with water
Filter the maleic acid buffer through a 0.22 μm filter
Weigh 89.5 mg FaSSIF-V2 powder (Vendor: Biorelevant catalog #V2FAS01), dissolve in 50 mL maleic buffer
Dissolve the FaSSIF-V2 powder in maleic buffer and make up the volume to 50 mL
Store at 4° C. for future use.

Briefly, test compounds were prepared as 50 μM working solutions in DMSO, and 2 μL test compound working solution was added to 98 μL of each buffer respectively in six tubes (for six timepoints, 0, 5, 15, 30, 60, and 120 min) and gently mixed. At each corresponding timepoint, the reaction was stopped by addition of 300 μL of quenching solution (methanol solution of 5 ng/mL terfenadine and 10 ng/mL of tolbutamide). Each tube was vortexed for 1 min, centrifuged at 4,000 rpm at 4° C. for 15 min, then 200 μL of supernatant was removed. The half-life of each example compound was determined using LC-MS/MS analysis, using the internal standards present in the quenching solution.

TABLE 3
Stability of example compounds in human fasted-state simulated
gastric fluid, and fasted-state simulated intestinal fluid. Half-
lives in human FaSSGF and FaSSIF determined by LC/MS methods.

| Compound Number | FaSSGF t_{1/2} (min) | FaSSIF t_{1/2} (min) |
|---|---|---|
| 1 | 96.9 | >373 |
| 2 | >373 | >373 |
| 3 | 20.8 | >373 |
| 37 | >372.75 | >372.75 |

What is claimed is:

1. A compound of Formula (VIa), (VIb), or (VIc), or a pharmaceutically acceptable salt, solvate, or stereoisomer thereof: 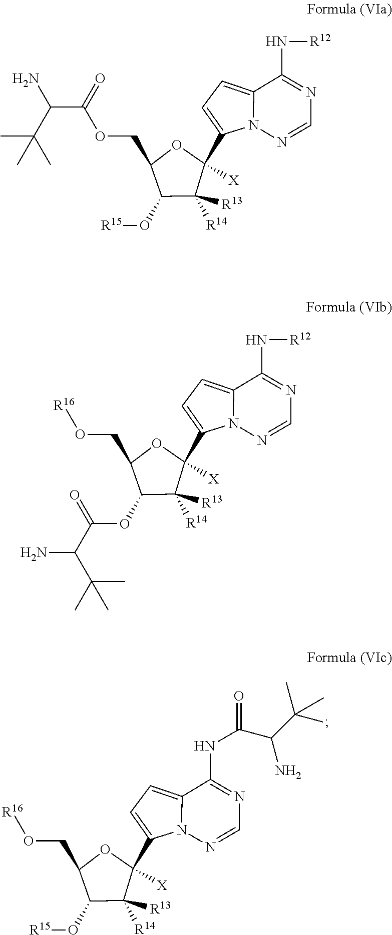
wherein:
X is hydrogen or —CN;
R¹² is hydrogen, —C(═O)R²², —C(═O)OR²², or C₁-C₆alkyl optionally substituted with one or more R^{12a};
each R^{12a} is independently halogen, —CN, —OH, —OR^{a}, —NR^{c}R^{d}, cycloalkyl, or heterocycloalkyl;
or two R^{12a} on the same atom are taken together to form an oxo;
R²² is C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{22a};
each R^{22a} is independently halogen, —CN, —NO₂, —OH, —OR^{a}, —OC(═O)R^{a}, —OC(═O)OR^{b}, —OC(═O)NR^{c}R^{d}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, cycloalkyl, heterocycloalkyl, aryl, and heteroaryl is optionally and independently substituted with one or more R;
or two R^{22a} on the same atom are taken together to form an oxo;
or two R^{22a} are taken together to form a cycloalkyl or heterocycloalkyl; each optionally and independently substituted with one or more R;
R¹³ is hydrogen or C₁-C₆alkyl;
R¹⁴ is —OH or fluoro;
R¹⁵ is hydrogen, —C(═O)R²⁵, —C(═O)OR²⁵, —CH₂—O—C(═O)R²⁵, or —CH₂—O—C(═O)OR²⁵;
R²⁵ is C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{25a};
each R^{25a} is independently halogen, —CN, —NO₂, —OH, —OR^{a}, —OC(═O)R^{a}, —OC(═O)OR^{b}, —OC(═O)NR^{c}R^{d}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, cycloalkyl, heterocycloalkyl, aryl, and heteroaryl is optionally and independently substituted with one or more R;
or two R^{25a} on the same atom are taken together to form an oxo;
or two R^{25a} are taken together to form a cycloalkyl or heterocycloalkyl; each optionally and independently substituted with one or more R;
R¹⁶ is hydrogen, —C(═O)R²⁶, —C(═O)OR²⁶, —CH₂—O—C(═O)R²⁶, —CH₂—O—C(═O)OR²⁶, or C₁-C₆alkyl optionally substituted with one or more R^{16a};
each R^{16a} is independently halogen, —CN, —OH, —OR^{a}, —NR^{c}R^{d}, cycloalkyl, or heterocycloalkyl;
or two R^{16a} on the same atom are taken together to form an oxo;
R²⁶ is C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{26a};
each R^{26a} is independently halogen, —CN, —NO₂, —OH, —OR^{a}, —OC(═O)R^{a}, —OC(═O)OR^{b}, —OC(═O)NR^{c}R^{d}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, cycloalkyl, heterocycloalkyl, aryl, and heteroaryl is optionally and independently substituted with one or more R;
or two R^{26a} on the same atom are taken together to form an oxo;
or two R^{26a} are taken together to form a cycloalkyl or heterocycloalkyl; each optionally and independently substituted with one or more R;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, and C₁-C₆heteroalkyl;
or two R on the same atom are taken together to form an oxo.

2. The compound of claim 1, wherein the compound is a compound of Formula (VIa):

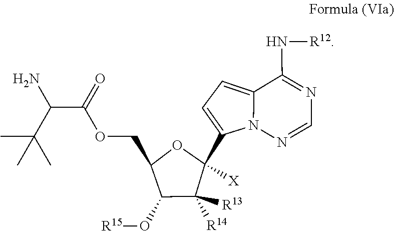

3. The compound of claim 1 or 2, wherein the compound is a compound of Formula (VIa-1):

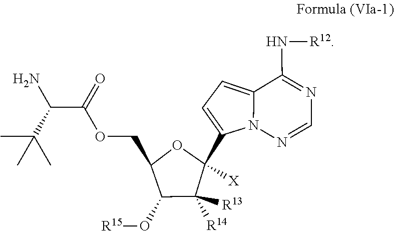

4. The compound of claim 1 or 2, wherein the compound is a compound of Formula (VIa-2):

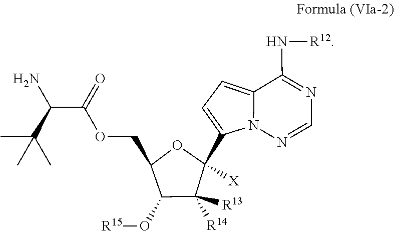

5. The compound of claim 1, wherein the compound is a compound of Formula (VIb):

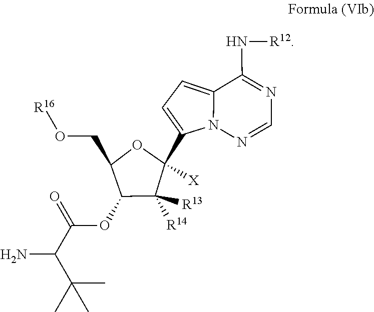

6. The compound of claim 1 or 5, wherein the compound is a compound of Formula (VIb-1):

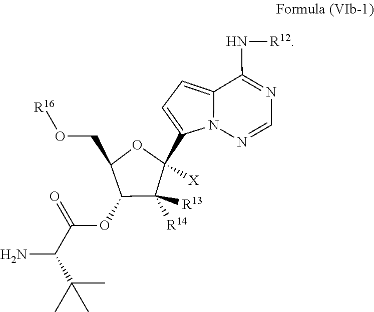

7. The compound of claim 1 or 5, wherein the compound is a compound of Formula (VIb-2):

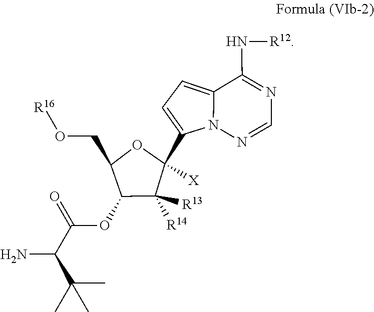

8. The compound of claim 1, wherein the compound is a compound of Formula (VIc):

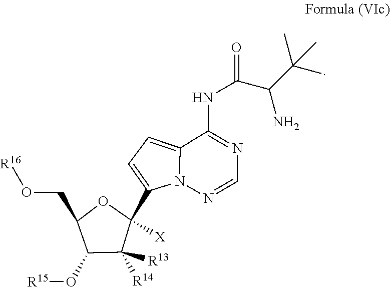

9. The compound of claim 1 or 8, wherein the compound is a compound of Formula (VIc-1):

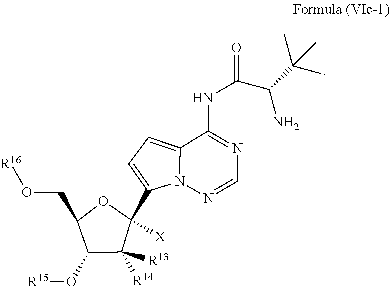

10. The compound of claim 1 or 8, wherein the compound is a compound of Formula (VIc-2):

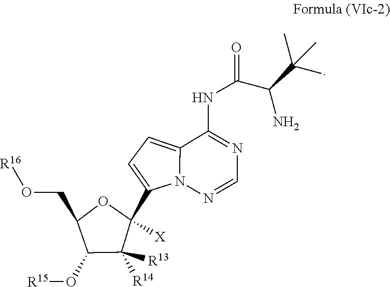

11. The compound of any one of claims 1-10, wherein X is hydrogen.

12. The compound of any one of claims 1-10, wherein X is —CN.

13. The compound of any one of claims 1-12, wherein R¹³ is hydrogen.

14. The compound of any one of claims 1-12, wherein R¹³ is C₁-C₆alkyl.

15. The compound of any one of claims 1-14, wherein R¹⁴ is —OH.

16. The compound of any one of claims 1-14, wherein R¹⁴ is fluoro.

17. The compound of any one of claims 1-7 or 11-16, wherein R¹² is hydrogen, —C(═O)R²², —C(═O)OR²², or C₁-C₆alkyl.

18. The compound of any one of claims 1-7 or 11-16, wherein R¹² is —C(═O)R²², —C(═O)OR²², or C₁-C₆alkyl.

19. The compound of any one of claims 1-7 or 11-16, wherein R¹² is —C(═O)R²².

20. The compound of any one of claims 1-7 or 11-16, wherein R¹² is —C(═O)OR²².

21. The compound of any one of claims 1-7 or 11-16, wherein R¹² is C₁-C₆alkyl.

22. The compound of any one of claims 1-7 or 11-16, wherein R¹² is hydrogen.

23. The compound of any one of claims 1-7 or 11-22, wherein R²² is C₁-C₆alkyl, C₁-C₆aminoalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{22a}.

24. The compound of any one of claims 1-7 or 11-22, wherein R²² is C₁-C₆alkyl, C₁-C₆aminoalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl).

25. The compound of any one of claims 1-7 or 11-22, wherein R²² is C₁-C₆alkyl, C₁-C₆aminoalkyl, aryl, C₁-C₆alkylene(cycloalkyl), C₁-C₆alkylene(heterocycloalkyl), C₁-C₆alkylene(aryl), or C₁-C₆alkylene(heteroaryl).

26. The compound of any one of claims 1-7 or 11-22, wherein R²² is C₁-C₆alkyl, C₁-C₆aminoalkyl, aryl, C₁-C₆alkylene(cycloalkyl), or C₁-C₆alkylene(aryl).

27. The compound of any one of claims 1-7 or 11-22, wherein R²² is C₁-C₆alkyl or C₁-C₆alkylene(aryl); wherein the alkyl, alkylene, and aryl is optionally and independently substituted with one or more R^{22a}.

28. The compound of any one of claims 1-7 or 11-22, wherein R²² is C₁-C₆alkyl optionally and independently substituted with one or more R^{22a}.

29. The compound of any one of claims 1-7 or 11-28, wherein each R²² is independently halogen, —CN, —OH, —OR^{a}, —OC(═O)R^{a}, —OC(═O)OR^{b}, —OC(═O)NR^{c}R^{d}, —NR^{c}R^{d}, —C(═O)R^{a}, —C(═O)OR^{b}, —C(═O)NR^{c}R^{d}, C₁-C₆alkyl, or C₁-C₆haloalkyl.

30. The compound of any one of claims 1-7 or 11-28, wherein each R²² is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, or C₁-C₆haloalkyl.

31. The compound of any one of claims 1-7 or 11-28, wherein each R²² is independently halogen, —OH, —OR^{a}, —NR^{c}R^{d}, C₁-C₆alkyl, or C₁-C₆haloalkyl.

32. The compound of any one of claims 1-7 or 11-28, wherein each R²² is independently halogen, —OC(═O)R^{a}, or —NR^{c}R^{d}.

33. The compound of any one of claims 1-7 or 11-28, wherein each R²² is independently —OC(═O)R^{a} or —NR^{c}R^{d}.

34. The compound of any one of claims 1-7 or 11-28, wherein each R²² is independently —OC(═O)R^{a}.

35. The compound of any one of claims 1-4 or 8-34, wherein R¹⁵ is —C(═O)R²⁵ or —C(═O)OR²⁵, or —CH₂—O—C(═O)R²⁵.

36. The compound of any one of claims 1-4 or 8-34, wherein R¹⁵ is hydrogen.

37. The compound of any one of claims 1-4 or 8-34, wherein R¹⁵ is —C(═O)R²⁵.

38. The compound of any one of claims 1-4 or 8-34, wherein R¹⁵ is —C(═O)OR²⁵.

39. The compound of any one of claims 1-4 or 8-38, wherein R²⁵ is C₁-C₆alkylene(cycloalkyl) or C₁-C₆alkylene(aryl); wherein the alkylene, cycloalkyl, and aryl is optionally and independently substituted with one or more R^{25a}.

40. The compound of any one of claims 1-4 or 8-38, wherein R²⁵ is C₁-C₆alkylene(cycloalkyl);
wherein the alkylene and cycloalkyl is optionally and independently substituted with one or more R^{25a}.

41. The compound of any one of claims 1-4 or 8-38, wherein R²⁵ is C₁-C₆alkylene(aryl); wherein the alkylene, and aryl is optionally and independently substituted with one or more R^{25a}.

42. The compound of any one of claims 1-4 or 8-41, wherein each R^{25a} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, or C₁-C₆haloalkyl.

43. The compound of any one of claims 1-4 or 8-41, wherein each R^{25 a} is independently halogen, —OH, —OR^{a}, —NR^{c}R^{d}, C₁-C₆alkyl, or C₁-C₆haloalkyl.

44. The compound of any one of claims 1 or 5-43, wherein R¹⁶ is hydrogen, —C(═O)R²⁶, —C(═O)OR²⁶ or C₁-C₆alkyl optionally substituted with one or more R^{1a}.

45. The compound of any one of claims 1 or 5-43, wherein R¹⁶ is hydrogen, —C(═O)R²⁶, or —C(═O)OR²⁶.

46. The compound of any one of claims 1 or 5-43, wherein R¹⁶ is —C(═O)R²⁶ or —C(═O)OR²⁶.

47. The compound of any one of claims 1 or 5-43, wherein R¹⁶ is hydrogen.

48. The compound of any one of claims 1 or 5-43, wherein R¹⁶ is —C(═O)R²⁶.

49. The compound of any one of claims 1 or 5-43, wherein R¹⁶ is —C(═O)OR²⁶.

50. The compound of any one of claims 1 or 5-49, wherein R²⁶ is C₁-C₆alkyl, C₁-C₆aminoalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally and independently substituted with one or more R^{26a}.

51. The compound of any one of claims 1 or 5-49, wherein R²⁶ is C₁-C₆alkyl, C₁-C₆aminoalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl).

52. The compound of any one of claims 1 or 5-49, wherein R²⁶ is C₁-C₆alkyl, C₁-C₆aminoalkyl, aryl, C₁-C₆alkylene(cycloalkyl), C₁-C₆alkylene(heterocycloalkyl), C₁-C₆alkylene(aryl), or C₁-C₆alkylene(heteroaryl).

53. The compound of any one of claims 1 or 5-49, wherein R²⁶ is C₁-C₆alkyl, C₁-C₆aminoalkyl, aryl, C₁-C₆alkylene(cycloalkyl), or C₁-C₆alkylene(aryl).

54. The compound of any one of claims 1 or 5-49, wherein R²⁶ is C₁-C₆alkylene(cycloalkyl) or C₁-C₆alkylene(aryl).

55. The compound of any one of claims 1 or 5-49, wherein R²⁶ is C₁-C₆alkyl or C₁-C₆aminoalkyl.

56. The compound of any one of claims 1 or 5-55, wherein each R^{26a} is independently halogen, —CN, —OH, —OR^{a}, —OC(═O)R^{a}, —OC(═O)OR^{b}, —OC(═O)NR^{c}R^{d}, —NR^{c}R^{d}, —C(═O)R^{a}, —C(═O)OR^{b}, —C(═O)NR^{c}R^{d}, C₁-C₆alkyl, or C₁-C₆haloalkyl.

57. The compound of any one of claims 1 or 5-55, wherein each R^{26a} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, or C₁-C₆haloalkyl.

58. The compound of any one of claims 1 or 5-55, wherein each R^{26a} is independently halogen, —OH, —OR^{a}, —NR^{c}R^{d}, C₁-C₆alkyl, or C₁-C₆haloalkyl.

59. The compound of any one of claims 1 or 5-55, wherein each R^{26a} is independently —OC(═O)R^{a} or —NR^{c}R^{d}.

60. A compound selected from a compound found in table 1, or a pharmaceutically acceptable salt, solvate, or stereoisomer thereof.

61. A pharmaceutical composition comprising the compound of any one of claims 1-60, or a pharmaceutically acceptable salt, solvate, or stereoisomer thereof, and at least one pharmaceutically acceptable carrier.

62. A method of treating a viral infection, comprising administering to a subject in need thereof a therapeutically effective amount of the compound of any one of claims 1-60, or a pharmaceutically acceptable salt, solvate, or stereoisomer thereof, or the pharmaceutical composition of claim 61.

63. The method of claim 62, further comprising administering at least one antiviral agent in combination with the compound of any one of claims 1-60, or a pharmaceutically acceptable salt, solvate, or stereoisomer thereof, or the pharmaceutical composition of claim 61.

64. The method of claim 62 or 63, wherein the viral infection is caused by a virus selected from the group consisting of coronavirus disease 2019 (SARS-CoV-2), Yellow Fever, Eastern Equine Encephalitis virus, Human Immunodeficiency virus (HIV), “African Swine Fever Viruses,” Arbovirus, Adenoviridae, Arenaviridae, Arterivirus, Astroviridae, Baculoviridae, Bimaviridae, Bimaviridae, Bunyaviridae, Caliciviridae, Caulimoviridae, Circoviridae, Coronaviridae, Cystoviridae, Ebolavirus, Deltaviridae, Filviridae, Filoviridae, Flaviviridae, Iridoviridae, Mononegavirus, Myoviridae, Papiloma virus, Papovaviridae, Paramyxoviridae, Prions, Parvoviridae, Phycodnaviridae, Poxviridae, Potyviridae, Reoviridae, Retroviridae, Rhabdoviridae, Tectiviridae, Togaviridae, pox, papilloma, corona, influenza, sendai virus (SeV), sindbis virus (SINV), vaccinia viruses, West Nile, Hanta, viruses which cause the common cold, and any combination thereof.

65. The method of any one of claims 62-64, wherein the viral infection is caused by coronavirus disease 2019 (SARS-CoV-2).

66. The method of any one of claims 62-64, wherein the viral infection is caused by the Middle East respiratory syndrome coronavirus (MERS-CoV).

67. The method of any one of claims 62-64, wherein the viral infection is caused by an Ebolavirus.

68. The method of claim 67, wherein the Ebolavirus is Zaira Ebolavirus (Ebola virus).

69. The method of any one of claims 63-68, wherein the at least one antiviral agent is abacavir, acyclovir, adefovir, brivudine, cidofovir, didanosine, edoxudine, emtricitabine, entecavir, famciclovir, favipiravir, ganciclovir, idoxuridine, lamivudine, molnupiravir, penciclovir, remdesivir, ribavirin, sofosbuvir, ST-193, stavudine, T-705 diphosphate,T-705 monophosphate, T-705 triphosphate, telbivudine, tenofovir alafenamide, tenofovir disoproxil, trifluridine, valaciclovir, valganciclovir, vidarabine, zalcitabine, zidovudine, or any combinations thereof.

70. The method of any one of claims 63-68, wherein the at least one antiviral agent is molnupiravir.

71. The method of any one of claims 63-68, wherein the at least one antiviral agent is a ribonucleic acid (RNA)-dependent RNA polymerase inhibitor, a checkpoint inhibitor or PD-1/PD-L1 inhibitor, a therapeutic vaccine, an RNA interference (RNAi) therapeutic, an antisense-based therapeutic, an coronavirus entry inhibitor, a TLR agonist, an RIG-I agonist, or an interferon.
